(19) United States
(12) Patent Application Publication
(10) Pub. No.: US 2025/0282735 A1
(43) Pub. Date: Sep. 11, 2025

(54) BICYCLE COMPOUNDS AS TEAD INHIBITOR

(71) Applicant: DONG-A ST CO., LTD., Seoul (KR)

(72) Inventors: Sunho Choi, Seoul (KR); Younggyu Kong, Gyeonggi-do (KR); Haneol Kim, Gyeonggi-do (KR); Gyerim Baek, Gyeonggi-do (KR); Hyeyeon Soh, Gyeonggi-do (KR); Seoyun Yang, Gyeonggi-do (KR); Jaewook Yoo, Gyeonggi-do (KR); Okyoung Lee, Gyeonggi-do (KR); Youngmin Jeong, Seoul (KR); Hyeonseok Jung, Gyeonggi-do (KR); Taedong Han, Gyeonggi-do (KR); Youngjun Park, Gyeonggi-do (KR)

(21) Appl. No.: 18/861,369

(22) PCT Filed: Apr. 28, 2023
(86) PCT No.: PCT/IB2023/054410

§ 371 Date: Oct. 29, 2024

(30) Foreign Application Priority Data
Apr. 29, 2022 (KR) 10-2022-0053712

(51) Int. Cl. C07D 231/56; A61K 31/404; A61K 31/416; A61K 31/437; A61K 31/444; A61K 31/4545; A61P 35/00; C07D 209/14; C07D 209/34; C07D 471/04

(52) U.S. Cl. CPC C07D 231/56; A61K 31/404; A61K 31/416; A61K 31/437; A61K 31/444; A61K 31/4545; A61P 35/00; C07D 209/14; C07D 209/34; C07D 471/04

(57) ABSTRACT

The present invention relates to bicyclic heterocycle compounds, to their preparation and to their therapeutic use. Specifically, the present invention relates to the compounds represented by formula (I), a pharmaceutically acceptable salt thereof, a stereoisomer thereof, a tautomer thereof, a solvate thereof, a hydrate thereof, or a prodrug analog thereof, to their preparation and to their therapeutic use.

### TECHNICAL FIELD

The present invention relates to bicyclic heterocycle compounds, to their preparation and to their therapeutic use. The bicyclic heterocycle compounds herein are Transcriptional Enhancer Associate Domain (TEAD) binders and/or inhibitors of YAP/TAZ-TEAD or TEAD-dependent gene transcription, and are therefore useful in the treatment of disease related to the activity of YAP/TAZ-TEAD or TEAD-dependent gene transcription including, e.g. cancers and other diseases.

### BACKGROUND ART

It is known that the Hippo signaling pathway could control organ size in animals by regulating cell proliferation and apoptosis. Organ growth is regulated by relying on several processes that occur at the cellular level, including cell division and programmed cell death (Zhao et al. 2008; Kango-Singh and Singh 2009). The regulating mechanism is as follows; When the Hippo signaling pathways is activated, it is involved in inhibiting cell proliferation and promoting apoptosis. On the other hand, when the hippo signaling pathway is inactivated, it promotes cell proliferation and inhibits apoptosis. The Hippo pathway also plays a critical role in the self-renewal and expansion of stem cells and tissue-specific progenitors involved in tissue repair and regeneration.

In mammals, the Hippo signaling system contains two transcriptional coactivators that are homologous to Yki and partially overlapping each other; YAP (Yes-associated protein) and TAZ (transcriptional coactivator with PDZ-binding motif, WWTR1) (Huang et al., 2005; Wang K et al., 2009). Hippo signaling regulates YAP proteins primarily through phosphorylation by LATS (large tumor suppressor) kinases (LATS1 and LATS2, Warts in Drosophila) (Dong et al., 2007; Huang et al., 2005; Oh and Irvine, 2008; Zhao et al., 2007). LATS kinases are activated through phosphorylation by Ste-20 family of protein kinases, principally MST1/2 (mammalian STE-like 1 and 2, Hpo in Drosophila) or MAP4K4 (mitogen-activated protein kinase 4, Msn in Drosophilla) (Misra and Irvine, 2018; Zheng and Pan, 2019). This highly conserved group of serine/threonine kinases regulates multiple cellular processes, including cell proliferation, apoptosis, and various stress responses (Dan et al., 2001). YAP protein phosphorylated by LATS binds 14-3-3 proteins and promotes cytoplasmic localization (Dong et al., 2007; Oh and Irvine, 2008; Zhao et al., 2007). The cytoplasmic inactive-YAP protein is degraded through recruitment of the SCFb-TRCP E3 ubiquitin ligase (Liu et al., 2010; Zhao et al., 2010). This is the process that the hippo pathway is turns “ON”.

Conversely, if the hippo pathway is turns “OFF”, the unphosphorylated active form of YAP protein can accumulate in the nucleus. As a result, it induces gene expression through interaction with the DNA-binding TEAD (TEA domain) transcription factors (Wu et al., 2008; Zhang et al., 2008; Zhao et al., 2008). YAP-TEAD complex stimulates expression of target genes, such as CTGF (connective tissue growth factor), CYR61 (Cysteine-rich angiogenic inducer 61), AMOTL2 (Angiomotin-like 2), and ANKRD1 (Ankyrin Repeat Domain 1) (Yu et al. 2015). Although TEAD binding appears to be the most important in induction of YAP target genes, they YAP-TEAD complex can further cooperate with other DNA-binding partners (Totaro et al. 2018).

Many of the genes involved in the Hippo signaling pathway are recognized as tumor suppressors, while YAP/TAZ is identified as an oncogene. Therefore, the hippo signaling pathway is becoming increasingly significant in human cancer research as many cancers are marked by unchecked cell division.

YAP has been found to be elevated in some human cancers, including breast cancer, colorectal cancer, and liver cancer (Kango-Singh et al., 2009; Zender et al., 2006; Steinhardt et al., 2008). YAP/TAZ is a widely activated transcriptional regulators in human malignancies and is essential for cancer initiation and tumor growth. In addition, YAP/TAZ proteins also control other cellular behaviors and can promote maintenance of stem cell or progenitor cell fates while inhibiting differentiation. YAP/TAZ activation leads to induction of cancer stem cell properties as well as cell proliferation and metastasis (Camargo et al., 2007; Chan et al., 2008; Dong et al., 2007; Zhao et al., 2007). The mechanisms have been investigated in cancer cells highlighting a broad transcriptional program linked to cell cycle progression downstream of YAP/TAZ (Kapoor et al., 2014; Zanconato et al., 2015). Accordingly, YAP/TAZ research is becoming very important in the field of cancer.

TEADs are essential transcription factors in regulating the transcriptional output of the Hippo pathway. Palmitoylation of TEAD has been studied to play an important role in regulating the hippo pathway transcription complexes. Protein S-palmitoylation is a reversible post-translational modification. It attaches a 16-carbon fatty acid, palmitate, to cysteine residues (Smotrys et al., 2004; Wan et at., 2007; Martin et al., 2009) and regulates protein trafficking, membrane localization, signaling activities and differential stability (Resh et al., 2006; Linder et al., 2007; Yount et al., 2010). Therefore, even in cancer, S-palmitoylation is essential for the function of both oncogenes and tumor suppressors.

The TEAD transcription factors are palmitoylated at evolutionarily conserved cysteine residues and undergo autopalmitoylation at physiological concentrations of palmitoyl-CoA (Chan et al., 2016). Palmitoylation is required for TEAD stability, it consequently plays an important role in regulating YAP-TEAD association and their physiological functions in the Hippo signaling pathway (Noland et al, 2016).

Although targeting the YAP-TEAD interaction could be a promising therapeutic approach for diseases related to Hippo pathway regulation, direct inhibition of transcription factors with small molecules remains a challenge.

Here, regulating TEAD itself may reveal new therapeutic opportunities for drug discovery. One of the ways to regulate it could be to target the TEAD-palmitoylation sites. As structural studies on TEAD proteins preceded, binding pockets were revealed, and now the possibility of accessing them as small molecules has increased. Therefore, an approach to develop a TEAD inhibitor should be attempted, and furthermore, it is expected that it will also inhibit the interaction with the YAP/TAZ protein.

### DISCLOSURE

### Definition

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule.

The term “substituted” refers to the fact that at least one of the hydrogen atoms of that moiety is replaced by another substituent or moiety.

In the present invention, “Cx-Cy” means having carbon atoms in a range of x to y.

In the present invention, a symbol “ [symbol] ” represents a single bond or a double bond. Whether [symbol] is a single bond or a double may be determined by a permitted valency of atoms connected to each other through [symbol] .

The term “alkyl” refers to an aliphatic unbranched (straight)-chain or branched-chain saturated hydrocarbon moiety having 1 to 20 carbon atoms, such as 1 to 12 carbon atoms, or 1 to 6 carbon atoms. Alkyl groups may be optionally substituted.

The term “cycloalkyl” means a saturated carbocyclic moiety having mono- or bicyclic (including bridged bicyclic) rings and 3 to 10 carbon atoms in the ring. In particular aspects, cycloalkyl may contain from 3 to 8 carbon atoms (i.e., (C3-C8) cycloalkyl). Examples of cycloalkyl moieties include, but are not limited to, cyclopropyl, cyclobutyl, cyclopentyl, cyclohexyl, cycloheptyl, etc. The cycloalkyl moiety may be attached in a spirocycle fashion such as spirocyclopropyl.

The term “cycloalkenyl” means a partially unsaturated (cycloalkenyl) carbocyclic moiety (e.g. cyclopentenyl, cyclohexenyl, and cycloheptenyl). Cycloalkenyl may include one double bond of two carbons which form the ring. The cycloalkenyl moiety may be attached in a spirocycle fashion such as spirocyclopropyl.

The term “haloalkyl” refers to an alkyl group wherein one or more of the hydrogen atoms of the alkyl group has been replaced by the same or different halogen atoms, such as fluoro atoms. Examples of haloalkyl include monofluoro-, difluoro-, or trifluoro-methyl, -ethyl, or -propyl, for example 3,3,3-trifluoropropyl, 2-fluoroethyl, 2,2,2-trifluoroethyl, fluoromethyl, or trifluoromethyl. Haloalkyl groups may be optionally substituted.

The term “alkenyl” refers to a straight or branched chain alkyl or substituted alkyl group as defined elsewhere herein having at least one carbon-carbon double bond. Alkenyl groups may be optionally substituted.

The term “alkynyl” refers to a straight or branched chain alkyl or substituted alkyl group as defined elsewhere herein having at least one carbon-carbon triple bond. Alkynyl groups may be optionally substituted.

The term “alkylene” means a divalent functional group which is induced from the alkyl group as defined above.

The term “alkenylene” means a divalent functional group which is induced from the alkenyl group. Alkenylene may be represented by —CH═CH—, —CH₂—CH═CH—, —CH(CH₃)—CH═CH—, —CH═CH(CH₃)—, —CH═CH—CH₂—, etc.

The term “alkynylene” means a divalent functional group which is induced the alkylyl group. Alkynylene may be represented by —C≡C—, —CH₂—C≡C—, —C≡C—CH₂—, —CH(CH₃)—C≡C—, etc.

The terms “heterocyclyl” and “heterocycle” refer to a 4, 5, 6 and 7-membered monocyclic or 7, 8, 9 and 10-membered bicyclic (including bridged bicyclic) heterocyclic moiety that is saturated or partially unsaturated, and has one or more (e.g., 1, 2, 3 or 4) heteroatoms selected from oxygen, nitrogen, and sulfur in the ring with the remaining ring atoms being carbon. When used in reference to a ring atom of a heterocycle, a nitrogen or sulfur may also be in an oxidized form, and a nitrogen may be substituted. The heterocycle can be attached to its pendant group at any heteroatom or carbon atom that results in a stable structure and any of the ring atoms can be optionally substituted. Examples of such saturated or partially unsaturated heterocycles include, without limitation, tetrahydrofuranyl, tetrahydropyranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. Other examples of such saturated or partially unsaturated heterocycles include, without limitation, oxiranyl and oxetanyl. The term of heterocycle also includes groups in which a heterocycle is fused to one or more aryl, heteroaryl, or cycloalkyl rings, such as indolinyl, 3H-indolyl, chromanyl, 2-azabicyclo[2.2.1]heptanyl, octahydroindolyl, or tetrahydroquinolinyl. Heterocyclyl groups may be optionally substituted.

The term “heterocycloalkyl” refers to a saturated carbocyclic moiety having mono- or bicyclic (including bridged bicyclic) rings. Heterocycloalkyl may be a 3 to 10-membered ring with at least one heteroatom selected from oxygen, nitrogen, and sulfur inside the ring and may be a ring structure in which at least two rings share at least one carbon atom (for example, spiro ring, bridged ring, etc.). Examples of heterocycloalkyl may include tetrahydrofuranyl, tetrahydropyranyl, tetrahydrothienyl, etc.

The term “aryl” refers to a cyclic aromatic hydrocarbon moiety having a mono-, bi- or tricyclic aromatic ring of 5 to 20 carbon ring atoms. Examples of aryl moieties include, but are not limited to, phenyl, naphthyl, benzyl, and the like. The term “aryl” also includes partially hydrogenated derivatives of the cyclic aromatic hydrocarbon moiety provided that at least one ring of the cyclic aromatic hydrocarbon moiety is aromatic, each being optionally substituted. In some aspects, monocyclic aryl rings may have 5 or 6 carbon ring atoms. Aryl groups may be optionally substituted. Aryl groups may be optionally fused.

The term “heteroaryl” refers to an aromatic heterocyclic mono- or bicyclic ring system of 1 to 20 ring atoms, comprising 1, 2, 3 or 4 heteroatoms selected from N, O and S, the remaining ring atoms being carbon. Examples of heteroaryl moieties include, but are not limited to, pyrrolyl, furanyl, thienyl, imidazolyl, oxazolyl, thiazolyl, triazolyl, oxadiazolyl, thiadiazolyl, tetrazolyl, pyridnyl, pyrazinyl, pyrazolyl, pyridazinyl, pyrimidinyl, triazinyl, isoxazolyl, bezofuranyl, isthiazolyl, beznothienyl, indolyl, isoindolyl, isobenzofuranyl, benzimidazolyl, benzoxazolyl, benzoisoxazolyl, benzothiazolyl, benzooxadiazolyl, benzothiadiazolyl, benzotriazolyl, purinyl, puinolinyl, isoquinolinyl, quinazolinyl, or quinoxalinyl. Heteroaryl groups may be optionally substituted. Heteroaryl groups may be optionally fused. Example of fused heteroaryl include, but are not limited to, -benzodioxanyl.

The terms “halogen” refer to fluoro, chloro, bromo, and iodo. In some aspects, halogen is fluoro or chloro.

The terms “oxo” refers to the ═O moiety.

The term “cyano” refers to the —C≡N moiety.

The terms “spirocycle” and “spirocyclyl” refers to carbogenic bicyclic ring systems comprising between 5 and 15 carbon atoms with both rings connected through a single atom. The rings can be different in size and nature, or identical in size and nature. Examples include, but are not limited to, spiropentane, spirohexane, spiroheptane, spirooctane, spriononane, or spirodecane. One or more of the carbon atoms in the spirocycle can be substituted with a heteroatom (e.g., O, N, S, or P), wherein in such aspects the spirocycle may comprise between 3 and 14 carbon atoms. Spirocycle groups may be optionally substituted.

The term “annular” refers to a moiety that is a member of a ring, including, but not limited to, a cycloalkyl ring, a cycloalkenyl ring, an aryl ring, a heteroaryl ring, a heterocyclyl ring, or a spirocyclyl ring. For example, if a heteroaryl ring is described as “comprising two or more annular heteroatoms”, two or more of the ring members of the heteroaryl ring will be heteroatoms.

The term “pharmaceutical acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably, hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, enthanesulfonic acid, p-toluenesulfonic acid, N-acetylcystein and the like. In addition, salts may be prepared by the addition of an inorganic base or an organic base to the free acid. Salts derived from an inorganic base include, but not are limited to, the sodium, potassium, lithium, ammonium, calcium, and magnesium salts and the like. Salts derived from organic base include, but are not limited to salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, lysine, arginine, N-ethylpiperidine, piperidine, polyamine resins and the like. In addition to salt forms, the present disclosure provides compounds which are in a prodrug form. As used herein the term “prodrug” refers to those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The present disclosure provides for metabolites of compounds of the disclosure. As used herein, a “metabolite” refers to a product produced through metabolism in the body of a specified compound or salt thereof. Such products can result for example from the oxidation, reduction, hydrolysis, amidation, deamidation, esterification, de-esterification, enzymatic cleavage, and the like, of the administered compound.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure can exist in multiple crystalline or amorphous forms. In general, all physical forms are equivalent for the uses contemplated by the present disclosure and are intended to be within the scope of the present disclosure.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelong, or by the manner in which the molecule rotates the plane of polarized light and designated as dextrorotatory or levorotatory (i.e., (+) or (−)-isomers respectively). A chiral compound can exist as either individual enantiomer or as a mixture thereof. A mixture containing equal proportions of the enantiomers is called a “racemic mixture”. In certain aspects the compound is enriched by at least about 90% by weight with a single diastereomer or enantiomer. In other aspects the compound is enriched by at least about 95%, 98%, or 99% by weight with a single diastereomer or enantiomer.

Certain compounds of the present disclosure possess asymmetric carbon atoms (optical centers) or double bonds; the racemates, diastereomers, geometric isomers, regioisomers, and individual isomers (e.g., separate enantiomers) are all intended to be encompassed within the scope of the present disclosure.

The compounds of the present disclosure may also exist in different tautomeric forms, and all such forms are embraced within the scope of the disclosure. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Unless otherwise indicated, the term “a compound of the formula” or “a compound of formula” or “compounds of the formula” refers to any compound selected from the genus of compounds as defined by the formula. In some embodiments or aspects, the term also includes a pharmaceutically acceptable salt or ester of any such compound, a stereoisomer, or a tautomer of such compound.

### Compound Description

(1) In some aspects, the present disclosure is directed to the compounds represented by formula (I), a pharmaceutically acceptable salt thereof, a stereoisomer thereof, a tautomer thereof, a solvate thereof, a hydrate thereof, or a prodrug analog thereof.

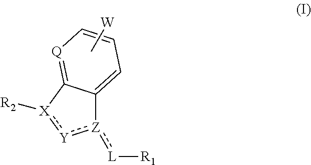

In formula (I),
each [symbol] of formula (I) is independently a single bond or a double bond,
L is absent, —N(R_{L})—, —N(R_{L})C(R_{L})₂—, —N(R_{L})C(═O)—, —C(R_{L})₂O—, —OC(R_{L})₂—, —C(R_{L})₂N(R_{L})—, —C(═O)—, —CH(OR_{L})—, ═CH— or is unbranched or branched C1-C3 alkylene or C2-C3 alkenylene, wherein at least one H of the unbranched or branched C1-C3 alkylene or C2-C3 alkenylene is independently unsubstituted or substituted with halogen, C1-C6 haloalkyl OR_{L} or N(R_{L})₂;
R_{L} is H or C1-C6 alkyl, wherein at least one H of the C1-C6 alkyl is independently unsubstituted or substituted with halogen;
Q is CH or N, wherein H of the CH is unsubstituted or substituted with halogen;
X is C or N;
Y is CR_{N}, C(═O) or N;
R_{N} is H or C1-C6 alkyl;
Z is C, N or CH;
W is

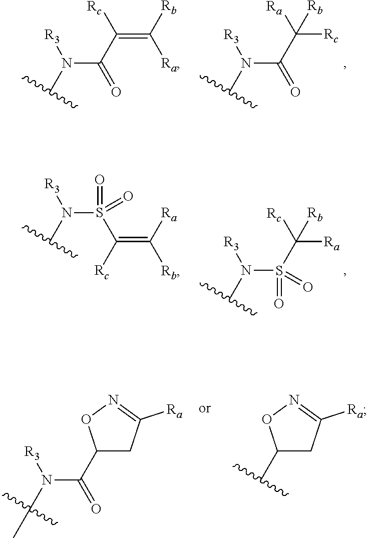

Rₐ, R_{b}, and R_{c} are each independently H, halogen, cyano, hydroxyl, C1-C3 alkyl-N(R₄)₂, or C1-C6 alkyl;
R₁ is H, C1-C3 alkyl, C3-C6 cycloalkyl, 4 to 6-membered heterocycloalkyl, C3-C6 cycloalkenyl,

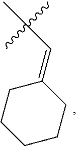 [SMILES: *C=C1CCCCC1]

aryl or heteroaryl, wherein at least one H of the C1-C3 alkyl, C3-C6 cycloalkyl, 4 to 6-membered heterocycloalkyl, C3-C6 cycloalkenyl, aryl or heteroaryl is independently unsubstituted or substituted with halogen, CN, —CH═CH—R₄, —(CH₂)₂—R₄, OR₄, SR₄, CH₂N(R₄)₂, C(═O)—R₄, COOR₄, CON(R₄)₂. C1-C10 alkyl, C1-C6 haloalkyl, C3-C6 cycloalkyl, 4 to 6-membered heterocycloalkyl, phenyl or phenyl substituted with halogen;
R₂ is H, unbranched or branched C1-C6 alkyl or C3-C6 cycloalkyl, wherein at least one H of the unbranched or branched C1-C6 alkyl or C3-C6 cycloalkyl is independently unsubstituted or substituted with deuterium, halogen or ═O;
R₃ is H, unbranched or branched C1-C6 alkyl, C3-C6 cycloalkyl, C1-C6 alkyl-aryl or C1-C6 alkyl-heteroaryl, wherein at least one H of the C1-C6 alkyl, C3-C6 cycloalkyl, C1-C6 alkyl-aryl or C1-C6 alkyl-heteroaryl is independently unsubstituted or substituted with halogen, OR_{M}, N(R_{M})₂, COOH, COOR_{M}, C(═O)R_{M} or C(═O)N(R_{M})₂;
R_{M} is H, C1-C6 alkyl or C3-C6 cycloalkyl; and
R₄ is H, C1-C6 alkyl, C3-C6 cycloalkyl, aryl or C1-C6 alkyl-aryl, wherein at least one H of the C1-C6 alkyl, C3-C6 cycloalkyl, aryl or C1-C6 alkyl-aryl is independently unsubstituted or substituted with halogen.

In formula (I), Z is N or CH, when [symbol] connecting Y to Z is a single bond and L is absent, —N(R_{L})—, —N(R_{L})C(R_{L})₂—, —N(R_{L})C(═O)—, —C(R_{L})₂O—, —OC(R_{L})₂—, —C(R_{L})₂N(R_{L})—, —C(═O)—, —CH(OR_{L})—, unbranched or branched C1-C3 alkylene, or C2-C3 alkenylene,
Z is C, when [symbol] connecting Y to Z is a double bond and L is absent, —N(R_{L})—, —N(R_{L})C(R_{L})₂—, —N(R_{L})C(═O)—, —C(R_{L})₂O—, —OC(R_{L})₂—, —C(R_{L})₂N(R_{L})—, —C(═O)—, —CH(OR_{L})—, unbranched or branched C1-C3 alkylene, C2-C3 alkenylene or C2-C3 alkynylene, and
Z is C when [symbol] connecting Y to Z is a single bond and L is ═CH—.

In formula (I), when L is absent, Z and R₁ are directly connected each other.

In formula (I), [symbol] connecting X to Y is a single bond and connecting Y to Z is a double bond when X is N, Y is CR_{N} and Z is C.

In formula (I), when X is N, Y is N and Z is C, [symbol] connecting X to Y is a single bond and [symbol] connecting Y to Z is a double bond.

In formula (I), when X is C, Y is CR_{N} and Z is N, [symbol] connecting X to Y is a double bond and [symbol] connecting Y to Z is a single bond.

In formula (I), when X is N, Y is C(═O) and Z is C, [symbol] connecting X to Y is a single bond and [symbol] connecting Y to Z is a single bond. In this case, [symbol] connecting Z to L is a double bond. For example, L may be ═CH—.

(2) In above (1), there may be provided a compound, a pharmaceutically acceptable salt thereof, a stereoisomer thereof, a tautomer thereof, a solvate thereof, a hydrate thereof, or a prodrug analog thereof according to the present invention, in which:
L is absent or ═CH— or is unbranched or branched C1-C3 alkylene or C2-C3 alkenylene, wherein at least one H of the unbranched or branched C1-C3 alkylene or C2-C3 alkenylene is independently unsubstituted or substituted with C1-C6 haloalkyl;
Q is CH or N, wherein H of the CH is unsubstituted or substituted with halogen;
X is C or N;
Y is CR_{N}, C(═O) or N;
R_{N} is H or C1-C6 alkyl;
Z is C, N or CH,
when [symbol] connecting Y to Z is a single bond and L is absent, unbranched or branched C1-C3 alkylene, C2-C3 alkenylene or C2-C3 alkynylene, Z is N or CH,
when [symbol] connecting Y to Z is a double bond and L is absent, unbranched or branched C1-C3 alkylene, C2-C3 alkenylene or C2-C3 alkynylene, Z is C, and
when [symbol] connecting Y to Z is a single bond and L is ═CH—, Z is C;
W is

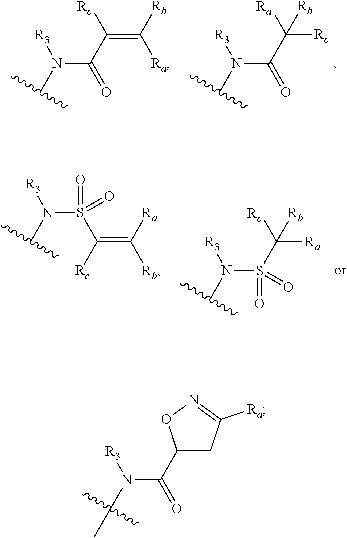

Rₐ, R_{b}, and R_{c} are each independently H, halogen, cyano, hydroxyl, C1-C3 alkyl-N(R₄)₂ or C1-C6 alkyl;
R₁ is H, C1-C3 alkyl, C3-C6 cycloalkyl, 4 to 6-membered heterocycloalkyl, C3-C6 cycloalkenyl,

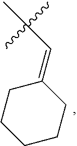 [SMILES: *C=C1CCCCC1]

aryl or heteroaryl, wherein at least one H of the C1-C3 alkyl, C3-C6 cycloalkyl, C3-C6 cycloalkenyl, aryl or heteroaryl is independently unsubstituted or substituted with halogen, CN, —CH═CH—R₄, —(CH₂)₂—R₄, OR₄, SR₄, CH₂N(R₄)₂, C(═O)—R₄, COOR₄, C1-C10 alkyl, C1-C6 haloalkyl, C3-C6 cycloalkyl, 4 to 6-membered heterocycloalkyl, phenyl or phenyl substituted with halogen;
R₂ is H or unbranched or branched C1-C6 alkyl or C3-C6 cycloalkyl, wherein at least one H of the unbranched or branched C1-C6 alkyl or C3-C6 cycloalkyl is independently unsubstituted or substituted with deuterium, halogen or ═O;
R₃ is H, unbranched or branched C1-C6 alkyl, C3-C6 cycloalkyl, benzyl or C1-C6 alkyl-benzyl, wherein at least one H of the unbranched or branched C1-C6 alkyl, C3-C6 cycloalkyl, benzyl or C1-C6 alkyl-benzyl is independently unsubstituted or substituted with COOH;
R₄ is H, C1-C6 alkyl, C3-C6 cycloalkyl, phenyl or benzyl, wherein at least one H of the C1-C6 alkyl, C3-C6 cycloalkyl, phenyl or benzyl is independently unsubstituted or substituted with halogen.

(3) In above (1) or (2), there may be provided a compound, a pharmaceutically acceptable salt thereof, a stereoisomer thereof, a tautomer thereof, a solvate thereof, a hydrate thereof, or a prodrug analog thereof according to the present invention, in which:
L is absent, unbranched or branched C1-C3 alkylene or C2-C3 alkenylene;
X, Y, Z, Q, R₁, R₂ and W are the same as defined above (1) or (2).

(4) In above any one of (1), (2) and (3), there may be provided a compound, a pharmaceutically acceptable salt thereof, a stereoisomer thereof, a tautomer thereof, a solvate thereof, a hydrate thereof, or a prodrug analog thereof according to the present invention, in which:
(i) X is N, Y is CR_{N} and Z is C,
(ii) X is N, Y is N and Z is C,
(iii) X is C, Y is CR_{N} and Z, or
(iv) X is N, Y is C(═O) and Z is C,
L, Q, R₁, R₂ and W are the same as defined above (1), (2) or (3).

(5) In above any one of (1), (2), (3) and (4), there may be provided a compound, a pharmaceutically acceptable salt thereof, a stereoisomer thereof, a tautomer thereof, a solvate thereof, a hydrate thereof, or a prodrug analog thereof according to the present invention, in which: the compound by represented by formula (I) is a compound represented by formula (II):

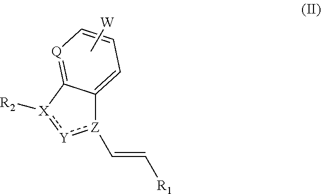

wherein:
Q is CH or N, wherein H of the CH is unsubstituted or substituted with halogen;
X is N;
Y is CR_{N} or N;
R_{N} is H or C1-C3 alkyl;
Z is C;
W is

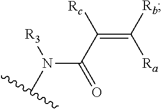

Rₐ, R_{b}, and R_{c} are each independently H, halogen, cyano, or C1-C6 alkyl;
R₁ is C1-C3 alkyl, C3-C6 cycloalkyl, 4 to 6-membered heterocycloalkyl, C3-C6 cycloalkenyl, aryl or heteroaryl, wherein at least one H of the C1-C3 alkyl, C3-C6 cycloalkyl, 4 to 6-membered heterocycloalkyl, C3-C6 cycloalkenyl, aryl or heteroaryl is independently unsubstituted or substituted with halogen, OR₄, unbranched or branched C1-C6 alkyl, COOR₄, C1-C6 haloalkyl, or phenyl;
R₂ is unbranched or branched C1-C6 alkyl or C3-C6 cycloalkyl, wherein at least one H of the unbranched or branched C1-C6 alkyl is independently unsubstituted or substituted with deuterium, halogen or ═O;
R₃ is H, C1-C3 alkyl, benzyl, C1-C3 alkyl-heteroaryl or C1-C3 alkyl substituted with COOH; and
R₄ is C1-C3 alkyl.

(6) In above any one of (1), (2), (3) and (4), there may be provided a compound, a pharmaceutically acceptable salt thereof, a stereoisomer thereof, a tautomer thereof, a solvate thereof, a hydrate thereof, or a prodrug analog thereof according to the present invention, in which: the compound represented by formula (I) is a compound represented by formula (III):

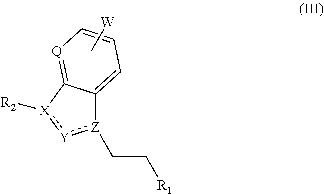

wherein:
Q is CH or N, wherein the CH is optionally substituted with halogen;
X is N;
Y is CR_{N};
R_{N} is H;
Z is C;
W is

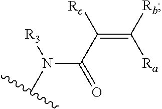

Rₐ, R_{b}, and R_{c} are H;
R₁ is C3-C6 cycloalkyl, C3-C6 cycloalkenyl or aryl, wherein at least one H of the C3-C6 cycloalkyl, C3-C6 cycloalkenyl or aryl is independently unsubstituted or substituted with halogen, OR₄, C1-C10 alkyl or C1-C6 haloalkyl;
R₂ is H or unbranched or branched C1-C6 alkyl or C3-C6 cycloalkyl;
R₃ is H, C1-C3 alkyl or benzyl;
R₄ is C1-C3 alkyl or benzyl, wherein at least one H of the benzyl is independently unsubstituted or substituted with halogen.

(7) In above any one of (1), (2), (3) and (4), there may be provided a compound, a pharmaceutically acceptable salt thereof, a stereoisomer thereof, a tautomer thereof, a solvate thereof, a hydrate thereof, or a prodrug analog thereof according to the present invention, in which: the compound represented by formula (I) is a compound represented by formula (IV):

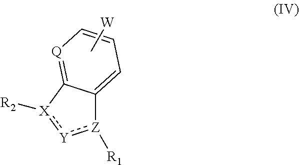

wherein:
Q is CH or N, wherein H of the CH is unsubstituted or substituted with halogen;
X is N;
Y is CR_{N} or N;
R_{N} is H;
Z is C;
W is

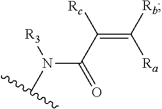

Rₐ, R_{b}, and R_{c} are each independently H or C1-C3 alkyl-N(R₄)₂;
R₁ is H, C3-C6 cycloalkyl, C3-C6 cycloalkenyl,

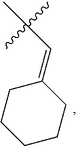 [SMILES: *C=C1CCCCC1]

or aryl, wherein at least one H of the C3-C6 cycloalkyl, C3-C6 cycloalkenyl or aryl is independently unsubstituted or substituted with halogen, CN, —CH═CH—R₄, —(CH₂)₂—R₄, OR₄, SR₄, CH₂N(R₄)₂, C(═O)—R₄, C1-C10 alkyl C1-C6 haloalkyl, C3-C6 cycloalkyl, 4 to 6-membered heterocycloalkyl, phenyl or phenyl substituted with halogen;
R₂ is H or unbranched or branched C1-C6 alkyl;
R₃ is H;
R₄ is H, C1-C3 alkyl, C3-C6 cycloalkyl, phenyl or benzyl, wherein at least one H of the C3-C6 cycloalkyl or benzyl is independently unsubstituted or substituted with halogen.

(8) In above any one of (1) to (7), there may be provided a compound, a pharmaceutically acceptable salt thereof, a stereoisomer thereof, a tautomer thereof, a solvate thereof, a hydrate thereof, or a prodrug analog thereof according to the present invention, in which: the compound is one selected from the group consisting of compounds represented by following 1 to 138:

| Compound | |
|---|---|
| No. | Structure |
| 1 | 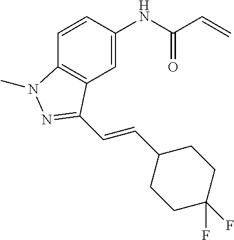 [SMILES: C=CC(=O)Nc1ccc2c(c1)c(/C=C/C1CCC(F)(F)CC1)nn2C] |
| 2 | 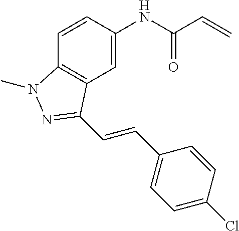 [SMILES: C=CC(=O)Nc1ccc2c(c1)c(/C=C/c1ccc(Cl)cc1)nn2C] |
| 3 | 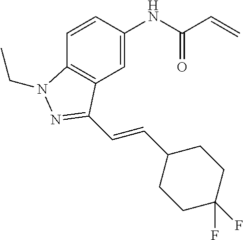 [SMILES: C=CC(=O)Nc1ccc2c(c1)c(/C=C/C1CCC(F)(F)CC1)nn2CC] |
| 4 | 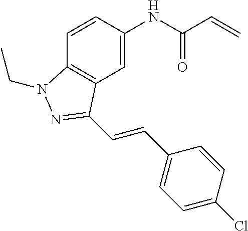 [SMILES: C=CC(=O)Nc1ccc2c(c1)c(/C=C/c1ccc(Cl)cc1)nn2CC] |
| 5 | 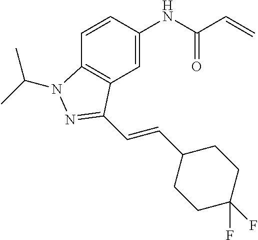 [SMILES: C=CC(=O)Nc1ccc2c(c1)c(/C=C/C1CCC(F)(F)CC1)nn2C(C)C] |
| 6 | 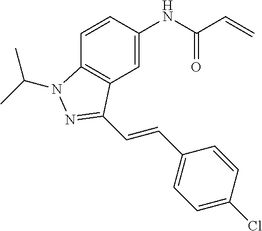 [SMILES: C=CC(=O)Nc1ccc2c(c1)c(/C=C/c1ccc(Cl)cc1)nn2C(C)C] |
| 7 | 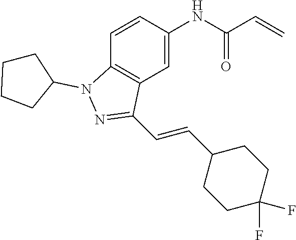 [SMILES: C=CC(=O)Nc1ccc2c(c1)c(/C=C/C1CCC(F)(F)CC1)nn2C1CCCC1] |
| 8 | 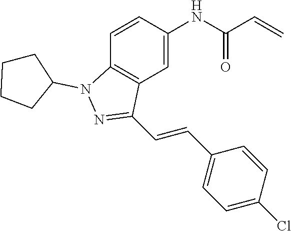 [SMILES: C=CC(=O)Nc1ccc2c(c1)c(/C=C/c1ccc(Cl)cc1)nn2C1CCCC1] |
| 9 | 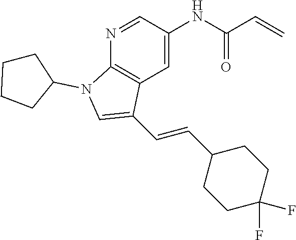 [SMILES: C=CC(=O)Nc1cnc2c(c1)c(/C=C/C1CCC(F)(F)CC1)cn2C1CCCC1] |
| 10 | 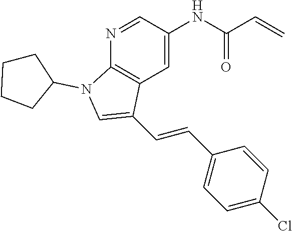 [SMILES: C=CC(=O)Nc1cnc2c(c1)c(/C=C/c1ccc(Cl)cc1)cn2C1CCCC1] |
| 11 | 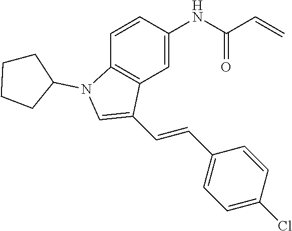 [SMILES: C=CC(=O)Nc1ccc2c(c1)c(/C=C/c1ccc(Cl)cc1)cn2C1CCCC1] |
| 12 | 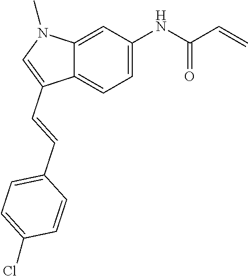 [SMILES: C=CC(=O)Nc1ccc2c(/C=C/c3ccc(Cl)cc3)cn(C)c2c1] |
| 13 | 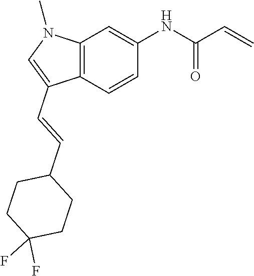 [SMILES: C=CC(=O)Nc1ccc2c(/C=C/C3CCC(F)(F)CC3)cn(C)c2c1] |
| 14 | 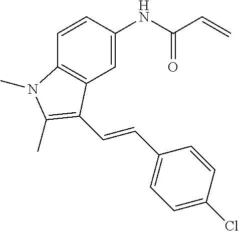 [SMILES: C=CC(=O)Nc1ccc2c(c1)c(/C=C/c1ccc(Cl)cc1)c(C)n2C] |
| 15 | 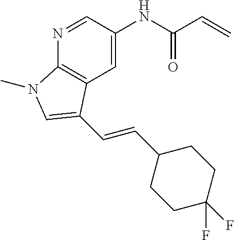 [SMILES: C=CC(=O)Nc1cnc2c(c1)c(/C=C/C1CCC(F)(F)CC1)cn2C] |
| 16 | 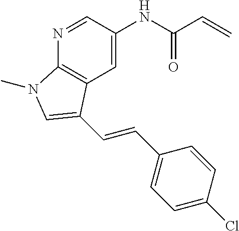 [SMILES: C=CC(=O)Nc1cnc2c(c1)c(/C=C/c1ccc(Cl)cc1)cn2C] |
| 17 | 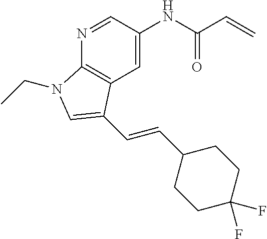 [SMILES: C=CC(=O)Nc1cnc2c(c1)c(/C=C/C1CCC(F)(F)CC1)cn2CC] |
| 18 | 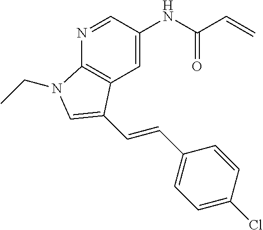 [SMILES: C=CC(=O)Nc1cnc2c(c1)c(/C=C/c1ccc(Cl)cc1)cn2CC] |
| 19 | 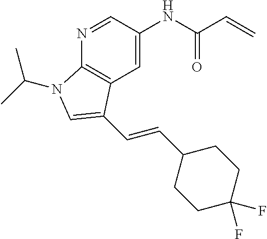 [SMILES: C=CC(=O)Nc1cnc2c(c1)c(/C=C/C1CCC(F)(F)CC1)cn2C(C)C] |
| 20 | 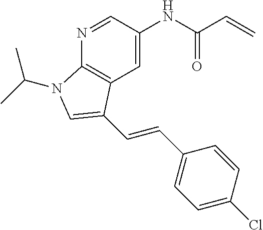 [SMILES: C=CC(=O)Nc1cnc2c(c1)c(/C=C/c1ccc(Cl)cc1)cn2C(C)C] |
| 21 | 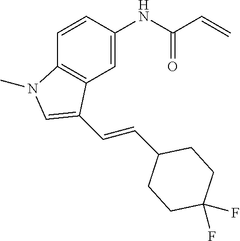 [SMILES: C=CC(=O)Nc1ccc2c(c1)c(/C=C/C1CCC(F)(F)CC1)cn2C] |
| 22 | 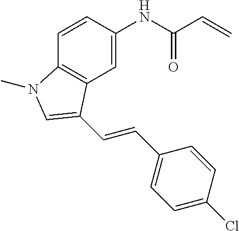 [SMILES: C=CC(=O)Nc1ccc2c(c1)c(/C=C/c1ccc(Cl)cc1)cn2C] |
| 23 | 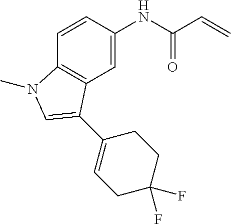 [SMILES: C=CC(=O)Nc1ccc2c(c1)c(C1=CCC(F)(F)CC1)cn2C] |
| 24 | 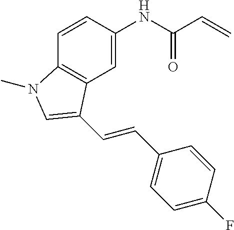 [SMILES: C=CC(=O)Nc1ccc2c(c1)c(/C=C/c1ccc(F)cc1)cn2C] |
| 25 | 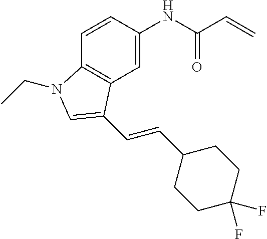 [SMILES: C=CC(=O)Nc1ccc2c(c1)c(/C=C/C1CCC(F)(F)CC1)cn2CC] |
| 26 | 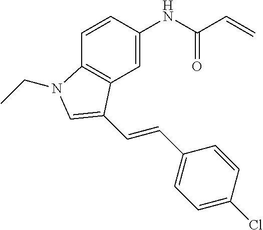 [SMILES: C=CC(=O)Nc1ccc2c(c1)c(/C=C/c1ccc(Cl)cc1)cn2CC] |
| 27 | 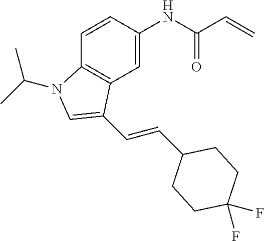 [SMILES: C=CC(=O)Nc1ccc2c(c1)c(/C=C/C1CCC(F)(F)CC1)cn2C(C)C] |
| 28 | 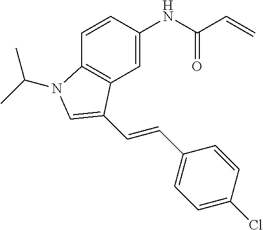 [SMILES: C=CC(=O)Nc1ccc2c(c1)c(/C=C/c1ccc(Cl)cc1)cn2C(C)C] |
| 29 | 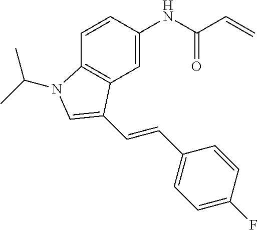 [SMILES: C=CC(=O)Nc1ccc2c(c1)c(/C=C/c1ccc(F)cc1)cn2C(C)C] |
| 30 | 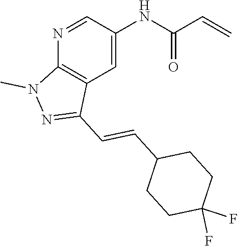 [SMILES: C=CC(=O)Nc1cnc2c(c1)c(/C=C/C1CCC(F)(F)CC1)nn2C] |
| 31 | 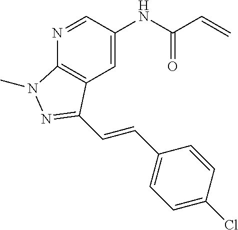 [SMILES: C=CC(=O)Nc1cnc2c(c1)c(/C=C/c1ccc(Cl)cc1)nn2C] |
| 32 | 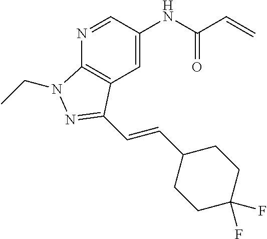 [SMILES: C=CC(=O)Nc1cnc2c(c1)c(/C=C/C1CCC(F)(F)CC1)nn2CC] |
| 33 | 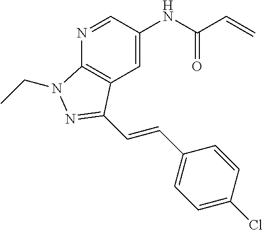 [SMILES: C=CC(=O)Nc1cnc2c(c1)c(/C=C/c1ccc(Cl)cc1)nn2CC] |
| 34 | 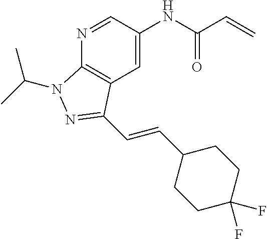 [SMILES: C=CC(=O)Nc1cnc2c(c1)c(/C=C/C1CCC(F)(F)CC1)nn2C(C)C] |
| 35 | 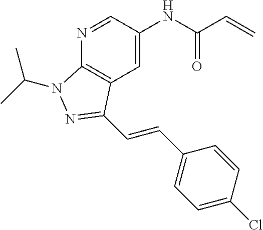 [SMILES: C=CC(=O)Nc1cnc2c(c1)c(/C=C/c1ccc(Cl)cc1)nn2C(C)C] |
| 36 | 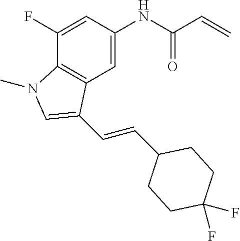 [SMILES: C=CC(=O)Nc1cc(F)c2c(c1)c(/C=C/C1CCC(F)(F)CC1)cn2C] |
| 37 | 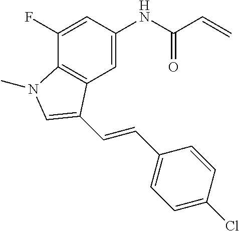 [SMILES: C=CC(=O)Nc1cc(F)c2c(c1)c(/C=C/c1ccc(Cl)cc1)cn2C] |
| 38 | 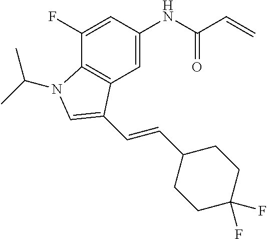 [SMILES: C=CC(=O)Nc1cc(F)c2c(c1)c(/C=C/C1CCC(F)(F)CC1)cn2C(C)C] |
| 39 | 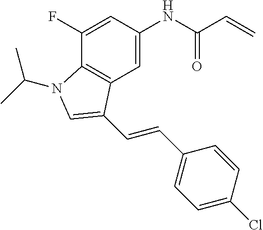 [SMILES: C=CC(=O)Nc1cc(F)c2c(c1)c(/C=C/c1ccc(Cl)cc1)cn2C(C)C] |
| 40 | 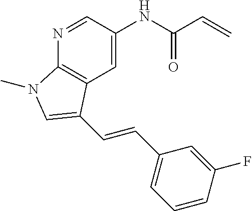 [SMILES: C=CC(=O)Nc1cnc2c(c1)c(/C=C/c1cccc(F)c1)cn2C] |
| 41 | 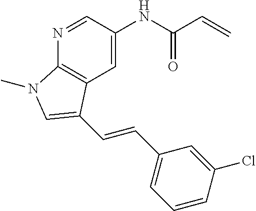 [SMILES: C=CC(=O)Nc1cnc2c(c1)c(/C=C/c1cccc(Cl)c1)cn2C] |
| 42 | 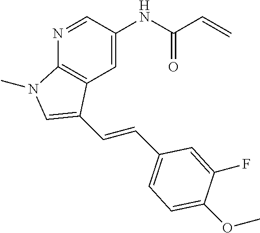 |
| 43 | 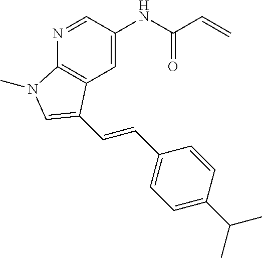 [SMILES: C=CC(=O)Nc1cnc2c(c1)c(/C=C/c1ccc(C(C)C)cc1)cn2C] |
| 44 | 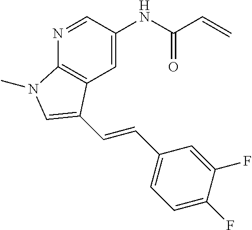 [SMILES: C=CC(=O)Nc1cnc2c(c1)c(/C=C/c1ccc(F)c(F)c1)cn2C] |
| 45 | 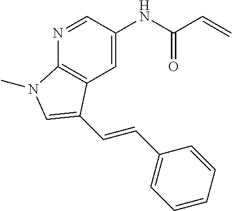 [SMILES: C=CC(=O)Nc1cnc2c(c1)c(/C=C/c1ccccc1)cn2C] |
| 46 | 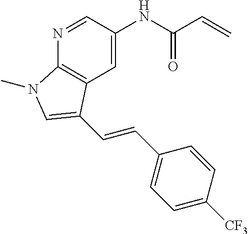 [SMILES: C=CC(=O)Nc1cnc2c(c1)c(/C=C/c1ccc(C(F)(F)F)cc1)cn2C] |
| 47 | 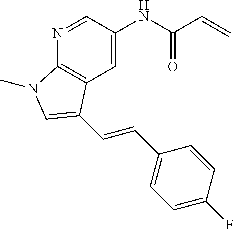 [SMILES: C=CC(=O)Nc1cnc2c(c1)c(/C=C/c1ccc(F)cc1)cn2C] |
| 48 | 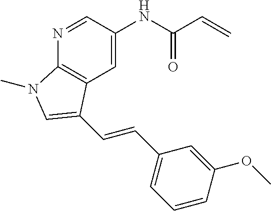 |
| 49 | 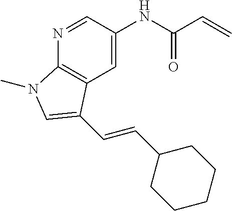 [SMILES: C=CC(=O)Nc1cnc2c(c1)c(/C=C/C1CCCCC1)cn2C] |
| 50 | 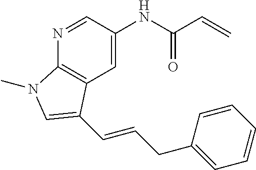 [SMILES: C=CC(=O)Nc1cnc2c(c1)c(/C=C/Cc1ccccc1)cn2C] |
| 51 | 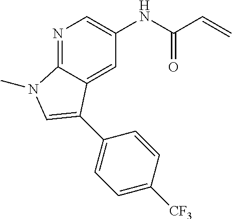 [SMILES: C=CC(=O)Nc1cnc2c(c1)c(-c1ccc(C(F)(F)F)cc1)cn2C] |
| 52 | 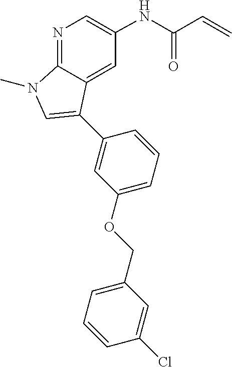 [SMILES: C=CC(=O)Nc1cnc2c(c1)c(-c1cccc(OCc3cccc(Cl)c3)c1)cn2C] |
| 53 | 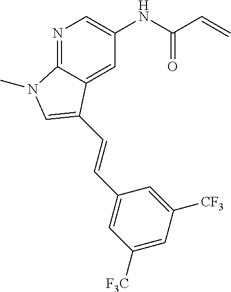 [SMILES: C=CC(=O)Nc1cnc2c(c1)c(/C=C/c1cc(C(F)(F)F)cc(C(F)(F)F)c1)cn2C] |
| 54 | 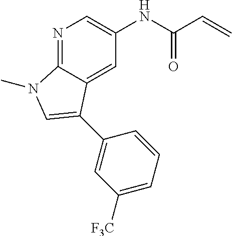 [SMILES: C=CC(=O)Nc1cnc2c(c1)c(-c1cccc(C(F)(F)F)c1)cn2C] |
| 55 | 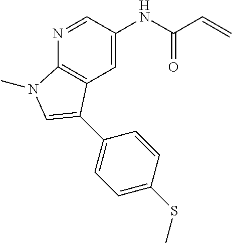 [SMILES: C=CC(=O)Nc1cnc2c(c1)c(-c1ccc(SC)cc1)cn2C] |
| 56 | 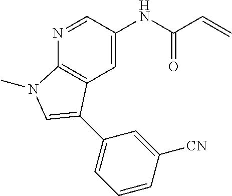 [SMILES: C=CC(=O)Nc1cnc2c(c1)c(-c1cccc(C#N)c1)cn2C] |
| 57 | 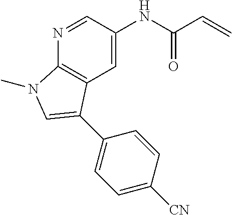 [SMILES: C=CC(=O)Nc1cnc2c(c1)c(-c1ccc(C#N)cc1)cn2C] |
| 58 | 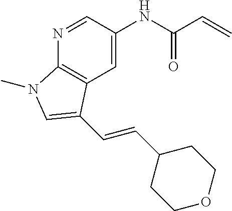 [SMILES: C=CC(=O)Nc1cnc2c(c1)c(/C=C/C1CCOCC1)cn2C] |
| 59 | 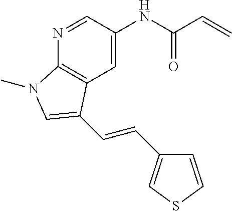 [SMILES: C=CC(=O)Nc1cnc2c(c1)c(/C=C/c1ccsc1)cn2C] |
| 60 | 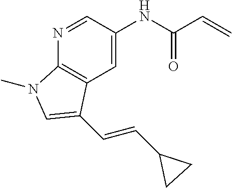 [SMILES: C=CC(=O)Nc1cnc2c(c1)c(/C=C/C1CC1)cn2C] |
| 61 | 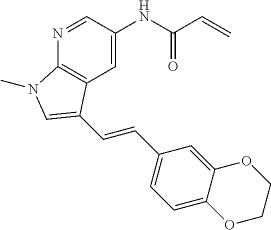 [SMILES: C=CC(=O)Nc1cnc2c(c1)c(/C=C/c1ccc3c(c1)OCCO3)cn2C] |
| 62 | 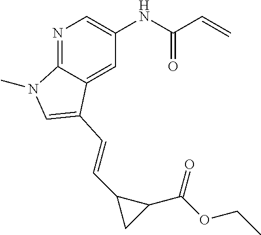 [SMILES: C=CC(=O)Nc1cnc2c(c1)c(/C=C/C1CC1C(=O)OCC)cn2C] |
| 63 | 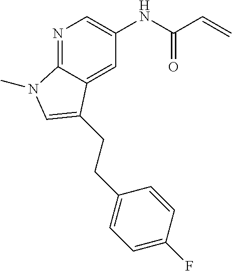 [SMILES: C=CC(=O)Nc1cnc2c(c1)c(CCc1ccc(F)cc1)cn2C] |
| 64 | 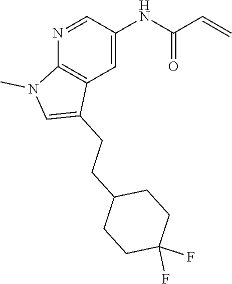 [SMILES: C=CC(=O)Nc1cnc2c(c1)c(CCC1CCC(F)(F)CC1)cn2C] |
| 65 | 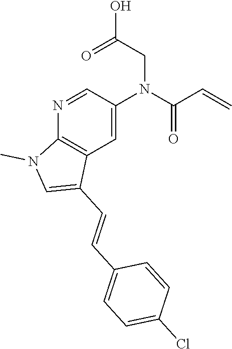 [SMILES: C=CC(=O)N(CC(=O)O)c1cnc2c(c1)c(/C=C/c1ccc(Cl)cc1)cn2C] |
| 66 | 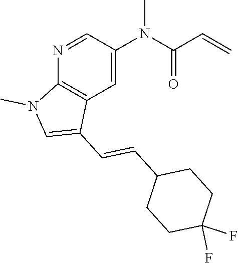 [SMILES: C=CC(=O)N(C)c1cnc2c(c1)c(/C=C/C1CCC(F)(F)CC1)cn2C] |
| 67 | 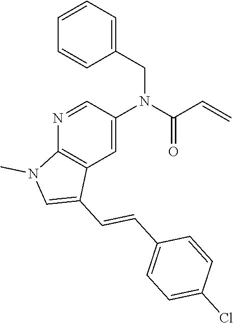 [SMILES: C=CC(=O)N(Cc1ccccc1)c1cnc2c(c1)c(/C=C/c1ccc(Cl)cc1)cn2C] |
| 68 | 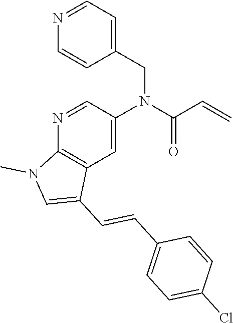 [SMILES: C=CC(=O)N(Cc1ccncc1)c1cnc2c(c1)c(/C=C/c1ccc(Cl)cc1)cn2C] |
| 69 | 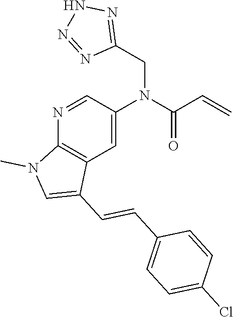 [SMILES: C=CC(=O)N(Cc1nn[nH]n1)c1cnc2c(c1)c(/C=C/c1ccc(Cl)cc1)cn2C] |
| 70 | 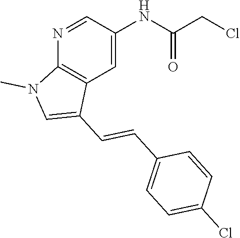 [SMILES: Cn1cc(/C=C/c2ccc(Cl)cc2)c2cc(NC(=O)CCl)cnc21] |
| 71 | 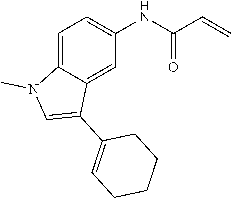 [SMILES: C=CC(=O)Nc1ccc2c(c1)c(C1=CCCCC1)cn2C] |
| 72 | 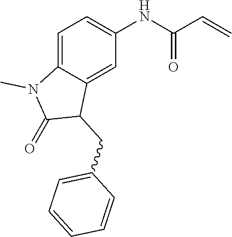 [SMILES: C=CC(=O)Nc1ccc2c(c1)C(Cc1ccccc1)C(=O)N2C] |
| 73 | 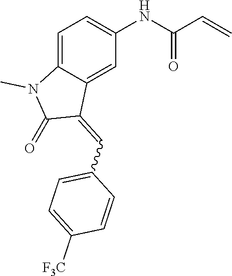 [SMILES: C=CC(=O)Nc1ccc2c(c1)C(=Cc1ccc(C(F)(F)F)cc1)C(=O)N2C] |
| 74 | 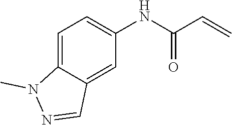 |
| 75 | 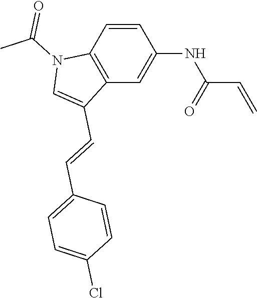 [SMILES: C=CC(=O)Nc1ccc2c(c1)c(/C=C/c1ccc(Cl)cc1)cn2C(C)=O] |
| 76 | 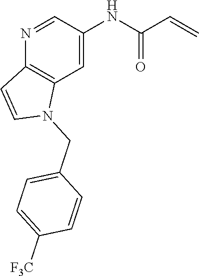 [SMILES: C=CC(=O)Nc1cnc2ccn(Cc3ccc(C(F)(F)F)cc3)c2c1] |
| 77 | 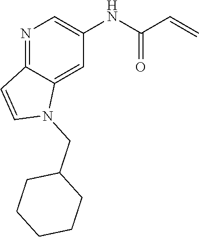 [SMILES: C=CC(=O)Nc1cnc2ccn(CC3CCCCC3)c2c1] |
| 78 | 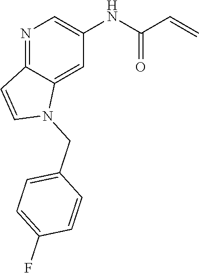 [SMILES: C=CC(=O)Nc1cnc2ccn(Cc3ccc(F)cc3)c2c1] |
| 79 | 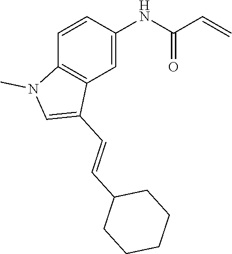 [SMILES: C=CC(=O)Nc1ccc2c(c1)c(/C=C/C1CCCCC1)cn2C] |
| 80 | 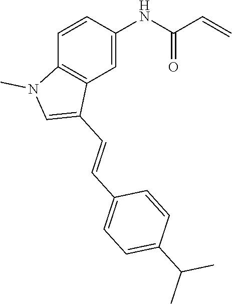 [SMILES: C=CC(=O)Nc1ccc2c(c1)c(/C=C/c1ccc(C(C)C)cc1)cn2C] |
| 81 | 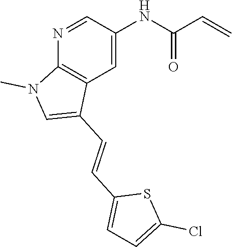 [SMILES: C=CC(=O)Nc1cnc2c(c1)c(/C=C/c1ccc(Cl)s1)cn2C] |
| 82 | 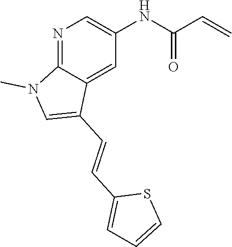 [SMILES: C=CC(=O)Nc1cnc2c(c1)c(/C=C/c1cccs1)cn2C] |
| 83 | 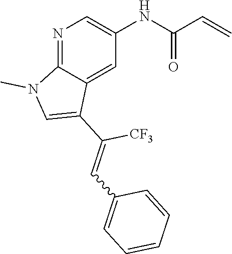 [SMILES: C=CC(=O)Nc1cnc2c(c1)c(C(=Cc1ccccc1)C(F)(F)F)cn2C] |
| 84 | 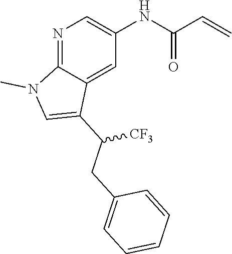 [SMILES: C=CC(=O)Nc1cnc2c(c1)c(C(Cc1ccccc1)C(F)(F)F)cn2C] |
| 85 | 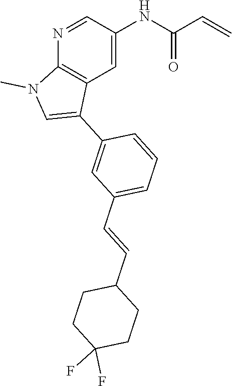 [SMILES: C=CC(=O)Nc1cnc2c(c1)c(-c1cccc(/C=C/C3CCC(F)(F)CC3)c1)cn2C] |
| 86 | 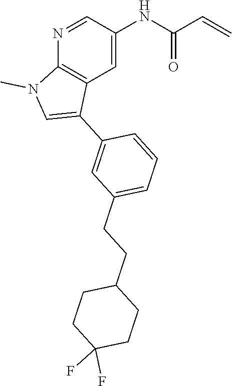 [SMILES: C=CC(=O)Nc1cnc2c(c1)c(-c1cccc(CCC3CCC(F)(F)CC3)c1)cn2C] |
| 87 | 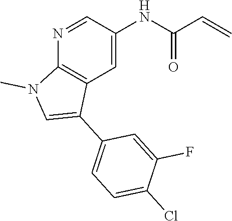 [SMILES: C=CC(=O)Nc1cnc2c(c1)c(-c1ccc(Cl)c(F)c1)cn2C] |
| 88 | 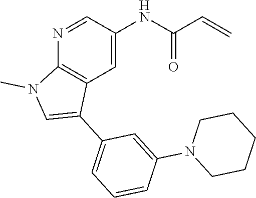 [SMILES: C=CC(=O)Nc1cnc2c(c1)c(-c1cccc(N3CCCCC3)c1)cn2C] |
| 89 | 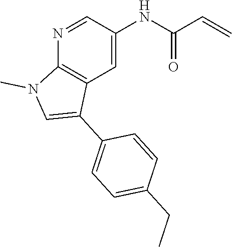 [SMILES: C=CC(=O)Nc1cnc2c(c1)c(-c1ccc(CC)cc1)cn2C] |
| 90 | 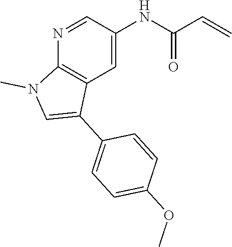 [SMILES: C=CC(=O)Nc1cnc2c(c1)c(-c1ccc(OC)cc1)cn2C] |
| 91 | 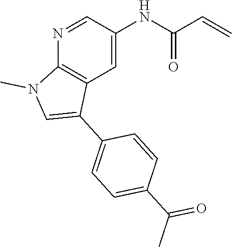 [SMILES: C=CC(=O)Nc1cnc2c(c1)c(-c1ccc(C(C)=O)cc1)cn2C] |
| 92 | 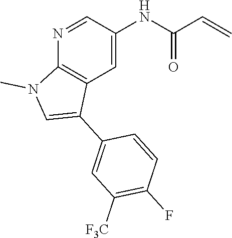 [SMILES: C=CC(=O)Nc1cnc2c(c1)c(-c1ccc(F)c(C(F)(F)F)c1)cn2C] |
| 93 | 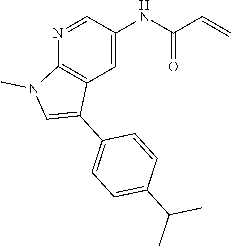 [SMILES: C=CC(=O)Nc1cnc2c(c1)c(-c1ccc(C(C)C)cc1)cn2C] |
| 94 | 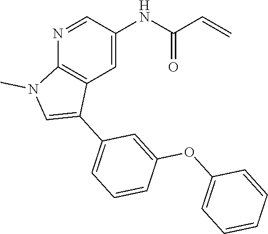 [SMILES: C=CC(=O)Nc1cnc2c(c1)c(-c1cccc(Oc3ccccc3)c1)cn2C] |
| 95 | 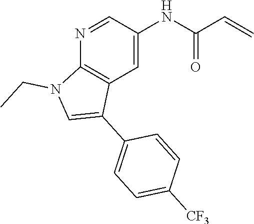 [SMILES: C=CC(=O)Nc1cnc2c(c1)c(-c1ccc(C(F)(F)F)cc1)cn2CC] |
| 96 | 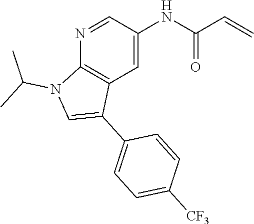 [SMILES: C=CC(=O)Nc1cnc2c(c1)c(-c1ccc(C(F)(F)F)cc1)cn2C(C)C] |
| 97 | 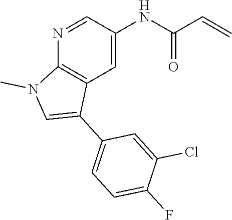 [SMILES: C=CC(=O)Nc1cnc2c(c1)c(-c1ccc(F)c(Cl)c1)cn2C] |
| 98 | 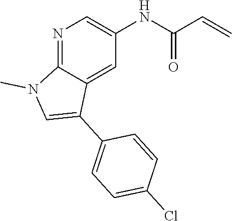 [SMILES: C=CC(=O)Nc1cnc2c(c1)c(-c1ccc(Cl)cc1)cn2C] |
| 99 | 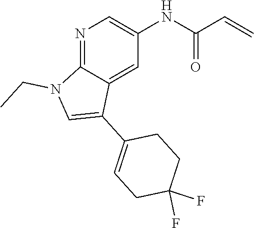 [SMILES: C=CC(=O)Nc1cnc2c(c1)c(C1=CCC(F)(F)CC1)cn2CC] |
| 100 | 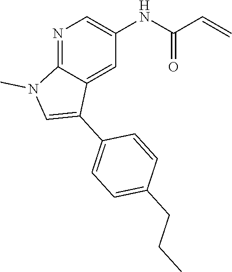 [SMILES: C=CC(=O)Nc1cnc2c(c1)c(-c1ccc(CCC)cc1)cn2C] |
| 101 | 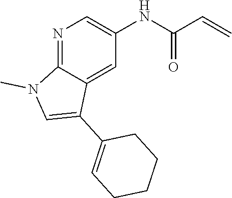 [SMILES: C=CC(=O)Nc1cnc2c(c1)c(C1=CCCCC1)cn2C] |
| 102 | 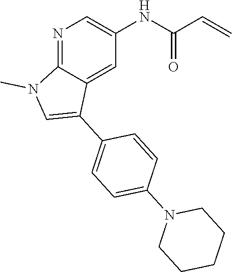 [SMILES: C=CC(=O)Nc1cnc2c(c1)c(-c1ccc(N3CCCCC3)cc1)cn2C] |
| 103 | 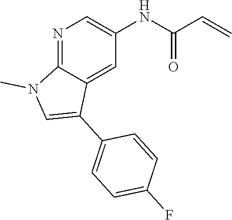 [SMILES: C=CC(=O)Nc1cnc2c(c1)c(-c1ccc(F)cc1)cn2C] |
| 104 | 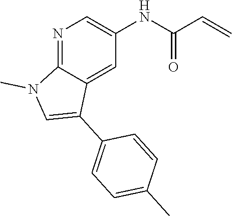 [SMILES: C=CC(=O)Nc1cnc2c(c1)c(-c1ccc(C)cc1)cn2C] |
| 105 | 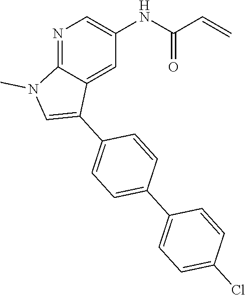 [SMILES: C=CC(=O)Nc1cnc2c(c1)c(-c1ccc(-c3ccc(Cl)cc3)cc1)cn2C] |
| 106 | 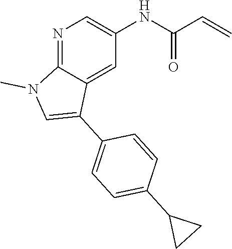 [SMILES: C=CC(=O)Nc1cnc2c(c1)c(-c1ccc(C3CC3)cc1)cn2C] |
| 107 | 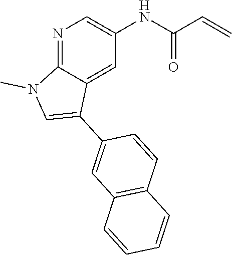 [SMILES: C=CC(=O)Nc1cnc2c(c1)c(-c1ccc3ccccc3c1)cn2C] |
| 108 | 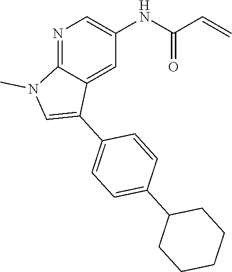 [SMILES: C=CC(=O)Nc1cnc2c(c1)c(-c1ccc(C3CCCCC3)cc1)cn2C] |
| 109 | 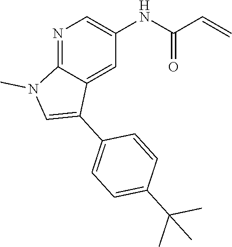 [SMILES: C=CC(=O)Nc1cnc2c(c1)c(-c1ccc(C(C)(C)C)cc1)cn2C] |
| 110 | 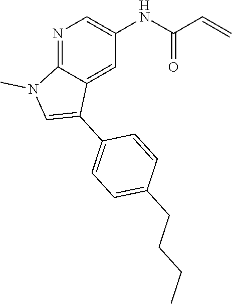 [SMILES: C=CC(=O)Nc1cnc2c(c1)c(-c1ccc(CCCC)cc1)cn2C] |
| 111 | 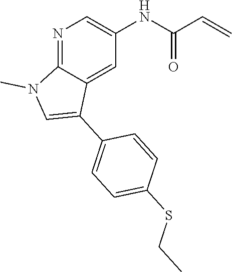 [SMILES: C=CC(=O)Nc1cnc2c(c1)c(-c1ccc(SCC)cc1)cn2C] |
| 112 | 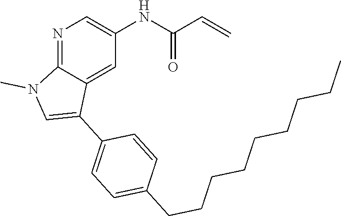 [SMILES: C=CC(=O)Nc1cnc2c(c1)c(-c1ccc(CCCCCCCCC)cc1)cn2C] |
| 113 | 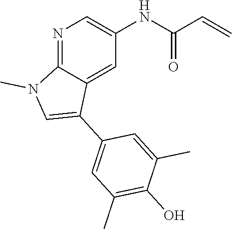 [SMILES: C=CC(=O)Nc1cnc2c(c1)c(-c1cc(C)c(O)c(C)c1)cn2C] |
| 114 | 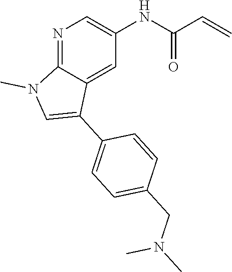 [SMILES: C=CC(=O)Nc1cnc2c(c1)c(-c1ccc(CN(C)C)cc1)cn2C] |
| 115 | 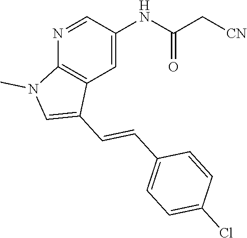 [SMILES: Cn1cc(/C=C/c2ccc(Cl)cc2)c2cc(NC(=O)CC#N)cnc21] |
| 116 | 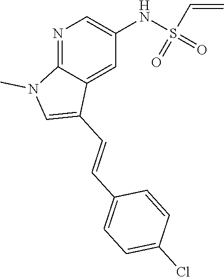 [SMILES: C=CS(=O)(=O)Nc1cnc2c(c1)c(/C=C/c1ccc(Cl)cc1)cn2C] |
| 117 | 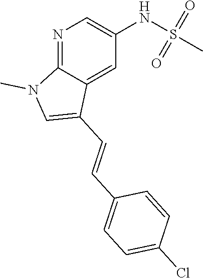 |
| 118 | 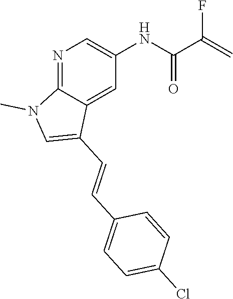 [SMILES: C=C(F)C(=O)Nc1cnc2c(c1)c(/C=C/c1ccc(Cl)cc1)cn2C] |
| 119 | 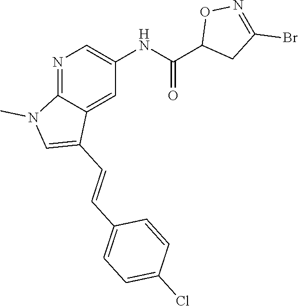 [SMILES: Cn1cc(/C=C/c2ccc(Cl)cc2)c2cc(NC(=O)C3CC(Br)=NO3)cnc21] |
| 120 | 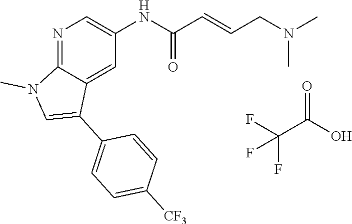 |
| 121 | 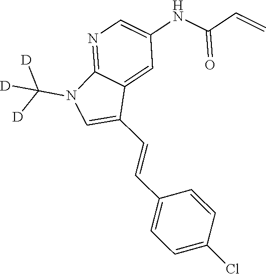 [SMILES: [2H]C([2H])([2H])n1cc(/C=C/c2ccc(Cl)cc2)c2cc(NC(=O)C=C)cnc21] |
| 122 | 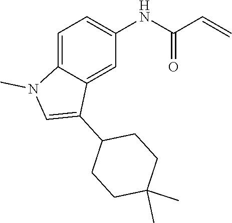 [SMILES: C=CC(=O)Nc1ccc2c(c1)c(C1CCC(C)(C)CC1)cn2C] |
| 123 | 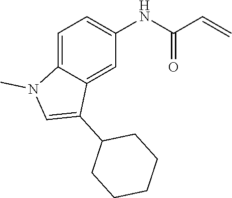 [SMILES: C=CC(=O)Nc1ccc2c(c1)c(C1CCCCC1)cn2C] |
| 124 | 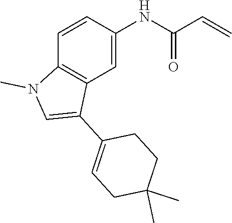 [SMILES: C=CC(=O)Nc1ccc2c(c1)c(C1=CCC(C)(C)CC1)cn2C] |
| 125 | 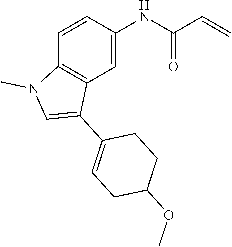 [SMILES: C=CC(=O)Nc1ccc2c(c1)c(C1=CCC(OC)CC1)cn2C] |
| 126 | 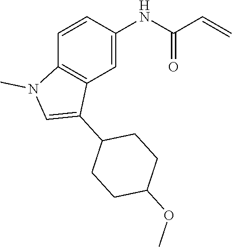 [SMILES: C=CC(=O)Nc1ccc2c(c1)c(C1CCC(OC)CC1)cn2C] |
| 127 | 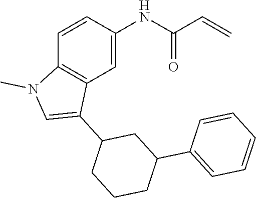 [SMILES: C=CC(=O)Nc1ccc2c(c1)c(C1CCCC(c3ccccc3)C1)cn2C] |
| 128 | 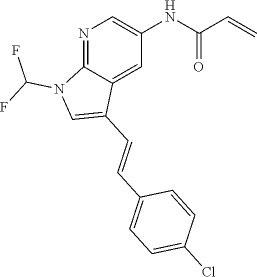 [SMILES: C=CC(=O)Nc1cnc2c(c1)c(/C=C/c1ccc(Cl)cc1)cn2C(F)F] |
| 129 | 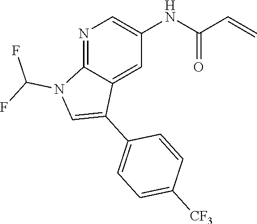 [SMILES: C=CC(=O)Nc1cnc2c(c1)c(-c1ccc(C(F)(F)F)cc1)cn2C(F)F] |
| 130 | 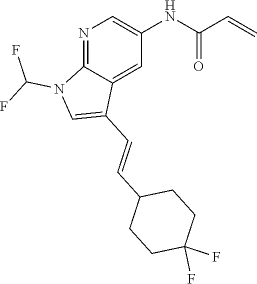 [SMILES: C=CC(=O)Nc1cnc2c(c1)c(/C=C/C1CCC(F)(F)CC1)cn2C(F)F] |
| 131 | 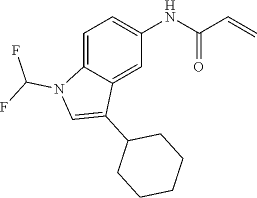 [SMILES: C=CC(=O)Nc1ccc2c(c1)c(C1CCCCC1)cn2C(F)F] |
| 132 | 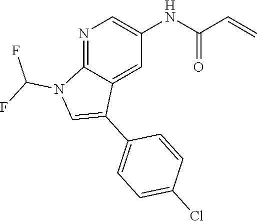 [SMILES: C=CC(=O)Nc1cnc2c(c1)c(-c1ccc(Cl)cc1)cn2C(F)F] |
| 133 | 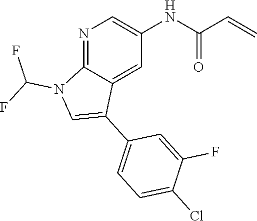 [SMILES: C=CC(=O)Nc1cnc2c(c1)c(-c1ccc(Cl)c(F)c1)cn2C(F)F] |
| 134 | 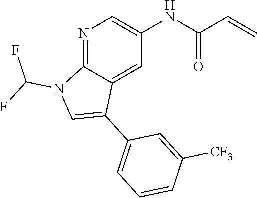 [SMILES: C=CC(=O)Nc1cnc2c(c1)c(-c1cccc(C(F)(F)F)c1)cn2C(F)F] |
| 135 | 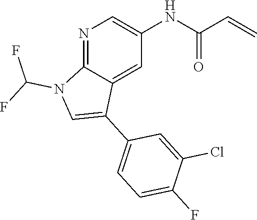 [SMILES: C=CC(=O)Nc1cnc2c(c1)c(-c1ccc(F)c(Cl)c1)cn2C(F)F] |
| 136 | 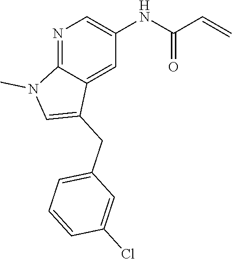 [SMILES: C=CC(=O)Nc1cnc2c(c1)c(Cc1cccc(Cl)c1)cn2C] |
| 137 | 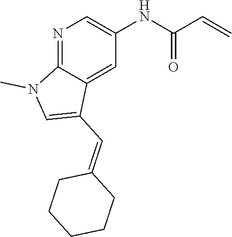 [SMILES: C=CC(=O)Nc1cnc2c(c1)c(C=C1CCCCC1)cn2C] |
| 138 | 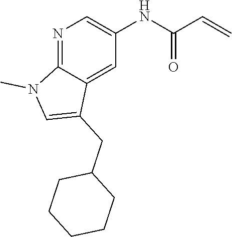 [SMILES: C=CC(=O)Nc1cnc2c(c1)c(CC1CCCCC1)cn2C] |

(9) In some aspects, the present disclosure is directed to a compound, a pharmaceutically acceptable salt thereof, a stereoisomer thereof, a tautomer thereof, a solvate thereof, a hydrate thereof, or a prodrug analog thereof, in which the compound may be one selected from the group consisting of compounds represented by 1 to 138.

### Pharmaceutical Composition, Method of Treatment or Prevention, and Use

In some aspects, the present disclosure is directed to a pharmaceutical composition including the compound, a pharmaceutically acceptable salt thereof, stereoisomer thereof, a tautomer thereof, a solvate thereof, a hydrate thereof, or a prodrug analog thereof according to above any one of (1) to (9), as an effective component.

For administration, the pharmaceutical composition of the present invention may further include at least one type of a pharmaceutically acceptable carrier, in addition to the compound, a pharmaceutically acceptable salt thereof, stereoisomer thereof, a tautomer thereof, a solvate thereof, a hydrate thereof, or a prodrug analog thereof according to above any one of (1) to (9). The pharmaceutically acceptable carrier used may include saline solution, sterilized water, Ringer's solution, buffered saline, dextrose solution, maltodextrin solution, glycerol, ethanol and a mixture of at least one component thereof, and other conventional additives such as antioxidants, buffer solutions, bacteriostatic agents, etc., may be added thereto, if needed. Also, such pharmaceutical composition may be formulated into injectable dosage forms such as aqueous solutions, suspensions, emulsions, etc., pills, capsules, granules or tablets in such a way that diluents, dispersing agents, surfactants, binders and lubricants are additionally added thereto. Thus, the composition of the present invention may be patches, liquids and solutions, pills, capsules, granules, tablets, suppositories, etc. Such preparations may be prepared according to a conventional method used for formulation in the art or a method disclosed in Remington's Pharmaceutical Science (latest edition), Mack Publishing Company, Easton PA, and such composition may be formulated into various preparations depending on each disease or component.

In some aspects, the pharmaceutical composition may be used for preventing or treating YAP/TAZ-TEAD-mediated diseases or TEAD-dependent gene transcription-mediated diseases.

In some aspects, the YAP/TAZ-TEAD-mediated diseases or TEAD-dependent gene transcription-mediated diseases may include cancer.

In some aspects, the present disclosure is directed to a method for preventing or treating YAP/TAZ-TEAD-mediated diseases or TEAD-dependent gene transcription-mediated diseases, and the method includes administering a therapeutically effective amount of the compound, a pharmaceutically acceptable salt thereof, stereoisomer thereof, a tautomer thereof, a solvate thereof, a hydrate thereof, or a prodrug analog thereof according to above any one of (1) to (9).

In some aspects, the present disclosure is directed to use of the compound, a pharmaceutically acceptable salt thereof, stereoisomer thereof, a tautomer thereof, a solvate thereof, a hydrate thereof, or a prodrug analog thereof according to above any one of (1) to (9) for preventing or treating YAP/TAZ-TEAD-mediated diseases or TEAD-dependent gene transcription-mediated diseases.

In some aspects, the present disclosure is directed to use of the compound, a pharmaceutically acceptable salt thereof, stereoisomer thereof, a tautomer thereof, a solvate thereof, a hydrate thereof, or a prodrug analog thereof according to above any one of (1) to (9) in preparation of a medicament for preventing or treating YAP/TAZ-TEAD-mediated diseases or TEAD-dependent gene transcription-mediated diseases.

Disclosed compounds are provided as shown in the following embodiments.

### General Process

The following generalized schemes are used to prepare the disclosed compounds, intermediates, and pharmaceutically acceptable salts thereof. Disclosed compounds and intermediates may be prepared using standard organic synthetic techniques and from commercially available starting materials and reagents. It will be appreciated that synthetic procedures employed in the preparation of disclosed compounds and intermediates will depend on the particular substituents present in the compound or intermediate and that various protection, deprotection, and conversion steps that are standard in organic synthesis may be required, but may not be illustrated in the following general schemes. It is also to be understood that any of the steps shown in any of the following general schemes may be used in any combination and in any order that is chemically feasible to achieve a desired intermediate or disclosed compound. It is further to be understood that when the stereochemistry of a particular compound or intermediate is not disclosed in any given example, the stereochemistry of said compound or intermediate may be determined by any known means common in the art.

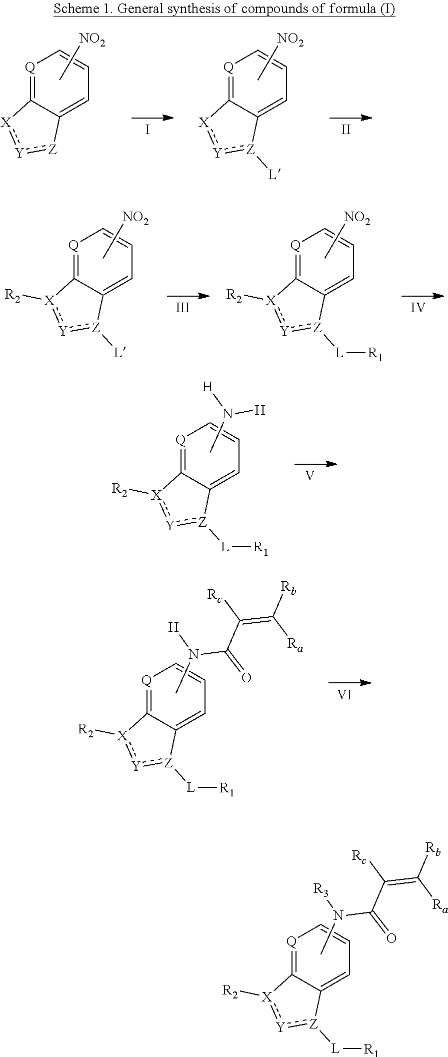

Step I consists in a halogenation procedure using molecular halogen or N-halosuccinimide. L′ can be a halogen such as bromide or iodide.

Step II consists in an alkylation procedure by use the corresponding halogen-alkyl or halogen-cycloalkyl derivatives, which themselves can be functionalized in further steps.

The cross-coupling of the halide R′ in step III is underwent in presence of Pd catalyst and base, by the use of boronic acid derivatives or boronic acid pinacol ester derivatives.

The boronic acid pinacol ester derivatives can be prepared by the use of appropriate aldehyde derivatives and aryl halide derivatives.

Step IV represents a reduction of the nitro group followed in step V by an amide formation using the appropriate acryloyl chloride derivatives.

The acylation or alkylation of the amide in step VI is presented by the use of appropriate acyl chloride or alkyl halide.

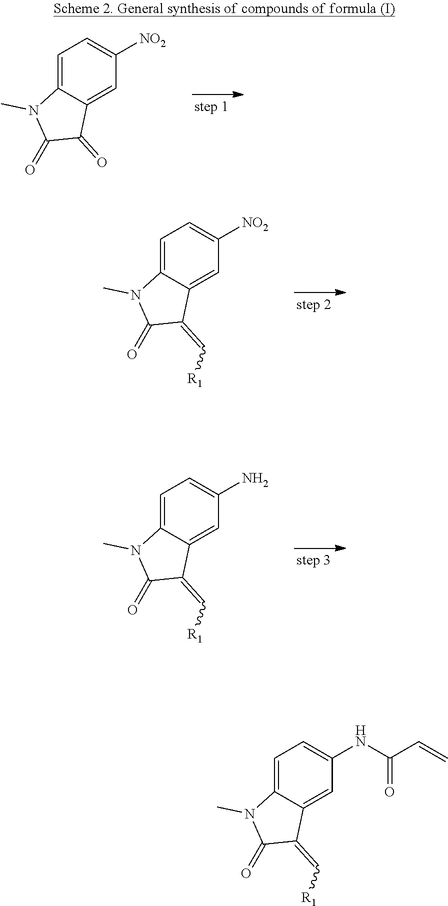

Step 1 consists in a olefination procedure using Wittig reagents.

Step 2 represents a reduction of the nitro group followed in step 3 by an amide formation using the appropriate acryloyl chloride derivatives.

The examples that follow describe the preparation of certain compounds of formula (I). The examples are not limiting but serve merely to illustrate in this application.

### Advantageous Effects

According to the present inventions, the bicyclic heterocycle compounds represented by formula (I), pharmaceutically acceptable salts thereof, stereoisomers thereof, tautomers thereof, solvates thereof, hydrates thereof or prodrug analogs thereof bind to TEAD and/or effectively inhibit YAP/TAZ-TEAD or TEAD-dependent gene transcription, and thus the compounds of the present invention are useful in the treatment of disease related to the activity of YAP/TAZ-TEAD or TEAD-dependent gene transcription including, e.g. cancers and other diseases.

### MODE FOR INVENTION

### Abbreviation

The following abbreviations and empirical formulae are used:
CD₃OD Methanol-d4
CDCl₃ Chloroform-d
CHCl₃ Chloroform
Cs₂CO₃ Cesium carbonate
DCM Dichloromethane
DIPEA N,N-Diisopropylethylamine
DMAP 4-Dimethylaminopyridine
DMF N,N-Dimethylformamide
DMSO Dimethyl sulfoxide
DMSO-d6 Dimethyl sulfoxide-d6
EDCI 1-Ethyl-3-(3-dimethylaminopropyl) carbodiimide hydrochloride
ESI Electrospray ionization
EtOH Ethanol
EtOAc Ethyl acetate
HCl Hydrogen chloride
Hex n-Hexane
HPLC High-Performance Liquid Chromatography
Hz Hertz
IC₅₀ The half maximal inhibitory concentration
KHCO₃ Potassium bicarbonate
KOAc Potassium acetate
KOH Potassium hydroxide
L liter(s)
LCMS Liquid chromatography mass spectrometry
MeCN Acetonitrile
MeOH Methanol
mL milliliter(s)
mmol millimole(s)
min minute(s)
N Normal concentration
NaBH₃ CN Sodium cyanoborohydride
Na₂CO₃ Sodium carbonate
NaH Sodium hydride
NaOH Sodium hydroxide
NaOMe Sodium methoxide
Na₂SO₄ Sodium sulfate
Na₂S₂O₃ Sodium thiosulfate
n-BuLi n-Butyllithium
NBS N-Bromosuccinimide
NH₄Cl Ammonium chloride
nm nanometer(s)
NMR Nuclear magnetic resolution
t-BuOK Potassium tert-butoxide
TEA Triethylamine
TFA Trifluoroacetic acid
THF Tetrahydrofuran® C. degree of Celsius
r.t. room temperature
h hour(s)
Pd/C Palladium on carbon
Pd(dppf)Cl₂-DCM [1,1′-Bis(diphenylphosphino)ferrocene]palladium(II) dichloride dichloromethane complex
Pd(PPh₃)₂Cl₂ Bis(triphenylphosphine)palladium(II)dichloride
Pd(PPh₃)₄ Tetrakis(triphenylphosphine)palladium(0)
Pd₂dba₃ Tris(dibenzylideneacetone)dipalladium(0)
PBS Phosphate-Buffered Saline
PPh₃ Triphenylphosphine
prep Preparative
WT wild type
Xantphos 4,5-Bis(diphenylphosphino)-9,9-dimethylxanthene
μM micromolarity

### EXAMPLES

The following are examples of methods and compositions of the disclosure. It is understood that various other embodiments may be practiced, given the general description provided above. The disclosure will be more fully understood by reference to the following examples. The claims should not, however, be construed as limited to the scope of the examples.

### Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times were approximate and were not optimized. Column chromatography and thin layer chromatography (TLC) were performed on silica gel unless otherwise noted.

### Intermediate A

### Preparation of 3-bromo-1-methyl-5-nitro-1H-indazole

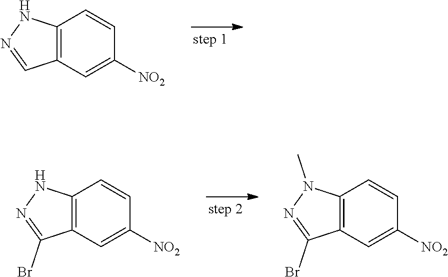

### Step 1: 3-Bromo-5-nitro-1H-indazole

5-Nitro-1H-indazole (30.0 g, 184 mmol) and NBS (36.0 g, 202 mmol) were dissolved in MeCN (300 mL) and heated to reflux for 30 min, then concentrated. To the resultant mixture was diluted with EtOAc (1.00 L), washed with water (1.00 L) twice, the organic phase was separated and washed successively with 10% Na₂S₂O₃ solution (500 mL), water (500 mL), saturated brine (500 mL), then the organic phase was separated, dried over anhydrous Na₂SO₄, filtered, concentrated to obtain 3-bromo-5-nitro-1H-indazole (45.4 g, quantitative). ¹H NMR (600 MHz, CDCl₃) δ 10.37 (s, 1H), 8.67 (d, J=2.0 Hz, 1H), 8.36 (dd, J=9.2, 2.1 Hz, 1H), 7.58 (d, J=9.1 Hz, 1H). LCMS (ESI) m/z=242 (M+H)⁺.

### Step 2: 3-Bromo-1-methyl-5-nitro-1H-indazole

To the solution of 3-bromo-5-nitro-1H-indazole (0.500 g, 2.07 mmol) in DMF (8 mL) was added Cs₂CO₃ (1.34 g, 4.13 mmol), iodomethane (0.193 mL, 3.10 mmol) and stirred at 80° C. for 16 h. After reaction completion, water is poured into the reaction mixture. The mixture was filtered, and the resulting solid was dried in vacuo to give 3-bromo-1-methyl-5-nitro-1H-indazole (0.484 g, 91%), which was used next step without further purification. ¹H NMR (600 MHz, CDCl₃) δ 8.61 (d, J=2.0 Hz, 1H), 8.32 (dd, J=9.3, 2.1 Hz, 1H), 7.46 (d, J=9.2 Hz, 1H), 4.13 (s, 3H). LCMS (ESI) m/z=256 (M+H)⁺.

### Intermediate B

### Preparation of 3-bromo-1-ethyl-5-nitro-1H-indazole

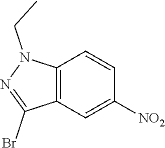

The title compound was prepared from 3-bromo-5-nitro-1H-indazole and ethyl iodide following the procedure outlined for step 2 of Intermediate A. ¹H NMR (600 MHz, CDCl₃) δ 8.62 (d, J=2.0 Hz, 1H), 8.31 (dd, J=9.2, 2.0 Hz, 1H), 7.47 (d, J=9.3 Hz, 1H), 4.46 (q, J=7.3 Hz, 2H), 1.56 (t, J=7.3 Hz, 3H). LCMS (ESI) m/z=271 (M+H)⁺.

### Intermediate C

### Preparation of 3-bromo-1-isopropyl-5-nitro-1H-indazole

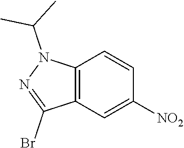 [SMILES: CC(C)n1nc(Br)c2cc([N+](=O)[O-])ccc21]

The title compound was prepared from 3-bromo-5-nitro-1H-indazole and isopropyl iodide following the procedure outlined for step 2 of Intermediate A. ¹H NMR (600 MHZ, CDCl₃) δ 8.61 (d, J=2.0 Hz, 1H), 8.29 (dd, J=9.3, 2.1 Hz, 1H), 7.49 (d, J=9.4 Hz, 1H), 4.85 (hept, J=6.7 Hz, 1H), 1.61 (d, J=6.7 Hz, 6H). LCMS (ESI) m/z=285 (M+H)⁺.

### Intermediate D

### Preparation of 3-bromo-1-cyclopentyl-5-nitro-1H-indazole

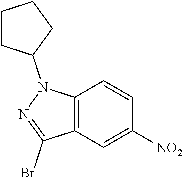 [SMILES: O=[N+]([O-])c1ccc2c(c1)c(Br)nn2C1CCCC1]

The title compound was prepared from 3-bromo-5-nitro-1H-indazole and cyclopentyl chloride following the procedure outlined for step 2 of Intermediate A. ¹H NMR (600 MHZ, CDCl₃) δ 8.54 (d, J=2.0 Hz, 1H), 8.25 (dd, J=9.2, 2.1 Hz, 1H), 7.50 (d, J=9.2 Hz, 1H), 4.97 (p, J=7.3 Hz, 1H), 2.24-2.11 (m, 4H), 2.03-1.93 (m, 2H), 1.79-1.69 (m, 2H). LCMS (ESI) m/z=311 (M+H)⁺.

### Intermediate E

### Preparation of 3-bromo-1-methyl-5-nitro-1H-pyrrolo[2,3-b]pyridine

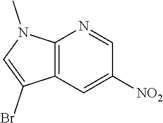

The title compound was prepared from 3-bromo-5-nitro-1H-pyrrolo[2,3-b]pyridine and iodomethane following the procedure outlined for step 2 of Intermediate A. ¹H NMR (600 MHz, CDCl₃) δ 9.24 (d, J=2.4 Hz, 1H), 8.72 (d, J=2.4 Hz, 1H), 7.40 (s, 1H), 3.94 (s, 3H). LCMS (ESI) m/z=256 (M+H)⁺.

### Intermediate F

### Preparation of 3-bromo-1-ethyl-5-nitro-1H-pyrrolo[2,3-b]pyridine

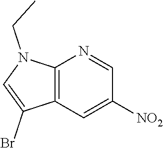

The title compound was prepared from 3-bromo-5-nitro-1H-pyrrolo[2,3-b]pyridine and ethyl iodide following the procedure outlined for step 2 of Intermediate A. ¹H NMR (600 MHz, CDCl₃) δ 9.24 (d, J=2.4 Hz, 1H), 8.72 (d, J=2.4 Hz, 1H), 7.45 (s, 1H), 4.40 (q, J=7.3 Hz, 2H), 1.51 (t, J=7.3 Hz, 3H). LCMS (ESI) m/z=271 (M+H)⁺.

### Intermediate G

### Preparation of 3-bromo-1-isopropyl-5-nitro-1H-pyrrolo[2,3-b]pyridine

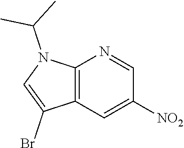 [SMILES: CC(C)n1cc(Br)c2cc([N+](=O)[O-])cnc21]

The title compound was prepared from 3-bromo-5-nitro-1H-pyrrolo[2,3-b]pyridine and isopropyl iodide following the procedure outlined for step 2 of Intermediate A. ¹H NMR (600 MHz, CDCl₃) δ 9.22 (d, J=2.4 Hz, 1H), 8.65 (d, J=2.4 Hz, 1H), 7.55 (s, 1H), 5.32-5.18 (m, 1H), 1.56-1.54 (d, J=6.0 Hz 6H). LCMS (ESI) m/z=285 (M+H)⁺.

### Intermediate H

### Preparation of 3-bromo-1-cyclopentyl-5-nitro-1H-pyrrolo[2,3-b]pyridine

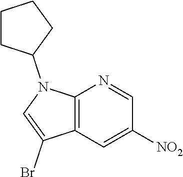 [SMILES: O=[N+]([O-])c1cnc2c(c1)c(Br)cn2C1CCCC1]

The title compound was prepared from 3-bromo-5-nitro-1H-pyrrolo[2,3-b]pyridine and cyclopentyl chloride following the procedure outlined for step 2 of Intermediate A. ¹H NMR (600 MHz, CDCl₃) δ 9.22 (d, J=2.4 Hz, 1H), 8.71 (d, J=2.4 Hz, 1H), 7.49 (s, 1H), 5.34 (p, J=7.5 Hz, 1H), 2.31-2.27 (m, 2H), 1.97-1.77 (m, 6H). LCMS (ESI) m/z=311 (M+H)⁺.

### Intermediate I

### Preparation of 3-bromo-1-methyl-5-nitro-1H-pyrazolo[3,4-b]pyridine

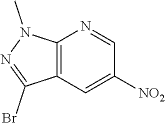

The title compound was prepared from 3-bromo-5-nitro-1H-pyrazolo[3,4-b]pyridine and iodomethane following the procedure outlined for step 2 of Intermediate A. ¹H NMR (600 MHz, CDCl₃) δ 9.44 (d, J=2.3 Hz, 1H), 8.85 (d, J=2.3 Hz, 1H), 4.21 (s, 3H). LCMS (ESI) m/z=257 (M+H)⁺.

### Intermediate J

### Preparation of 3-bromo-1-ethyl-5-nitro-1H-pyrazolo[3,4-b]pyridine

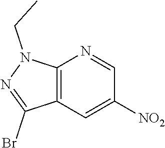

The title compound was prepared from 3-bromo-5-nitro-1H-pyrazolo[3,4-b]pyridine and ethyl iodide following the procedure outlined for step 2 of Intermediate A. ¹H NMR (600 MHz, CDCl₃) δ 9.42 (d, J=2.4 Hz, 1H), 8.84 (d, J=2.4 Hz, 1H), 4.62-4.55 (q, J=6.0 Hz, 2H), 1.59-1.56 (t, J=6.0 Hz, 3H). LCMS (ESI) m/z=272 (M+H)⁺.

### Intermediate K

### Preparation of 3-bromo-1-isopropyl-5-nitro-1H-pyrazolo[3,4-b]pyridine

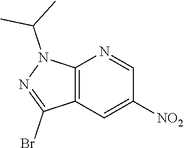 [SMILES: CC(C)n1nc(Br)c2cc([N+](=O)[O-])cnc21]

The title compound was prepared from 3-bromo-5-nitro-1H-pyrazolo[3,4-b]pyridine and isopropyl iodide following the procedure outlined for step 2 of Intermediate A. ¹H NMR (600 MHz, CDCl₃) δ 9.41 (d, J=2.4 Hz, 1H), 8.83 (d, J=2.4 Hz, 1H), 5.33 (hept, J=6.7 Hz, 1H), 1.61 (d, J=6.7 Hz, 6H). LCMS (ESI) m/z=286 (M+H)⁺.

### Intermediate L

### Preparation of 3-bromo-1-ethyl-5-nitro-1H-indole

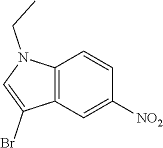

The title compound was prepared from 3-bromo-5-nitro-1H-indole and ethyl iodide following the procedure outlined for step 2 of Intermediate A. ¹H NMR (400 MHZ, CDCl₃) δ 8.54 (s, 1H), 8.15 (d, J=9.2 Hz, 1H), 7.37 (d, J=9.2 Hz, 1H), 7.30 (s, 1H), 4.22 (q, J=7.4 Hz, 2H), 1.51 (t, J=7.4 Hz, 3H). LCMS (ESI) m/z=270 (M+H)⁺.

### Intermediate M

### Preparation of 3-bromo-1-isopropyl-5-nitro-1H-indole

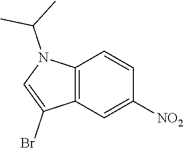 [SMILES: CC(C)n1cc(Br)c2cc([N+](=O)[O-])ccc21]

The title compound was prepared from 3-bromo-5-nitro-1H-indole and isopropyl iodide following the procedure outlined for step 2 of Intermediate A. ¹H NMR (600 MHZ, CDCl₃) δ 8.55 (d, J=2.2 Hz, 1H), 8.16 (dd, J=9.1, 2.2 Hz, 1H), 7.40 (dd, J=8.7, 2.8 Hz, 2H), 4.75-4.69 (m, 1H), 1.56 (d, J=6.0 Hz, 6H). LCMS (ESI) m/z=284 (M+H)⁺.

### Intermediate N

### Preparation of 3-bromo-1-cyclopentyl-5-nitro-1H-indole

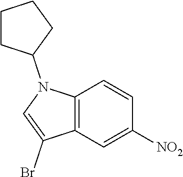 [SMILES: O=[N+]([O-])c1ccc2c(c1)c(Br)cn2C1CCCC1]

The title compound was prepared from 3-bromo-5-nitro-1H-indole and cyclopentyl chloride following the procedure outlined for step 2 of Intermediate A. ¹H NMR (600 MHZ, CDCl₃) δ 8.53 (d, J=2.2 Hz, 1H), 8.14 (dd, J=9.1, 2.2 Hz, 1H), 7.43 (d, J=9.1 Hz, 1H), 7.36 (s, 1H), 4.83 (p, J=6.8 Hz, 1H), 2.31-2.23 (m, 2H), 1.96-1.87 (m, 4H), 1.84-1.80 (m, 2H). LCMS (ESI) m/z=310 (M+H)⁺.

### Intermediate O

### Preparation of 3-bromo-7-fluoro-1-methyl-5-nitro-1H-indole

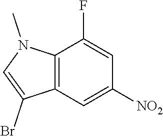

The title compound was prepared from 7-fluoro-5-nitro-1H-indole and iodomethane following the procedure outlined for Intermediate A. ¹H NMR (600 MHZ, CDCl₃) δ 8.34 (d, J=1.9 Hz, 1H), 7.85 (dd, J=12.2, 1.9 Hz, 1H), 7.20 (s, 1H), 4.04 (s, 3H). LCMS (ESI) m/z=273 (M+H)⁺.

### Intermediate P

### Preparation of 3-bromo-7-fluoro-1-isopropyl-5-nitro-1H-indole

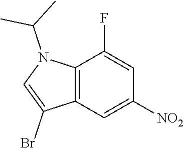 [SMILES: CC(C)n1cc(Br)c2cc([N+](=O)[O-])cc(F)c21]

The title compound was prepared from 7-fluoro-5-nitro-1H-indole and isopropyl iodide chloride following the procedure outlined for Intermediate A. ¹H NMR (600 MHZ, CDCl₃) δ 8.21 (d, J=2.0 Hz, 1H), 7.85 (dd, J=12.6, 2.0 Hz, 1H), 7.43 (s, 1H), 5.05 (m, 1H), 1.56 (d, J=6.6 Hz, 6H). LCMS (ESI) m/z=302 (M+H)⁺.

### Intermediate Q

### Preparation of 3-bromo-1-methyl-6-nitro-1H-indole

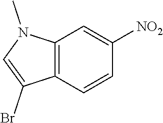

The title compound was prepared from 3-bromo-6-nitro-1H-indole and iodomethane following the procedure outlined for step 2 of Intermediate A. ¹H NMR (600 MHZ, CDCl₃) δ 8.33 (t, J=2.2 Hz, 1H), 8.09 (dd, J=8.8, 2.0 Hz, 1H), 7.64-7.60 (m, 1H), 7.38 (s, 1H), 3.90 (s, 3H). LCMS (ESI) m/z=256 (M+H)⁺.

### Intermediate R

### Preparation of 3-bromo-1,2-dimethyl-5-nitro-1H-indole

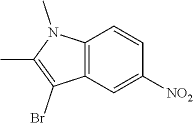

The title compound was prepared from 3-bromo-2-methyl-5-nitro-1H-indole and iodomethane following the procedure outlined for step 2 of Intermediate A. ¹H NMR (600 MHZ, CDCl₃) δ 8.44 (d, J=2.2 Hz, 1H), 8.10 (dd, J=9.0, 2.2 Hz, 1H), 7.28 (d, J=9.0 Hz, 1H), 3.77 (s, 3H), 2.48 (s, 3H). LCMS (ESI) m/z=270 (M+H)⁺.

### Example 1

### (E)-N-(3-(2-(4,4-Difluorocyclohexyl)vinyl)-1-methyl-1H-indazol-5-yl)acrylamide

The overall reaction scheme was as follows:

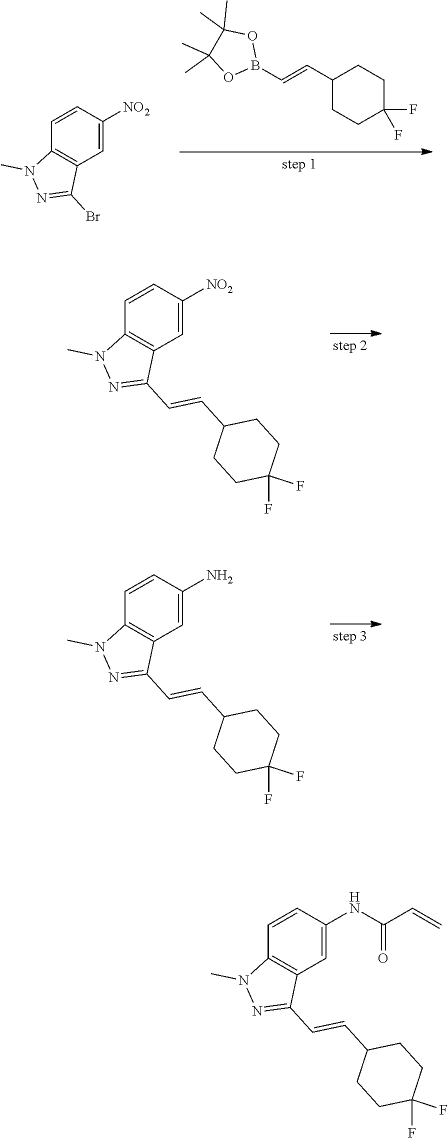 [SMILES: Cn1nc(Br)c2cc([N+](=O)[O-])ccc21 ; CC1(C)OB(/C=C/C2CCC(F)(F)CC2)OC1(C)C ; Cn1nc(/C=C/C2CCC(F)(F)CC2)c2cc([N+](=O)[O-])ccc21 ; Cn1nc(/C=C/C2CCC(F)(F)CC2)c2cc(N)ccc21 ; C=CC(=O)Nc1ccc2c(c1)c(/C=C/C1CCC(F)(F)CC1)nn2C]

### Step 1: (E)-3-(2-(4,4-Difluorocyclohexyl)vinyl)-1-methyl-5-nitro-1H-indazole

The mixture of intermediate A (200 mg, 0.781 mmol), (E)-2-(2-(4,4-difluorocyclohexyl)vinyl)-4,4,5,5-tetramethyl-1,3,2-dioxaborolane (234 mg, 0.859 mmol), Pd(PPh₃)⁺ (90.2 mg, 10 mol %), Na₂CO₃ (248 mg, 2.34 mmol) in dioxane/water (5:1, 8 mL) was stirred at 120° C. under microwave radiation for 2 h then cooled to r.t. The mixture was filtered to remove insoluble and rinsed with EtOAc. The filtrate was concentrated and the residue was purified by flash chromatography (0%-20% EtOAc/DCM) to obtain the titled compound (222 mg, 88%). ¹H NMR (600 MHZ, CDCl₃) δ 8.86 (d, J=1.9 Hz, 1H), 8.19 (dd, J=9.1, 1.9 Hz, 1H), 7.95 (d, J=8.6 Hz, 1H), 6.49 (d, J=15.7 Hz, 1H), 6.22 (dd, J=15.6, 7.0 Hz, 1H), 3.73 (s, 3H), 2.63-2.52 (m, 1H), 2.19-2.03 (m, 2H), 1.99-1.83 (m, 2H), 1.78-1.59 (m, 4H). LCMS (ESI) m/z=322 (M+H)⁺.

### Step 2: (E)-3-(2-(4,4-Difluorocyclohexyl)vinyl)-1-methyl-1H-indazol-5-amine

(E)-3-(2-(4,4-Difluorocyclohexyl)vinyl)-1-methyl-5-nitro-1H-indazole (111 mg, 0.361 mmol), iron powder (60.5 mg, 1.08 mmol), and HCl solution (3 N aqueous solution, 0.240 mL, 0.722 mmol) were suspended in EtOH (3 mL). The reaction mixture was stirred at 80° C. for 15 h then cooled to r.t. The mixture was diluted in EtOAc and filtered to remove insoluble. The filtrate was washed with 1 N NaOH aqueous solution then concentrated. The residue was purified by flash chromatography (0%-10% MeOH/EtOAc) to obtain the titled compound (97.0 mg, 97%). LCMS (ESI) m/z=292 (M+H)⁺.

### Step 3: (E)-N-(3-(2-(4,4-Difluorocyclohexyl)vinyl)-1-methyl-1H-indazol-5-yl)acrylamide

(E)-3-(2-(4,4-Difluorocyclohexyl)vinyl)-1-methyl-1H-indazol-5-amine (188 mg, 0.645 mmol) and TEA (0.18 mL, 1.29 mmol) were diluted in DCM (4 mL) and cooled to 0° C. Acryloyl chloride (0.057 mL, 0.710 mmol) was added at once and the reaction mixture was stirred for 2 h and quenched by adding water. The resultant mixture was separated and the aqueous layer was washed with DCM twice. The organic layer was concentrated and the residue was purified by prep. HPLC (MeCN/water) to obtain the titled compound (148 mg, 66%). ¹H NMR (600 MHz, CD₃OD) δ 8.41 (d, J=2.0 Hz, 1H), 7.54 (dd, J=9.0, 1.9 Hz, 1H), 7.48 (d, J=9.0 Hz, 1H), 6.70 (dd, J=16.4, 1.3 Hz, 1H), 6.54 (dd, J=16.3, 7.0 Hz, 1H), 6.46 (dd, J=16.9, 10.0 Hz, 1H), 6.39 (dd, J=17.0, 1.8 Hz, 1H), 5.79 (dd, J=10.0, 1.8 Hz, 1H), 4.00 (s, 3H), 2.38 (s, 1H), 2.12-2.08 (m, 2H), 1.97-1.82 (m, 6H), 1.65-1.58 (m, 2H). LCMS (ESI) m/z=346 (M+H)⁺.

### Example 2

### (E)-N-(3-(4-Chlorostyryl)-1-methyl-1H-indazol-5-yl)acrylamide

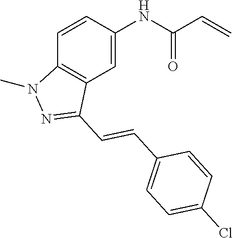 [SMILES: C=CC(=O)Nc1ccc2c(c1)c(/C=C/c1ccc(Cl)cc1)nn2C]

The title compound was prepared from Intermediate A and (E)-(4-chlorostyryl) boronic acid following the procedure outlined for Example 1. ¹H NMR (400 MHZ, CD₃OD) δ 8.57 (s, 1H), 7.62 (d, J=8.1 Hz, 2H), 7.55 (s, 2H), 7.43 (d, J=3.4 Hz, 2H), 7.39 (d, J=8.1 Hz, 2H), 6.54-6.37 (m, 2H), 5.82 (s, 1H), 4.07 (s, 3H). LCMS (ESI) m/z=338 (M+H)⁺.

### Example 3

### (E)-N-(3-(2-(4,4-Difluorocyclohexyl)vinyl)-1-ethyl-1H-indazol-5-yl)acrylamide

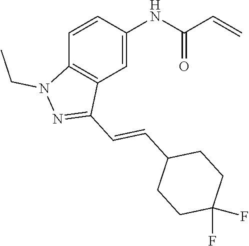 [SMILES: C=CC(=O)Nc1ccc2c(c1)c(/C=C/C1CCC(F)(F)CC1)nn2CC]

The title compound was prepared from Intermediate B and (E)-2-(2-(4,4-difluorocyclohexyl)vinyl)-4,4,5,5-tetramethyl-1,3,2-dioxaborolane following the procedure outlined for Example 1. ¹H NMR (400 MHZ, CD₃OD) δ 8.41 (s, 1H), 7.52 (s, 2H), 6.71 (d, J=16.5 Hz, 1H), 6.54 (dd, J=16.5, 7.1 Hz, 1H), 6.45 (d, J=9.6 Hz, 1H), 6.38 (d, J=17.0 Hz, 1H), 5.78 (d, J=9.8 Hz, 1H), 4.40 (q, J=7.2 Hz, 2H), 2.43-2.35 (m, 1H), 2.15-2.07 (m, 2H), 2.03-1.79 (m, 5H), 1.67-1.58 (m, 2H), 1.44 (t, J=7.2 Hz, 3H). LCMS (ESI) m/z=360 (M+H)⁺.

### Example 4

### (E)-N-(3-(4-Chlorostyryl)-1-ethyl-1H-indazol-5-yl)acrylamide

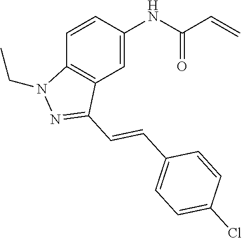 [SMILES: C=CC(=O)Nc1ccc2c(c1)c(/C=C/c1ccc(Cl)cc1)nn2CC]

The title compound was prepared from Intermediate B and (E)-(4-chlorostyryl) boronic acid following the procedure outlined for Example 1. ¹H NMR (400 MHZ, CD₃OD) δ 8.57 (s, 1H), 7.62 (d, J=7.9 Hz, 2H), 7.56 (s, 2H), 7.43 (d, J=2.0 Hz, 2H), 7.41-7.35 (m, 2H), 6.49 (dd, J=17.1, 9.8 Hz, 1H), 6.40 (d, J=16.7 Hz, 1H), 5.80 (d, J=9.8 Hz, 1H), 4.45 (q, J=7.3 Hz, 2H), 1.49 (t, J=7.3 Hz, 3H). LCMS (ESI) m/z=352 (M+H)⁺.

### Example 5

### (E)-N-(3-(2-(4,4-Difluorocyclohexyl)vinyl)-1-isopropyl-1H-indazol-5-yl)acrylamide

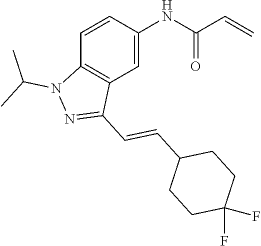 [SMILES: C=CC(=O)Nc1ccc2c(c1)c(/C=C/C1CCC(F)(F)CC1)nn2C(C)C]

The title compound was prepared from Intermediate C and (E)-2-(2-(4,4-difluorocyclohexyl)vinyl)-4,4,5,5-tetramethyl-1,3,2-dioxaborolane following the procedure outlined for Example 1. ¹H NMR (600 MHZ, CD₃OD) δ 8.41 (d, J=1.9 Hz, 1H), 7.54 (d, J=9.0 Hz, 2H), 7.51 (dd, J=9.0, 1.9 Hz, 2H), 6.74 (dd, J=16.3, 1.4 Hz, 1H), 6.53 (dd, J=16.4, 7.0 Hz, 1H), 6.47 (dd, J=16.9, 10.1 Hz, 1H), 6.38 (dd, J=16.9, 1.8 Hz, 1H), 5.78 (dd, J=10.0, 1.8 Hz, 1H), 2.44-2.35 (m, 1H), 2.15-2.08 (m, 2H), 1.99-1.83 (m, 4H), 1.68-1.59 (m, 2H), 1.54 (d, J=6.7 Hz, 6H). LCMS (ESI) m/z=374 (M+H)⁺.

### Example 6

### (E)-N-(3-(4-Chlorostyryl)-1-isopropyl-1H-indazol-5-yl)acrylamide

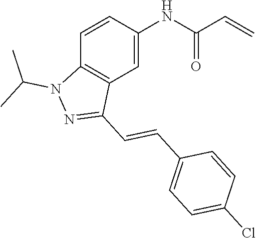 [SMILES: C=CC(=O)Nc1ccc2c(c1)c(/C=C/c1ccc(Cl)cc1)nn2C(C)C]

The title compound was prepared from Intermediate C and (E)-(4-chlorostyryl) boronic acid following the procedure outlined for Example 1. ¹H NMR (600 MHZ, CD₃OD) δ 8.57 (d, J=1.8 Hz, 1H), 7.62 (d, J=8.5 Hz, 2H), 7.59 (d, J=9.0 Hz, 1H), 7.53 (dd, J=9.0, 1.8 Hz, 1H), 7.45 (d, J=1.9 Hz, 2H), 7.38 (d, J=8.4 Hz, 2H), 6.49 (dd, J=16.9, 10.0 Hz, 1H), 6.41 (dd, J=16.9, 1.6 Hz, 1H), 5.80 (dd, J=9.7, 1.7 Hz, 1H), 4.95 (m, 1H), 1.58 (d, J=6.4 Hz, 7H). LCMS (ESI) m/z=366 (M+H)⁺.

### Example 7

### (E)-N-(1-Cyclopentyl-3-(2-(4,4-difluorocyclohexyl)vinyl)-1H-indazol-5-yl)acrylamide

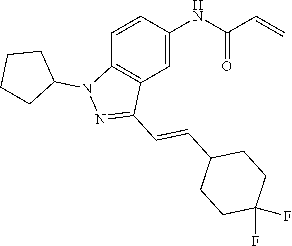 [SMILES: C=CC(=O)Nc1ccc2c(c1)c(/C=C/C1CCC(F)(F)CC1)nn2C1CCCC1]

The title compound was prepared from Intermediate D and (E)-2-(2-(4,4-difluorocyclohexyl)vinyl)-4,4,5,5-tetramethyl-1,3,2-dioxaborolane following the procedure outlined for Example 1. ¹H NMR (600 MHZ, CD₃OD) δ 8.40 (d, J=1.9 Hz, 1H), 7.54 (d, J=9.0 Hz, 1H), 7.50 (dd, J=9.0, 1.9 Hz, 1H), 6.73 (dd, J=16.4, 1.3 Hz, 1H), 6.52 (dd, J=16.3, 7.1 Hz, 1H), 6.47 (dd, J=16.9, 10.0 Hz, 1H), 6.38 (dd, J=17.0, 1.8 Hz, 1H), 5.78 (dd, J=10.0, 1.8 Hz, 1H), 5.06 (p, J=7.8 Hz, 1H), 2.44-2.34 (m, 1H), 2.18-2.07 (m, 7H), 1.99-1.83 (m, 7H), 1.81-1.72 (m, 1H), 1.67-1.55 (m, 1H). LCMS (ESI) m/z=400 (M+H)⁺.

### Example 8

### (E)-N-(3-(4-Chlorostyryl)-1-cyclopentyl-1H-indazol-5-yl)acrylamide

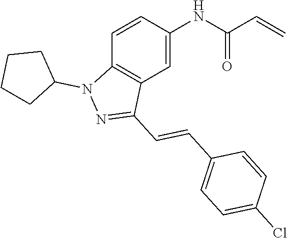 [SMILES: C=CC(=O)Nc1ccc2c(c1)c(/C=C/c1ccc(Cl)cc1)nn2C1CCCC1]

The title compound was prepared from Intermediate D and (E)-(4-chlorostyryl) boronic acid following the procedure outlined for Example 1. ¹H NMR (600 MHZ, CD₃OD) δ 8.56 (d, J=2.0 Hz, 1H), 7.60 (dd, J=15.7, 8.8 Hz, 3H), 7.52 (dd, J=9.0, 2.0 Hz, 1H), 7.44 (d, J=4.8 Hz, 2H), 7.38 (d, J=8.4 Hz, 2H), 6.49 (dd, J=17.0, 10.0 Hz, 1H), 6.41 (dd, J=17.0, 1.8 Hz, 1H), 5.80 (dd, J=10.0, 1.2 Hz, 1H), 5.11 (p, J=7.4 Hz, 1H), 2.24-2.10 (m, 4H), 2.04-1.94 (m, 2H), 1.84-1.75 (m, 2H). LC/MS (ESI) m/z=392 (M+H)⁺.

### Example 9

### (E)-N-(1-Cyclopentyl-3-(2-(4,4-difluorocyclohexyl)vinyl)-1H-pyrrolo[2,3-b]pyridin-5-yl)acrylamide

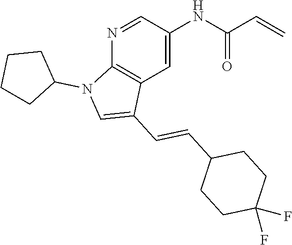 [SMILES: C=CC(=O)Nc1cnc2c(c1)c(/C=C/C1CCC(F)(F)CC1)cn2C1CCCC1]

The title compound was prepared from Intermediate H and (E)-2-(2-(4,4-difluorocyclohexyl)vinyl)-4,4,5,5-tetramethyl-1,3,2-dioxaborolane following the procedure outlined for Example 1. ¹H NMR (400 MHZ, CD₃OD) δ 8.61 (s, 1H), 8.39 (s, 1H), 7.51 (s, 1H), 6.55 (d, J=16.2 Hz, 1H), 6.49-6.35 (m, 2H), 6.11 (dd, J=16.0, 7.0 Hz, 1H), 5.80 (d, J=9.8 Hz, 1H), 5.15 (p, J=7.0, 6.2 Hz, 1H), 2.09-2.04 (m, 2H), 1.93-1.86 (m, 9H), 1.81-1.76 (m, 3H), 1.61-1.55 (m, 3H). LCMS (ESI) m/z=400 (M+H)⁺.

### Example 10

### (E)-N-(3-(4-Chlorostyryl)-1-cyclopentyl-1H-pyrrolo[2,3-b]pyridin-5-yl)acrylamide

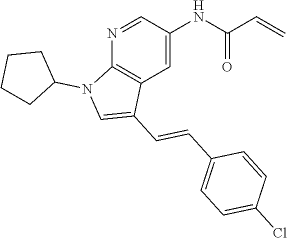 [SMILES: C=CC(=O)Nc1cnc2c(c1)c(/C=C/c1ccc(Cl)cc1)cn2C1CCCC1]

The title compound was prepared from Intermediate H and (E)-(4-chlorostyryl) boronic acid following the procedure outlined for Example 1. ¹H NMR (400 MHZ, CD₃OD) δ 8.79 (s, 1H), 8.43 (s, 1H), 7.76 (s, 1H), 7.53 (d, J=8.2 Hz, 2H), 7.37-7.28 (m, 3H), 7.07 (d, J=16.4 Hz, 1H), 6.56-6.44 (m, 1H), 6.42 (d, J=16.7 Hz, 1H), 5.82 (d, J=9.9 Hz, 1H), 5.20 (s, 1H), 4.09 (d, J=7.3 Hz, 1H), 2.28-2.18 (m, 2H), 2.01-1.92 (m, 6H), 1.86-1.80 (m, 2H). LCMS (ESI) m/z=392 (M+H)⁺.

### Example 11

### (E)-N-(3-(4-Chlorostyryl)-1-cyclopentyl-1H-indol-5-yl)acrylamide

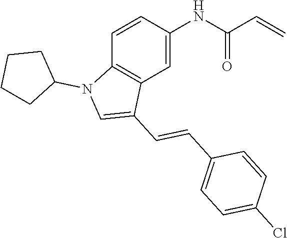 [SMILES: C=CC(=O)Nc1ccc2c(c1)c(/C=C/c1ccc(Cl)cc1)cn2C1CCCC1]

The title compound was prepared from Intermediate N and (E)-(4-chlorostyryl) boronic acid following the procedure outlined for Example 1. ¹H NMR (400 MHZ, CD₃OD) δ 8.38 (s, 1H), 7.73 (d, J=37.5 Hz, 1H), 7.59-7.41 (m, 4H), 7.37 (d, J=13.8 Hz, 1H), 7.32-7.25 (m, 2H), 7.12-7.03 (m, 1H), 7.04-6.93 (m, 1H), 6.54-6.43 (m, 1H), 6.38 (d, J=17.4 Hz, 1H), 5.77 (d, J=9.9 Hz, 1H), 5.21-5.03 (m, 1H), 2.28-2.20 (m, 2H), 1.97-1.91 (m, 4H), 1.84-1.77 (m, 2H). LCMS (ESI) m/z=391 (M+H)⁺.

### Example 12

### (E)-N-(3-(4-Chlorostyryl)-1-methyl-1H-indol-6-yl)acrylamide

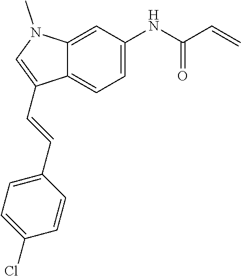 [SMILES: C=CC(=O)Nc1ccc2c(/C=C/c3ccc(Cl)cc3)cn(C)c2c1]

The title compound was prepared from Intermediate Q and (E)-(4-chlorostyryl) boronic acid following the procedure outlined for Example 1. ¹H NMR (400 MHZ, CD₃OD) δ 8.03 (s, 1H), 7.91 (s, 1H), 7.51 (s, 2H), 7.42 (s, 1H), 7.32 (s, 3H), 7.24 (s, 1H), 7.05 (d, J=16.2 Hz, 1H), 6.57-6.46 (m, 1H), 6.40 (d, J=16.5 Hz, 1H), 5.80 (d, J=9.4 Hz, 1H), 3.81 (s, 3H). LCMS (ESI) m/z=337 (M+H)⁺.

### Example 13

### (E)-N-(3-(2-(4,4-Difluorocyclohexyl)vinyl)-1-methyl-1H-indol-6-yl)acrylamide

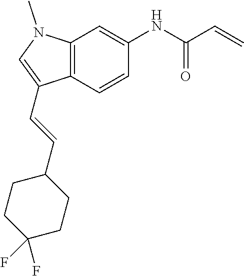 [SMILES: C=CC(=O)Nc1ccc2c(/C=C/C3CCC(F)(F)CC3)cn(C)c2c1]

The title compound was prepared from Intermediate Q and (E)-2-(2-(4,4-difluorocyclohexyl)vinyl)-4,4,5,5-tetramethyl-1,3,2-dioxaborolane following the procedure outlined for Example 1. ¹H NMR (600 MHZ, CD₃OD) δ 7.94 (d, J=1.7 Hz, 1H), 7.70 (d, J=8.5 Hz, 1H), 7.16 (s, 1H), 7.12 (dd, J=8.5, 1.8 Hz, 1H), 6.55 (d, J=16.0 Hz, 1H), 6.47 (dd, J=16.9, 10.1 Hz, 1H), 6.37 (dd, J=16.9, 1.7 Hz, 1H), 6.06 (dd, J=16.1, 7.1 Hz, 1H), 5.76 (dd, J=10.2, 1.7 Hz, 1H), 3.73 (s, 3H), 2.31-2.23 (m, 1H), 2.12-2.03 (m, 2H), 1.93-1.78 (m, 4H), 1.59-1.51 (m, 2H). LCMS (ESI) m/z=345 (M+H)⁺.

### Example 14

### (E)-N-(3-(4-Chlorostyryl)-1,2-dimethyl-1H-indol-5-yl)acrylamide

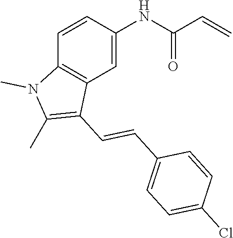 [SMILES: C=CC(=O)Nc1ccc2c(c1)c(/C=C/c1ccc(Cl)cc1)c(C)n2C]

The title compound was prepared from Intermediate R and (E)-(4-chlorostyryl) boronic acid following the procedure outlined for Example 1. ¹H NMR (600 MHZ, CD₃OD) δ 8.36 (d, J=1.7 Hz, 1H), 7.52 (d, J=8.5 Hz, 2H), 7.39-7.28 (m, 5H), 7.04-6.99 (m, 1H), 6.49 (d, J=17.0, 10.1 Hz, 1H), 6.37 (dd, J=16.9, 1.6 Hz, 1H), 5.76 (dd, J=10.2, 1.6 Hz, 1H), 3.70 (s, 3H), 2.52 (s, 3H). LCMS (ESI) m/z=351 (M+H)⁺.

### Example 15

### (E)-N-(3-(2-(4,4-Difluorocyclohexyl)vinyl)-1-methyl-1H-pyrrolo[2,3-b]pyridin-5-yl)acrylamide

The overall reaction scheme was as follows:

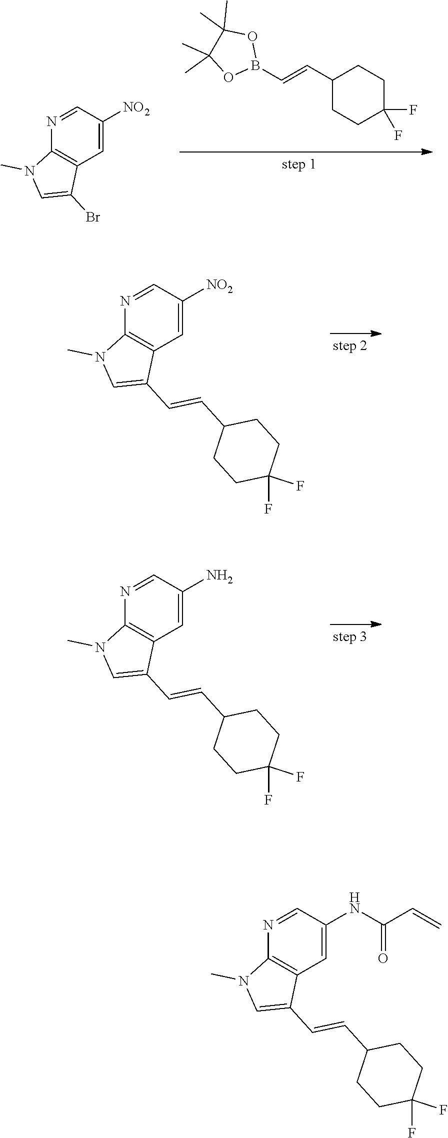 [SMILES: Cn1cc(Br)c2cc([N+](=O)[O-])cnc21 ; CC1(C)OB(/C=C/C2CCC(F)(F)CC2)OC1(C)C ; Cn1cc(/C=C/C2CCC(F)(F)CC2)c2cc([N+](=O)[O-])cnc21 ; Cn1cc(/C=C/C2CCC(F)(F)CC2)c2cc(N)cnc21 ; C=CC(=O)Nc1cnc2c(c1)c(/C=C/C1CCC(F)(F)CC1)cn2C]

### Step 1: (E)-3-(2-(4,4-Difluorocyclohexyl)vinyl)-1-methyl-5-nitro-1H-pyrrolo[2,3-b]pyridine

A mixture of intermediate E (130 mg, 0.510 mmol), (E)-2-(2-(4,4-difluorocyclohexyl)vinyl)-4,4,5,5-tetramethyl-1,3,2-dioxaborolane (166 mg, 0.612 mmol), Na₂CO₃ (92.0 mg, 0.866 mmol), KOAc (92.0 mg, 0.933 mmol), Pd(dppf)Cl₂-DCM (21 mg, 0.0255 mmol) were suspended in a mixed solution of MeCN (6 mL) and water (1.5 mL). The reaction mixture was stirred at 120° C. for 1 h under microwave condition. To the resultant mixture was diluted with EtOAc and water, and extracted with EtOAc twice. The combined organic phase was washed with brine, dried over anhydrous Na₂SO₄, filtered and concentrated. The residue was purified by flash chromatography to obtain the titled compound (77.0 mg, 47%). ¹H NMR (600 MHz, CDCl₃) δ 9.23 (d, J=2.3 Hz, 1H), 8.91 (d, J=2.4 Hz, 1H), 7.33 (s, 1H), 6.50 (d, J=16.2 Hz, 1H), 6.16 (dd, J=16.2, 6.9 Hz, 1H), 3.92 (s, 3H), 2.32-2.25 (m, 1H), 2.20-2.13 (m, 2H), 1.94-1.91 (m, 2H), 1.88-1.75 (m, 3H), 1.65-1.59 (m, 2H). LCMS (ESI) m/z=322 (M+H)⁺.

### Step 2: (E)-3-(2-(4,4-Difluorocyclohexyl)vinyl)-1-methyl-1H-pyrrolo[2,3-b]pyridin-5-amine

To a stirred solution of (E)-3-(2-(4,4-difluorocyclohexyl)vinyl)-1-methyl-5-nitro-1H-pyrrolo[2,3-b]pyridine (159 mg, 0.494 mmol) in THF/Water (4:1, 10 mL) was added zinc (323 mg, 4.94 mmol) and NH₄Cl (264 mg, 4.94 mmol). The reaction mixture was stirred at 70° C. for 1 h. The resultant mixture was filtered to remove insoluble and extracted with EtOAc. The organic layer was dried over anhydrous Na₂SO₄, filtered and concentrated. The residue was purified by flash chromatography to obtain the titled compound (42.0 mg, 29%). ¹H NMR (600 MHz, CDCl₃) δ 7.93 (d, J=2.5 Hz, 1H), 7.47 (d, J=2.5 Hz, 1H), 7.09 (s, 1H), 6.45 (d, J=16.7 Hz, 1H), 5.97 (dd, J=16.1, 7.1 Hz, 1H), 3.79 (s, 3H), 2.28-2.19 (m, 1H), 2.19-2.11 (m, 2H), 1.93-1.87 (m, 2H), 1.87-1.74 (m, 2H), 1.63-1.53 (m, 2H). LCMS (ESI) m/z=292 (M+H)⁺.

### Step 3: (E)-N-(3-(2-(4,4-Difluorocyclohexyl)vinyl)-7-fluoro-1-methyl-1H-indol-5-yl(acrylamide

(E)-3-(2-(4,4-Difluorocyclohexyl)vinyl)-1-methyl-1H-pyrrolo[2,3-b]pyridin-5-amine (188 mg, 0.645 mmol) and TEA (0.18 mL, 1.29 mmol) were diluted in DCM (4 mL) and cooled to 0° C. Acryloyl chloride (0.057 mL, 0.710 mmol) was added at once and the reaction mixture was stirred for 1 h then quenched by adding water. The resultant mixture was separated and the aqueous layer was extracted with DCM twice. The collected organic layer was concentrated and the residue was purified by flash column chromatography to obtain the titled compound (148 mg, 66%). ¹H NMR (600 MHZ, CD₃OD) δ 8.62 (d, J=2.3 Hz, 1H), 8.41 (d, J=2.3 Hz, 1H), 7.38 (s, 1H), 6.56-6.35 (m, 3H), 6.11 (dd, J=16.2, 7.1 Hz, 1H), 5.84-5.76 (m, 1H), 3.82 (s, 3H), 2.35-2.26 (m, 1H), 2.14-2.03 (m, 2H), 1.95-1.77 (m, 4H), 1.62-1.51 (m, 2H). LCMS (ESI) m/z=346 (M+H)⁺.

### Example 16

### (E)-N-(3-(4-Chlorostyryl)-1-methyl-1H-pyrrolo[2,3-b]pyridin-5-yl)acrylamide

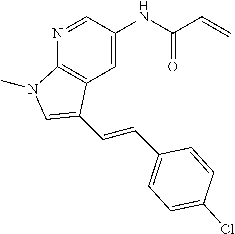 [SMILES: C=CC(=O)Nc1cnc2c(c1)c(/C=C/c1ccc(Cl)cc1)cn2C]

The title compound was prepared from Intermediate E and (E)-(4-chlorostyryl) boronic acid following the procedure outlined for Example 15. ¹H NMR (600 MHZ, CD₃OD) δ 8.79 (d, J=2.3 Hz, 1H), 8.44 (d, J=2.2 Hz, 1H), 7.60 (s, 1H), 7.54-7.48 (m, 2H), 7.34-7.30 (m, 2H), 7.28 (d, J=16.5 Hz, 1H), 7.04 (d, J=16.5 Hz, 1H), 6.49 (dd, J=16.9, 10.0 Hz, 1H), 6.42 (dd, J=17.0, 1.8 Hz, 1H), 5.82 (dd, J=10.0, 1.8 Hz, 1H), 3.85 (s, 3H). LCMS (ESI) m/z=338 (M+H)⁺.

### Example 17

### (E)-N-(3-(2-(4,4-Difluorocyclohexyl)vinyl)-1-ethyl-1H-pyrrolo[2,3-b]pyridin-5-yl)acrylamide

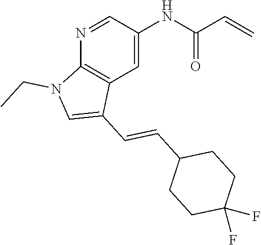 [SMILES: C=CC(=O)Nc1cnc2c(c1)c(/C=C/C1CCC(F)(F)CC1)cn2CC]

The title compound was prepared from Intermediate F and (E)-2-(2-(4,4-difluorocyclohexyl)vinyl)-4,4,5,5-tetramethyl-1,3,2-dioxaborolane following the procedure outlined for Example 15. ¹H NMR (400 MHZ, CD₃OD) δ 8.61 (s, 1H), 8.40 (s, 1H), 7.45 (s, 1H), 6.55 (d, J=16.0 Hz, 1H), 6.49-6.36 (m, 2H), 6.12 (dd, J=16.2, 7.1 Hz, 1H), 5.80 (d, J=9.9 Hz, 1H), 4.27 (q, J=8.0 Hz, 2H), 2.37-2.24 (m, 1H), 2.16-2.02 (m, 2H), 1.95-1.76 (m, 4H), 1.65-1.54 (m, 2H), 1.42 (t, J=7.0 Hz, 3H). LCMS (ESI) m/z=360 (M+H)⁺.

### Example 18

### (E)-N-(3-(4-Chlorostyryl)-1-ethyl-1H-pyrrolo[2,3-b]pyridin-5-yl)acrylamide

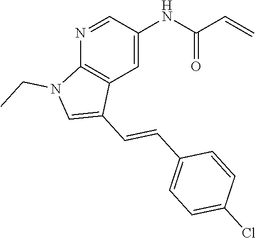 [SMILES: C=CC(=O)Nc1cnc2c(c1)c(/C=C/c1ccc(Cl)cc1)cn2CC]

The title compound was prepared from Intermediate F and (E)-(4-chlorostyryl) boronic acid following the procedure outlined for Example 15. ¹H NMR (600 MHZ, CD₃OD) δ 8.78 (d, J=2.2 Hz, 1H), 8.42 (d, J=2.2 Hz, 1H), 7.67 (s, 1H), 7.51 (d, J=8.5 Hz, 2H), 7.30 (dd, J=12.4, 9.3 Hz, 3H), 7.05 (d, J=16.5 Hz, 1H), 6.45 (dd, J=17.0, 5.8 Hz, 2H), 5.81 (dd, J=10.0, 1.7 Hz, 1H), 4.31 (q, J=7.3 Hz, 2H), 1.45 (t, J=7.3 Hz, 3H). LCMS (ESI) m/z=352 (M+H)⁺.

### Example 19

### (E)-N-(3-(2-(4,4-Difluorocyclohexyl)vinyl)-1-isopropyl-1H-pyrrolo[2,3-b]pyridin-5-yl)acrylamide

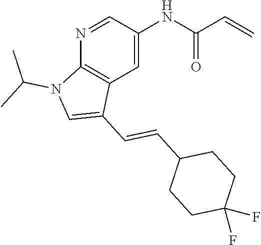 [SMILES: C=CC(=O)Nc1cnc2c(c1)c(/C=C/C1CCC(F)(F)CC1)cn2C(C)C]

The title compound was prepared from Intermediate G and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 8.59 (d, J=2.3 Hz, 1H), 8.38 (d, J=2.2 Hz, 1H), 7.54 (s, 1H), 6.55 (dd, J=16.1, 1.3 Hz, 1H), 6.47 (dd, J=17.0, 10.0 Hz, 1H), 6.38 (dd, J=17.0, 1.8 Hz, 1H), 6.10 (dd, J=16.1, 7.1 Hz, 1H), 5.79 (dd, J=10.1, 1.7 Hz, 1H), 5.03 (p, J=6.7 Hz, 1H), 2.32-2.24 (m, 1H), 2.12-2.02 (m, 2H), 1.94-1.75 (m, 4H), 1.61-1.55 (m, 2H), 1.50 (d, J=6.8 Hz, 6H). LC/MS (ESI) m/z=374 (M+H)⁺.

### Example 20

### (E)-N-(3-(4-Chlorostyryl)-1-isopropyl-1H-pyrrolo[2,3-b]pyridin-5-yl)acrylamide

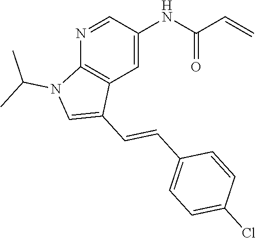 [SMILES: C=CC(=O)Nc1cnc2c(c1)c(/C=C/c1ccc(Cl)cc1)cn2C(C)C]

The title compound was prepared from Intermediate G and (E)-(4-chlorostyryl) boronic acid following the procedure outlined for Example 15. ¹H NMR (600 MHZ, CD₃OD) δ 8.78 (d, J=2.3 Hz, 1H), 8.42 (d, J=2.2 Hz, 1H), 7.78 (s, 1H), 7.52 (d, J=8.5 Hz, 2H), 7.31 (dd, J=12.5, 3.9 Hz, 3H), 7.06 (d, J=16.5 Hz, 1H), 6.45 (dd, J=17.0, 5.8 Hz, 2H), 5.81 (dd, J=10.0, 1.7 Hz, 1H), 5.08 (p, J=6.7 Hz, 1H), 1.53 (d, J=6.8 Hz, 6H). LC/MS (ESI) m/z=366 (M+H)⁺.

### Example 21

### (E)-N-(3-(2-(4,4-Difluorocyclohexyl)vinyl)-1-methyl-1H-indol-5-yl)acrylamide

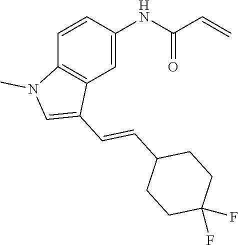 [SMILES: C=CC(=O)Nc1ccc2c(c1)c(/C=C/C1CCC(F)(F)CC1)cn2C]

The title compound was prepared from 3-bromo-1-methyl-5-nitro-1H-indole and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 8.17 (d, J=1.9 Hz, 1H), 7.38-7.26 (m, 2H), 7.18 (d, J=3.5 Hz, 1H), 6.55 (d, J=16.1 Hz, 1H), 6.46 (dd, J=10.1, 3.1 Hz, 1H), 6.35 (dd, J=17.0, 1.7 Hz, 1H), 6.07 (dd, J=16.1, 7.1 Hz, 1H), 5.74 (dd, J=10.2, 1.7 Hz, 1H), 3.74 (s, 3H), 2.31-2.23 (m, 1H), 2.13-2.02 (m, 2H), 1.93-1.86 (m, 3H), 1.86-1.77 (m, 1H), 1.61-1.50 (m, 2H). LC/MS (ESI) m/z=345 (M+H)⁺.

### Example 22

### (E)-N-(3-(4-Chlorostyryl)-1-methyl-1H-indol-5-yl)acrylamide

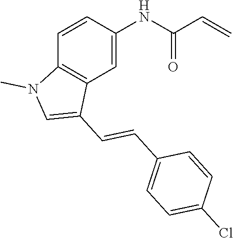 [SMILES: C=CC(=O)Nc1ccc2c(c1)c(/C=C/c1ccc(Cl)cc1)cn2C]

The title compound was prepared from 3-bromo-1-methyl-5-nitro-1H-indole and (E)-(4-chlorostyryl) boronic acid following the procedure outlined for Example 15. ¹H NMR (600 MHz, DMSO-d6) δ 10.11 (s, 1H), 8.37 (d, J=1.5 Hz, 1H), 7.63 (s, 1H), 7.55 (d, J=8.5 Hz, 2H), 7.48-7.42 (m, 2H), 7.39 (dd, J=12.4, 10.6 Hz, 3H), 6.96 (d, J=16.5 Hz, 1H), 6.48 (dd, J=16.9, 10.1 Hz, 1H), 6.27 (dd, J=16.9, 1.9 Hz, 1H), 5.74 (dd, J=10.1, 1.9 Hz, 1H), 3.79 (s, 3H). LC/MS (ESI) m/z=337 (M+H)⁺.

### Example 23

### N-(3-(4,4-Difluorocyclohex-1-en-1-yl)-1-methyl-1H-indol-5-yl)acrylamide

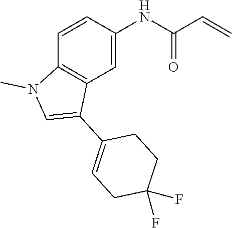 [SMILES: C=CC(=O)Nc1ccc2c(c1)c(C1=CCC(F)(F)CC1)cn2C]

The title compound was prepared from 3-bromo-1-methyl-5-nitro-1H-indole and 2-(4,4-difluorocyclohex-1-en-1-yl)-4,4,5,5-tetramethyl-1,3,2-dioxaborolane following the procedure outlined for Example 15. ¹H NMR (400 MHZ, CD₃OD) δ 8.23 (s, 1H), 7.38-7.29 (m, 2H), 7.22 (s, 1H), 6.46 (dd, J=16.9, 10.0 Hz, 1H), 6.34 (d, J=16.9 Hz, 1H), 6.02 (s, 1H), 5.74 (d, J=10.0 Hz, 1H), 3.75 (s, 3H), 2.24-2.12 (m, 2H), 1.26 (d, J=12.1 Hz, 4H). LCMS (ESI) m/z=317 (M+H)⁺.

### Example 24

### (E)-N-(3-(4-Fluorostyryl)-1-methyl-1H-indol-5-yl)acrylamide

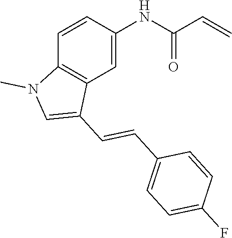 [SMILES: C=CC(=O)Nc1ccc2c(c1)c(/C=C/c1ccc(F)cc1)cn2C]

The title compound was prepared from 3-bromo-1-methyl-5-nitro-1H-indole and (E)-2-(4-fluorostyryl)-4,4,5,5-tetramethyl-1,3,2-dioxaborolane following the procedure outlined for Example 15. ¹H NMR (600 MHZ, DMSO-d6) δ 10.10 (s, 1H), 8.36 (d, J=1.5 Hz, 1H), 7.60 (s, 1H), 7.57 (dd, J=8.6, 5.6 Hz, 2H), 7.47 (dd, J=8.8, 1.9 Hz, 1H), 7.43 (d, J=8.7 Hz, 1H), 7.29 (d, J=16.5 Hz, 1H), 7.18 (t, J=8.8 Hz, 2H), 6.97 (d, J=16.5 Hz, 1H), 6.48 (dd, J=16.9, 10.2 Hz, 1H), 6.27 (dd, J=16.9, 1.9 Hz, 1H), 5.74 (dd, J=10.1, 1.9 Hz, 1H), 3.79 (s, 3H). LCMS (ESI) m/z=321 (M+H)⁺.

### Example 25

### (E)-N-(3-(2-(4,4-Difluorocyclohexyl)vinyl)-1-ethyl-1H-indol-5-yl)acrylamide

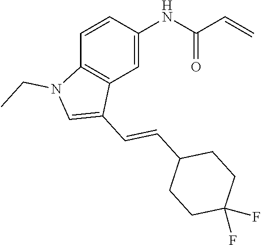 [SMILES: C=CC(=O)Nc1ccc2c(c1)c(/C=C/C1CCC(F)(F)CC1)cn2CC]

The title compound was prepared from Intermediate L and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 8.19 (s, 1H), 7.35 (d, J=1.6 Hz, 2H), 7.27 (s, 1H), 6.57 (d, J=16.1 Hz, 1H), 6.48 (dd, J=17.0, 10.2 Hz, 1H), 6.36 (dd, J=17.0, 1.6 Hz, 1H), 6.08 (dd, J=16.1, 7.1 Hz, 1H), 5.76 (dd, J=10.2, 1.6 Hz, 1H), 4.17 (q, J=7.2 Hz, 2H), 2.32-2.25 (m, J=7.1 Hz, 1H), 2.12-2.05 (m, 2H), 1.94-1.79 (m, 4H), 1.63-1.54 (m, 2H), 1.41 (t, J=7.3 Hz, 3H). LCMS (ESI) m/z=359 (M+H)⁺.

### Example 26

### (E)-N-(3-(4-Chlorostyryl)-1-ethyl-1H-indol-5-yl)acrylamide

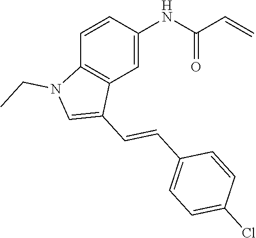 [SMILES: C=CC(=O)Nc1ccc2c(c1)c(/C=C/c1ccc(Cl)cc1)cn2CC]

The title compound was prepared from Intermediate L and (E)-(4-chlorostyryl) boronic acid following the procedure outlined for Example 15. ¹H NMR (400 MHZ, CDCl₃) δ 8.24 (s, 1H), 7.45-7.35 (m, J=7.7 Hz, 4H), 7.32-7.27 (m, 3H), 7.24 (d, J=14.6 Hz, 1H), 6.97 (d, J=16.6 Hz, 1H), 6.47 (d, J=17.0 Hz, 1H), 6.29 (dd, J=17.0, 10.1 Hz, 1H), 5.77 (d, J=10.2 Hz, 1H), 4.15 (q, J=14.6, 7.2 Hz, 2H), 1.48 (t, J=7.0 Hz, 3H). LCMS (ESI) m/z=351 (M+H)⁺.

### Example 27

### (E)-N-(3-(2-(4,4-Difluorocyclohexyl)vinyl)-1-isopropyl-1H-indol-5-yl)acrylamide

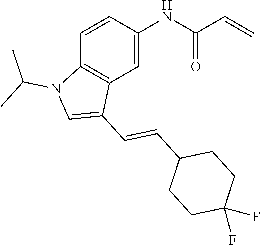 [SMILES: C=CC(=O)Nc1ccc2c(c1)c(/C=C/C1CCC(F)(F)CC1)cn2C(C)C]

The title compound was prepared from Intermediate M and (E)-2-(2-(4,4-difluorocyclohexyl)vinyl)-4,4,5,5-tetramethyl-1,3,2-dioxaborolane following the procedure outlined for Example 15. ¹H NMR (600 MHz, DMSO-d6) δ 10.03 (s, 1H), 8.19 (d, J=1.8 Hz, 1H), 7.57 (s, 1H), 7.45 (d, J=8.9 Hz, 1H), 7.40 (dd, J=8.8, 1.9 Hz, 1H), 6.52 (d, J=15.3 Hz, 1H), 6.45 (dd, J=16.9, 10.1 Hz, 1H), 6.24 (dd, J=17.0, 2.0 Hz, 1H), 6.01 (dd, J=16.1, 7.0 Hz, 1H), 5.72 (dd, J=10.1, 2.0 Hz, 1H), 4.68 (p, J=6.7 Hz, 1H), 2.35-2.27 (m, 1H), 2.09-2.01 (m, 2H), 2.01-1.89 (m, 2H), 1.89-1.85 (m, 2H), 1.43 (d, J=6.7 Hz, 6H). LCMS (ESI) m/z=373 (M+H)⁺.

### Example 28

### (E)-N-(3-(4-Chlorostyryl)-1-isopropyl-1H-indol-5-yl)acrylamide

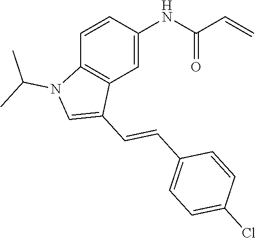 [SMILES: C=CC(=O)Nc1ccc2c(c1)c(/C=C/c1ccc(Cl)cc1)cn2C(C)C]

The title compound was prepared from Intermediate M and (E)-(4-chlorostyryl) boronic acid following the procedure outlined for Example 15. ¹H NMR (600 MHz, DMSO-d6) § 10.10 (s, 1H), 8.35 (d, J=2.1 Hz, 1H), 7.83 (s, 1H), 7.56-7.51 (m, 3H), 7.46-7.43 (m, 1H), 7.41-7.36 (m, 3H), 6.99 (d, J=16.5 Hz, 1H), 6.48 (dd, J=16.9, 10.1 Hz, 1H), 6.27 (dd, J=17.0, 2.0 Hz, 1H), 5.77-5.72 (d, J=10.1, 1.9 Hz, 1H), 4.74 (p, J=6.7 Hz, 1H), 1.47 (d, J=6.6 Hz, 6H). LCMS (ESI) m/z=365 (M+H)⁺.

### Example 29

### (E)-N-(3-(4-Fluorostyryl)-1-isopropyl-1H-indol-5-yl)acrylamide

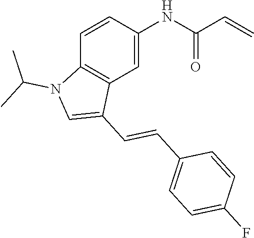 [SMILES: C=CC(=O)Nc1ccc2c(c1)c(/C=C/c1ccc(F)cc1)cn2C(C)C]

The title compound was prepared from Intermediate M and (E)-2-(4-fluorostyryl)-4,4,5,5-tetramethyl-1,3,2-dioxaborolane following the procedure outlined for Example 15. ¹H NMR (600 MHz, DMSO-d6) § 10.09 (s, 1H), 8.34 (dd, J=3.6, 1.9 Hz, 1H), 7.80 (s, 1H), 7.58-7.53 (m, 2H), 7.51 (d, J=8.9 Hz, 1H), 7.45-7.42 (m, 1H), 7.30 (d, J=16.5 Hz, 1H), 7.21-7.16 (m, 2H), 7.00 (d, J=16.5 Hz, 1H), 6.48 (dd, J=17.0, 10.1 Hz, 1H), 6.27 (dd, J=17.0, 2.0 Hz, 1H), 5.77-5.71 (m, 1H), 7.73 (hept, J=6.6 Hz, 1H), 1.47 (d, J=6.7 Hz, 6H). LCMS (ESI) m/z=349 (M+H)⁺.

### Example 30

### (E)-N-(3-(2-(4,4-Difluorocyclohexyl)vinyl)-1-methyl-1H-pyrazolo[3,4-b]pyridin-5-yl)acrylamide

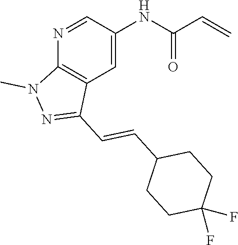 [SMILES: C=CC(=O)Nc1cnc2c(c1)c(/C=C/C1CCC(F)(F)CC1)nn2C]

The title compound was prepared from Intermediate I and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 8.83 (d, J=2.3 Hz, 1H), 8.61 (d, J=2.2 Hz, 1H), 6.68 (dd, J=16.4, 1.1 Hz, 1H), 6.55 (dd, J=16.4, 7.0 Hz, 1H), 6.49-6.37 (m, 2H), 5.82 (dd, J=9.7, 2.0 Hz, 1H), 4.04 (s, 3H), 2.44-2.35 (m, 1H), 2.16-2.04 (m, 2H), 1.99-1.79 (m, 4H), 1.65-1.56 (m, 2H). LCMS (ESI) m/z=347 (M+H)⁺.

### Example 31

### (E)-N-(3-(4-Chlorostyryl)-1-methyl-1H-pyrazolo[3,4-b]pyridin-5-yl)acrylamide

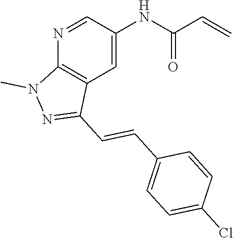 [SMILES: C=CC(=O)Nc1cnc2c(c1)c(/C=C/c1ccc(Cl)cc1)nn2C]

The title compound was prepared from Intermediate I and (E)-(4-chlorostyryl) boronic acid following the procedure outlined for Example 15. ¹H NMR (600 MHZ, CD₃OD) δ 8.97 (d, J=2.3 Hz, 1H), 8.63 (d, J=2.3 Hz, 1H), 7.61 (d, J=8.5 Hz, 2H), 7.47-7.31 (m, 4H), 6.49 (dd, J=17.0, 9.7 Hz, 1H), 6.43 (dd, J=17.0, 2.1 Hz, 1H), 5.84 (dd, J=9.7, 2.0 Hz, 1H), 4.09 (s, 3H). LCMS (ESI) m/z=339 (M+H)⁺.

### Example 32

### (E)-N-(3-(2-(4,4-Difluorocyclohexyl)vinyl)-1-ethyl-1H-pyrazolo[3,4-b]pyridin-5-yl)acrylamide

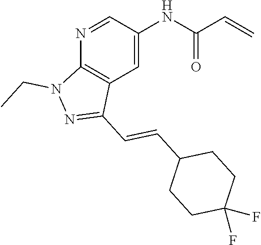 [SMILES: C=CC(=O)Nc1cnc2c(c1)c(/C=C/C1CCC(F)(F)CC1)nn2CC]

The title compound was prepared from Intermediate J and (E)-2-(2-(4,4-difluorocyclohexyl)vinyl)-4,4,5,5-tetramethyl-1,3,2-dioxaborolane following the procedure outlined for Example 15. ¹H NMR (600 MHz, CDCl₃) δ 8.80 (d, J=2.3 Hz, 1H), 8.40 (d, J=2.3 Hz, 1H), 7.48 (s, 1H), 6.68 (dd, J=16.3, 1.4 Hz, 1H), 6.54-6.47 (m, 2H), 6.31 (dd, J=16.8, 10.3 Hz, 1H), 5.86 (dd, J=10.3, 1.0 Hz, 1H), 4.51 (q, J=7.2 Hz, 2H), 2.35-2.27 (m, 1H), 2.20-2.11 (m, 2H), 1.98-1.90 (m, 2H), 1.89-1.75 (m, 2H), 1.68-1.60 (m, 2H), 1.51 (t, J=7.2 Hz, 3H). LCMS (ESI) m/z=361 (M+H)⁺.

### Example 33

### (E)-N-(3-(4-Chlorostyryl)-1-ethyl-1H-pyrazolo[3,4-b]pyridin-5-yl)acrylamide

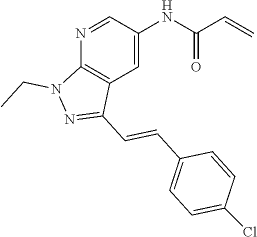 [SMILES: C=CC(=O)Nc1cnc2c(c1)c(/C=C/c1ccc(Cl)cc1)nn2CC]

The title compound was prepared from Intermediate J and (E)-(4-chlorostyryl) boronic acid following the procedure outlined for Example 15. ¹H NMR (600 MHZ, CDCl₃) δ 8.96 (d, J=2.3 Hz, 1H), 8.43 (d, J=2.3 Hz, 1H), 7.53-7.47 (m, 3H), 7.41-7.30 (m, 4H), 6.53 (dd, J=16.8, 1.1 Hz, 1H), 6.33 (dd, J=16.8, 10.2 Hz, 1H), 5.87 (dd, J=10.2, 1.1 Hz, 1H), 4.56 (q, J=7.3 Hz, 2H), 1.55 (t, J=7.3 Hz, 3H). LCMS (ESI) m/z=353 (M+H)⁺.

### Example 34

### (E)-N-(3-(2-(4,4-Difluorocyclohexyl)vinyl)-1-isopropyl-1H-pyrazolo[3,4-b]pyridin-5-yl)acrylamide

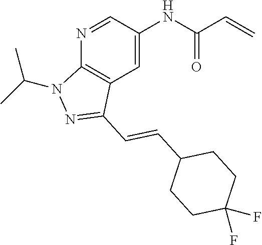 [SMILES: C=CC(=O)Nc1cnc2c(c1)c(/C=C/C1CCC(F)(F)CC1)nn2C(C)C]

The title compound was prepared from Intermediate K and (E)-2-(2-(4,4-difluorocyclohexyl)vinyl)-4,4,5,5-tetramethyl-1,3,2-dioxaborolane following the procedure outlined for Example 15. ¹H NMR (400 MHZ, CDCl₃) δ 8.79 (s, 1H), 8.41 (s, 1H), 7.66 (s, 1H), 6.70 (d, J=16.4 Hz, 1H), 6.53-6.42 (m, 2H), 6.31 (dd, J=16.8, 10.3 Hz, 1H), 5.83 (d, J=10.2 Hz, 1H), 5.20 (p, J=6.7 Hz, 1H), 2.36-2.23 (m, 1H), 2.21-2.09 (m, 2H), 1.95-1.88 (m, 2H), 1.86-1.71 (m, 2H), 1.69-1.59 (m, 2H), 1.55 (d, J=6.7 Hz, 6H). LCMS (ESI) m/z=375 (M+H)⁺.

### Example 35

### (E)-N-(3-(4-Chlorostyryl)-1-isopropyl-1H-pyrazolo[3,4-b]pyridin-5-yl)acrylamide

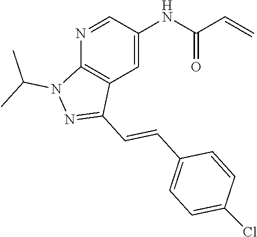 [SMILES: C=CC(=O)Nc1cnc2c(c1)c(/C=C/c1ccc(Cl)cc1)nn2C(C)C]

The title compound was prepared from Intermediate K and (E)-(4-chlorostyryl) boronic acid following the procedure outlined for Example 15. ¹H NMR (400 MHZ, CDCl₃) δ 8.96 (s, 1H), 8.44 (s, 1H), 7.51 (d, J=8.3 Hz, 2H), 7.38-7.32 (m, 3H), 6.53 (d, J=16.9 Hz, 1H), 6.34 (dd, J=16.9, 10.2 Hz, 1H), 5.87 (d, J=10.1 Hz, 1H), 5.27 (p, J=6.8 Hz, 1H), 1.61 (d, J=6.8 Hz, 6H). LCMS (ESI) m/z=367 (M+H)⁺.

### Example 36

### (E)-N-(3-(2-(4,4-Difluorocyclohexyl)vinyl)-7-fluoro-1-methyl-1H-indol-5-yl)acrylamide

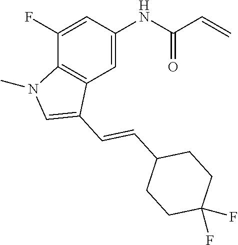 [SMILES: C=CC(=O)Nc1cc(F)c2c(c1)c(/C=C/C1CCC(F)(F)CC1)cn2C]

The title compound was prepared from Intermediate O and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 7.88 (d, J=1.6 Hz, 1H), 7.28 (dd, J=14.3, 1.5 Hz, 1H), 7.19 (s, 1H), 6.53 (d, J=16.1 Hz, 1H), 6.44 (dd, J=17.0, 10.0 Hz, 2H), 6.07 (dd, J=16.1, 7.1 Hz, 1H), 5.77 (dd, J=10.0, 1.8 Hz, 1H), 3.93 (s, 3H), 2.32-2.25 (m, 1H), 2.12-2.05 (m, 2H), 1.94-1.80 (m, 4H), 1.61-1.53 (m, 2H). LCMS (ESI) m/z=363 (M+H)⁺.

### Example 37

### (E)-N-(3-(4-Chlorostyryl)-7-fluoro-1-methyl-1H-indol-5-yl)acrylamide

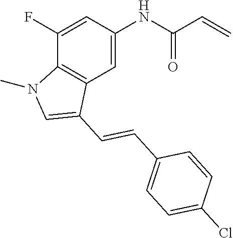 [SMILES: C=CC(=O)Nc1cc(F)c2c(c1)c(/C=C/c1ccc(Cl)cc1)cn2C]

The title compound was prepared from Intermediate O and (E)-(4-chlorostyryl) boronic acid following the procedure outlined for Example 15. ¹H NMR (600 MHZ, CD₃OD) δ 8.07 (d, J=1.7 Hz, 1H), 7.51 (d, J=8.5 Hz, 2H), 7.42 (s, 2H), 7.34-7.28 (m, 3H), 7.03 (d, J=16.5 Hz, 1H), 6.47 (dd, J=16.9, 9.9 Hz, 1H), 6.39 (dd, J=17.0, 1.8 Hz, 1H), 5.79 (dd, J=10.0, 1.8 Hz, 1H), 3.99 (s, 3H). LCMS (ESI) m/z=355 (M+H)⁺.

### Example 38

### (E)-N-(3-(2-(4,4-Difluorocyclohexyl)vinyl)-7-fluoro-1-isopropyl-1H-indol-5-yl)acrylamide

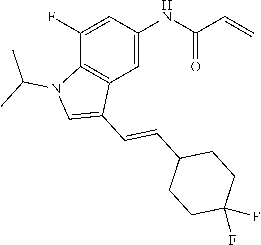 [SMILES: C=CC(=O)Nc1cc(F)c2c(c1)c(/C=C/C1CCC(F)(F)CC1)cn2C(C)C]

The title compound was prepared from Intermediate P and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 8.42 (s, 1H), 7.50 (d, J=9.2 Hz, 1H), 7.46 (d, J=8.8 Hz, 1H), 6.73 (d, J=16.4 Hz, 1H), 6.56 (dd, J=16.4, 7.1 Hz, 1H), 6.46 (dd, J=16.9, 10.0 Hz, 1H), 6.38 (dd, J=16.9, 1.8 Hz, 1H), 5.78 (dd, J=10.0, 1.7 Hz, 1H), 5.36-5.30 (m, 1H), 2.44-2.35 (m, 1H), 2.14-2.06 (m, 2H), 1.99-1.83 (m, 4H), 1.68-1.60 (m, 3H), 1.31 (d, J=18.5 Hz, 6H). LCMS (ESI) m/z=391 (M+H)⁺.

### Example 39

### (E)-N-(3-(4-Chlorostyryl)-7-fluoro-1-isopropyl-1H-indol-5-yl)acrylamide

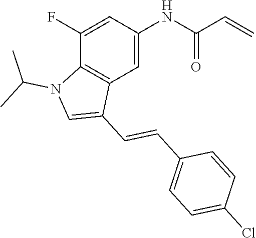 [SMILES: C=CC(=O)Nc1cc(F)c2c(c1)c(/C=C/c1ccc(Cl)cc1)cn2C(C)C]

The title compound was prepared from Intermediate P and (E)-(4-chlorostyryl) boronic acid following the procedure outlined for Example 15. ¹H NMR (600 MHZ, CD₃OD) δ 8.09 (d, J=1.7 Hz, 1H), 7.68 (s, 1H), 7.52 (d, J=8.5 Hz, 2H), 7.34 (d, J=7.2 Hz, 2H), 7.31 (s, 2H), 7.04 (d, J=16.5 Hz, 1H), 6.47 (dd, J=17.0, 10.0 Hz, 1H), 6.39 (dd, J=16.9, 1.8 Hz, 1H), 5.79 (dd, J=10.0, 1.8 Hz, 1H), 4.99 (p, J=6.7 Hz, 1H), 1.56 (d, J=6.7 Hz, 6H). LCMS (ESI) m/z=383 (M+H)⁺.

### Example 40

### (E)-N-(3-(3-Fluorostyryl)-1-methyl-1H-pyrrolo[2,3-b]pyridin-5-yl)acrylamide

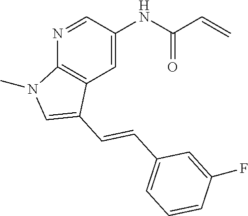 [SMILES: C=CC(=O)Nc1cnc2c(c1)c(/C=C/c1cccc(F)c1)cn2C]

The title compound was prepared from Intermediate E and (E)-2-(3-fluorostyryl)-4,4,5,5-tetramethyl-1,3,2-dioxaborolane following the procedure outlined for Example 15. ¹H NMR (600 MHZ, CD₃OD) δ 8.79 (d, J=2.2 Hz, 1H), 8.45 (d, J=2.2 Hz, 1H), 7.61 (s, 1H), 7.36-7.30 (m, 3H), 7.30-7.26 (m, 1H), 7.06 (d, J=16.5 Hz, 1H), 6.94-6.90 (m, 1H), 6.50 (dd, J=16.9, 10.0 Hz, 1H), 6.43 (dd, J=16.9, 1.8 Hz, 1H), 5.83 (dd, J=9.9, 1.8 Hz, 1H), 3.85 (s, 3H). LCMS (ESI) m/z=322 (M+H)⁺.

### Example 41

### (E)-N-(3-(3-Chlorostyryl)-1-methyl-1H-pyrrolo[2,3-b]pyridin-5-yl)acrylamide

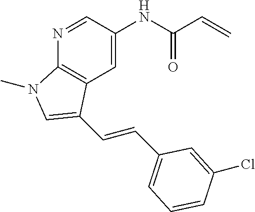 [SMILES: C=CC(=O)Nc1cnc2c(c1)c(/C=C/c1cccc(Cl)c1)cn2C]

The title compound was prepared from Intermediate E and (E)-2-(3-chlorostyryl)-4,4,5,5-tetramethyl-1,3,2-dioxaborolane following the procedure outlined for Example 15. ¹H NMR (600 MHZ, CD₃OD) δ 8.81 (d, J=2.3 Hz, 1H), 8.45 (d, J=2.2 Hz, 1H), 7.62 (s, 1H), 7.55 (t, J=1.8 Hz, 1H), 7.46 (d, J=8.0 Hz, 1H), 7.35-7.29 (m, 2H), 7.20-7.17 (m, 1H), 7.03 (d, J=16.5 Hz, 1H), 6.50 (dd, J=17.0, 9.9 Hz, 1H), 6.43 (dd, J=17.0, 1.8 Hz, 1H), 5.83 (dd, J=9.9, 1.8 Hz, 1H), 3.86 (s, 3H). LCMS (ESI) m/z=338 (M+H)⁺.

### Example 42

### (E)-N-(3-(3-Fluoro-4-methoxystyryl)-1-methyl-1H-pyrrolo[2,3-b]pyridin-5-yl)acrylamide

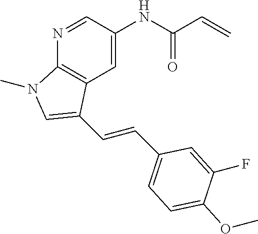

The title compound was prepared from Intermediate E and 2-[(E)-2-(3-fluoro-4-methoxy-phenyl) vinyl]-4,4,5,5-tetramethyl-1,3,2-dioxaborolane following the procedure outlined for Example 15. ¹H NMR (600 MHZ, CD₃OD) δ 8.79 (d, J=2.3 Hz, 1H), 8.45 (d, J=2.3 Hz, 1H), 7.59 (s, 1H), 7.34 (m, 2H), 7.26 (d, J=8.4 Hz, 1H), 7.18 (d, J=16.5 Hz, 1H), 7.07 (t, J=8.7 Hz, 1H), 7.01 (d, J=16.5 Hz, 1H), 6.51 (dd, J=17.0, 10.0 Hz, 1H), 6.43 (dd, J=17.0, 1.8 Hz, 1H), 5.83 (dd, J=10.0, 1.8 Hz, 1H), 3.89 (s, 3H), 3.86 (s, 3H). LCMS (ESI) m/z=352 (M+H)⁺.

### Example 43

### (E)-N-(3-(4-Isopropylstyryl)-1-methyl-1H-pyrrolo[2,3-b]pyridin-5-yl)acrylamide

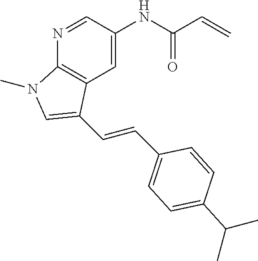 [SMILES: C=CC(=O)Nc1cnc2c(c1)c(/C=C/c1ccc(C(C)C)cc1)cn2C]

The title compound was prepared from Intermediate E and 2-[(E)-2-(4-isopropylphenyl) vinyl]-4,4,5,5-tetramethyl-1,3,2-dioxaborolane following the procedure outlined for Example 15. ¹H NMR (600 MHZ, CD₃OD) δ 8.78 (d, J=1.3 Hz, 1H), 8.46 (s, 1H), 7.57 (s, 1H), 7.48-7.44 (m, 2H), 7.25-7.20 (m, 3H), 7.06 (d, J=16.2 Hz, 1H), 6.53-6.39 (m, 2H), 5.83 (d, J=10.0 Hz, 1H), 3.89-3.83 (m, 3H), 2.90 (m, 1H), 1.27 (d, J=7.1 Hz, 6H). LCMS (ESI) m/z=346 (M+H)⁺.

### Example 44

### (E)-N-(3-(3,4-Difluorostyryl)-1-methyl-1H-pyrrolo[2,3-b]pyridin-5-yl)acrylamide

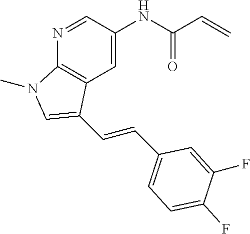 [SMILES: C=CC(=O)Nc1cnc2c(c1)c(/C=C/c1ccc(F)c(F)c1)cn2C]

The title compound was prepared from Intermediate E and 2-[(E)-2-(3,4-difluorophenyl) vinyl]-4,4,5,5-tetramethyl-1,3,2-dioxaborolane following the procedure outlined for Example 15. ¹H NMR (600 MHZ, CD₃OD) δ 8.77 (d, J=2.2 Hz, 1H), 8.42 (d, J=2.2 Hz, 1H), 7.58 (s, 1H), 7.44 (dd, J=12.2, 7.8, Hz, 1H), 7.31-7.27 (m, 1H), 7.21 (dd, J=17.0, 13.4, Hz, 2H), 7.00 (d, J=16.5 Hz, 1H), 6.46 (dd, J=16.9, 5.8 Hz, 2H), 5.84-5.81 (m, 1H), 3.84 (s, 3H). LCMS (ESI) m/z=340 (M+H)⁺.

### Example 45

### (E)-N-(1-Methyl-3-styryl-1H-pyrrolo[2,3-b]pyridin-5-yl)acrylamide

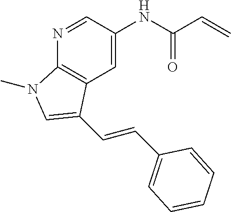 [SMILES: C=CC(=O)Nc1cnc2c(c1)c(/C=C/c1ccccc1)cn2C]

The title compound was prepared from Intermediate E and 2-[(E)-styryl]-4,4,5,5-tetramethyl-1,3,2-dioxaborolane following the procedure outlined for Example 15. ¹H NMR (600 MHz, CD₃OD) δ 8.79 (d, J=1.8 Hz, 1H), 8.46 (s, 1H), 7.59 (s, 1H), 7.53 (d, J=7.8 Hz, 2H), 7.33 (t, J=7.6 Hz, 2H), 7.28 (d, J=16.5 Hz, 1H), 7.19 (t, J=7.3 Hz, 1H), 7.08 (d, J=16.5 Hz, 1H), 6.50 (dd, J=17.2, 10.1 Hz, 1H), 6.43 (d, J=17.0 Hz, 1H), 5.83 (d, J=10.0 Hz, 1H), 3.85 (s, 3H). LCMS (ESI) m/z=304 (M+H)⁺.

### Example 46

### (E)-N-(1-Methyl-3-(4-(trifluoromethyl) styryl)-1H-pyrrolo[2,3-b]pyridin-5-yl)acrylamide

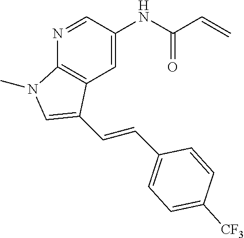 [SMILES: C=CC(=O)Nc1cnc2c(c1)c(/C=C/c1ccc(C(F)(F)F)cc1)cn2C]

The title compound was prepared from Intermediate E and [(E)-2-[4-(trifluoromethyl)phenyl]vinyl]boronic acid following the procedure outlined for Example 15. ¹H NMR (600 MHZ, CD₃OD) δ 8.85 (s, 1H), 8.46 (s, 1H), 7.72 (d, J=8.2 Hz, 2H), 7.68 (s, 1H), 7.62 (d, J=7.6 Hz, 2H), 7.46 (d, J=16.8 Hz, 2H), 7.15 (d, J=16.0 Hz, 1H), 6.46 (d, J=13.9 Hz, 2H), 5.84 (d, J=10.0 Hz, 1H), 3.88 (s, 3H). LCMS (ESI) m/z=372 (M+H)⁺.

### Example 47

### (E)-N-(3-(4-Fluorostyryl)-1-methyl-1H-pyrrolo[2,3-b]pyridin-5-yl)acrylamide

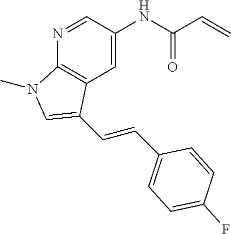 [SMILES: C=CC(=O)Nc1cnc2c(c1)c(/C=C/c1ccc(F)cc1)cn2C]

The title compound was prepared from Intermediate E and (E)-2-(4-fluorostyryl)-4,4,5,5-tetramethyl-1,3,2-dioxaborolane following the procedure outlined for Example 15. ¹H NMR (600 MHz, CD₃OD) δ 8.90 (s, 1H), 8.56 (s, 1H), 7.69 (s, 1H), 7.68-7.63 (m, 2H), 7.33 (d, J=16.7 Hz, 1H), 7.21-7.15 (m, 3H), 6.62 (dd, J=17.0, 10.0 Hz, 1H), 6.54 (d, J=17.0 Hz, 1H), 5.95 (d, J=10.0 Hz, 1H), 3.97 (s, 3H). LCMS (ESI) m/z=322 (M+H)⁺.

### Example 48

### (E)-N-(3-(3-Methoxystyryl)-1-methyl-1H-pyrrolo[2,3-b]pyridin-5-yl)acrylamide

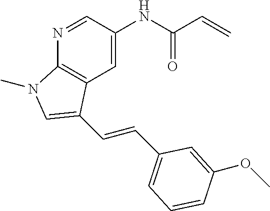

The title compound was prepared from Intermediate E and 2-[(E)-2-(3-methoxyphenyl) vinyl]-4,4,5,5-tetramethyl-1,3,2-dioxaborolane following the procedure outlined for Example 15. ¹H NMR (600 MHZ, CD₃OD) δ 8.80 (s, 1H), 8.47 (s, 1H), 7.60 (s, 1H), 7.29 (d, J=16.4 Hz, 1H), 7.24 (t, J=8.0 Hz, 1H), 7.14-7.04 (m, 3H), 6.78 (d, J=7.8 Hz, 1H), 6.51 (dd, J=17.5, 10.5 Hz, 1H), 6.43 (d, J=16.8 Hz, 1H), 5.83 (d, J=10.0 Hz, 1H), 3.87 (s, 3H), 3.84 (s, 3H). LCMS (ESI) m/z=334 (M+H)⁺.

### Example 49

### (E)-N-(3-(2-Cyclohexylvinyl)-1-methyl-1H-pyrrolo[2,3-b]pyridin-5-yl)acrylamide

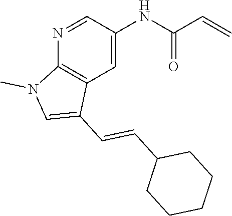 [SMILES: C=CC(=O)Nc1cnc2c(c1)c(/C=C/C1CCCCC1)cn2C]

The title compound was prepared from Intermediate E and 2-[(E)-2-cyclohexylvinyl]-4,4,5,5-tetramethyl-1,3,2-dioxaborolane following the procedure outlined for Example 15. ¹H NMR (600 MHZ, CD₃OD) δ 8.59 (d, J=1.9 Hz, 1H), 8.42 (d, J=1.8 Hz, 1H), 7.34 (s, 1H), 6.51-6.38 (m, 3H), 6.11 (dd, J=16.2, 7.1 Hz, 1H), 5.81 (d, J=10.0 Hz, 1H), 3.80 (s, 3H), 2.18-2.08 (m, 1H), 1.89-1.75 (m, 4H), 1.74-1.68 (m, 1H), 1.41-1.33 (m, 2H), 1.29-1.20 (m, 3H). LCMS (ESI) m/z=310 (M+H)⁺.

### Example 50

### (E)-N-(1-Methyl-3-(3-phenylprop-1-en-1-yl)-1H-pyrrolo[2,3-b]pyridin-5-yl)acrylamide

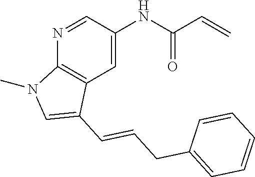 [SMILES: C=CC(=O)Nc1cnc2c(c1)c(/C=C/Cc1ccccc1)cn2C]

The title compound was prepared from Intermediate E and 4,4,5,5-tetramethyl-2-[(E)-3-phenylprop-1-enyl]-1,3,2-dioxaborolane following the procedure outlined for Example 15. ¹H NMR (600 MHz, CD₃OD) δ 8.56 (s, 1H), 8.44 (s, 1H), 7.40 (s, 1H), 7.23 (d, J=58.5 Hz, 5H), 6.58-6.27 (m, 5H), 5.79 (s, 1H), 3.81 (d, J=2.0 Hz, 2H), 3.56 (s, 3H). LCMS (ESI) m/z=318 (M+H)⁺.

### Example 51

### N-(1-Methyl-3-(4-(trifluoromethyl)phenyl)-1H-pyrrolo[2,3-b]pyridin-5-yl)acrylamide

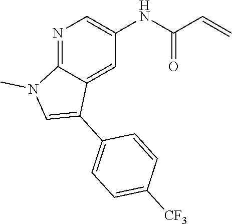 [SMILES: C=CC(=O)Nc1cnc2c(c1)c(-c1ccc(C(F)(F)F)cc1)cn2C]

The title compound was prepared from Intermediate E and [4-(trifluoromethyl)phenyl]boronic acid following the procedure outlined for Example 15. ¹H NMR (600 MHz, CDCl₃) δ 8.77 (s, 1H), 8.34 (s, 1H), 7.71-7.65 (m, 4H), 7.46 (s, 1H), 6.49 (d, J=16.8 Hz, 1H), 6.36-6.29 (m, 1H), 5.81 (d, J=10.1 Hz, 1H), 3.94 (s, 3H). LCMS (ESI) m/z=346 (M+H)⁺.

### Example 52

### N-(3-(3-((3-Chlorobenzyl)oxy)phenyl)-1-methyl-1H-pyrrolo[2,3-b]pyridin-5-yl)acrylamide

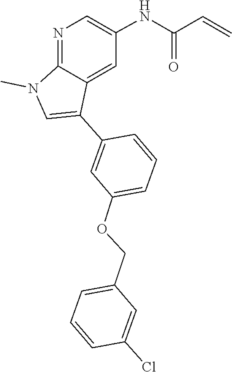 [SMILES: C=CC(=O)Nc1cnc2c(c1)c(-c1cccc(OCc3cccc(Cl)c3)c1)cn2C]

The title compound was prepared from Intermediate E and (3-((3-chlorophenyl) methoxy)phenyl) boronic acid following the procedure outlined for Example 15. ¹H NMR (600 MHz, CDCl₃) δ 8.58 (s, 1H), 8.41 (s, 1H), 7.61 (br s, 1H), 7.49 (s, 1H), 7.37-7.30 (m, 4H), 7.26 (s, 1H), 7.22 (d, J=7.2 Hz, 1H), 7.17 (s, 1H), 6.88 (d, J=8.0 Hz, 1H), 6.48 (d, J=16.8 Hz, 1H), 6.33 (d, J=17.0, 10.4 Hz, 1H), 5.80 (d, J=10.0 Hz, 1H), 5.11 (s, 2H), 3.91 (s, 3H). LCMS (ESI) m/z=418 (M+H)⁺.

### Example 53

### (E)-N-(3-(3,5-Bis(trifluoromethyl) styryl)-1-methyl-1H-pyrrolo[2,3-b]pyridin-5-yl)acrylamide

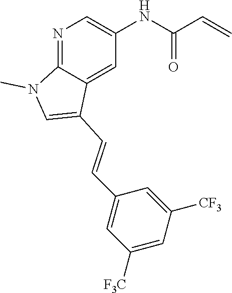 [SMILES: C=CC(=O)Nc1cnc2c(c1)c(/C=C/c1cc(C(F)(F)F)cc(C(F)(F)F)c1)cn2C]

The title compound was prepared from Intermediate E and (E)-(3,5-bis(trifluoromethyl) styryl) boronic acid following the procedure outlined for Example 15. ¹H NMR (400 MHZ, CD₃OD) δ 8.87 (s, 1H), 8.41 (s, 1H), 8.09 (s, 2H), 7.71 (d, J=15.9 Hz, 2H), 7.54 (d, J=17.0 Hz, 1H), 7.17 (d, J=16.7 Hz, 1H), 6.47 (t, J=16.6 Hz, 2H), 5.85 (d, J=9.7 Hz, 1H), 3.87 (s, 3H). LCMS (ESI) m/z=439 (M+H)⁺.

### Example 54

### N-(1-Methyl-3-(3-(trifluoromethyl)phenyl)-1H-pyrrolo[2,3-b]pyridin-5-yl)acrylamide

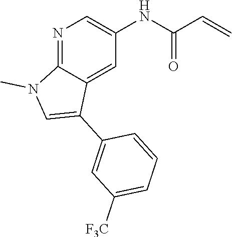 [SMILES: C=CC(=O)Nc1cnc2c(c1)c(-c1cccc(C(F)(F)F)c1)cn2C]

The title compound was prepared from Intermediate E and (3-(trifluoromethyl)phenyl) boronic acid following the procedure outlined for Example 15. ¹H NMR (600 MHZ, CD₃OD) δ 8.70 (s, 1H), 8.53 (s, 1H), 7.93 (d, J=12.6 Hz, 2H), 7.83 (s, 1H), 7.64 (t, J=7.8 Hz, 1H), 7.56 (d, J=7.2 Hz, 1H), 6.49 (dd, J=17.1, 10.1 Hz, 1H), 6.41 (d, J=16.9 Hz, 1H), 5.81 (d, J=10.2 Hz, 1H), 3.93 (s, 3H). LCMS (ESI) m/z=346 (M+H)⁺.

### Example 55

### N-(1-Methyl-3-(4-(methylthio)phenyl)-1H-pyrrolo[2,3-b]pyridin-5-yl)acrylamide

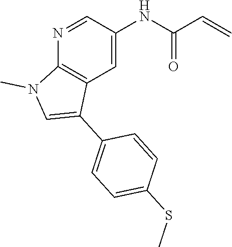 [SMILES: C=CC(=O)Nc1cnc2c(c1)c(-c1ccc(SC)cc1)cn2C]

The title compound was prepared from Intermediate E and 4-(methylthio)phenylboronic acid following the procedure outlined for Example 15. ¹H NMR (600 MHz, CDCl₃) δ 8.70 (dd, J=6.7, 2.3 Hz, 1H), 8.31 (d, J=2.3 Hz, 1H), 7.53 (d, J=7.9 Hz, 2H), 7.37 (s, 1H), 7.33 (d, J=8.0 Hz, 2H), 6.48 (d, J=16.8 Hz, 1H), 6.31 (dd, J=16.8, 10.2 Hz, 1H), 5.80 (d, J=10.2 Hz, 1H), 3.90 (s, 3H), 2.52 (s, 3H). LCMS (ESI) m/z=324 (M+H)⁺.

### Example 56

### N-(3-(3-Cyanophenyl)-1-methyl-1H-pyrrolo[2,3-b]pyridin-5-yl)acrylamide

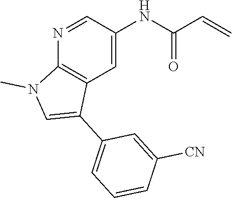 [SMILES: C=CC(=O)Nc1cnc2c(c1)c(-c1cccc(C#N)c1)cn2C]

The title compound was prepared from Intermediate E and (3-cyanophenyl) boronic acid following the procedure outlined for Example 15. ¹H NMR (600 MHZ, CD₃OD) δ 8.70 (d, J=2.2 Hz, 1H), 8.50 (d, J=2.2 Hz, 1H), 8.01-7.94 (m, 2H), 7.85-7.79 (m, 1H), 7.63-7.57 (m, 2H), 6.48 (dd, J=16.9, 10.0 Hz, 1H), 6.40 (dd, J=17.0, 1.7 Hz, 1H), 5.81 (dd, J=10.0, 1.7 Hz, 1H), 3.91 (s, 3H). LCMS (ESI) m/z=303 (M+H)⁺.

### Example 57

### N-(3-(4-Cyanophenyl)-1-methyl-1H-pyrrolo[2,3-b]pyridin-5-yl)acrylamide

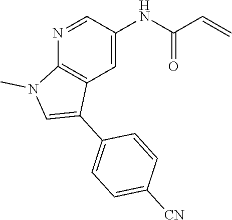 [SMILES: C=CC(=O)Nc1cnc2c(c1)c(-c1ccc(C#N)cc1)cn2C]

The title compound was prepared from Intermediate E and (4-cyanophenyl) boronic acid following the procedure outlined for Example 15. ¹H NMR (600 MHZ, CD₃OD) δ 8.75 (d, J=2.2 Hz, 1H), 8.45 (d, J=2.2 Hz, 1H), 7.86 (s, 1H), 7.82-7.80 (m, 2H), 7.75-7.72 (m, 2H), 6.48 (dd, J=17.0, 9.9 Hz, 1H), 6.40 (dd, J=17.0, 1.8 Hz, 1H), 5.82 (dd, J=9.9, 1.8 Hz, 1H), 3.90 (s, 3H). LCMS (ESI) m/z=303 (M+H)⁺.

### Example 58

### (E)-N-(1-Methyl-3-(2-(tetrahydro-2H-pyran-4-yl) vinyl)-1H-pyrrolo[2,3-b]pyridin-5-yl)acrylamide

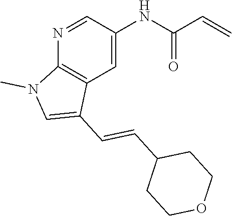 [SMILES: C=CC(=O)Nc1cnc2c(c1)c(/C=C/C1CCOCC1)cn2C]

The title compound was prepared from Intermediate E and 4,4,5,5-tetramethyl-2-[(E)-2-tetrahydropyran-4-ylvinyl]-1,3,2-dioxaborolane following the procedure outlined for Example 15. ¹H NMR (600 MHz, CD₃OD) δ 8.60 (d, J=2.3 Hz, 1H), 8.41 (d, J=2.2 Hz, 1H), 7.37 (s, 1H), 6.52-6.44 (m, 2H), 6.43-6.36 (m, 1H), 6.11 (dd, J=16.2, 6.9 Hz, 1H), 5.80 (dd, J=10.0, 1.7 Hz, 1H), 3.98 (dd, J=11.4, 2.7 Hz, 2H), 3.80 (s, 3H), 3.50 (td, J=11.8, 2.0 Hz, 2H), 3.30 (p, J=1.7 Hz, 2H), 2.43-2.36 (m, 1H), 1.76-1.72 (m, 2H), 1.60-1.52 (m, 2H). LCMS (ESI) m/z=312 (M+H)⁺.

### Example 59

### (E)-N-(1-Methyl-3-(2-(thiophen-3-yl) vinyl)-1H-pyrrolo[2,3-b]pyridin-5-yl)acrylamide

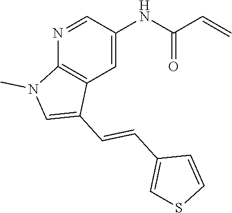 [SMILES: C=CC(=O)Nc1cnc2c(c1)c(/C=C/c1ccsc1)cn2C]

The title compound was prepared from Intermediate E and 4,4,5,5-tetramethyl-2-[(E)-2-(3-thienyl) vinyl]-1,3,2-dioxaborolane following the procedure outlined for Example 15. ¹H NMR (600 MHz, CD₃OD) δ 8.75 (d, J=2.2 Hz, 1H), 8.43 (d, J=2.2 Hz, 1H), 7.54 (s, 1H), 7.41-7.37 (m, 2H), 7.30 (dd, J=2.9, 1.3 Hz, 1H), 7.12 (s, 2H), 6.49 (dd, J=17.0, 10.0 Hz, 1H), 6.42 (dd, J=17.0, 1.8 Hz, 1H), 5.82 (dd, J=10.0, 1.7 Hz, 1H), 3.84 (s, 3H). LCMS (ESI) m/z=310 (M+H)⁺.

### Example 60

### (E)-N-(3-(2-Cyclopropylvinyl)-1-methyl-1H-pyrrolo[2,3-b]pyridin-5-yl)acrylamide

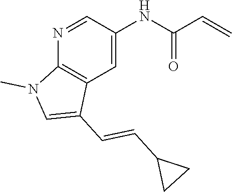 [SMILES: C=CC(=O)Nc1cnc2c(c1)c(/C=C/C1CC1)cn2C]

The title compound was prepared from Intermediate E and 2-[(E)-2-cyclopropylvinyl]4,4,5,5-tetramethyl-1,3,2-dioxaborolane following the procedure outlined for Example 15. ¹H NMR (600 MHZ, CD₃OD) δ 8.56 (d, J=2.2 Hz, 1H), 8.41 (d, J=2.2 Hz, 1H), 7.33 (s, 1H), 6.56-6.38 (m, 3H), 5.81 (dd, J=10.0, 1.7 Hz, 1H), 5.72 (dd, J=16.0, 8.6 Hz, 1H), 3.81 (s, 3H), 1.59-1.52 (m, 1H), 0.81-0.77 (m, 2H), 0.52-0.48 (m, 2H). LCMS (ESI) m/z=268 (M+H)⁺.

### Example 61

### (E)-N-(3-(2-(2,3-Dihydrobenzo[b][1,4]dioxin-6-yl) vinyl)-1-methyl-1H-pyrrolo[2,3-b]pyridin-5-yl)acrylamide

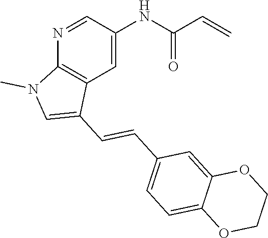 [SMILES: C=CC(=O)Nc1cnc2c(c1)c(/C=C/c1ccc3c(c1)OCCO3)cn2C]

The title compound was prepared from Intermediate E and 2-[(E)-2-(2,3-dihydro-1,4-benzodioxin-6-yl) vinyl]4,4,5,5-tetramethyl-1,3,2-dioxaborolane following the procedure outlined for Example 15. ¹H NMR (600 MHz, CD₃OD) δ 8.73 (d, J=2.2 Hz, 1H), 8.46 (d, J=2.2 Hz, 1H), 7.53 (s, 1H), 7.09 (d, J=16.5 Hz, 1H), 7.02-6.98 (m, 2H), 6.95 (d, J=16.5 Hz, 1H), 6.79 (d, J=8.3 Hz, 1H), 6.46 (dd, J=17.0, 5.9 Hz, 2H), 5.82 (dd, J=10.0, 1.7 Hz, 1H), 5.50 (s, 1H), 4.26-4.23 (m, 4H), 3.84 (s, 3H). LCMS (ESI) m/z=362 (M+H)⁺.

### Example 62

### Ethyl (E)-2-(2-(5-acrylamido-1-methyl-1H-pyrrolo[2,3-b]pyridin-3-yl) vinyl)cyclopropane-1-carboxylate

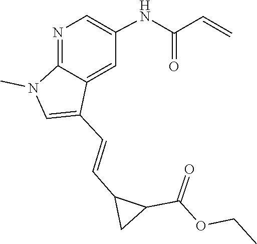 [SMILES: C=CC(=O)Nc1cnc2c(c1)c(/C=C/C1CC1C(=O)OCC)cn2C]

The title compound was prepared from Intermediate E and ethyl 2-[(E)-2-(4,4,5,5-tetramethyl-1,3,2-dioxaborolan-2-yl) vinyl]cyclopropanecarboxylate following the procedure outlined for Example 15. ¹H NMR (600 MHZ, CD₃OD) δ 8.58 (d, J=2.2 Hz, 1H), 8.39 (d, J=2.3 Hz, 1H), 7.39 (s, 1H), 6.66 (d, J=15.9 Hz, 1H), 6.48 (dd, J=17.0, 10.0 Hz, 1H), 6.40 (dd, J=17.0, 1.8 Hz, 1H), 5.81 (dd, J=10.0, 1.8 Hz, 1H), 5.77 (dd, J=15.9, 8.5 Hz, 1H), 4.15 (q, J=7.1 Hz, 2H), 3.81 (s, 3H), 2.15-2.08 (m, 1H), 1.82-1.77 (m, 1H), 1.43-1.38 (m, 1H), 1.27 (t, J=7.2 Hz, 3H), 1.20-1.15 (m, 1H). LCMS (ESI) m/z=340 (M+H)⁺.

### Example 63

### N-(3-(4-Fluorophenethyl)-1-methyl-1H-pyrrolo[2,3-b]pyridin-5-yl)acrylamide

The overall reaction scheme was as follows:

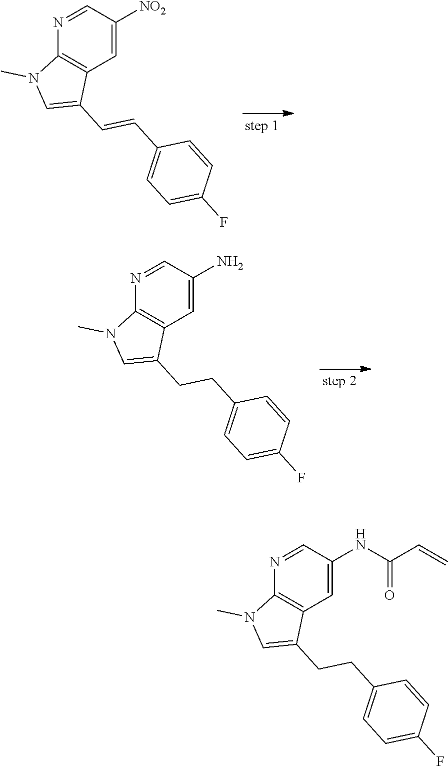

### Step 1: (E)-3-(4-Fluorophenetyl)-1-methyl-1H-pyrrolo[2,3-b]pyridin-5-amine

To a stirred solution of (E)-3-(4-fluorostyryl)-1-methyl-5-nitro-1H-pyrrolo[2,3-b]pyridine (200 mg, 0.673 mmol) in MeOH (5 mL) were added Pd/C (10 wt %, 20 mg, 0.306 mmol) at r.t. The reaction mixture was stirred for 16 h under hydrogen atmosphere. After completion of the reaction, the mixture was filtered to remove insolubles and extracted with EtOAc. The collected organic layer was dried over anhydrous Na₂SO₄, filtered and concentrated. The residue was purified by flash chromatography to obtain the titled compound (150 mg, 83%). ¹H NMR (600 MHZ, CDCl₃) δ 7.93 (s, 1H), 7.16 (s, 1H), 7.13-7.08 (m, 2H), 6.95 (t, J=8.6 Hz, 2H), 6.80 (s, 1H), 3.76 (s, 3H), 2.92 (s, 4H). LCMS (ESI) m/z=270 (M+H)⁺.

### Step 2: (E)-N-(3-(4-Fluorophenethyl)-1-methyl-1H-pyrrolo[2,3-b]pyridin-5-yl)acrylamide

A solution of (E)-3-(4-fluorophenethyl)-1-methyl-1H-pyrrolo[2,3-b]pyridin-5-amine (150 mg, 0.557 mmol) and TEA (0.155 mL, 1.11 mmol) in DCM (3 mL) was stirred at r.t. The mixture was cooled to 0° C. and then acryloyl chloride (0.0619 mL, 0.780 mmol) was added dropwise at 0° C. The resultant mixture was stirred at 0° C. for 1 h. The reaction was quenched by adding water and extracted with DCM twice. The combined organic layers were dried over Na₂SO₄, filtered and concentrated. The residue was purified by flash chromatography to obtain the titled compound (5.8 mg, 3%). ¹H NMR (600 MHZ, CD₃OD) δ 8.34 (s, 1H), 8.29 (s, 1H), 7.19-7.14 (m, 2H), 7.08 (s, 1H), 6.95 (t, J=8.3 Hz, 2H), 6.47 (dd, J=16.7, 10.2 Hz, 1H), 6.39 (d, J=17.0 Hz, 1H), 5.80 (d, J=10.0 Hz, 1H), 3.77 (s, 3H), 3.01-2.94 (m, 4H). LCMS (ESI) m/z=324 (M+H)⁺.

### Example 64

### N-(3-(2-(4,4-Difluorocyclohexyl)ethyl)-1-methyl-1H-pyrrolo[2,3-b]pyridin-5-yl)acrylamide

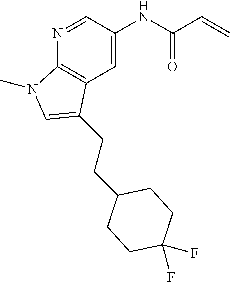 [SMILES: C=CC(=O)Nc1cnc2c(c1)c(CCC1CCC(F)(F)CC1)cn2C]

The title compound was prepared from (E)-3-(2-(4,4-difluorocyclohexyl)vinyl)-1-methyl-5-nitro-1H-pyrrolo[2,3-b]pyridine following the procedure outlined for Example 63. ¹H NMR (600 MHZ, CD₃OD) δ 8.34 (dd, J=9.9, 2.3 Hz, 2H), 7.14 (s, 1H), 6.46 (dd, J=17.0, 10.0 Hz, 1H), 6.38 (dd, J=17.0, 1.7 Hz, 1H), 5.79 (dd, J=10.0, 1.7 Hz, 1H), 3.77 (s, 3H), 2.75-2.70 (m, 2H), 2.05-1.96 (m, 2H), 1.86 (d, J=13.5 Hz, 2H), 1.78-1.60 (m, 4H), 1.46-1.37 (m, 1H), 1.31-1.21 (m, 2H). LCMS (ESI) m/z=347 (M+H)⁺.

### Example 65

### (E)-N-Acryloyl-N-(3-(4-chlorostyryl)-1-methyl-1H-pyrrolo[2,3-b]pyridin-5-yl)glycine

The overall reaction scheme was as follows:

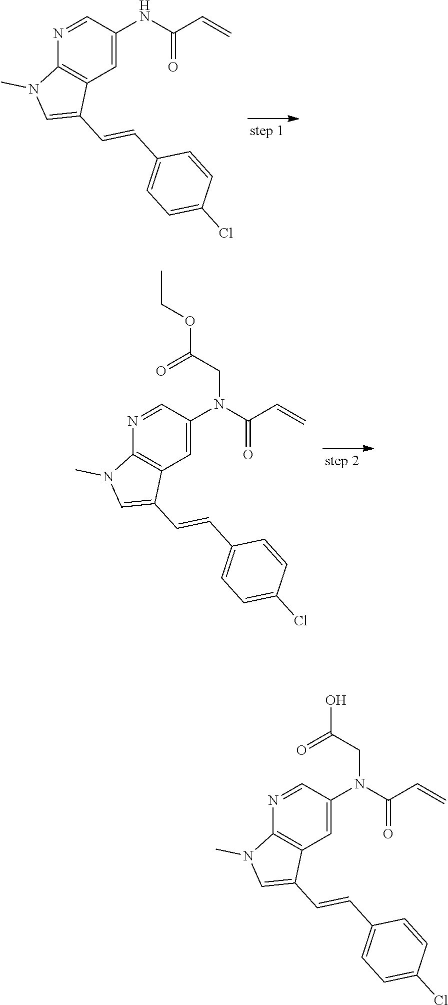 [SMILES: C=CC(=O)Nc1cnc2c(c1)c(/C=C/c1ccc(Cl)cc1)cn2C ; C=CC(=O)N(CC(=O)OCC)c1cnc2c(c1)c(/C=C/c1ccc(Cl)cc1)cn2C ; C=CC(=O)N(CC(=O)O)c1cnc2c(c1)c(/C=C/c1ccc(Cl)cc1)cn2C]

### Step 1: Ethyl (E)-N-acryloyl-N-(3-(4-chlorostyryl)-1-methyl-1H-pyrrolo[2,3-b]pyridin-5-yl)glycinate

To a solution of Example 16 (60.0 mg, 0.178 mmol) in DMF (2 mL) was added NaH (60% suspension in mineral oil, 14.0 mg, 0.355 mmol) portionwise at 0° C. After stirring for 30 min at 0° C., ethyl 2-bromoacetate (44.0 mg, 0.266 mmol) was added to the mixture, and stirred at r.t. for 3 h. The reaction was quenched by adding water, and the resultant mixture was extracted with EtOAc. The combined organic layers were washed with water and brine, dried over anhydrous Na₂SO₄, then concentrated. The residue was purified by flash chromatography to obtain the titled compound (50.0 mg, 67%). ¹H NMR (600 MHZ, CD₃OD) δ 8.38 (s, 1H), 8.29 (s, 1H), 7.67 (s, 1H), 7.46 (d, J=7.6 Hz, 2H), 7.26 (t, J=11.1 Hz, 3H), 7.02 (d, J=16.5 Hz, 1H), 6.32 (d, J=16.8 Hz, 1H), 6.10 (dd, J=16.8, 10.4 Hz, 1H), 5.62 (d, J=10.5 Hz, 1H), 4.54 (s, 2H), 4.21 (q, J=7.0 Hz, 2H), 3.87 (s, 2H), 1.27 (t, J=7.1 Hz, 2H). LCMS (ESI) m/z=424 (M+H)⁺.

### Step 2: (E)-N-Acryloyl-N-(3-(4-chlorostyryl)-1-methyl-1H-pyrrolo[2,3-b]pyridin-5-yl)glycine

To a solution of ethyl (E)-N-acryloyl-N-(3-(4-chlorostyryl)-1-methyl-1H-pyrrolo[2,3-b]pyridin-5-yl)glycinate (50.0 mg, 0.118 mmol) in THF (2 mL) was added 2 N NaOH solution (0.18 mL, 0.355 mmol) and the reaction mixture was stirred at r.t. for 2 h then acidified by 1 N HCl solution. The resultant mixture was extracted with DCM, dried over anhydrous Na₂SO₄, then concentrated. The residue was purified by flash chromatography to obtain the titled compound (14.0 mg, 30%). ¹H NMR (600 MHZ, CD₃OD) δ 8.45 (d, J=1.7 Hz, 1H), 8.31 (d, J=1.9 Hz, 1H), 7.68 (s, 1H), 7.51 (d, J=8.5 Hz, 2H), 7.33-7.24 (m, 3H), 7.05 (d, J=16.5 Hz, 1H), 6.29 (dd, J=16.8, 1.9 Hz, 1H), 6.11 (dd, J=16.8, 10.4 Hz, 1H), 5.59 (dd, J=10.5, 1.9 Hz, 1H), 4.45 (s, 1H), 3.87 (s, 1H). LCMS (ESI) m/z=396 (M+H)⁺.

### Example 66

### (E)-N-(3-(2-(4,4-Difluorocyclohexyl)vinyl)-1-methyl-1H-pyrrolo[2,3-b]pyridin-5-yl)-N-methylacrylamide

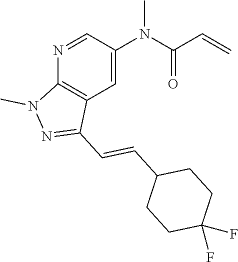 [SMILES: C=CC(=O)N(C)c1cnc2c(c1)c(/C=C/C1CCC(F)(F)CC1)nn2C]

The title compound was prepared from Example 15 and iodomethane following the procedure outlined for Example 65, step 1. ¹H NMR (400 MHZ, CD₃OD) δ 7.88 (s, 2H), 7.26 (s, 1H), 6.30 (d, J=16.2 Hz, 1H), 6.02 (d, J=16.8 Hz, 1H), 5.89 (d, J=16.6 Hz, 1H), 5.85-5.73 (m, 1H), 5.31 (d, J=10.5 Hz, 1H), 3.16 (s, 3H), 3.06 (s, 3H), 2.10-1.96 (m, 1H), 1.88-1.76 (m, 2H), 1.69-1.60 (m, 3H), 1.59-1.49 (m, 1H), 1.38-1.24 (m, 2H). LCMS (ESI) m/z=360 (M+H)⁺.

### Example 67

### (E)-N-Benzyl-N-(3-(4-chlorostyryl)-1-methyl-1H-pyrrolo[2,3-b]pyridin-5-yl)acrylamide

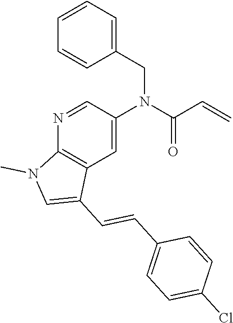 [SMILES: C=CC(=O)N(Cc1ccccc1)c1cnc2c(c1)c(/C=C/c1ccc(Cl)cc1)cn2C]

The title compound was prepared from Example 16 and benzyl chloride following the procedure outlined for Example 65, step 1. ¹H NMR (600 MHZ, CDCl₃) δ 7.98 (d, J=2.2 Hz, 1H), 7.75 (d, J=2.2 Hz, 1H), 7.39 (s, 1H), 7.38-7.34 (m, 2H), 7.33-7.26 (m, 7H), 7.08 (d, J=16.4 Hz, 1H), 6.72 (d, J=16.4 Hz, 1H), 6.47 (dd, J=16.7, 1.9 Hz, 1H), 6.00 (dd, J=16.7, 10.3 Hz, 1H), 5.55 (dd, J=10.3, 1.9 Hz, 1H), 5.05 (s, 2H), 3.89 (s, 3H). LCMS (ESI) m/z=428 (M+H)⁺.

### Example 68

### (E)-N-(3-(4-Chlorostyryl)-1-methyl-1H-pyrrolo[2,3-b]pyridin-5-yl)-N-(pyridin-4-ylmethyl)acrylamide

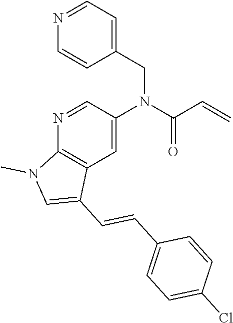 [SMILES: C=CC(=O)N(Cc1ccncc1)c1cnc2c(c1)c(/C=C/c1ccc(Cl)cc1)cn2C]

The title compound was prepared from Example 16 and 4-(chloromethyl)pyridine following the procedure outlined for Example 65, step 1. ¹H NMR (600 MHZ, CDCl₃) δ 7.98 (d, J=2.2 Hz, 1H), 7.75 (d, J=2.2 Hz, 1H), 7.39 (s, 1H), 7.38-7.34 (m, 2H), 7.33-7.26 (m, 7H), 7.08 (d, J=16.4 Hz, 1H), 6.72 (d, J=16.4 Hz, 1H), 6.47 (dd, J=16.7, 1.9 Hz, 1H), 6.00 (dd, J=16.7, 10.3 Hz, 1H), 5.55 (dd, J=10.3, 1.9 Hz, 1H), 5.05 (s, 2H), 3.89 (s, 3H). LCMS (ESI) m/z=429 (M+H)⁺.

### Example 69

### (E)-N-((2H-Tetrazol-5-yl)methyl)-N-(3-(4-chlorostyryl)-1-methyl-1H-pyrrolo[2,3-b]pyridin-5-yl)acrylamide

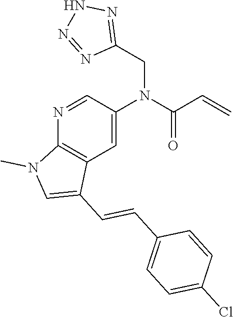 [SMILES: C=CC(=O)N(Cc1nn[nH]n1)c1cnc2c(c1)c(/C=C/c1ccc(Cl)cc1)cn2C]

The title compound was prepared from Example 16 and 5-(chloromethyl)-2H-tetrazole following the procedure outlined for Example 65, step 1. ¹H NMR (600 MHZ, CD₃OD) δ 8.38 (d, J=2.2 Hz, 1H), 8.15 (d, J=2.2 Hz, 1H), 7.64 (s, 1H), 7.45 (d, J=8.5 Hz, 2H), 7.24 (dd, J=27.1, 12.5 Hz, 3H), 6.99 (d, J=16.6 Hz, 1H), 6.34 (dd, J=16.8, 1.7 Hz, 1H), 6.08 (dd, J=16.8, 10.5 Hz, 1H), 5.62 (dd, J=10.5, 1.7 Hz, 1H), 5.34 (s, 2H), 3.82 (s, 3H). LCMS (ESI) m/z=420 (M+H)⁺.

### Example 70

### (E)-2-Chloro-N-(3-(4-chlorostyryl)-1-methyl-1H-pyrrolo[2,3-b]pyridin-5-yl) acetamide

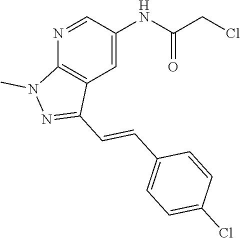 [SMILES: Cn1nc(/C=C/c2ccc(Cl)cc2)c2cc(NC(=O)CCl)cnc21]

The title compound was prepared from (E)-3-(4-chlorostyryl)-1-methyl-1H-pyrrolo[2,3-b]pyridin-5-amine and 2-chloroacetyl chloride following the procedure outlined for Example 1, step 3. ¹H NMR (600 MHz, CDCl₃) δ 8.61 (d, J=2.3 Hz, 1H), 8.37 (br s, 1H), 8.34 (d, J=2.3 Hz, 1H), 7.44-7.41 (m, 2H), 7.38 (s, 1H), 7.33-7.29 (m, 2H), 7.17 (d, J=16.4 Hz, 1H), 6.98 (d, J=16.4 Hz, 1H), 4.28 (s, 2H), 3.89 (s, 3H). LCMS (ESI) m/z=360 (M+H)⁺.

### Example 71

### N-(3-(Cyclohex-1-en-1-yl)-1-methyl-1H-indol-5-yl)acrylamide

The overall reaction scheme was as follows:

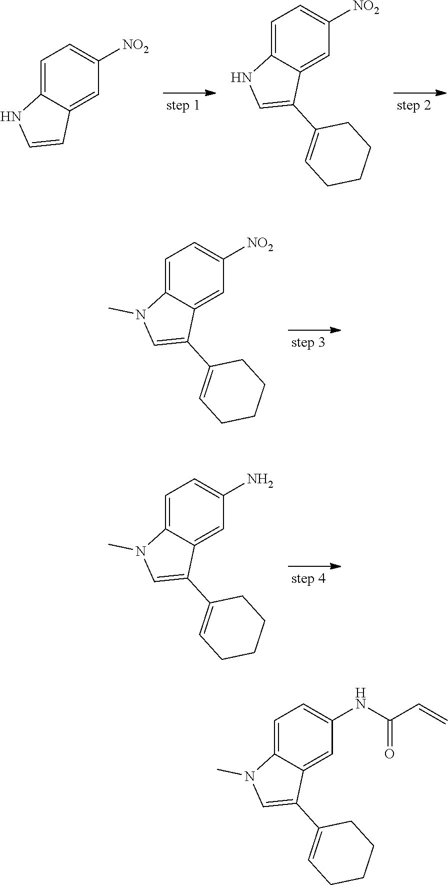 [SMILES: O=[N+]([O-])c1ccc2[nH]ccc2c1 ; O=[N+]([O-])c1ccc2[nH]cc(C3=CCCCC3)c2c1 ; Cn1cc(C2=CCCCC2)c2cc([N+](=O)[O-])ccc21 ; Cn1cc(C2=CCCCC2)c2cc(N)ccc21 ; C=CC(=O)Nc1ccc2c(c1)c(C1=CCCCC1)cn2C]

### Step 1: 3-(Cyclohex-1-en-1-yl)-5-nitro-1H-indole

To a mixture of 5-nitro-1H-indole (300 mg, 1.85 mmol) and cyclohexanone (0.38 mL, 3.70 mmol) in MeOH (3 mL), NaOMe solution (30 wt %, 2.5 mL, 11.1 mmol) was added. The reaction mixture was stirred at 90° C. for 15 h then cooled to r.t. The reaction mixture was diluted with EtOAc and water then extracted with EtOAc. The collected organic layer was dried over anhydrous Na₂SO₄, filtered, and concentrated. The residue was purified by flash chromatography (0-30% EtOAc/DCM) to obtain the titled compound (339 mg, 76%). ¹H NMR (400 MHZ, CDCl₃) δ 8.86 (s, 1H), 8.40 (s, 1H), 8.12 (d, J=9.0 Hz, 1H), 7.39 (d, J=9.3 Hz, 1H), 6.30 (s, 1H), 2.47-2.41 (m, 2H), 2.33-2.28 (m, 2H), 1.86-1.79 (m, 2H), 1.78-1.68 (m, 2H). LCMS (ESI) m/z=243 (M+H)⁺.

### Step 2: 3-(Cyclohex-1-en-1-yl)-1-methyl-5-nitro-1H-indole

To a mixture of 3-(cyclohex-1-en-1-yl)-5-nitro-1H-indole (339 mg, 1.40 mmol) and Cs₂CO₃ (2.00 eq, 912 mg, 2.80 mmol) in DMF (3 mL), iodoethane (0.13 mL, 1.68 mmol) was added. The reaction mixture was stirred at 120° C. for 5 h then cooled to r.t. The resultant mixture was diluted in EtOAc and washed with water 3 times then brine. The organic layer was dried over anhydrous Na₂SO₄, filtered, and concentrated. The residue was purified by flash chromatography (0%-30% EtOAc/Hex) to obtain the titled compound (315 mg, 88%). ¹H NMR (600 MHZ, CDCl₃) δ 8.84 (d, J=2.2 Hz, 1H), 8.13 (dd, J=9.1, 2.3 Hz, 1H), 7.29 (d, J=9.0 Hz, 1H), 7.11 (s, 1H), 6.31-6.28 (m, 1H), 3.81 (s, 3H), 2.44-2.39 (m, 2H), 2.31-2.27 (m, 2H), 1.85-1.80 (m, 2H), 1.75-1.70 (m, 2H). LCMS (ESI) m/z=257 (M+H)⁺.

### Step 3: 3-(Cyclohex-1-en-1-yl)-1-methyl-1H-indol-5-amine

A mixture of 3-(cyclohex-1-en-1-yl)-1-methyl-5-nitro-1H-indole (150 mg, 0.585 mmol), iron powder (98 mg, 1.76 mmol), and HCl solution (3 N aqueous solution, 0.39 mL, 1.17 mmol) were suspended in EtOH (5 mL). The reaction mixture was stirred at 80° C. for 15 h then cooled to r.t. The resultant mixture was filtered to remove insolubles. The filtrate was concentrated and the residue was purified by flash chromatography (0%-30% EtOAc/DCM) to obtain the titled compound (108 mg, 82%). LCMS (ESI) m/z=227 (M+H)⁺.

### Step 4: N-(3-(Cyclohex-1-en-1-yl)-1-methyl-1H-indol-5-yl)acrylamide

To a mixture of 3-(cyclohex-1-en-1-yl)-1-methyl-1H-indol-5-amine (108 mg, 0.477 mmol), acrylic acid (0.049 mL, 0.716 mmol), DIPEA (0.29 mL, 1.67 mmol), and DMAP (4.8 mg, 0.0477 mmol) in DCM (4 mL), EDCI (126 mg, 0.811 mmol) was added. The reaction mixture was stirred at r.t. for 16 h. The reaction was quenched by adding water and extracted with DCM. The collected organic layer was dried over anhydrous Na₂SO₄, filtered, and concentrated. The residue was purified by prep. HPLC (MeCN/water) to obtain the titled compound (20 mg, 15%). ¹H NMR (600 MHZ, CD₃OD) δ 8.21 (d, J=1.9 Hz, 1H), 7.35 (dd, J=8.7, 2.0 Hz, 1H), 7.30-7.27 (m, 1H), 7.12 (s, 1H), 6.46 (dd, J=17.0, 10.2 Hz, 1H), 6.34 (dd, J=17.0, 1.7 Hz, 1H), 6.21-6.17 (m, 1H), 5.74 (dd, J=10.2, 1.7 Hz, 1H), 3.73 (s, 3H), 2.45-2.40 (m, 2H), 2.28-2.23 (m, 2H), 1.83-1.78 (m, 2H), 1.73-1.68 (m, 2H). LCMS (ESI) m/z=281 (M+H)⁺.

### Example 72

### N-(3-Benzyl-1-methyl-2-oxoindolin-5-yl)acrylamide

The overall reaction scheme was as follows:

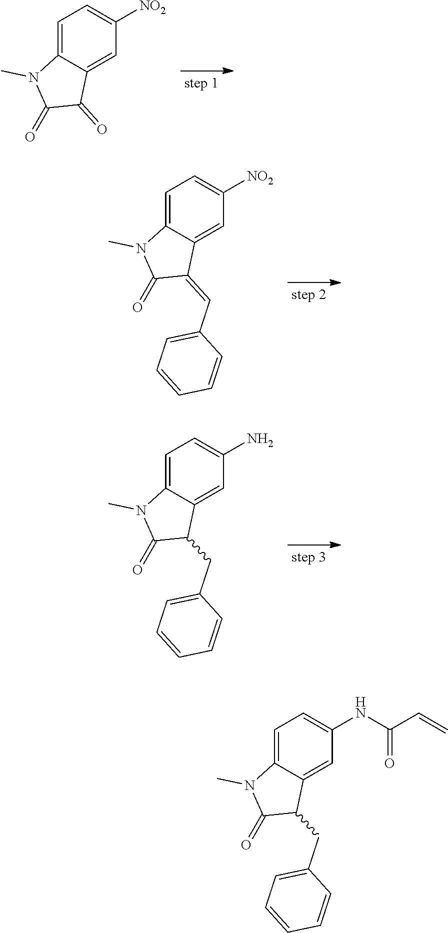

### Step 1: 3-Benzylidene-1-methyl-5-nitro-indolin-2-one

To a mixture of benzyltriphenylphosphonium chloride (278 mg, 0.715 mmol) in dry THF (3 mL), n-BuLi (2.5 M in Hex, 0.31 mL, 0.780 mmol) was added as dropwise. The reaction mixture was stirred for 30 min then a solution of 1-methyl-5-nitro-indoline-2,3-dione (134 mg, 0.650 mmol) in dry THF (9 mL) was added as dropwise. The resultant mixture was stirred at r.t. for 18 h then quenched by adding water. The resultant mixture was extracted with EtOAc twice. The collected organic layer was washed with brine, dried over Na₂SO₄, filtered, and concentrated. The residue was purified by flash chromatography (0%-20% EtOAc/Hex) to obtain the titled compound (78 mg, E/Z=1:4, 43%). ¹H NMR (400 MHZ, CDCl₃) δ 8.57 (s, 1H), 8.45 (s, 1H, E-form), 8.35 (s, 1H, E-form), 8.26 (d, J=8.8 Hz, 1H), 8.16 (s, 1H, E-form), 8.04 (s, 1H), 7.74 (s, 1H, E-form), 7.67 (s, 2H), 7.54 (s, 3H), 7.38 (d, J=16.3 Hz, 2H), 6.99-6.89 (m, 2H), 3.37 (s, 3H), 3.28 (s, 3H, E-form). LCMS (ESI) m/z=281 (M+H)⁺.

### Step 2: 5-Amino-3-benzyl-1-methyl-indolin-2-one

A mixture of 3-benzylidene-1-methyl-5-nitro-indolin-2-one (40 mg, 0.143 mmol) and Pd/C (30 mg, 0.0285 mmol) were suspended in EtOAc (3 mL). The reaction mixture was stirred under hydrogen atmosphere for 18 h then filtered to remove insolubles. The filtrate was concentrated and the residue was purified by flash chromatography (0%-30% EtOAc/DCM) to obtain the titled compound (21 mg, 58%). ¹H NMR (400 MHZ, CDCl₃) δ 7.25-7.11 (m, 5H), 6.68 (d, J=8.6 Hz, 1H), 6.58 (d, J=8.1 Hz, 1H), 6.26 (s, 1H), 3.65 (dd, J=9.5, 4.4 Hz, 1H), 3.47 (dd, J=13.7, 4.5 Hz, 1H), 3.11 (s, 3H), 2.85 (dd, J=13.7, 9.5 Hz, 1H). LCMS (ESI) m/z=253 (M+H)⁺.

### Step 3: N-(3-Benzyl-1-methyl-2-oxoindolin-5-yl)acrylamide

To a mixture of 5-amino-3-benzyl-1-methyl-indolin-2-one (21 mg, 0.0832 mmol), acrylic acid (0.0086 mL, 0.125 mmol), DIPEA (0.051 mL, 0.291 mmol), and DMAP (0.84 mg, 0.00832 mmol) in DCM (0.8323 mL), EDCI (22 mg, 0.141 mmol) was added. The reaction mixture was stirred at r.t. for 16 h. The reaction was quenched by adding water and extracted with DCM. The collected organic layer was dried over anhydrous Na₂SO₄, filtered, and concentrated. The residue was purified by prep. HPLC (MeCN/water) to obtain the titled compound. (1.0 mg, 4%). ¹H NMR (600 MHz, CD₃OD) δ 7.40-7.37 (m, 1H), 7.31 (s, 1H), 7.16-7.10 (m, 3H), 7.09-7.05 (m, 2H), 6.79 (d, J=8.4 Hz, 1H), 6.38 (dd, J=17.4, 1.5 Hz, 1H), 6.15 (dd, J=17.3, 10.5 Hz, 1H), 5.88 (dd, J=10.5, 1.5 Hz, 1H), 4.47 (t, J=6.2 Hz, 2H), 3.07 (s, 3H), 2.72 (t, J=6.2 Hz, 2H). LCMS (ESI) m/z=307 (M+H)⁺.

### Example 73

### N-(1-Methyl-2-oxo-3-(4-(trifluoromethyl)benzylidene) indolin-5-yl)acrylamide

The overall reaction scheme was as follows:

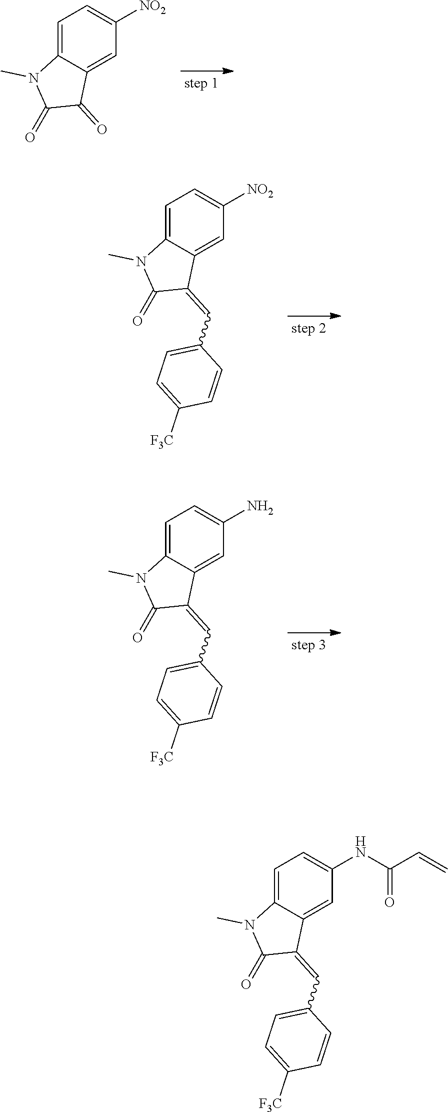 [SMILES: CN1C(=O)C(=O)c2cc([N+](=O)[O-])ccc21 ; CN1C(=O)C(=Cc2ccc(C(F)(F)F)cc2)c2cc([N+](=O)[O-])ccc21 ; CN1C(=O)C(=Cc2ccc(C(F)(F)F)cc2)c2cc(N)ccc21 ; C=CC(=O)Nc1ccc2c(c1)C(=Cc1ccc(C(F)(F)F)cc1)C(=O)N2C]

### Step 1: 1-Methyl-5-nitro-3-[[4-(trifluoromethyl)phenyl]methylene]indolin-2-one

A mixture of p-(trifluoromethyl)benzyl bromide (0.051 mL, 0.331 mmol) and PPh₃ (87 mg, 0.331 mmol) in dry THF (2 mL) was stirred at 75° C. for 12 h until precipitate observed then cooled to r.t. n-BuLi (2.5 M in Hex, 0.13 mL, 0.331 mmol) was added as dropwise and stirred for additional 10 min. A solution of 1-methyl-5-nitro-indoline-2,3-dione (62 mg, 0.301 mmol) in dry THF (4 mL) was added as dropwise and the reaction mixture was stirred at r.t. for 7 h. The reaction was quenched by adding water and extracted with EtOAc twice. The collected organic layer was washed with brine, dried over Na₂SO₄, filtered, and concentrated. The residue was purified by flash chromatography (0%-30% EtOAc/Hex) to obtain the titled compound (23 mg, 22%). ¹H NMR (600 MHZ, CDCl₃) δ 8.46 (d, J=2.2 Hz, 1H), 8.38 (d, J=8.1 Hz, 2H), 8.31 (dd, J=8.6, 2.2 Hz, 1H), 7.73 (d, J=8.1 Hz, 3H), 6.93 (d, J=8.6 Hz, 1H), 3.36 (s, 3H). LCMS (ESI) m/z=349 (M+H)⁺.

### Step 2: 5-Amino-1-methyl-3-[[4-(trifluoromethyl)phenyl]methylene]indolin-2-one

A mixture of 1-methyl-5-nitro-3-[[4-(trifluoromethyl)phenyl]methylene]indolin-2-one (23 mg, 0.0660 mmol), iron powder (11 mg, 0.198 mmol), and HCl solution (3 N aqueous solution, 0.044 mL, 0.132 mmol) were suspended in EtOH (2 mL). The reaction mixture was stirred at 80° C. for 15 h then cooled to rt. The resultant mixture was filtered to remove insolubles. The filtrate was concentrated and the residue was purified by flash chromatography (0%-30% EtOAc/DCM) to obtain the titled compound (12 mg, 57%). LCMS (ESI) m/z=319 (M+H)⁺.

### Step 3: N-(1-Methyl-2-oxo-3-(4-(trifluoromethyl)benzylidene) indolin-5-yl)acrylamide

A mixture of 5-amino-1-methyl-3-[[4-(trifluoromethyl)phenyl]methylene]indolin-2-one (12 mg, 0.0377 mmol), N-acryloxysuccinimide (13 mg, 0.0754 mmol), and TEA (0.011 mL, 0.0754 mmol) were suspended in CHCl₃ (1 mL). The reaction mixture was stirred at r.t. for 16 h then quenched by adding water. The resultant mixture was extracted with DCM. The collected organic layer was dried over Na₂SO₄, filtered, and concentrated. The residue was purified by prep. HPLC (MeCN/water) to obtain the titled compound (1.8 mg, 13%). ¹H NMR (600 MHZ, CD₃OD) δ 7.98 (d, J=2.1 Hz, 1H), 7.89 (d, J=8.2 Hz, 2H), 7.85 (d, J=8.3 Hz, 2H), 7.80 (s, 1H), 7.76-7.71 (m, 1H), 7.62 (dd, J=8.4, 2.1 Hz, 1H), 7.00 (d, J=8.5 Hz, 1H), 6.36 (dd, J=17.0, 9.5 Hz, 1H), 6.31 (dd, J=17.0, 2.4 Hz, 1H), 5.74 (dd, J=9.4, 2.4 Hz, 1H), 3.28 (s, 3H). LCMS (ESI) m/z=373 (M+H)⁺.

### Example 74

### N-(1-Methyl-1H-indazol-5-yl)acrylamide

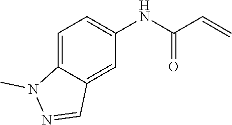

The title compound was prepared from 1-methylindazol-5-amine and acryloyl chloride following the procedure outlined for Example 1, step 3. ¹H NMR (400 MHZ, CD₃OD) δ 8.16 (s, 1H), 7.96 (s, 1H), 7.56-7.50 (m, 2H), 6.46 (dd, J=17.0, 9.8 Hz, 1H), 6.37 (dd, J=17.0, 2.1 Hz, 1H), 5.77 (dd, J=9.8, 2.1 Hz, 1H), 4.05 (s, 3H). LCMS (ESI) m/z=202 (M+H)⁺.

### Example 75

### (E)-N-(1-Acetyl-3-(4-chlorostyryl)-1H-indol-5-yl)acrylamide

The overall reaction scheme was as follows:

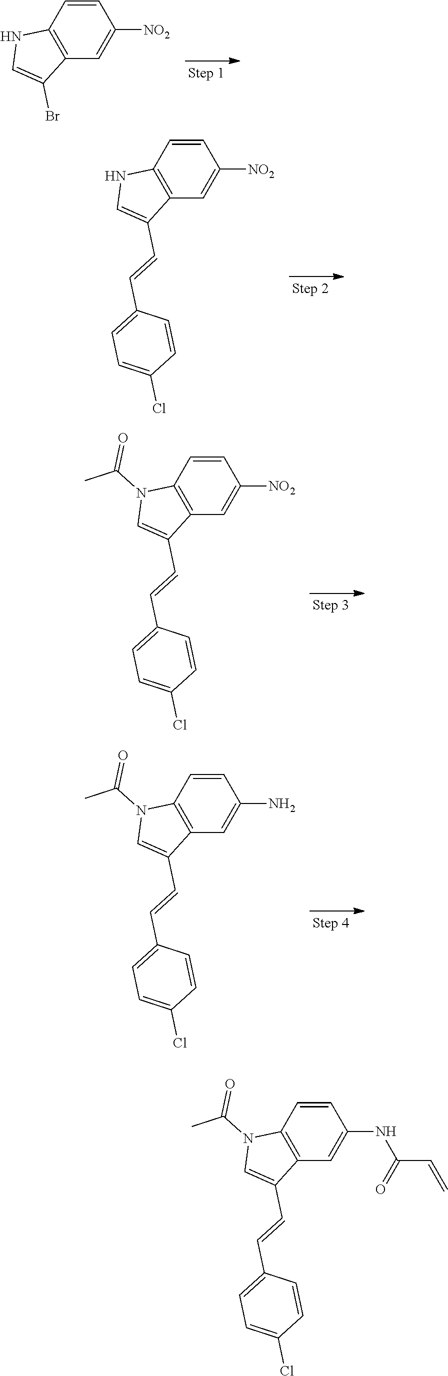

### Step 1: 3-[(E)-2-(4-Chlorophenyl) vinyl]-5-nitro-1H-indole

A mixture of 3-bromo-5-nitro-1H-indole (300 mg, 1.24 mmol), Pd(PPh₃)₂Cl₂ (87 mg, 0.124 mmol), Na₂CO₃ (264 mg, 2.49 mmol) and [(E)-2-(4-chlorophenyl) vinyl]boronic acid (272 mg, 1.49 mmol) in THF (12 mL) and water (4 mL) was stirred at 80° C. for 16 h under nitrogen atmosphere. The reaction mixture was quenched by adding water and extracted with EtOAc twice. The combined organic layers were dried over anhydrous Na₂SO₄, filtered, and concentrated. The residue was purified by flash chromatography to obtain the titled compound (160 mg, 43%). ¹H NMR (600 MHz, CDCl₃) δ 8.90 (d, J=2.1 Hz, 1H), 8.55 (s, 1H), 8.17 (dd, J=8.9, 2.2 Hz, 1H), 7.52 (d, J=2.5 Hz, 1H), 7.49-7.46 (m, 2H), 7.44 (d, J=8.9 Hz, 1H), 7.36-7.32 (m, 2H), 7.27-7.22 (m, 1H), 7.10 (d, J=16.5 Hz, 1H). LCMS (ESI) m/z=299 (M+H)⁺.

### Step 2: (E)-1-(3-(4-Chlorostyryl)-5-nitro-1H-indol-1-yl)ethan-1-one

A solution of 3-[(E)-2-(4-chlorophenyl) vinyl]-5-nitro-1H-indole (100 mg, 0.335 mmol) and TEA (0.12 mL, 0.837 mmol) in DCM (3 mL) was stirred at r.t. The mixture was cooled to 0° C. and then acetyl chloride (0.036 mL, 0.502 mmol) was added as dropwise. The resultant mixture was stirred at 0° C. for 1 h. The reaction was quenched by adding water and extracted with DCM twice. The combined organic layers were dried over anhydrous Na₂SO₄, filtered and concentrated. The residue was purified by flash chromatography to obtain the titled compound (71 mg, 62%). ¹H NMR (600 MHz, CDCl₃) δ 8.76 (d, J=2.3 Hz, 1H), 8.62 (d, J=9.1 Hz, 1H), 8.30 (dd, J=9.1, 2.3 Hz, 1H), 7.70 (s, 1H), 7.50-7.47 (m, 2H), 7.39-7.35 (m, 2H), 7.18 (d, J=5.3 Hz, 2H), 2.71 (s, 3H). LCMS (ESI) m/z=341 (M+H)⁺.

### Step 3: (E)-1-(5-Amino-3-(4-chlorostyryl)-1H-indol-1-yl)ethan-1-one

To a stirred solution of (E)-1-(3-(4-chlorostyryl)-5-nitro-1H-indol-1-yl)ethan-1-one (71 mg, 0.207 mmol) in a mixed solution of THF/water (4:1, 5 mL) were added zinc (136 mg, 2.07 mmol) and NH₄Cl (111 mg, 2.07 mmol) at r.t. The reaction mixture was stirred for 2 h. After completion of the reaction, the mixture was filtered to remove insolubles and extracted with EtOAc. The combined organic layer was dried over anhydrous Na₂SO₄, filtered and concentrated. The residue was purified by flash chromatography to obtain the titled compound (26 mg, 40%). ¹H NMR (600 MHz, CDCl₃) δ 7.45-7.41 (m, 2H), 7.34-7.31 (m, 2H), 7.25 (s, 2H), 7.13 (d, J=2.3 Hz, 1H), 7.10 (d, J=1.7 Hz, 2H), 6.78 (dd, J=8.7, 2.3 Hz, 1H), 2.61 (s, 3H). LCMS (ESI) m/z=311 (M+H)⁺.

### Step 4: (E)-N-(1-Acetyl-3-(4-chlorostyryl)-1H-indol-5-yl)acrylamide

A solution of (E)-1-(5-amino-3-(4-chlorostyryl)-1H-indol-1-yl)ethan-1-one (23 mg, 0.0753 mmol) and TEA (0.026 mL, 0.188 mmol) in DCM (3 mL) was stirred at r.t. The mixture was cooled to 0° C. and then acryloyl chloride (0.0092 mL, 0.113 mmol) was added as dropwise. The resultant mixture was stirred at 0° C. for 1 h. The reaction was quenched by adding water and extracted with DCM twice. The combined organic layers were dried over Na₂SO₄, filtered and concentrated. The residue was purified by flash chromatography to obtain the titled compound (12 mg, 43%). ¹H NMR (600 MHZ, CD₃OD) δ 8.35 (d, J=1.9 Hz, 1H), 8.26 (d, J=8.8 Hz, 1H), 7.73 (s, 1H), 7.46 (d, J=8.5 Hz, 2H), 7.39 (dd, J=8.9, 2.0 Hz, 1H), 7.31-7.26 (m, 2H), 7.16-7.09 (m, 2H), 6.44 (dd, J=17.0, 9.8 Hz, 1H), 6.37 (dd, J=16.9, 1.9 Hz, 1H), 5.77 (dd, J=9.8, 1.9 Hz, 1H), 2.56 (s, 3H). LCMS (ESI) m/z=365 (M+H)⁺.

### Example 76

### N-(1-(4-(Trifluoromethyl)benzyl)-1H-pyrrolo[3,2-b]pyridin-6-yl)acrylamide

The overall reaction scheme was as follows:

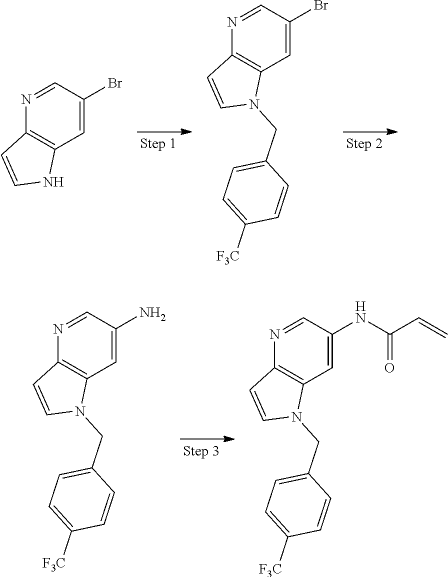 [SMILES: Brc1cnc2cc[nH]c2c1 ; FC(F)(F)c1ccc(Cn2ccc3ncc(Br)cc32)cc1 ; Nc1cnc2ccn(Cc3ccc(C(F)(F)F)cc3)c2c1 ; C=CC(=O)Nc1cnc2ccn(Cc3ccc(C(F)(F)F)cc3)c2c1]

### Step 1: 6-Bromo-1-(4-(trifluoromethyl)benzyl)-1H-pyrrolo[3,2-b]pyridine

A mixture of 6-bromo-1H-pyrrolo[3,2-b]pyridine (200 mg, 1.02 mmol), NaH (60% suspension in mineral oil, 81 mg, 3.38 mmol) and 1-(bromomethyl)-4-(trifluoromethyl)benzene (364 mg, 1.52 mmol) in DMF (4 mL) was stirred at 110° C. for 5 h. The reaction mixture was diluted in EtOAc and washed with brine 4 times. The combined organic layers were dried over anhydrous Na₂SO₄ and concentrated under reduced pressure. The residue was purified by flash chromatography to obtain the titled compound (253 mg, 70%). ¹H NMR (600 MHz, CDCl₃) δ 8.52 (d, J=1.9 Hz, 1H), 7.75 (s, 1H), 7.60 (d, J=8.2 Hz, 2H), 7.41 (d, J=3.3 Hz, 1H), 7.18 (d, J=8.1 Hz, 2H), 6.84 (d, J=3.2 Hz, 1H), 5.38 (s, 2H). LCMS (ESI) m/z=356 (M+H)⁺.

### Step 2: 1-(4-(Trifluoromethyl)benzyl)-1H-pyrrolo[3,2-b]pyridin-6-amine

A mixture of 6-bromo-1-(4-(trifluoromethyl)benzyl)-1H-pyrrolo[3,2-b]pyridine (253 mg, 0.712 mmol), Pd₂dba₃ (65 mg, 0.0712 mmol), xantphos (82 mg, 0.142 mmol), Cs₂CO₃ (696 mg, 2.14 mmol) and benzophenone imine (0.18 mL, 1.07 mmol) in dry toluene (4 mL) was stirred at 110° C. for 16 h under nitrogen atmosphere. The reaction was quenched by adding water and extracted with EtOAc twice. The combined organic layers were dried over anhydrous Na₂SO₄ and concentrated. The residue was suspended in 3 N HCl (2 mL) and stirred at r.t. for 16 h. The reaction mixture was diluted in water and extracted with EtOAc twice. The combined organic layers were dried over anhydrous Na₂SO₄ and concentrated. The residue was purified by flash chromatography to obtain the titled compound (20 mg, 9.6%). LCMS (ESI) m/z=292 (M+H)⁺.

### Step 3: N-(1-(4-(Trifluoromethyl)benzyl)-1H-pyrrolo[3,2-b]pyridin-6-yl)acrylamide

A solution of 1-(4-(trifluoromethyl)benzyl)-1H-pyrrolo[3,2-b]pyridin-6-amine (20 mg, 0.0687 mmol) and TEA (0.019 mL, 0.137 mmol) in DCM (3 mL) was stirred at r.t. The mixture was cooled to 0° C. then acryloyl chloride (0.0065 mL, 0.0824 mmol) was added as dropwise. The resultant mixture was stirred at 0° C. for 1 h. The reaction was quenched by adding water and extracted with DCM twice. The combined organic layers were dried over anhydrous Na₂SO₄, filtered and concentrated. The residue was purified by flash chromatography to obtain the titled compound (10.6 mg, 45%). ¹H NMR (600 MHZ, CD₃OD) δ 8.41 (s, 1H), 8.38 (s, 1H), 7.65 (d, J=3.3 Hz, 1H), 7.62 (d, J=8.2 Hz, 2H), 7.33 (d, J=8.1 Hz, 2H), 6.66 (d, J=3.2 Hz, 1H), 6.47-6.34 (m, 2H), 5.79 (d, J=10.0 Hz, 1H), 5.52 (s, 2H). LCMS (ESI) m/z=346 (M+H)⁺.

### Example 77

### N-(1-(Cyclohexylmethyl)-1H-pyrrolo[3,2-b]pyridin-6-yl)acrylamide

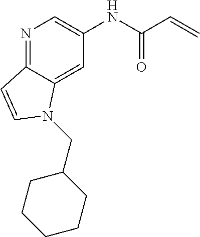 [SMILES: C=CC(=O)Nc1cnc2ccn(CC3CCCCC3)c2c1]

The title compound was prepared from 6-bromo-1-(4-(trifluoromethyl)benzyl)-1H-pyrrolo[3,2-b]pyridine and bromomethylcyclohexane following the procedure outlined for Example 76. ¹H NMR (600 MHZ, CD₃OD) δ 8.48 (dd, J=2.1, 0.7 Hz, 1H), 8.36 (d, J=2.1 Hz, 1H), 7.51 (d, J=3.3 Hz, 1H), 6.55 (dd, J=3.2, 0.8 Hz, 1H), 6.49 (dd, J=17.0, 10.0 Hz, 1H), 6.41 (dd, J=17.0, 1.8 Hz, 1H), 5.82 (dd, J=10.0, 1.8 Hz, 1H), 4.03 (d, J=7.3 Hz, 3H), 1.92-1.84 (m, 1H), 1.76-1.70 (m, 2H), 1.68-1.63 (m, 1H), 1.61-1.56 (m, 2H), 1.35-1.30 (m, 2H), 1.26-1.19 (m, 4H), 1.10-1.00 (m, 2H). LCMS (ESI) m/z=284 (M+H)⁺.

### Example 78

### N-(1-(4-Fluorobenzyl)-1H-pyrrolo[3,2-b]pyridin-6-yl)acrylamide

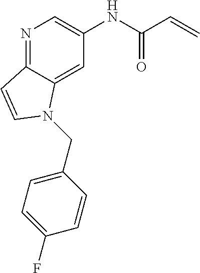 [SMILES: C=CC(=O)Nc1cnc2ccn(Cc3ccc(F)cc3)c2c1]

The title compound was prepared from 6-bromo-1-(4-(trifluoromethyl)benzyl)-1H-pyrrolo[3,2-b]pyridine and 4-fluorobenzyl bromide following the procedure outlined for Example 76. ¹H NMR (600 MHZ, CD₃OD) δ 8.41 (dd, J=2.2, 0.9 Hz, 1H), 8.39 (d, J=2.1 Hz, 1H), 7.63 (d, J=3.3 Hz, 1H), 7.27-7.22 (m, 2H), 7.08-7.03 (m, 2H), 6.62 (dd, J=3.3, 0.9 Hz, 1H), 6.45 (dd, J=17.0, 9.9 Hz, 1H), 6.38 (dd, J=17.0, 1.9 Hz, 1H), 5.80 (dd, J=9.9, 1.9 Hz, 1H), 5.40 (s, 2H). LCMS (ESI) m/z=296 (M+H)⁺.

### Intermediate U

### Preparation of 3-bromo-1-(difluoromethyl)-5-nitro-1H-pyrrolo[2,3-b]pyridine

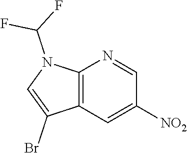 [SMILES: O=[N+]([O-])c1cnc2c(c1)c(Br)cn2C(F)F]

3-Bromo-5-nitro-1H-pyrrolo[2,3-b]pyridine (1.0 g, 4.13 mmol), ethyl 2-bromo-2,2-difluoro-acetate (0.59 mL, 4.54 mmol), and t-BuOK (0.93 g, 8.26 mmol) were suspended in MeCN (40 mL). The reaction mixture was stirred at 80° C. for 16 h then concentrated. The residue was diluted with EtOAc and water, extracted with EtOAc. The collected organic layer was dried over Na₂SO₄, filtered, and concentrated. The residue was purified by flash chromatography to obtain the titled compound (0.91 g, 75%). ¹H NMR (600 MHZ, DMSO-d6) 9.28 (t, J=1.9 Hz, 1H), 8.72 (d, J=2.4 Hz, 1H), 8.52 (s, 1H), 8.24 (t, J=58.7 Hz, 1H). LCMS (ESI) m/z=293 (M+H)⁺.

### Intermediate V

### Preparation of 3-iodo-1-methyl-5-nitro-1H-pyrrolo[2,3-b]pyridine

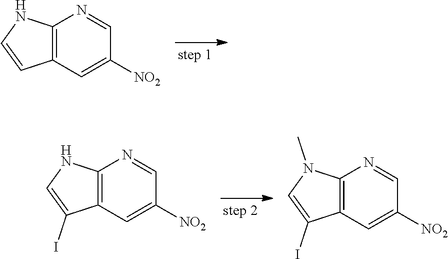

### Step 1: 3-Iodo-5-nitro-1H-pyrrolo[2,3-b]pyridine

5-Nitro-1H-pyrrolo[2,3-b]pyridine (1.00 g, 6.13 mmol) and KOH (2.12 g, 15.3 mmol) were dissolved in DMF (10 mL). To this solution molecular iodine (1.71 g, 6.74 mmol) dissolved in DMF (10 mL) were added dropwise. The resulting mixture was subsequently stirred for 1 h at r.t. Afterwards the reaction mixture was added excess water and stirred. The formed precipitate was collected by filtration and rinsed twice with water. The filtered precipitate was dried in vacuo to obtain the titled compound (1.5 g, 85%). ¹H NMR (600 MHZ, CDCl₃) δ 12.91 (s, 1H), 9.09 (d, J=2.0 Hz, 1H), 8.41 (d, J=2.1 Hz, 1H), 8.03 (s, 1H). LCMS (ESI) m/z=290 (M+H)⁺.

### Step 2: 3-Iodo-1-methyl-5-nitro-1H-pyrrolo[2,3-b]pyridine

To the solution of 3-iodo-5-nitro-1H-pyrrolo[2,3-b]pyridine (1.5 g, 5.19 mmol) in DMF (26 mL) was added Cs₂CO₃ (3.38 g, 10.38 mmol), iodomethane (0.485 mL, 7.78 mmol) and stirred at 80° C. for 16 h. After reaction completion, water is poured into the reaction mixture. The mixture was filtered, and the resulting solid was dried in vacuo to give the titled compound (1.5 g, 92%). ¹H NMR (400 MHZ, CDCl₃) δ 9.23 (d, J=2.3 Hz, 1H), 8.59 (d, J=2.4 Hz, 1H), 7.48 (s, 1H), 3.97 (s, 3H). LCMS (ESI) m/z=304 (M+H)⁺.

### Intermediate W

### Preparation of 1-(difluoromethyl)-3-iodo-5-nitro-1H-pyrrolo[2,3-b]pyridine

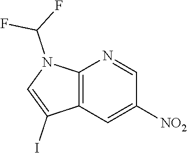 [SMILES: O=[N+]([O-])c1cnc2c(c1)c(I)cn2C(F)F]

The title compound was prepared from 3-iodo-5-nitro-1H-pyrrolo[2,3-b]pyridine following the procedure outlined for Intermediate U. ¹H NMR (400 MHZ, CDCl₃) δ 9.26 (d, J=2.5 Hz, 1H), 8.64 (d, J=2.1 Hz, 1H), 7.83 (d, J=2.3 Hz, 2H). LCMS (ESI) m/z=339 (M+H)⁺.

### Example 79

### (E)-N-(3-(2-Cyclohexylvinyl)-1-methyl-1H-indol-5-yl)acrylamide

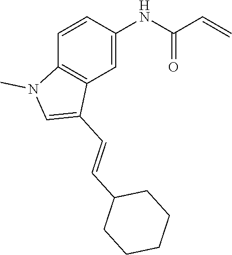 [SMILES: C=CC(=O)Nc1ccc2c(c1)c(/C=C/C1CCCCC1)cn2C]

The title compound was prepared from 3-bromo-1-methyl-5-nitro-1H-indole and (E)-2-(2-cyclohexylvinyl)-4,4,5,5-tetramethyl-1,3,2-dioxaborolane following the procedure outlined for Example 15. ¹H NMR (400 MHZ, CD₃OD) δ 8.17 (s, 1H), 7.42-7.28 (m, 2H), 7.17 (s, 1H), 6.48 (d, J=16.2 Hz, 2H), 6.37 (d, J=17.2 Hz, 1H), 6.09 (d, J=13.9 Hz, 1H), 5.76 (d, J=9.7 Hz, 1H), 3.76 (s, 3H), 2.12 (s, 1H), 1.89-1.77 (m, 4H), 1.72 (d, J=11.3 Hz, 1H), 1.43-1.33 (m, 1H), 1.32-1.20 (m, 4H). LCMS (ESI) m/z=309 (M+H)⁺.

### Example 80

### (E)-N-(3-(4-Isopropylstyryl)-1-methyl-1H-indol-5-yl)acrylamide

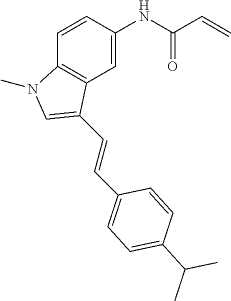 [SMILES: C=CC(=O)Nc1ccc2c(c1)c(/C=C/c1ccc(C(C)C)cc1)cn2C]

The title compound was prepared from 3-bromo-1-methyl-5-nitro-1H-indole and (E)-(4-isopropylstyryl) boronic acid following the procedure outlined for Example 15. ¹H NMR (400 MHZ, CD₃OD) δ 8.36 (s, 1H), 7.42 (s, 3H), 7.33 (s, 2H), 7.21 (dd, J=22.6, 11.2 Hz, 3H), 7.04 (d, J=16.4 Hz, 1H), 6.56-6.46 (m, 1H), 6.39 (d, J=16.2 Hz, 1H), 5.76 (t, J=10.6 Hz, 1H), 3.75 (s, 3H), 2.88 (s, 1H), 1.24 (t, J=11.4 Hz, 6H). LCMS (ESI) m/z=345 (M+H)⁺.

### Example 81

### (E)-N-(3-(2-(5-Chlorothiophen-2-yl) vinyl)-1-methyl-1H-pyrrolo[2,3-b]pyridin-5-yl)acrylamide

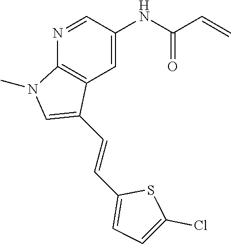 [SMILES: C=CC(=O)Nc1cnc2c(c1)c(/C=C/c1ccc(Cl)s1)cn2C]

The title compound was prepared from Intermediate E and 2-[(E)-2-(5-chloro-2-thienyl) vinyl]-4,4,5,5-tetramethyl-1,3,2-dioxaborolane following the procedure outlined for Example 15. ¹H NMR (400 MHZ, DMSO-d6) § 10.28 (s, 1H), 8.62 (d, J=2.2 Hz, 1H), 8.48 (d, J=2.1 Hz, 1H), 7.80 (s, 1H), 7.02 (ddd, J=20.4, 16.5, 5.2 Hz, 4H), 6.46 (dd, J=17.0, 10.1 Hz, 1H), 6.28 (dd, J=17.0, 2.0 Hz, 1H), 5.77 (dd, J=10.1, 1.9 Hz, 1H), 3.31 (s, 3H). LCMS (ESI) m/z=344 (M+H)⁺.

### Example 82

### (E)-N-(1-Methyl-3-(2-(thiophen-2-yl) vinyl)-1H-pyrrolo[2,3-b]pyridin-5-yl)acrylamide

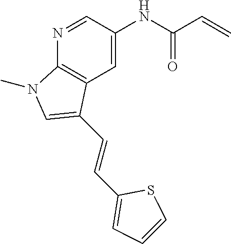 [SMILES: C=CC(=O)Nc1cnc2c(c1)c(/C=C/c1cccs1)cn2C]

The title compound was prepared from Intermediate E and (E)-2-thiophene boronic acid following the procedure outlined for Example 15. ¹H NMR (400 MHZ, DMSO-d6) & 10.29 (s, 1H), 8.65 (d, J=2.2 Hz, 1H), 8.49 (d, J=2.2 Hz, 1H), 7.79 (s, 1H), 7.36 (d, J=5.1 Hz, 1H), 7.08 (ddd, J=20.9, 15.4, 10.6 Hz, 4H), 6.47 (dd, J=17.0, 10.1 Hz, 1H), 6.28 (dd, J=17.0, 2.0 Hz, 1H), 5.78 (dd, J=10.1, 1.9 Hz, 1H), 3.79 (s, 3H). LCMS (ESI) m/z=310 (M+H)⁺.

### Example 83

### N-(1-Methyl-3-(2-phenyl-1-(trifluoromethyl) vinyl)-1H-pyrrolo[2,3-b]pyridin-5

The overall reaction scheme was as follows:

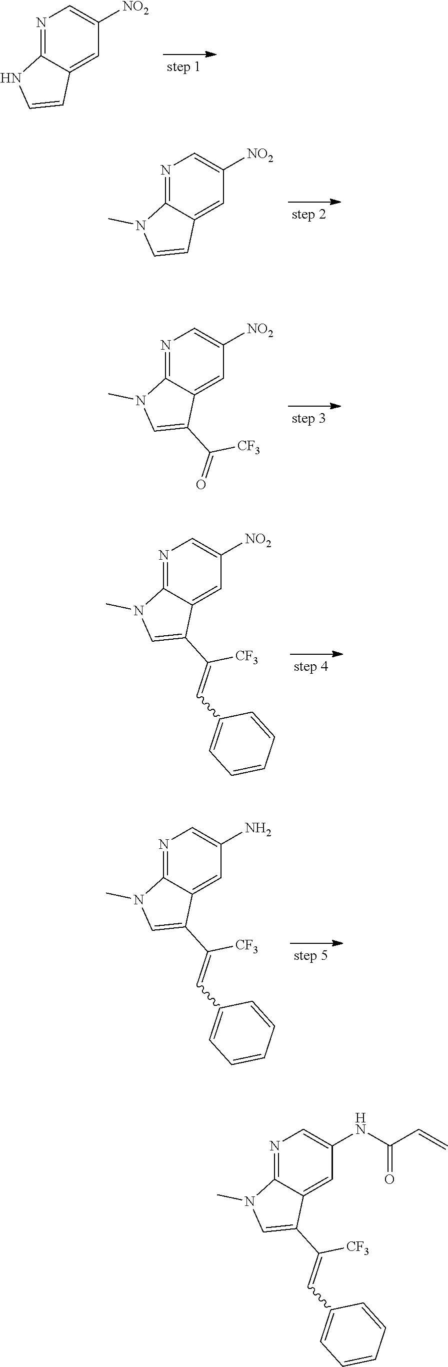 [SMILES: O=[N+]([O-])c1cnc2[nH]ccc2c1 ; Cn1ccc2cc([N+](=O)[O-])cnc21 ; Cn1cc(C(=O)C(F)(F)F)c2cc([N+](=O)[O-])cnc21 ; Cn1cc(C(=Cc2ccccc2)C(F)(F)F)c2cc([N+](=O)[O-])cnc21 ; Cn1cc(C(=Cc2ccccc2)C(F)(F)F)c2cc(N)cnc21 ; C=CC(=O)Nc1cnc2c(c1)c(C(=Cc1ccccc1)C(F)(F)F)cn2C]

### Step 1: 1-Methyl-5-nitro-1H-pyrrolo[2,3-b]pyridine

A mixture of 5-nitro-1H-pyrrolo[2,3-b]pyridine (1.0 g, 6.13 mmol) and Cs₂CO₃ (4.0 g, 12.3 mmol) were suspended in DMF (20 mL). Iodomethane (0.46 mL, 7.36 mmol) was added. The reaction mixture was stirred at r.t. for 5 h. The resultant mixture was diluted in EtOAc and washed with water 3 times then brine. The organic layer was dried over anhydrous Na₂SO₄, filtered, and concentrated. The residue was purified by flash chromatography (0%-30% EtOAc/Hex) to obtain the titled compound (961 mg, 88%). ¹H NMR (600 MHZ, CDCl₃) δ 9.24 (d, J=2.3 Hz, 1H), 8.76 (d, J=2.3 Hz, 1H), 7.36 (d, J=3.5 Hz, 1H), 6.67 (d, J=3.5 Hz, 1H), 3.96 (s, 3H). LCMS (ESI) m/z=178 (M+H)⁺.

### Step 2: 2,2,2-Trifluoro-1-(1-methyl-5-nitro-1H-pyrrolo[2,3-b]pyridin-3-yl)ethan-1-one

1-Methyl-5-nitro-1H-pyrrolo[2,3-b]pyridine (961 mg, 5.42 mmol) was diluted in DMF (10 mL). TFA (2.0 mL, 14.4 mmol) was added dropwise. The reaction mixture was stirred at 40° C. for 18 h then poured into ice. The resultant mixture was extracted with EtOAc three times. The organic layer was dried over Na₂SO₄, filtered and concentrated. The residue was purified by flash chromatography to obtain the titled compound (1.4 g, 95%). ¹H NMR (400 MHZ, CDCl₃) δ 9.44 (d, J=2.5 Hz, 1H), 9.36 (d, J=2.5 Hz, 1H), 8.25-8.20 (m, 2H), 4.09 (s, 3H). LCMS (ESI) m/z=274 (M+H)⁺.

### Step 3: 1-Methyl-5-nitro-3-(2-phenyl-1-(trifluoromethyl) vinyl)-1H-pyrrolo[2,3-b]pyridine

Benzyltriphenylphosphonium chloride (342 mg, 0.879 mmol) and NaH (60 wt % in oil, 35 mg, 0.879 mmol) were diluted in THF (7 mL). 2,2,2-trifluoro-1-(1-methyl-5-nitro-1H-pyrrolo[2,3-b]pyridin-3-yl)ethan-1-one (1.00 eq, 200 mg, 0.732 mmol) was added and the reaction mixture was stirred at rt for 18 h. The reaction mixture was quenched by adding water and extracted with EtOAc twice. The collected organic layer was dried over Na₂SO₄, filtered, and concentrated. The residue was purified by flash chromatography to obtain the titled compound (201 mg, 79%). ¹H NMR (400 MHZ, CDCl₃) δ 9.26 (d, J=2.4 Hz, 1H), 8.21 (d, J=2.4 Hz, 1H), 7.57 (s, 1H), 7.55-7.51 (m, 1H), 7.28-7.23 (m, 1H), 7.23-7.18 (m, 4H), 4.08 (s, 3H). LCMS (ESI) m/z=348 (M+H)⁺.

### Step 4: 1-Methyl-3-(2-phenyl-1-(trifluoromethyl) vinyl)-1H-pyrrolo[2,3-b]pyridin-5-amine

1-Methyl-5-nitro-3-(2-phenyl-1-(trifluoromethyl) vinyl)-1H-pyrrolo[2,3-b]pyridine (100 mg, 0.287 mmol), iron (48 mg, 0.864 mmol), and HCl 3N solution (0.19 mL, 0.576 mmol) were suspended in EtOH (3 mL). The reaction mixture was stirred at 80° C. for 15 h then cooled to rt. Activated charcoal was added to the mixture and filtered to remove insolubles. The filtrate was concentrated to obtain the crude compound (85 mg) and underwent to the next reaction without further purifications. LCMS (ESI) m/z=318 (M+H)⁺.

### Step 5: N-(1-Methyl-3-(2-phenyl-1-(trifluoromethyl) vinyl)-1H-pyrrolo[2,3-b]pyridin-5-yl)acrylamide

The crude 1-methyl-3-(2-phenyl-1-(trifluoromethyl) vinyl)-1H-pyrrolo[2,3-b]pyridin-5-amine (85 mg, 0.268 mmol) and TEA (0.11 mL, 0.804 mmol) were diluted in DCM (3 mL). Acryloyl chloride (0.019 mL, 0.241 mmol) was added at 0° C. and stirred for 1 h then concentrated. The residue was purified by flash chromatography (0-20% EtOAc/DCM) to obtain the titled compound (28 mg, 28%). ¹H NMR (400 MHZ, CD₃OD) δ 8.49 (d, J=2.1 Hz, 1H), 7.81 (d, J=2.2 Hz, 1H), 7.51 (s, 1H), 7.44 (s, 1H), 7.20-7.09 (m, 6H), 6.37 (dd, J=17.2, 9.4 Hz, 1H), 6.30 (, J=16.9, 2.3 Hz, 2H), 5.74 (dd, J=9.5, 2.2 Hz, 1H), 3.91 (s, 3H). LCMS (ESI) m/z=372 (M+H)⁺.

### Example 84

### N-(1-Methyl-3-(1-trifluoromethyl-2-phenylethan-1-yl)-1H-pyrrolo[2,3-b]pyridin-5-yl)acrylamide

The overall reaction scheme was as follows:

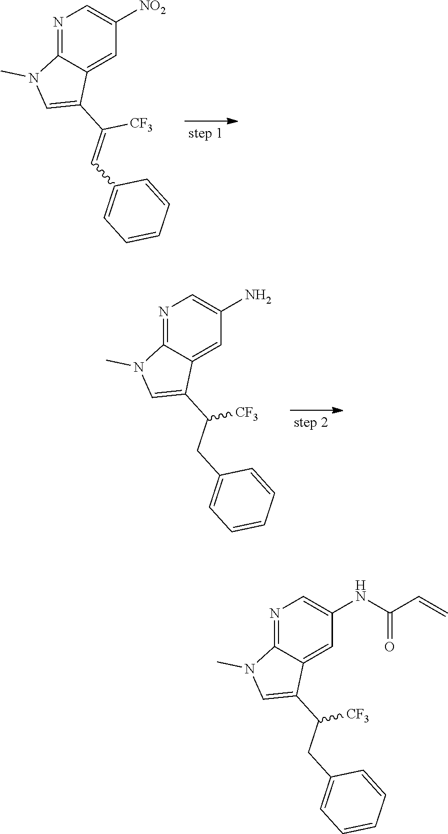 [SMILES: Cn1cc(C(=Cc2ccccc2)C(F)(F)F)c2cc([N+](=O)[O-])cnc21 ; Cn1cc(C(Cc2ccccc2)C(F)(F)F)c2cc(N)cnc21 ; C=CC(=O)Nc1cnc2c(c1)c(C(Cc1ccccc1)C(F)(F)F)cn2C]

### Step 1: 1-Methyl 3-(1-trifluoromethyl-2-phenylethan-1-yl)-1H-pyrrolo[2,3-b]pyridin-5-amine

1-Methyl-5-nitro-3-(2-phenyl-1-(trifluoromethyl) vinyl)-1H-pyrrolo[2,3-b]pyridine (100 mg, 0.288 mmol) was diluted in EtOAc (3 mL). Pd/C (31 mg, 0.0288 mmol) was added and stirred under hydrogen atmosphere for 18 h. The insolubles removed by filtration and concentrated to obtain the crude titled compound (78 mg) and underwent to the next reaction without further purifications. LCMS (ESI) m/z=320 (M+H)⁺.

### Step 2: N-(1-Methyl-3-(1-trifluoromethyl-2-phenylethan-1-yl)-1H-pyrrolo[2,3-b]pyridin-5-yl)acrylamide

The crude 1-Methyl 3-(1-trifluoromethyl-2-phenylethan-1-yl)-1H-pyrrolo[2,3-b]pyridin-5-amine (78 mg, 0.244 mmol) and TEA (0.10 mL, 0.733 mmol) were diluted in DCM (3 mL). Acryloyl chloride (0.018 mL, 0.220 mmol) was added at 0° C. and stirred for 1 h then concentrated. The residue was purified by flash chromatography (0-20% EtOAc/DCM) to obtain the titled compound (49 mg, 54%). ¹H NMR (400 MHZ, CD₃OD) δ 8.40 (d, J=2.2 Hz, 1H), 8.36 (d, J=2.1 Hz, 1H), 7.34 (s, 1H), 7.13-7.02 (m, 5H), 6.47 (dd, J=16.9, 9.7 Hz, 1H), 6.38 (dd, J=17.0, 2.1 Hz, 1H), 5.80 (dd, J=9.7, 2.1 Hz, 1H), 4.02-3.94 (m, 1H), 3.77 (s, 3H), 3.42 (dd, J=13.5, 4.0 Hz, 1H), 3.25 (dd, J=13.6, 11.4 Hz, 1H). LCMS (ESI) m/z=374 (M+H)⁺.

### Example 85

### (E)-N-(3-(3-(2-(4,4-Difluorocyclohexyl)vinyl)phenyl)-1-methyl-1H-pyrrolo[2,3-b]pyridin-5-yl)acrylamide

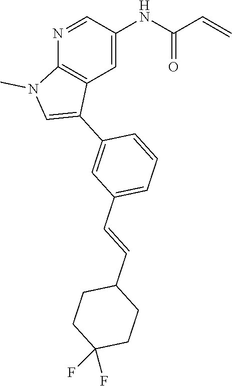 [SMILES: C=CC(=O)Nc1cnc2c(c1)c(-c1cccc(/C=C/C3CCC(F)(F)CC3)c1)cn2C]

The title compound was prepared from Intermediate E and 2-(3-(2-(4,4-difluorocyclohexyl)ethyl)phenyl)-4,4,5,5-tetramethyl-1,3,2-dioxaborolane following the procedure outlined for Example 1. ¹H NMR (600 MHZ, CD₃OD) δ 8.73 (d, J=2.3 Hz, 1H), 8.45 (d, J=2.3 Hz, 1H), 7.67 (s, 1H), 7.63 (t, J=1.6 Hz, 1H), 7.51-7.46 (m, 1H), 7.35 (t, J=7.6 Hz, 1H), 7.28 (d, J=7.8 Hz, 1H), 6.55-6.37 (m, 3H), 6.31 (dd, J=16.0, 7.0 Hz, 1H), 5.80 (dd, J=10.0, 1.7 Hz, 1H), 3.90 (d, J=4.1 Hz, 3H), 2.32 (dd, J=7.2, 3.5 Hz, 1H), 2.08 (m, 2H), 1.95-1.76 (m, 4H), 1.64-1.51 (m, 2H). LCMS (ESI) m/z=422 (M+H)⁺.

### Example 86

### N-(3-(3-(2-(4,4-Difluorocyclohexyl)ethyl)phenyl)-1-methyl-1H-pyrrolo[2,3-b]pyridin-5-yl)acrylamide

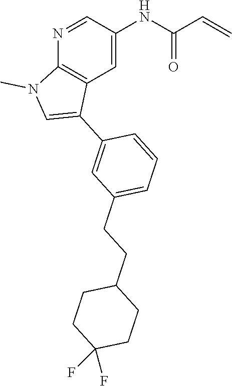 [SMILES: C=CC(=O)Nc1cnc2c(c1)c(-c1cccc(CCC3CCC(F)(F)CC3)c1)cn2C]

The title compound was prepared following the procedure outlined for Example 84 using a compound prepared according to the procedure outlined for step 2 of Example 85. ¹H NMR (600 MHz, CD₃OD) δ 8.72 (d, J=2.3 Hz, 1H), 8.43 (d, J=2.2 Hz, 1H), 7.64 (d, J=3.7 Hz, 1H), 7.48 (s, 1H), 7.46-7.44 (m, 1H), 7.33 (t, J=7.6 Hz, 1H), 7.10 (d, J=7.6 Hz, 1H), 6.43 (ddd, J=18.7, 17.0, 5.9 Hz, 2H), 5.80 (dd, J=10.1, 1.7 Hz, 1H), 3.90 (s, 3H), 2.74-2.70 (m, 2H), 2.04-2.01 (m, 2H), 1.89 (d, J=13.5 Hz, 2H), 1.80-1.69 (m, 2H), 1.65 (ddd, J=9.8, 8.8, 5.0 Hz, 2H), 1.44 (s, 1H), 1.34-1.26 (m, 2H). LCMS (ESI) m/z=424 (M+H)⁺.

### Example 87

### N-(3-(4-Chloro-3-fluorophenyl)-1-methyl-1H-pyrrolo[2,3-b]pyridin-5-yl)acrylamide

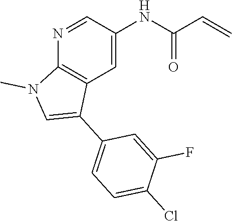 [SMILES: C=CC(=O)Nc1cnc2c(c1)c(-c1ccc(Cl)c(F)c1)cn2C]

The title compound was prepared from Intermediate E and (4-chloro-3-fluorophenyl) boronic acid following the procedure outlined for Example 15. ¹H NMR (600 MHZ, CD₃OD) δ 8.69 (d, J=2.2 Hz, 1H), 8.47 (d, J=2.3 Hz, 1H), 7.76 (s, 1H), 7.53-7.45 (m, 3H), 6.48 (dd, J=17.0, 10.0 Hz, 1H), 6.40 (dd, J=17.0, 1.8 Hz, 1H), 5.82 (dd, J=10.0, 1.8 Hz, 1H), 3.90 (s, 3H). LCMS (ESI) m/z=330 (M+H)⁺.

### Example 88

### N-(1-Methyl-3-(3-(piperidin-1-yl)phenyl)-1H-pyrrolo[2,3-b]pyridin-5-yl)acrylamide

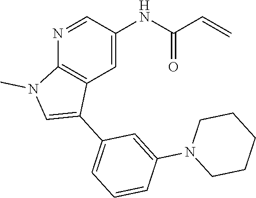 [SMILES: C=CC(=O)Nc1cnc2c(c1)c(-c1cccc(N3CCCCC3)c1)cn2C]

The title compound was prepared from Intermediate E and 1-[3-(4,4,5,5-tetramethyl-1,3,2-dioxaborolan-2-yl)phenyl]piperidine following the procedure outlined for Example 15. ¹H NMR (600 MHZ, CD₃OD) δ 8.69 (d, J=2.3 Hz, 1H), 8.49 (d, J=2.3 Hz, 1H), 7.65 (s, 1H), 7.32-7.29 (m, 1H), 7.25-7.24 (m, 1H), 7.15 (d, J=7.6 Hz, 1H), 6.93 (dd, J=8.2, 2.3 Hz, 1H), 6.48 (dd, J=17.0, 10.1 Hz, 1H), 6.39 (dd, J=17.0, 1.7 Hz, 1H), 5.81 (dd, J=10.1, 1.7 Hz, 1H), 3.91 (s, 3H), 3.25-3.20 (m, 4H), 1.79-1.75 (m, 4H), 1.65-1.61 (m, 2H). LCMS (ESI) m/z=361 (M+H)⁺.

### Example 89

### N-(3-(4-Ethylphenyl)-1-methyl-1H-pyrrolo[2,3-b]pyridin-5-yl)acrylamide

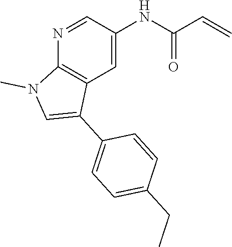 [SMILES: C=CC(=O)Nc1cnc2c(c1)c(-c1ccc(CC)cc1)cn2C]

The title compound was prepared from Intermediate E and (4-ethylphenyl) boronic acid following the procedure outlined for Example 15. ¹H NMR (600 MHZ, CDCl₃) δ 8.68 (d, J=2.3 Hz, 1H), 8.33 (d, J=2.3 Hz, 1H), 7.53 (d, J=8.1 Hz, 2H), 7.40 (s, 1H), 7.36 (s, 1H), 7.28 (s, 1H), 6.48 (dd, J=16.8, 1.1 Hz, 1H), 6.30 (dd, J=16.8, 10.2 Hz, 1H), 5.80 (dd, J=10.2, 1.0 Hz, 1H), 3.91 (s, 3H), 2.69 (q, J=7.6 Hz, 2H), 1.28 (t, J=7.6 Hz, 3H). LCMS (ESI) m/z=306 (M+H)⁺.

### Example 90

### N-(3-(4-Methoxyphenyl)-1-methyl-1H-pyrrolo[2,3-b]pyridin-5-yl)acrylamide

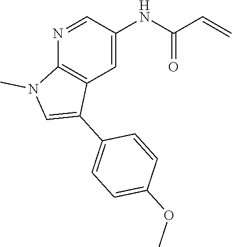 [SMILES: C=CC(=O)Nc1cnc2c(c1)c(-c1ccc(OC)cc1)cn2C]

The title compound was prepared from Intermediate E and (4-methoxyphenyl) boronic acid following the procedure outlined for Example 15. ¹H NMR (600 MHZ, CDCl₃) δ 8.68 (d, J=2.3 Hz, 1H), 8.31 (d, J=2.3 Hz, 1H), 7.56-7.52 (m, 2H), 7.38 (s, 1H), 7.32 (s, 1H), 7.01-6.97 (m, 2H), 6.48 (dd, J=16.8, 1.1 Hz, 1H), 6.30 (dd, J=16.8, 10.2 Hz, 1H), 5.81 (dd, J=10.2, 1.0 Hz, 1H), 3.91 (s, 3H), 3.85 (s, 3H). LCMS (ESI) m/z=308 (M+H)⁺.

### Example 91

### N-(3-(4-Acetylphenyl)-1-methyl-1H-pyrrolo[2,3-b]pyridin-5-yl)acrylamide

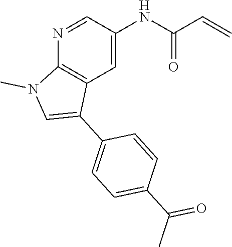 [SMILES: C=CC(=O)Nc1cnc2c(c1)c(-c1ccc(C(C)=O)cc1)cn2C]

The title compound was prepared from Intermediate E and (4-acetylphenyl) boronic acid following the procedure outlined for Example 15. ¹H NMR (600 MHZ, CD₃OD) δ 8.79 (d, J=2.3 Hz, 1H), 8.47 (d, J=2.2 Hz, 1H), 8.07-8.04 (m, 2H), 7.86 (s, 1H), 7.80 (d, J=8.4 Hz, 2H), 6.49 (dd, J=17.0, 10.0 Hz, 1H), 6.41 (dd, J=16.9, 1.8 Hz, 1H), 5.82 (dd, J=10.0, 1.8 Hz, 1H), 3.92 (s, 3H), 2.63 (s, 3H). LCMS (ESI) m/z=320 (M+H)⁺.

### Example 92

### N-(3-(4-Fluoro-3-(trifluoromethyl)phenyl)-1-methyl-1H-pyrrolo[2,3-b]pyridin-5-yl)acrylamide

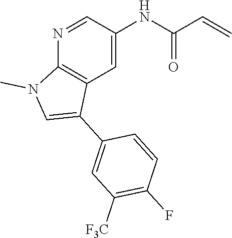 [SMILES: C=CC(=O)Nc1cnc2c(c1)c(-c1ccc(F)c(C(F)(F)F)c1)cn2C]

The title compound was prepared from Intermediate E and [4-fluoro-3-(trifluoromethyl)phenyl]boronic acid following the procedure outlined for Example 15. ¹H NMR (600 MHz, CD₃OD) δ 8.66 (d, J=2.2 Hz, 1H), 8.50 (d, J=2.2 Hz, 1H), 7.96-7.92 (m, 1H), 7.92-7.89 (m, 1H), 7.78 (s, 1H), 7.41 (dd, J=10.2, 8.8 Hz, 1H), 6.48 (dd, J=17.0, 10.0 Hz, 1H), 6.40 (dd, J=17.0, 1.8 Hz, 1H), 5.82 (dd, J=10.0, 1.8 Hz, 1H), 3.92 (s, 3H). LCMS (ESI) m/z=364 (M+H)⁺.

### Example 93

### N-(3-(4-Isopropylphenyl)-1-methyl-1H-pyrrolo[2,3-b]pyridin-5-yl)acrylamide

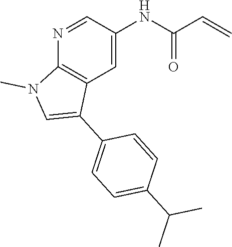 [SMILES: C=CC(=O)Nc1cnc2c(c1)c(-c1ccc(C(C)C)cc1)cn2C]

The title compound was prepared from Intermediate E and (4-isopropylphenyl) boronic acid following the procedure outlined for Example 15. ¹H NMR (600 MHZ, CD₃OD) δ 8.70 (d, J=2.3 Hz, 1H), 8.45 (d, J=2.2 Hz, 1H), 7.62 (s, 1H), 7.59-7.56 (m, 2H), 7.32-7.28 (m, 2H), 6.48 (dd, J=17.0, 10.1 Hz, 1H), 6.39 (dd, J=17.0, 1.7 Hz, 1H), 5.81 (dd, J=10.1, 1.7 Hz, 1H), 3.90 (s, 3H), 2.96-2.91 (m, 1H), 1.30 (s, 3H), 1.28 (s, 3H). LCMS (ESI) m/z=320 (M+H)⁺.

### Example 94

### N-(1-Methyl-3-(3-phenoxyphenyl)-1H-pyrrolo[2,3-b]pyridin-5-yl)acrylamide

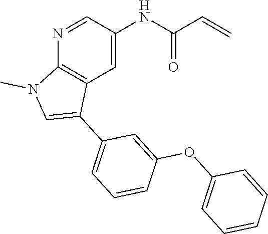 [SMILES: C=CC(=O)Nc1cnc2c(c1)c(-c1cccc(Oc3ccccc3)c1)cn2C]

The title compound was prepared from Intermediate E and (3-phenoxyphenyl) boronic acid following the procedure outlined for Example 15. ¹H NMR (600 MHz, CD₃OD) δ 8.66 (d, J=2.3 Hz, 1H), 8.46 (d, J=2.1 Hz, 1H), 7.67 (d, J=4.0 Hz, 1H), 7.44-7.39 (m, 2H), 7.39-7.35 (m, 2H), 7.29 (d, J=1.5 Hz, 1H), 7.11 (m, 1H), 7.07-7.04 (m, 2H), 6.89-6.85 (m, 1H), 6.48 (dd, J=17.0, 10.0 Hz, 1H), 6.40 (dd, J=17.0, 1.7 Hz, 1H), 5.81 (dd, J=10.1, 1.7 Hz, 1H), 3.90 (s, 3H). LCMS (ESI) m/z=370 (M+H)⁺.

### Example 95

### N-(1-Ethyl-3-(4-(trifluoromethyl)phenyl)-1H-pyrrolo[2,3-b]pyridin-5-yl)acrylamide

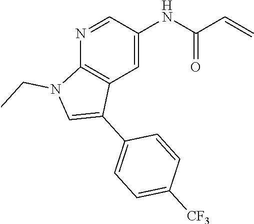 [SMILES: C=CC(=O)Nc1cnc2c(c1)c(-c1ccc(C(F)(F)F)cc1)cn2CC]

The title compound was prepared from Intermediate F and [4-(trifluoromethyl)phenyl]boronic acid following the procedure outlined for Example 15. ¹H NMR (600 MHz, CD₃OD) δ 8.76 (dd, J=2.3, 0.9 Hz, 1H), 8.47 (d, J=2.2 Hz, 1H), 7.91 (s, 1H), 7.86 (d, J=8.4 Hz, 2H), 7.73-7.71 (m, 2H), 6.49 (dd, J=17.0, 10.0 Hz, 1H), 6.41 (dd, J=17.0, 1.7 Hz, 1H), 5.82 (dd, J=10.0, 1.7 Hz, 1H), 4.39 (q, J=7.3 Hz, 2H), 1.51 (t, J=7.3 Hz, 3H). LCMS (ESI) m/z=360 (M+H)⁺.

### Example 96

### N-(1-isopropyl-3-(4-(trifluoromethyl)phenyl)-1H-pyrrolo[2,3-b]pyridin-5-yl)acrylamide

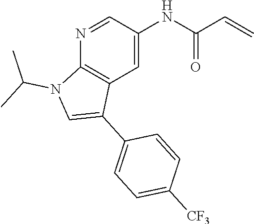 [SMILES: C=CC(=O)Nc1cnc2c(c1)c(-c1ccc(C(F)(F)F)cc1)cn2C(C)C]

The title compound was prepared from Intermediate G and [4-(trifluoromethyl)phenyl]boronic acid following the procedure outlined for Example 15. ¹H NMR (600 MHz, CD₃OD) δ 8.77 (d, J=2.3 Hz, 1H), 8.47 (d, J=2.3 Hz, 1H), 8.01 (s, 1H), 7.88 (d, J=8.1 Hz, 2H), 7.73 (d, J=8.2 Hz, 2H), 6.49 (dd, J=17.0, 10.0 Hz, 1H), 6.41 (dd, J=17.0, 1.7 Hz, 1H), 5.82 (dd, J=10.1, 1.7 Hz, 1H), 5.19-5.14 (m, 1H), 1.60 (s, 3H), 1.59 (s, 3H). LCMS (ESI) m/z=374 (M+H)⁺.

### Example 97

### N-(3-(3-Chloro-4-fluorophenyl)-1-methyl-1H-pyrrolo[2,3-b]pyridin-5-yl)acrylamide

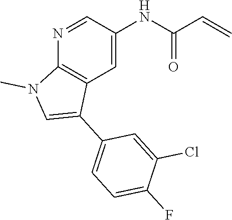 [SMILES: C=CC(=O)Nc1cnc2c(c1)c(-c1ccc(F)c(Cl)c1)cn2C]

The title compound was prepared from Intermediate E and (3-chloro-4-fluorophenyl) boronic acid following the procedure outlined for Example 15. ¹H NMR (600 MHz, CD₃OD) δ 8.66 (d, J=2.2 Hz, 1H), 8.49 (d, J=2.3 Hz, 1H), 7.74 (dd, J=7.1, 2.2 Hz, 1H), 7.71 (s, 1H), 7.61 (m, 1H), 7.34-7.29 (m, 1H), 6.52-6.39 (m, 2H), 5.81 (dd, J=10.1, 1.7 Hz, 1H), 3.91 (d, J=4.5 Hz, 3H). LCMS (ESI) m/z=330 (M+H)⁺.

### Example 98

### N-(3-(4-Chlorophenyl)-1-methyl-1H-pyrrolo[2,3-b]pyridin-5-yl)acrylamide

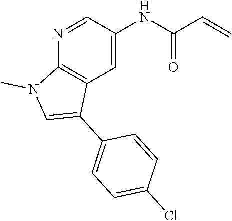 [SMILES: C=CC(=O)Nc1cnc2c(c1)c(-c1ccc(Cl)cc1)cn2C]

The title compound was prepared from Intermediate E and (4-chlorophenyl) boronic acid following the procedure outlined for Example 15. ¹H NMR (600 MHZ, CD₃OD) δ 8.69 (d, J=1.9 Hz, 1H), 8.46 (d, J=2.0 Hz, 1H), 7.71 (s, 1H), 7.65 (d, J=8.3 Hz, 2H), 7.43 (d, J=8.3 Hz, 2H), 6.51-6.36 (m, 2H), 5.81 (d, J=10.1 Hz, 1H), 3.91 (s, 3H). LCMS (ESI) m/z=312 (M+H)⁺.

### Example 99

### N-(3-(4,4-Difluorocyclohex-1-en-1-yl)-1-ethyl-1H-pyrrolo[2,3-b]pyridin-5-yl)acrylamide

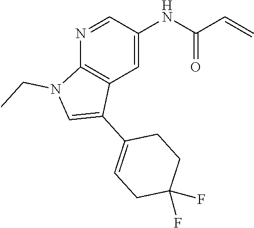 [SMILES: C=CC(=O)Nc1cnc2c(c1)c(C1=CCC(F)(F)CC1)cn2CC]

The title compound was prepared from Intermediate F and 2-(4,4-difluorocyclohexen-1-yl)-4,4,5,5-tetramethyl-1,3,2-dioxaborolane following the procedure outlined for Example 15. ¹H NMR (600 MHZ, CD₃OD) δ 8.65 (d, J=2.2 Hz, 1H), 8.40 (d, J=2.2 Hz, 1H), 7.53 (s, 1H), 6.48 (dd, J=17.0, 10.1 Hz, 1H), 6.39 (dd, J=17.0, 1.7 Hz, 1H), 6.05 (br s, 1H), 5.81 (dd, J=10.1, 1.7 Hz, 1H), 4.30 (q, J=7.2 Hz, 2H), 2.80-2.71 (m, 4H), 2.24-2.17 (m, 2H), 1.44 (t, J=7.3 Hz, 3H). LCMS (ESI) m/z=332 (M+H)⁺.

### Example 100

### N-(1-Methyl-3-(4-propylphenyl)-1H-pyrrolo[2,3-b]pyridin-5-yl)acrylamide

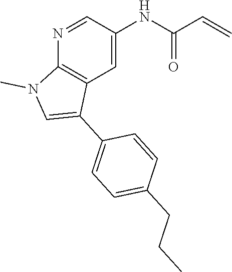 [SMILES: C=CC(=O)Nc1cnc2c(c1)c(-c1ccc(CCC)cc1)cn2C]

The title compound was prepared from Intermediate E and (4-propylphenyl) boronic acid following the procedure outlined for Example 15. ¹H NMR (600 MHZ, CD₃OD) δ 8.70 (d, J=2.2 Hz, 1H), 8.45 (d, J=2.1 Hz, 1H), 7.62 (s, 1H), 7.56 (d, J=8.0 Hz, 2H), 7.25 (d, J=8.0 Hz, 2H), 6.48 (dd, J=17.0, 10.1 Hz, 1H), 6.39 (dd, J=17.0, 1.5 Hz, 1H), 5.81 (dd, J=10.1, 1.5 Hz, 1H), 3.90 (s, 3H), 2.62 (t, J=7.6 Hz, 2H), 1.71-1.64 (m, 2H), 0.97 (t, J=7.3 Hz, 3H). LCMS (ESI) m/z=320 (M+H)⁺.

### Example 101

### N-(3-(Cyclohex-1-en-1-yl)-1-methyl-1H-pyrrolo[2,3-b]pyridin-5-yl)acrylamide

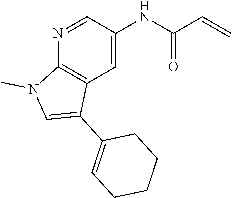 [SMILES: C=CC(=O)Nc1cnc2c(c1)c(C1=CCCCC1)cn2C]

The title compound was prepared from Intermediate E and 2-(cyclohexen-1-yl)-4,4,5,5-tetramethyl-1,3,2-dioxaborolane following the procedure outlined for Example 15. ¹H NMR (600 MHz, CD₃OD) δ 8.64 (d, J=2.2 Hz, 1H), 8.40 (d, J=2.2 Hz, 1H), 7.35 (s, 1H), 6.47 (dd, J=17.0, 10.1 Hz, 1H), 6.39 (dd, J=17.0, 1.6 Hz, 1H), 6.21 (t, J=3.9 Hz, 1H), 5.80 (dd, J=10.1, 1.6 Hz, 1H), 3.81 (s, 3H), 2.46-2.42 (m, 2H), 2.29-2.24 (m, 2H), 1.85-1.80 (m, 2H), 1.74-1.68 (m, 2H). LCMS (ESI) m/z=292 (M+H)⁺.

### Example 102

### N-(1-Methyl-3-(4-(piperidin-1-yl)phenyl)-1H-pyrrolo[2,3-b]pyridin-5-yl)acrylamide

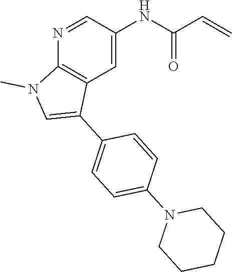 [SMILES: C=CC(=O)Nc1cnc2c(c1)c(-c1ccc(N3CCCCC3)cc1)cn2C]

The title compound was prepared from Intermediate E and 1-[4-(4,4,5,5-tetramethyl-1,3,2-dioxaborolan-2-yl)phenyl]piperidine following the procedure outlined for Example 15. ¹H NMR (600 MHZ, CD₃OD) δ 8.67 (d, J=2.2 Hz, 1H), 8.44 (d, J=2.1 Hz, 1H), 7.56-7.52 (m, 3H), 7.08 (d, J=8.6 Hz, 2H), 6.48 (dd, J=17.0, 10.1 Hz, 1H), 6.39 (dd, J=16.9, 1.3 Hz, 1H), 5.81 (dd, J=10.1, 1.2 Hz, 1H), 3.89 (s, 3H), 3.19-3.15 (m, 4H), 1.77-1.73 (m, J=11.0, 5.7 Hz, 4H), 1.64-1.59 (m, J=11.7, 6.0 Hz, 2H). LCMS (ESI) m/z=361 (M+H)⁺

### Example 103

### N-(3-(4-Fluorophenyl)-1-methyl-1H-pyrrolo[2,3-b]pyridin-5-yl)acrylamide

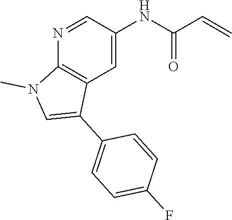 [SMILES: C=CC(=O)Nc1cnc2c(c1)c(-c1ccc(F)cc1)cn2C]

The title compound was prepared from Intermediate E and (4-fluorophenyl) boronic acid following the procedure outlined for Example 15. ¹H NMR (600 MHZ, CD₃OD) δ 8.67 (d, J=2.2 Hz, 1H), 8.45 (d, J=2.2 Hz, 1H), 7.67-7.63 (m, 3H), 7.19-7.15 (m, 2H), 6.48 (dd, J=17.0, 10.1 Hz, 1H), 6.40 (dd, J=17.0, 1.7 Hz, 1H), 5.81 (dd, J=10.1, 1.7 Hz, 1H), 3.91 (s, 3H). LCMS (ESI) m/z=296 (M+H)⁺.

### Example 104

### N-(1-Methyl-3-(p-tolyl)-1H-pyrrolo[2,3-b]pyridin-5-yl)acrylamide

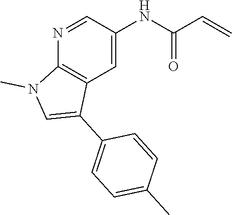 [SMILES: C=CC(=O)Nc1cnc2c(c1)c(-c1ccc(C)cc1)cn2C]

The title compound was prepared from Intermediate E and p-tolylboronic acid following the procedure outlined for Example 15. ¹H NMR (600 MHZ, CD₃OD) δ 8.68 (d, J=2.2 Hz, 1H), 8.45 (d, J=2.2 Hz, 1H), 7.62 (s, 1H), 7.54 (d, J=8.0 Hz, 2H), 7.25 (d, J=7.9 Hz, 2H), 6.48 (dd, J=17.0, 10.1 Hz, 1H), 6.39 (dd, J=17.0, 1.5 Hz, 1H), 5.81 (dd, J=10.1, 1.5 Hz, 1H), 3.90 (s, 3H), 2.37 (s, 3H). LCMS (ESI) m/z=292 (M+H)⁺.

### Example 105

### N-(3-(4′-Chloro-[1,1′-biphenyl]-4-yl)-1-methyl-1H-pyrrolo[2,3-b]pyridin-5-yl)acrylamide

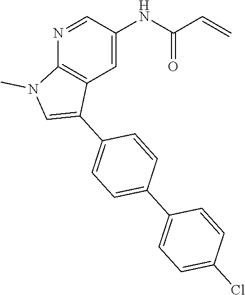 [SMILES: C=CC(=O)Nc1cnc2c(c1)c(-c1ccc(-c3ccc(Cl)cc3)cc1)cn2C]

The title compound was prepared from Intermediate E and [4-(4-chlorophenyl)phenyl]boronic acid following the procedure outlined for Example 15. ¹H NMR (600 MHz, CD₃OD) δ 8.77 (d, J=2.2 Hz, 1H), 8.46 (d, J=2.2 Hz, 1H), 7.77 (s, 1H), 7.75 (d, J=4.2 Hz, 2H), 7.70 (d, J=8.3 Hz, 2H), 7.66 (d, J=8.5 Hz, 2H), 7.45 (d, J=8.5 Hz, 2H), 6.49 (dd, J=17.0, 10.1 Hz, 1H), 6.40 (dd, J=17.0, 1.6 Hz, 1H), 5.81 (dd, J=10.1, 1.5 Hz, 1H), 3.92 (s, 3H). LCMS (ESI) m/z=388 (M+H)⁺.

### Example 106

### N-(3-(4-Cyclopropylphenyl)-1-methyl-1H-pyrrolo[2,3-b]pyridin-5-yl)acrylamide

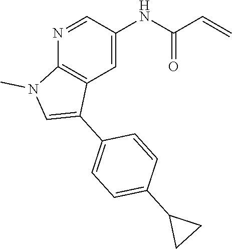 [SMILES: C=CC(=O)Nc1cnc2c(c1)c(-c1ccc(C3CC3)cc1)cn2C]

The title compound was prepared from Intermediate E and (4-cyclopropylphenyl) boronic acid following the procedure outlined for Example 15. ¹H NMR (600 MHz, CD₃OD) δ 8.68 (d, J=2.2 Hz, 1H), 8.45 (d, J=2.1 Hz, 1H), 7.62 (s, 1H), 7.54 (d, J=8.1 Hz, 2H), 7.15 (d, J=8.1 Hz, 2H), 6.48 (dd, J=17.0, 10.1 Hz, 1H), 6.39 (dd, J=17.0, 1.6 Hz, 1H), 5.81 (dd, J=10.1, 1.5 Hz, 1H), 3.90 (s, 3H), 1.97-1.91 (m, 1H), 1.00-0.96 (m, 2H), 0.73-0.69 (m, 2H). LCMS (ESI) m/z=318 (M+H)⁺.

### Example 107

### N-(1-Methyl-3-(naphthalen-2-yl)-1H-pyrrolo[2,3-b]pyridin-5-yl)acrylamide

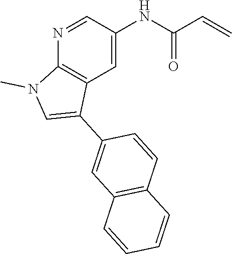 [SMILES: C=CC(=O)Nc1cnc2c(c1)c(-c1ccc3ccccc3c1)cn2C]

The title compound was prepared from Intermediate E and 2-naphthaleneboronic acid following the procedure outlined for Example 15. ¹H NMR (400 MHZ, CDCl₃) δ 8.82 (s, 1H), 8.44 (s, 1H), 8.05 (s, 1H), 7.94-7.86 (m, 2H), 7.85 (d, J=7.4 Hz, 1H), 7.79-7.72 (m, 1H), 7.54-7.41 (m, 3H), 6.50 (dd, J=16.6, 0.9 Hz, 1H), 6.34 (dd, J=16.7, 10.2 Hz, 1H), 5.82 (d, J=9.9 Hz, 1H), 3.98 (s, 3H). LCMS (ESI) m/z=328 (M+H)⁺.

### Example 108

### N-(3-(4-Cyclohexylphenyl)-1-methyl-1H-pyrrolo[2,3-b]pyridin-5-yl)acrylamide

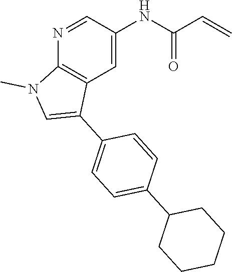 [SMILES: C=CC(=O)Nc1cnc2c(c1)c(-c1ccc(C3CCCCC3)cc1)cn2C]

The title compound was prepared from Intermediate E and (4-cyclohexylphenyl) boronic acid following the procedure outlined for Example 15. ¹H NMR (400 MHZ, CD₃OD) δ 8.68 (d, J=2.2 Hz, 1H), 8.44 (d, J=2.2 Hz, 1H), 7.60 (s, 1H), 7.55 (d, J=8.2 Hz, 2H), 7.27 (d, J=8.1 Hz, 2H), 6.48 (dd, J=17.0, 9.8 Hz, 1H), 6.38 (dd, J=17.0, 2.0 Hz, 1H), 5.80 (dd, J=9.8, 2.0 Hz, 1H), 3.89 (s, 3H), 2.53 (s, 1H), 1.88 (s, 4H), 1.77 (d, J=12.3 Hz, 1H), 1.56-1.38 (m, 5H). LCMS (ESI) m/z=360 (M+H)⁺.

### Example 109

### N-(3-(4-(tert-Butyl)phenyl)-1-methyl-1H-pyrrolo[2,3-b]pyridin-5-yl)acrylamide

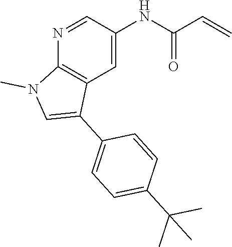 [SMILES: C=CC(=O)Nc1cnc2c(c1)c(-c1ccc(C(C)(C)C)cc1)cn2C]

The title compound was prepared from 3-bromo-1-methyl-1H-pyrrolo[2,3-b]pyridin-5-amine and (4-tert-butylphenyl) boronic acid following the procedure outlined for Example 15, step 1 and step 3. ¹H NMR (400 MHZ, CD₃OD) δ 8.71 (d, J=2.3 Hz, 1H), 8.44 (d, J=2.1 Hz, 1H), 7.63 (s, 1H), 7.60-7.56 (m, 2H), 7.47 (d, J=8.6 Hz, 2H), 6.48 (dd, J=16.9, 9.8 Hz, 1H), 6.39 (dd, J=16.9, 2.0 Hz, 1H), 5.80 (dd, J=9.9, 2.0 Hz, 1H), 3.90 (s, 3H), 1.36 (s, 9H). LCMS (ESI) m/z=334 (M+H)⁺.

### Example 110

### N-(3-(4-Butylphenyl)-1-methyl-1H-pyrrolo[2,3-b]pyridin-5-yl)acrylamide

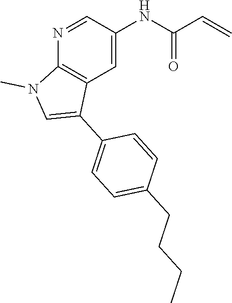 [SMILES: C=CC(=O)Nc1cnc2c(c1)c(-c1ccc(CCCC)cc1)cn2C]

The title compound was prepared from 3-bromo-1-methyl-1H-pyrrolo[2,3-b]pyridin-5-amine and (4-butylphenyl) boronic acid following the procedure outlined for Example 15, step 1 and step 3. ¹H NMR (600 MHZ, CD₃OD) δ 8.69 (d, J=2.3 Hz, 1H), 8.45 (d, J=2.3 Hz, 1H), 7.61 (s, 1H), 7.57-7.54 (m, 2H), 7.26-7.23 (m, 2H), 6.48 (dd, J=17.0, 10.1 Hz, 1H), 6.40-6.37 (m, 1H), 5.80 (dd, J=10.1, 1.7 Hz, 1H), 3.90 (s, 3H), 2.67-2.62 (m, 2H), 1.66-1.61 (m, 2H), 1.42-1.36 (m, 2H), 0.96 (t, J=7.4 Hz, 3H). LCMS (ESI) m/z=334 (M+H)⁺.

### Example 111

### N-(3-(4-(Ethylthio)phenyl)-1-methyl-1H-pyrrolo[2,3-b]pyridin-5-yl)acrylamide

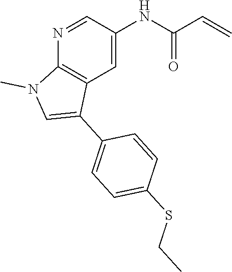 [SMILES: C=CC(=O)Nc1cnc2c(c1)c(-c1ccc(SCC)cc1)cn2C]

The title compound was prepared from Intermediate V and 4-ethylthiophenyl boronic acid following the procedure outlined for Example 1. ¹H NMR (400 MHZ, CD₃OD) δ 8.70 (d, J=2.2 Hz, 1H), 8.45 (d, J=2.3 Hz, 1H), 7.67 (s, 1H), 7.60 (d, J=8.5 Hz, 2H), 7.41 (d, J=8.5 Hz, 2H), 6.48 (dd, J=17.0, 9.9 Hz, 1H), 6.39 (dd, J=17.0, 2.0 Hz, 1H), 5.80 (dd, J=9.7, 2.0 Hz, 1H), 3.90 (s, 3H), 2.97 (q, J=7.3 Hz, 2H), 1.31 (t, J=7.3 Hz, 3H). LCMS (ESI) m/z=338 (M+H)⁺.

### Example 112

### N-(1-Methyl-3-(4-nonylphenyl)-1H-pyrrolo[2,3-b]pyridin-5-yl)acrylamide

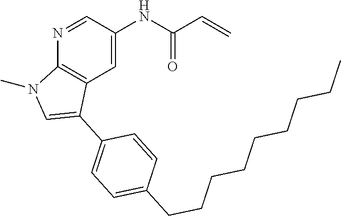 [SMILES: C=CC(=O)Nc1cnc2c(c1)c(-c1ccc(CCCCCCCCC)cc1)cn2C]

The title compound was prepared from Intermediate V and 4-nonylphenyl boronic acid following the procedure outlined for Example 1. ¹H NMR (600 MHZ, CD₃OD) δ 8.68 (s, 1H), 8.44 (s, 1H), 7.59 (d, J=2.9 Hz, 1H), 7.54 (dd, J=8.1, 2.8 Hz, 2H), 7.23 (dd, J=8.1, 2.8 Hz, 2H), 6.47 (ddd, J=17.0, 10.1, 2.9 Hz, 1H), 6.38 (d, J=17.0 Hz, 1H), 5.80 (d, J=10.0 Hz, 1H), 3.88 (s, 3H), 2.65-2.59 (m, 2H), 1.67-1.61 (m, 2H), 1.35 (d, J=6.5 Hz, 4H), 1.31-1.25 (m, 10H), 0.92-0.86 (m, 4H). LCMS (ESI) m/z=404 (M+H)⁺.

### Example 113

### N-(3-(4-Hydroxy-3,5-dimethylphenyl)-1-methyl-1H-pyrrolo[2,3-b]pyridin-5-yl)acrylamide

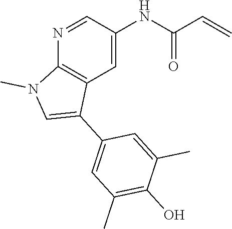 [SMILES: C=CC(=O)Nc1cnc2c(c1)c(-c1cc(C)c(O)c(C)c1)cn2C]

The title compound was prepared from Intermediate V and 3,5-dimethyl-4-hydroxylphenyl-boronic acid pinacol ester following the procedure outlined for Example 1. ¹H NMR (400 MHZ, CD₃OD) δ 8.64 (s, 1H), 8.56 (s, 1H), 7.53 (s, 1H), 7.21 (s, 2H), 6.48 (dd, J=17.0, 9.7 Hz, 1H), 6.43-6.36 (m, 1H), 5.84-5.78 (m, 1H), 3.90 (s, 3H), 2.29 (s, 6H). LCMS (ESI) m/z=322 (M+H)⁺.

### Example 114

### Preparation of N-(3-(4-((dimethylamino)methyl)phenyl)-1-methyl-1H-pyrrolo[2,3-b]pyridin-5-yl)acrylamide

The overall reaction scheme was as follows:

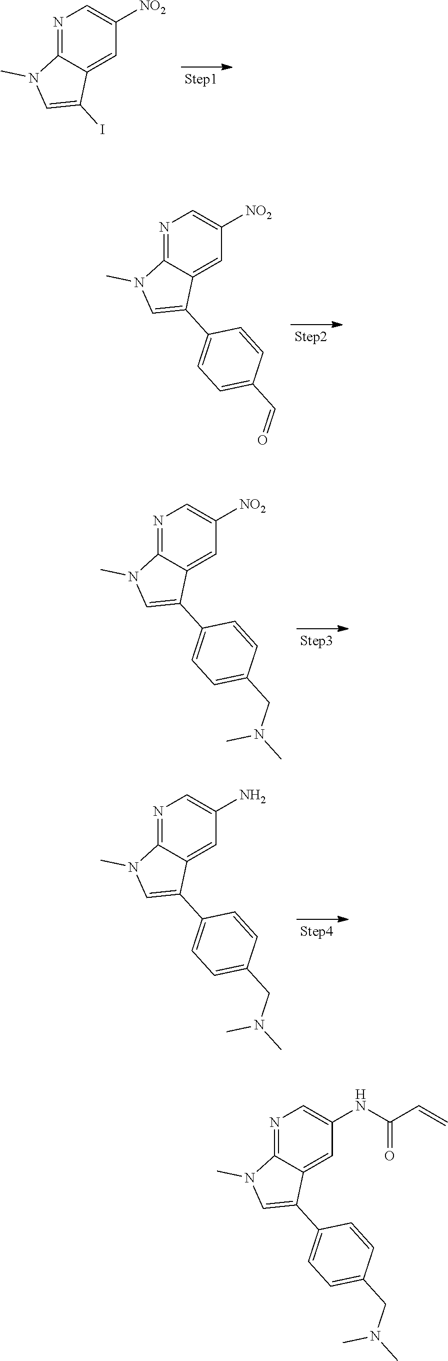

### Step 1: 4-(1-Methyl-5-nitro-1H-pyrrolo[2,3-b]pyridin-3-yl)benzaldehyde

The mixture of 3-iodo-1-methyl-5-nitro-1H-pyrrolo[2,3-b]pyridine (200 mg, 0.660 mmol), 4-formylphenylboronic acid (166 mg, 0.990 mmol), Na₂CO₃ (210 mg, 1.98 mmol) in a mixed solution of dioxane/water (5:1, 6 mL) was degassed and heated at 120° C. in a microwave-oven synthesizer for 2 h. The mixture was cooled to r.t. and filtered through a Celite pad to remove insolubles then rinsed with EtOAc. The solvent was removed and the crude was purified by silica gel chromatography (0%-20% EA/DCM) to obtain the titled compound (110 mg, 59%). ¹H NMR (400 MHZ, CDCl₃) δ 10.07 (s, 1H), 9.31 (d, J=2.4 Hz, 1H), 9.07 (d, J=2.3 Hz, 1H), 8.01 (d, J=8.2 Hz, 2H), 7.80 (d, J=8.1 Hz, 2H), 7.70 (s, 1H), 4.05 (s, 3H). LCMS (ESI) m/z=282 (M+H)⁺.

### Step 2: N,N-Dimethyl-1-(4-(1-methyl-5-nitro-1H-pyrrolo[2,3-b]pyridin-3-yl)phenyl) methanamine

A mixture of 4-(1-methyl-5-nitro-1H-pyrrolo[2,3-b]pyridin-3-yl)benzaldehyde (110 mg, 0.391 mmol) and dimethylamine hydrochloride (41 mg, 0.508 mmol) were suspended in DCM (4 mL). DIPEA (0.20 mL, 1.17 mmol) was added and stirred for 1 h. After then, NaBH₃CN (37 mg, 0.587 mmol) was added. The reaction mixture was stirred at r.t. for 18 h then quenched by adding water. The resultant mixture was extracted with DCM 3 times then concentrated. The residue was purified by reverse flash chromatography with TFA adductive (0.1%) to obtain semi-TFA salt of the titled compound (175 mg, 96%). The semi-TFA salt was underwent to the next reaction without further neutralization. LCMS (ESI) m/z=311 (M+H)⁺.

### Step 3: 3-(4-((Dimethylamino)methyl)phenyl)-1-methyl-1H-pyrrolo[2,3-b]pyridin-5-amine

To a solution of N,N-dimethyl-1-(4-(1-methyl-5-nitro-1H-pyrrolo[2,3-b]pyridin-3-yl)phenyl) methanamine (58 mg, 0.187 mmol) in EtOAc (2 mL), Pd/C (20 mg, 0.0187 mmol) was added and stirred under hydrogen atmosphere for 18 h. The insolubles was removed by filtration and concentrated to obtain the titled compound (52 mg, 99%). ¹H NMR (400 MHZ, CDCl₃) δ 7.97 (d, J=2.5 Hz, 1H), 7.60-7.52 (m, 3H), 7.38 (d, J=8.0 Hz, 2H), 7.32 (s, 1H), 3.88 (s, 3H), 3.54 (s, 2H), 2.34 (s, 6H). LCMS (ESI) m/z=281 (M+H)⁺.

### Step 4: N-(3-(4-((Dimethylamino)methyl)phenyl)-1-methyl-1H-pyrrolo[2,3-b]pyridin-5-yl)acrylamide

A mixture of 3-(4-((dimethylamino)methyl)phenyl)-1-methyl-1H-pyrrolo[2,3-b]pyridin-5-amine (52 mg, 0.185 mmol) and TEA (0.052 mL, 0.371 mmol) were diluted in DCM (2 mL). Acryloyl chloride (0.015 mL, 0.185 mmol) was added at 0° C. and stirred for 1 h then concentrated. The residue was purified by prep HPLC to obtain titled compound (24 mg, 39%). ¹H NMR (600 MHZ, CD₃OD) δ 8.88 (s, 1H), 8.38 (s, 1H), 7.85-7.78 (m, 3H), 7.59-7.54 (m, 2H), 6.49 (dd, J=17.0, 10.1 Hz, 1H), 6.40 (d, J=17.6 Hz, 1H), 5.82 (d, J=9.5 Hz, 1H), 4.35 (s, 2H), 3.93 (s, 3H), 2.90 (s, 6H). LCMS (ESI) m/z=335 (M+H)⁺.

### Example 115

### Preparation of (E)-N-(3-(4-chlorostyryl)-1-methyl-1H-pyrrolo[2,3-b]pyridin-5-yl)-2-cyanoacetamide

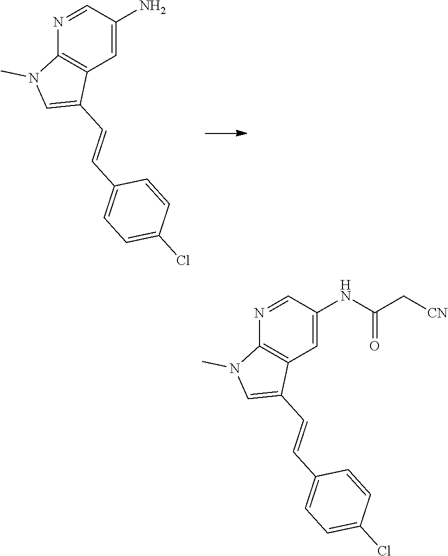 [SMILES: Cn1cc(/C=C/c2ccc(Cl)cc2)c2cc(N)cnc21 ; Cn1cc(/C=C/c2ccc(Cl)cc2)c2cc(NC(=O)CC#N)cnc21]

A mixture of the intermediate prepared according to the procedure outlined for step 2 of Example 16 (50 mg, 0.18 mmol), 2-cyanoacetic acid (22 mg, 0.26 mmol), DIPEA (0.11 mL, 0.62 mmol) and DMAP (2.2 mg, 0.02 mmol) were suspended in DCM (3.52 mL). EDCI (47 mg, 0.30 mmol) was added. The reaction mixture was stirred at r.t. for 16 h. The reaction was quenched by adding water and extracted with DCM. The collected organic layer was dried over anhydrous Na₂SO₄, filtered, and concentrated. The residue was purified by column chromatography to obtain the titled compound (40 mg, 65%). ¹H NMR (600 MHZ, CDCl₃) δ 8.59 (s, 1H), 8.36 (s, 1H), 7.45-7.41 (m, 2H), 7.40 (s, 1H), 7.34-7.29 (m, 2H), 7.16 (d, J=16.4 Hz, 1H), 6.97 (d, J=16.4 Hz, 1H), 3.95-3.87 (m, 3H), 3.66 (s, 2H). LCMS (ESI) m/z=351 (M+H)⁺.

### Example 116

### (E)-N-(3-(4-Chlorostyryl)-1-methyl-1H-pyrrolo[2,3-b]pyridin-5-yl) ethenesulfonamide

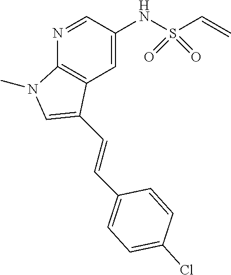 [SMILES: C=CS(=O)(=O)Nc1cnc2c(c1)c(/C=C/c1ccc(Cl)cc1)cn2C]

The title compound was prepared according to the procedure of Example 16 using ethenesulfonyl chloride in step 3. ¹H NMR (600 MHZ, CDCl₃) δ 8.19 (d, J=2.4 Hz, 2H), 7.45-7.41 (m, 2H), 7.40 (s, 1H), 7.34-7.30 (m, 2H), 7.15 (d, J=16.4 Hz, 1H), 6.96 (d, J=16.4 Hz, 1H), 6.60 (dd, J=16.5, 9.9 Hz, 1H), 6.40 (s, 1H), 6.19 (d, J=16.5 Hz, 1H), 5.96 (d, J=9.9 Hz, 1H), 3.89 (s, 3H). LCMS (ESI) m/z=374 (M+H)⁺.

### Example 117

### (E)-N-(3-(4-Chlorostyryl)-1-methyl-1H-pyrrolo[2,3-b]pyridin-5-yl) methanesulfonamide

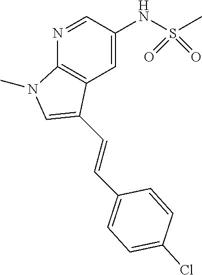

The title compound was prepared according to the procedure of Example 16 using methanesulfonyl chloride in step 3. ¹H NMR (600 MHZ, CDCl₃) δ 8.25 (dd, J=6.5, 2.3 Hz, 1H), 7.43 (d, J=2.5 Hz, 2H), 7.34-7.31 (m, 2H), 7.16 (d, J=16.4 Hz, 1H), 6.97 (d, J=16.4 Hz, 1H), 6.32 (s, 1H), 3.90 (s, 3H), 3.02 (s, 3H). LCMS (ESI) m/z=362 (M+H)⁺.

### Example 118

### (E)-N-(3-(4-Chlorostyryl)-1-methyl-1H-pyrrolo[2,3-b]pyridin-5-yl)-2-fluoroacrylamide

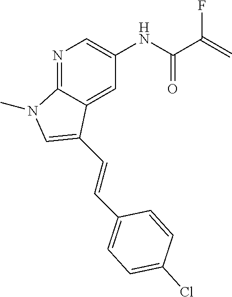 [SMILES: C=C(F)C(=O)Nc1cnc2c(c1)c(/C=C/c1ccc(Cl)cc1)cn2C]

The title compound was prepared from a compound prepared according to the procedure outlined for step 2 of Example 16 and 2-fluoroacrylic acid following the procedure outlined for Example 115 (i.e., the title compound was prepared following the procedure outlined for Example 115 using 2-fluoroacrylic acid and a compound prepared according to the procedure outlined for step 1 and step 2 of Example 15 using Intermediate E and (E)-(4-chlorostyryl) boronic acid). ¹H NMR (600 MHZ, CD₃OD) δ 8.54 (d, J=2.3 Hz, 1H), 8.41 (d, J=2.3 Hz, 1H), 7.72 (s, 1H), 7.09 (d, J=3.9 Hz, 1H), 6.93 (dd, J=4.3, 1.5 Hz, 1H), 5.76 (dd, J=46.5, 3.5 Hz, 1H), 5.34 (dd, J=15.1, 3.5 Hz, 1H), 3.88 (s, 3H). LCMS (ESI) m/z=356 (M+H)⁺.

### Example 119

### Preparation of (E)-3-bromo-N-(3-(4-chlorostyryl)-1-methyl-1H-pyrrolo[2,3-b]pyridin-5-yl)-4,5-dihydroisoxazole-5-carboxamide

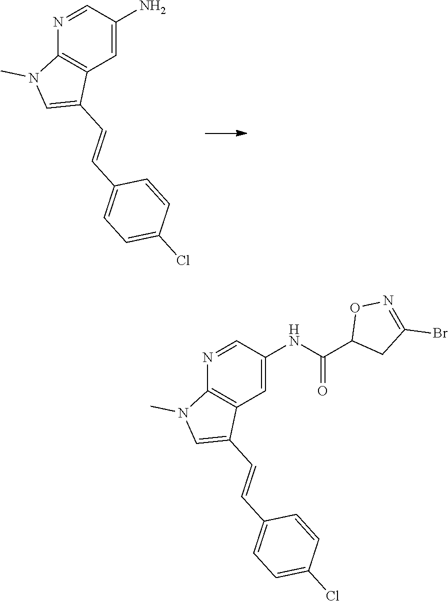 [SMILES: Cn1cc(/C=C/c2ccc(Cl)cc2)c2cc(N)cnc21 ; Cn1cc(/C=C/c2ccc(Cl)cc2)c2cc(NC(=O)C3CC(Br)=NO3)cnc21]

A solution of the intermediate prepared according to the procedure outlined for step 2 of Example 16 (100 mg, 0.3 mmol), dibromoformaldoxime (60 mg, 0.3 mmol) were dissolved in DMF (2 mL) at 0° C. Aqueous solution of KHCO₃ (0.59 mL, 0.59 mmol) was added dropwise to the solution for 30 min. The reaction mixture was warm to r.t. and stirred for 1 h. The reaction mixture was diluted in water and extracted with EtOAc. The combined organic layer was dried over anhydrous Na₂SO₄ and concentrated under reduced pressure. The residue was purified by column chromatography to obtain the titled compound (6.6 mg, 5%). ¹H NMR (600 MHz, CDCl₃) δ 8.65 (d, J=2.3 Hz, 1H), 8.51 (s, 1H), 8.39 (d, J=2.2 Hz, 1H), 7.44 (d, J=8.4 Hz, 2H), 7.38 (s, 1H), 7.31 (d, J=8.6 Hz, 2H), 7.17 (d, J=16.3 Hz, 1H), 6.98 (d, J=16.5 Hz, 1H), 5.25 (dd, J=9.7, 7.7 Hz, 1H), 3.89 (s, 3H). LCMS (ESI) m/z=459 (M+H)⁺.

### Example 120

### (E)-4-(Dimethylamino)-N-(1-methyl-3-(4-(trifluoromethyl)phenyl)-1H-pyrrolo[2,3-b]pyridin-5-yl) but-2-enamide 2,2,2-trifluoroacetate

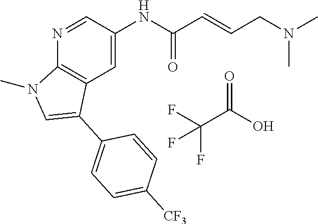

The title compound was prepared according to the procedure of Example 115, using (E)-4-(dimethylamino) but-2-enoic acid hydrochloride. ¹H NMR (600 MHz, DMSO-d6) & 10.54 (s, 1H), 9.78 (s, 1H), 8.76 (d, J=2.2 Hz, 1H), 8.52 (d, J=2.2 Hz, 1H), 8.13 (s, 1H), 7.85 (d, J=8.2 Hz, 2H), 7.80 (d, J=8.3 Hz, 2H), 6.79-6.71 (m, 1H), 6.48 (d, J=15.3 Hz, 1H), 3.99-3.94 (m, 2H), 3.86 (s, 3H), 2.80 (d, J=4.1 Hz, 6H). LCMS (ESI) m/z=403 (M+H)⁺.

### Example 121

### (E)-N-(3-(4-Chlorostyryl)-1-(methyl-d3)-1H-pyrrolo[2,3-b]pyridin-5-yl)acrylamide

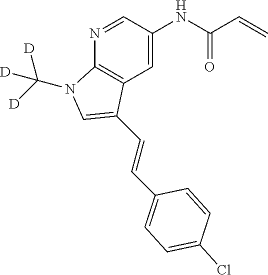 [SMILES: [2H]C([2H])([2H])n1cc(/C=C/c2ccc(Cl)cc2)c2cc(NC(=O)C=C)cnc21]

The title compound was prepared from 3-iodo-5-nitro-1-(trideuteriomethyl) pyrrolo[2,3-b]pyridine and (E)-(4-chlorostyryl) boronic acid following the procedure outlined for Example 15. ¹H NMR (400 MHZ, CD₃OD) δ 8.78 (d, J=2.3 Hz, 1H), 8.45 (d, J=2.1 Hz, 1H), 7.59 (s, 1H), 7.51 (d, J=8.5 Hz, 2H), 7.32 (d, J=8.5 Hz, 2H), 7.28 (d, J=16.6 Hz, 1H), 7.04 (d, J=16.5 Hz, 1H), 6.50 (dd, J=16.9, 9.6 Hz, 1H), 6.42 (dd, J=17.0, 2.1 Hz, 1H), 5.83 (dd, J=9.7, 2.2 Hz, 1H). LCMS (ESI) m/z=341 (M+H)⁺.

### Example 122

### Preparation of N-(3-(4,4-dimethylcyclohexyl)-1-methyl-1H-indol-5-yl)acrylamide

The overall reaction scheme was as follows:

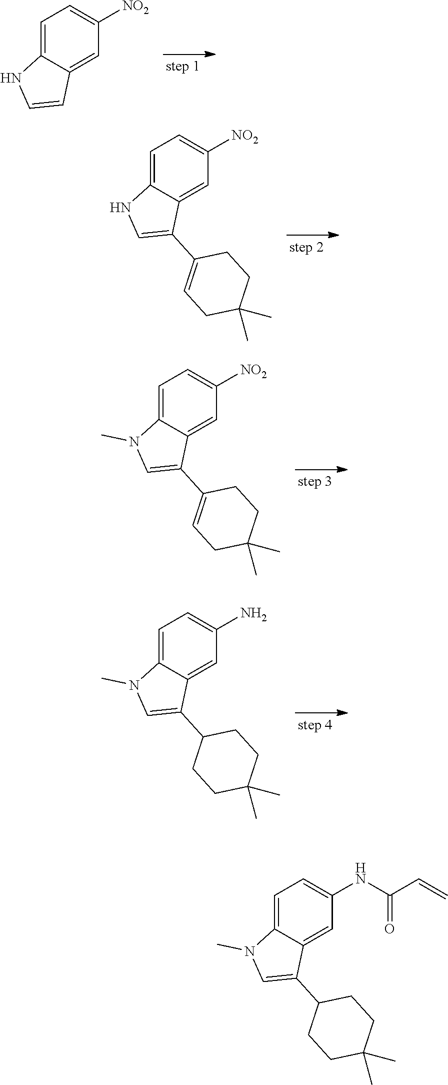

### Step 1: 3-(4,4-Dimethylcyclohex-1-en-1-yl)-5-nitro-1H-indole

To a mixture of 5-nitro-1H-indole (500 mg, 3.85 mmol) and 4,4-dimethylcyclohexanone (778 mg, 6.17 mmol) in MeOH (6 mL), NaOMe solution (30 wt %, 3.53 mL, 18.5 mmol) was added. The reaction mixture was stirred at 90° C. for 16 h then cooled to r.t. The reaction mixture was diluted with EtOAc and water then extracted with EtOAc. The collected organic layer was dried over anhydrous Na₂SO₄, filtered, and concentrated. The residue was purified by flash column chromatography (0-30% EtOAc/Hex) to obtain the titled compound (318 mg, 38.2%). ¹H NMR (600 MHZ, CDCl₃) δ 8.87 (d, J=2.2 Hz, 1H), 8.38 (s, 1H), 8.12 (dd, J=9.0, 2.2 Hz, 1H), 7.39 (d, J=9.0 Hz, 1H), 7.29 (d, J=2.4 Hz, 1H), 6.25-6.23 (m, 1H), 2.47-2.44 (m, 2H), 2.10-2.08 (m, 2H), 1.57 (t, J=6.4 Hz, 2H), 1.02 (s, 6H). LCMS (ESI) m/z=271 (M+H)⁺.

### Step 2: 3-(4,4-Dimethylcyclohex-1-en-1-yl)-1-methyl-5-nitro-1H-indole

To a mixture of 3-(4,4-dimethylcyclohex-1-en-1-yl)-5-nitro-1H-indole (318 mg, 1.18 mmol) and Cs₂CO₃ (767 mg, 2.35 mmol) in DMF (3 mL), iodoethane (0.11 mL, 1.76 mmol) was added. The reaction mixture was stirred at 80° C. for 16 h then cooled to r.t. The resultant mixture was diluted in EtOAc and washed with water 3 times then brine. The organic layer was dried over anhydrous Na₂SO₄, filtered, and concentrated. The residue was purified by flash column chromatography (0%-30% EtOAc/Hex) to obtain the titled compound (320 mg, 96%). ¹H NMR (600 MHz, CDCl₃) δ 8.85 (d, J=2.2 Hz, 1H), 8.13 (dd, J=9.0, 2.2 Hz, 1H), 7.30 (d, J=9.0 Hz, 1H), 7.13 (s, 1H), 6.22-6.20 (m, 1H), 3.82 (s, 3H), 2.45-2.41 (m, 2H), 2.10-2.06 (m, 2H), 1.56 (t, J=6.4 Hz, 2H), 1.01 (s, 6H). LCMS (ESI) m/z=285 (M+H)⁺.

### Step 3: 3-(4,4-Dimethylcyclohexyl)-1-methyl-1H-indol-5-amine

To a mixture of 3-(4,4-dimethylcyclohex-1-en-1-yl)-1-methyl-5-nitro-1H-indole (160 mg, 0.563 mmol) and Pd/C (17 mg, 0.45 mmol) were suspended in MeOH (3 mL). The reaction mixture was stirred at r.t. for 15 h. The resultant mixture was filtered to remove insolubles. The filtrate was concentrated and the residue was purified by flash column chromatography (0%-100% EtOAc/Hex) to obtain the titled compound (80 mg, 55%). ¹H NMR (600 MHZ, CDCl₃) δ 7.08 (d, J=8.5 Hz, 1H), 6.94 (d, J=2.1 Hz, 1H), 6.74 (s, 1H), 6.68 (dd, J=8.5, 2.2 Hz, 1H), 3.68 (s, 3H), 3.50 (s, 2H), 2.67-2.59 (m, 2H), 1.90-1.84 (m, 2H), 1.66-1.59 (m, 2H), 1.49 (d, J=13.1 Hz, 2H), 1.41-1.35 (m, 2H), 0.97 (d, J=3.6 Hz, 6H). LCMS (ESI) m/z=255 (M+H)⁺.

### Step 4: N-(3-(4,4-Dimethylcyclohexyl)-1-methyl-1H-indol-5-yl)acrylamide

To a mixture of 3-(4,4-dimethylcyclohexyl)-1-methyl-1H-indol-5-amine (80 mg, 0.312 mmol), acryloyl chloride (0.045 mL, 0.562 mmol) and TEA (0.087 mL, 0.624 mmol) in DCM (3 mL), was added. The reaction mixture was stirred at 0° C. for 1 h. The collected organic layer was dried over anhydrous Na₂SO₄, filtered, and concentrated. The residue was purified by flash column chromatography (0%-50% EtOAc/Hex) to obtain the titled compound (23 mg, 24%). ¹H NMR (600 MHz, CD₃OD) δ 7.96 (d, J=1.6 Hz, 1H), 7.30-7.25 (m, 2H), 6.94 (s, 1H), 6.47 (dd, J=17.0, 10.2 Hz, 1H), 6.34 (dd, J=17.0, 1.6 Hz, 1H), 5.74 (dd, J=10.2, 1.7 Hz, 1H), 3.73 (s, 3H), 2.72-2.63 (m, J=12.0, 3.6 Hz, 1H), 1.91-1.84 (m, 2H), 1.74-1.66 (m, 2H), 1.52 (d, J=13.0 Hz, 2H), 1.46-1.40 (m, 2H), 1.02 (s, 3H), 0.99 (s, 3H), LCMS (ESI) m/z=309 (M+H)⁺.

### Example 123

### N-(3-Cyclohexyl-1-methyl-1H-indol-5-yl)acrylamide

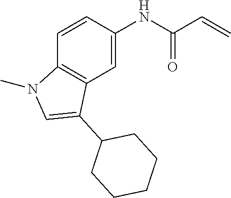 [SMILES: C=CC(=O)Nc1ccc2c(c1)c(C1CCCCC1)cn2C]

The title compound was prepared from cyclohexanone following the procedure outlined for Example 122. ¹H NMR (600 MHZ, CD₃OD) δ 7.95 (d, J=1.9 Hz, 1H), 7.29-7.27 (m, 1H), 7.26-7.24 (m, 1H), 6.90 (d, J=0.8 Hz, 1H), 6.46 (dd, J=17.0, 10.2 Hz, 1H), 6.34 (dd, J=17.0, 1.7 Hz, 1H), 5.74 (dd, J=10.2, 1.7 Hz, 1H), 3.72 (s, 3H), 2.81-2.74 (m, 1H), 2.06 (dd, J=8.7, 3.9 Hz, 2H), 1.89-1.82 (m, 2H), 1.81-1.75 (m, 1H), 1.50-1.45 (m, 4H), 1.37-1.29 (m, 1H). LCMS (ESI) m/z=283 (M+H)⁺.

### Example 124

### N-(3-(4,4-dimethylcyclohex-1-en-1-yl)-1-methyl-1H-indol-5-yl)acrylamide

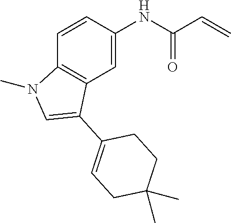 [SMILES: C=CC(=O)Nc1ccc2c(c1)c(C1=CCC(C)(C)CC1)cn2C]

The title compound was prepared from 4,4-dimethylcyclohexanone following the procedure outlined for Example 122 without step 3. ¹H NMR (600 MHZ, CD₃OD) δ 8.22 (d, J=1.8 Hz, 1H), 7.37 (dd, J=8.7, 1.9 Hz, 1H), 7.30 (d, J=8.8 Hz, 1H), 7.16 (s, 1H), 6.47 (dd, J=17.0, 10.2 Hz, 1H), 6.34 (dd, J=17.0, 1.7 Hz, 1H), 6.14-6.11 (m, 1H), 5.75 (dd, J=10.2, 1.7 Hz, 1H), 3.76 (s, 3H), 2.49-2.44 (m, 2H), 2.06 (dd, J=3.9, 2.0 Hz, 2H), 1.56 (t, J=6.5 Hz, 2H), 1.24 (t, J=7.2 Hz, 2H), 1.01 (s, 6H). LCMS (ESI) m/z=309 (M+H)⁺.

### Example 125

### N-(3-(4-Methoxycyclohex-1-en-1-yl)-1-methyl-1H-indol-5-yl)acrylamide

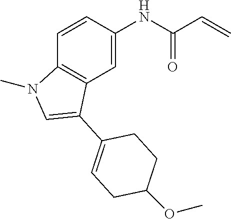 [SMILES: C=CC(=O)Nc1ccc2c(c1)c(C1=CCC(OC)CC1)cn2C]

The title compound was prepared from 4-methoxycyclohexanone following the procedure outlined for Example 122 without step 3. ¹H NMR (600 MHZ, CD₃OD) δ 8.22 (d, J=1.8 Hz, 1H), 7.36 (dd, J=8.7, 1.9 Hz, 1H), 7.31 (d, J=8.7 Hz, 1H), 7.18 (s, 1H), 6.47 (dd, J=17.0, 10.2 Hz, 1H), 6.35 (dd, J=17.0, 1.6 Hz, 1H), 6.09 (d, J=1.2 Hz, 1H), 5.75 (dd, J=10.2, 1.6 Hz, 1H), 3.76 (s, 3H), 3.63-3.57 (m, 1H), 3.42 (s, 3H), 2.67-2.60 (m, 2H), 2.55-2.47 (m, 1H), 2.25-2.18 (m, 1H), 2.14-2.08 (m, 1H), 1.80-1.73 (m, 1H). LCMS (ESI) m/z=311 (M+H)⁺.

### Example 126

### N-(3-(4-Methoxycyclohexyl)-1-methyl-1H-indol-5-yl)acrylamide

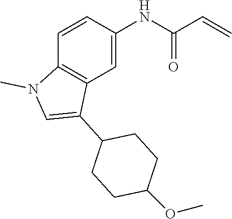 [SMILES: C=CC(=O)Nc1ccc2c(c1)c(C1CCC(OC)CC1)cn2C]

The title compound was prepared from 4-methoxycyclohexanone following the procedure outlined for Example 122. ¹H NMR (600 MHZ, CD₃OD) δ 7.93 (d, J=1.6 Hz, 1H), 7.28 (m, 2H), 6.93 (s, 1H), 6.47 (dd, J=17.0, 10.2 Hz, 1H), 6.34 (dd, J=17.0, 1.7 Hz, 1H), 5.74 (dd, J=10.2, 1.7 Hz, 1H), 3.73 (s, 3H), 3.55 (t, J=2.9 Hz, 1H), 3.35 (s, 3H), 2.88-2.81 (m, 1H), 2.07-2.01 (m, 2H), 1.83-1.77 (m, 4H), 1.68-1.61 (m, 2H). LCMS (ESI) m/z=313 (M+H)⁺.

### Example 127

### N-(1-Methyl-3-(3-phenylcyclohexyl)-1H-indol-5-yl)acrylamide

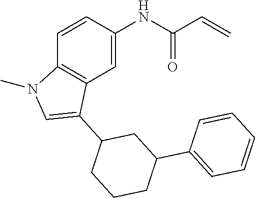 [SMILES: C=CC(=O)Nc1ccc2c(c1)c(C1CCCC(c3ccccc3)C1)cn2C]

The title compound was prepared from 3-phenylcyclohexanone following the procedure outlined for Example 122. ¹H NMR (600 MHZ, CD₃OD) δ 7.96 (dd, J=37.8, 1.7 Hz, 1H), 7.33-7.28 (m, 1.5H, enantiomeric mixture), 7.27 (s, 0.5H, enantiomer), 7.25 (t, J=5.9 Hz, 4H), 7.15-7.10 (m, 1H, 1.5H, enantiomeric mixture), 6.95 (s, 0.5H, enantiomer), 6.49-6.42 (m, 1H), 6.37-6.30 (m, 1H), 5.75-5.71 (m, 1H), 3.75 (d, J=36.0 Hz, 3H), 3.41-3.37 (m, 0.5H, enantiomer), 3.03-2.91 (m, 1H), 2.80-2.72 (m, 0.5H, enantiomer), 2.27-2.06 (m, 3H), 2.03-1.97 (m, 1H), 1.97-1.85 (m, 1.5H, enantiomeric mixture), 1.85-1.76 (m, 1H), 1.76-1.64 (m, 2H), 1.59-1.50 (m, 1.5H, enantiomeric mixture), 1.35-1.29 (m, 1H). LCMS (ESI) m/z=359 (M+H)⁺.

### Example 128

### (E)-N-(3-(4-Chlorostyryl)-1-(difluoromethyl)-1H-pyrrolo[2,3-b]pyridin-5-yl)acrylamide

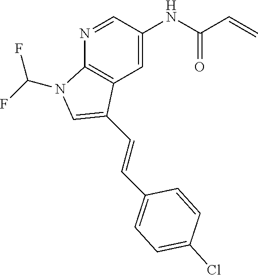 [SMILES: C=CC(=O)Nc1cnc2c(c1)c(/C=C/c1ccc(Cl)cc1)cn2C(F)F]

The title compound was prepared from Intermediate U and (E)-(4-chlorostyryl) boronic acid following the procedure outlined for Example 15. ¹H NMR (600 MHZ, CD₃OD) δ 8.86 (d, J=1.8 Hz, 1H), 8.30 (d, J=2.2 Hz, 1H), 7.79 (t, J=60.5 Hz, 1H), 7.64 (s, 1H), 7.45 (d, J=8.5 Hz, 2H), 7.39 (s, 1H), 7.34 (dd, J=6.3, 4.5 Hz, 2H), 7.19-7.06 (m, 2H), 6.52 (dd, J=16.9, 0.8 Hz, 1H), 6.32 (dd J=16.8, 10.3 Hz, 1H), 5.87 (d, J=10.3 Hz, 1H). LCMS (ESI) m/z=374 (M+H)⁺.

### Example 129

### N-(1-(Difluoromethyl)-3-(4-(trifluoromethyl)phenyl)-1H-pyrrolo[2,3-b]pyridin-5-yl)acrylamide

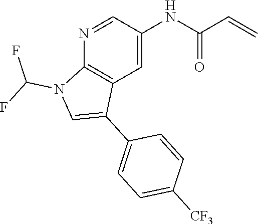 [SMILES: C=CC(=O)Nc1cnc2c(c1)c(-c1ccc(C(F)(F)F)cc1)cn2C(F)F]

The title compound was prepared from Intermediate U and [4-(trifluoromethyl)phenyl]boronic acid following the procedure outlined for Example 15. ¹H NMR (600 MHz, CD₃OD) δ 8.81 (d, J=2.2 Hz, 1H), 8.54 (d, J=2.2 Hz, 1H), 8.09 (d, J=7.4 Hz, 1H), 7.92 (dd, J=34.1, 26.0 Hz, 3H), 7.79 (d, J=8.2 Hz, 2H), 6.45 (m, 2H), 5.83 (dd, J=9.9, 1.8 Hz, 1H). LCMS (ESI) m/z=382 (M+H)⁺.

### Example 130

### (E)-N-(3-(2-(4,4-Difluorocyclohexyl)vinyl)-1-(difluoromethyl)-1H-pyrrolo[2,3-b]pyridin-5-yl)acrylamide

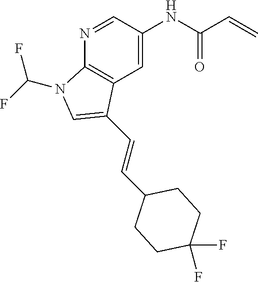 [SMILES: C=CC(=O)Nc1cnc2c(c1)c(/C=C/C1CCC(F)(F)CC1)cn2C(F)F]

The title compound was prepared from Intermediate U and (E)-2-(2-(4,4-difluorocyclohexyl)vinyl)-4,4,5,5-tetramethyl-1,3,2-dioxaborolane following the procedure outlined for Example 15. ¹H NMR (600 MHZ, CD₃OD) δ 8.72 (d, J=2.2 Hz, 1H), 8.48 (d, J=2.2 Hz, 1H), 7.86 (t, J=60.3 Hz, 2H), 7.66 (s, 1H), 6.58 (d, J=16.8 Hz, 1H), 6.45 (m, 2H), 6.28 (dd, J=16.2, 7.1 Hz, 1H), 5.83 (dd, J=9.8, 1.9 Hz, 1H), 2.33 (s, 1H), 2.14-2.05 (m, 2H), 1.98-1.80 (m, 4H), 1.60 (dd, J=26.2, 11.5 Hz, 2H). LCMS (ESI) m/z=382 (M+H)⁺.

### Example 131

### Preparation of N-(3-cyclohexyl-1-(difluoromethyl)-1H-indol-5-yl)acrylamide

The overall reaction scheme was as follows:

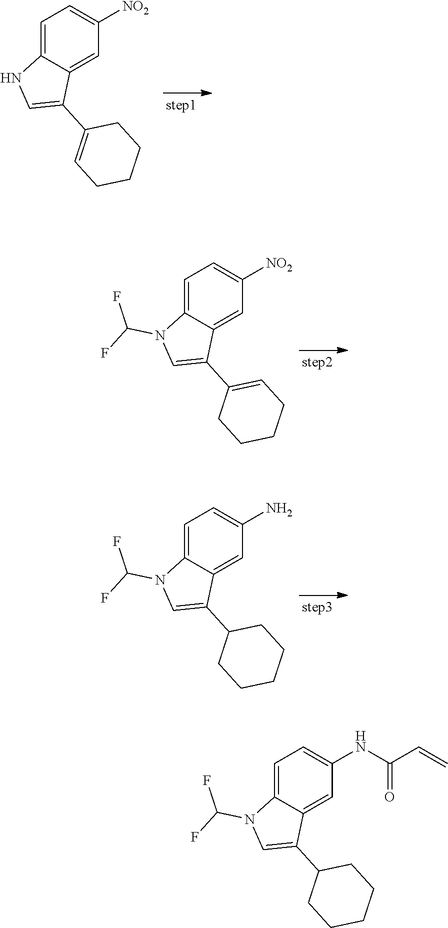 [SMILES: O=[N+]([O-])c1ccc2[nH]cc(C3=CCCCC3)c2c1 ; O=[N+]([O-])c1ccc2c(c1)c(C1=CCCCC1)cn2C(F)F ; Nc1ccc2c(c1)c(C1CCCCC1)cn2C(F)F ; C=CC(=O)Nc1ccc2c(c1)c(C1CCCCC1)cn2C(F)F]

### Step 1: 3-(Cyclohexen-1-yl)-1-(difluoromethyl)-5-nitro-1H-indole

A mixture of 3-(cyclohexen-1-yl)-5-nitro-1H-indole (500 mg, 2.06 mmol), ethyl 2-bromo-2,2-difluoro-acetate (0.29 mL, 2.27 mmol), and t-BuOK (463 mg, 4.13 mmol) were suspended in MeCN (20 mL). The reaction mixture was stirred at 80° C. for 18 h then concentrated. The residue was diluted with EtOAc and water, extracted with EtOAc. The collected organic layer was dried over Na₂SO₄, filtered, and concentrated. The residue was purified by flash chromatography to obtain the titled compound (183 mg, 30%). ¹H NMR (400 MHz, CDCl₃) δ 8.82 (d, J=2.0 Hz, 1H), 8.22 (dd, J=9.0, 2.3 Hz, 1H), 7.60 (d, J=9.1 Hz, 1H), 7.32 (s, 1H), 6.35-6.28 (m, 1H), 2.45-2.37 (m, 2H), 2.35-2.26 (m, 2H), 1.89-1.78 (m, 2H), 1.73 (qd, J=5.8, 2.0 Hz, 2H). LCMS (ESI) m/z=293 (M+H)⁺.

### Step 2: 3-Cyclohexyl-1-(difluoromethyl)-1H-indol-5-amine

To a solution of 3-(cyclohexen-1-yl)-1-(difluoromethyl)-5-nitro-1H-indole (59 mg, 0.202 mmol) in EtOAc (2 mL), Pd/C (21 mg, 0.0202 mmol) was added. The reaction mixture was stirred under hydrogen atmosphere for 18 h. The insolubles removed by filtration and concentrated to obtain the titled compound and was underwent to the next reaction without further purifications. LCMS (ESI) m/z=265 (M+H)⁺.

### Step 3: N-(3-Cyclohexyl-1-(difluoromethyl)-1H-indol-5-yl)acrylamide

A mixture of 3-cyclohexyl-1-(difluoromethyl)-1H-indol-5-amine (59 mg, 0.223 mmol) and TEA (0.062 mL, 0.446 mmol) were diluted in DCM (2 mL). Acryloyl chloride (0.013 mL, 0.156 mmol) was added at 0° C. and stirred for 30 min. The resultant mixture was concentrated and purified by flash chromatography (0-30% EtOAc/DCM) to obtain the titled compound (16 mg, 23%). ¹H NMR (400 MHZ, CD₃OD) δ 8.07 (d, J=1.8 Hz, 1H), 7.57-7.50 (m, 2H), 7.38 (dd, J=8.8, 1.5 Hz, 1H), 7.16 (s, 1H), 6.46 (dd, J=16.9, 9.9 Hz, 1H), 6.36 (dd, J=17.0, 1.9 Hz, 1H), 5.76 (dd, J=10.0, 1.9 Hz, 1H), 2.81-2.74 (m, 1H), 2.12-2.06 (m, 2H), 1.91-1.76 (m, 5H), 1.53-1.44 (m, 6H), 1.37-1.27 (m, 2H). LCMS (ESI) m/z=319 (M+H)⁺.

### Example 132

### N-(3-(4-Chlorophenyl)-1-(difluoromethyl)-1H-pyrrolo[2,3-b]pyridin-5-yl)acrylamide

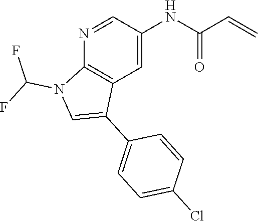 [SMILES: C=CC(=O)Nc1cnc2c(c1)c(-c1ccc(Cl)cc1)cn2C(F)F]

The title compound was prepared from Intermediate U and (4-chlorophenyl) boronic acid following the procedure outlined for Example 15. ¹H NMR (400 MHz, CD₃OD) δ 8.75 (d, J=2.3 Hz, 1H), 8.53 (d, J=2.2 Hz, 1H), 8.14-7.77 (m, 1H), 7.70 (d, J=8.6 Hz, 2H), 7.49 (d, J=8.6 Hz, 2H), 6.44 (m, 3H), 5.82 (dd, J=9.5, 2.3 Hz, 1H). LCMS (ESI) m/z=348 (M+H)⁺.

### Example 133

### N-(3-(4-Chloro-3-fluorophenyl)-1-(difluoromethyl)-1H-pyrrolo[2,3-b]pyridin-5-yl)acrylamide

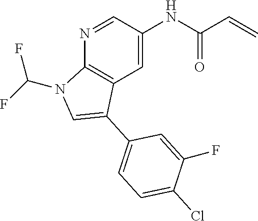 [SMILES: C=CC(=O)Nc1cnc2c(c1)c(-c1ccc(Cl)c(F)c1)cn2C(F)F]

The title compound was prepared from Intermediate U and (4-chloro-3-fluorophenyl) boronic acid following the procedure outlined for Example 15. ¹H NMR (400 MHZ, CD₃OD) δ 8.76 (d, J=2.2 Hz, 1H), 8.54 (d, J=2.2 Hz, 1H), 8.15-7.79 (m, 2H), 7.64-7.51 (m, 3H), 6.44 (m, 2H), 5.83 (dd, J=9.5, 2.3 Hz, 1H). LCMS (ESI) m/z=366 (M+H)⁺.

### Example 134

### N-(1-(Difluoromethyl)-3-(3-(trifluoromethyl)phenyl)-1H-pyrrolo[2,3-b]pyridin-5-yl)acrylamide

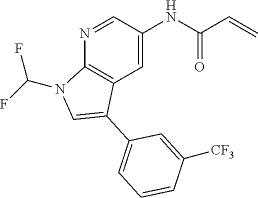 [SMILES: C=CC(=O)Nc1cnc2c(c1)c(-c1cccc(C(F)(F)F)c1)cn2C(F)F]

The title compound was prepared from Intermediate U and (3-(trifluoromethyl)phenyl) boronic acid following the procedure outlined for Example 15. ¹H NMR (400 MHZ, CD₃OD) δ 8.73 (d, J=2.2 Hz, 1H), 8.59 (d, J=2.2 Hz, 1H), 8.15-7.80 (m, 4H), 7.74-7.63 (m, 2H), 6.44 (m, 2H), 5.82 (dd, J=9.5, 2.3 Hz, 1H). LCMS (ESI) m/z=382 (M+H)⁺.

### Example 135

### N-(3-(3-Chloro-4-fluorophenyl)-1-(difluoromethyl)-1H-pyrrolo[2,3-b]pyridin-5-yl)acrylamide

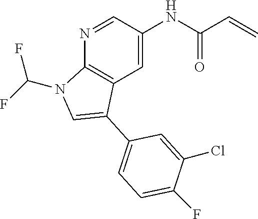 [SMILES: C=CC(=O)Nc1cnc2c(c1)c(-c1ccc(F)c(Cl)c1)cn2C(F)F]

The title compound was prepared from Intermediate U and (3-chloro-4-fluorophenyl) boronic acid following the procedure outlined for Example 15. ¹H NMR (400 MHZ, CD₃OD) δ 8.70 (d, J=2.2 Hz, 1H), 8.55 (d, J=2.2 Hz, 1H), 8.12-7.78 (m, 3H), 7.66 (m, 1H), 7.37 (t, J=8.9 Hz, 1H), 6.44 (m, 2H), 5.82 (dd, J=9.5, 2.3 Hz, 1H). LCMS (ESI) m/z=366 (M+H)⁺.

### Example 136

### N-(3-(3-Chlorobenzyl)-1-methyl-1H-pyrrolo[2,3-b]pyridin-5-yl)acrylamide

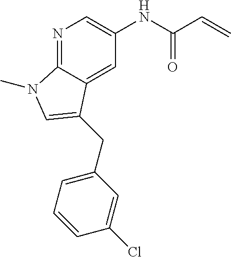 [SMILES: C=CC(=O)Nc1cnc2c(c1)c(Cc1cccc(Cl)c1)cn2C]

The title compound was prepared from Intermediate E and (3-chlorobenzyl) boronic acid following the procedure outlined for Example 15. ¹H NMR (600 MHZ, CD₃OD) δ 8.41 (d, J=2.2 Hz, 1H), 8.15 (d, J=2.2 Hz, 1H), 7.24 (d, J=1.9 Hz, 1H), 7.22 (d, J=7.7 Hz, 1H), 7.20-7.14 (m, 3H), 6.43 (dd, J=17.0, 10.0 Hz, 1H), 6.36 (dd, J=17.0, 1.7 Hz, 1H), 5.77 (dd, J=10.0, 1.7 Hz, 1H), 4.03 (s, 2H), 3.80 (s, 3H). LCMS (ESI) m/z=326 (M+H)⁺.

### Example 137

### N-(3-(Cyclohexylidenemethyl)-1-methyl-1H-pyrrolo[2,3-b]pyridin-5-yl)acrylamide

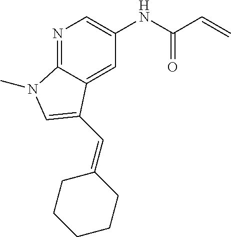 [SMILES: C=CC(=O)Nc1cnc2c(c1)c(C=C1CCCCC1)cn2C]

The title compound was prepared from Intermediate E and (cyclohexylidenemethyl) boronic acid pinacol ester following the procedure outlined for Example 15. ¹H NMR (400 MHZ, CD₃OD) δ 8.38 (d, J=2.3 Hz, 1H), 8.34 (d, J=2.3 Hz, 1H), 7.27 (s, 1H), 6.48 (dd, J=17.0, 9.8 Hz, 1H), 6.38 (dd, J=17.0, 2.0 Hz, 1H), 6.19 (s, 1H), 5.80 (dd, J=9.8, 2.0 Hz, 1H), 3.84 (s, 3H), 2.46-2.41 (m, 2H), 2.33 (d, J=5.8 Hz, 2H), 1.70-1.55 (m, 6H). LCMS (ESI) m/z=296 (M+H)⁺.

### Example 138

### N-(3-(Cyclohexylmethyl)-1-methyl-1H-pyrrolo[2,3-b]pyridin-5-yl)acrylamide

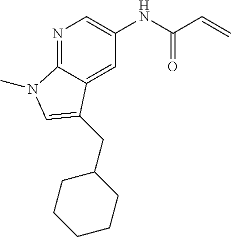 [SMILES: C=CC(=O)Nc1cnc2c(c1)c(CC1CCCCC1)cn2C]

The title compound was prepared from a compound prepared according to the procedure outlined for step 1 of Example 137 following the procedure outlined for Example 84. ¹H NMR (600 MHZ, CD₃OD) δ 8.35 (d, J=2.2 Hz, 1H), 8.32 (d, J=2.3 Hz, 1H), 7.11 (s, 1H), 6.47 (dd, J=17.0, 10.1 Hz, 1H), 6.38 (dd, J=17.0, 1.7 Hz, 1H), 5.79 (dd, J=10.1, 1.7 Hz, 1H), 3.79 (s, 3H), 2.58 (d, J=7.0 Hz, 2H), 1.77-1.67 (m, 4H), 1.66-1.55 (m, 2H), 1.26-1.14 (m, 2H), 1.03-0.93 (m, 2H). LCMS (ESI) m/z=298 (M+H)⁺.

### Biological Evaluation

### Example A1: Reporter Assay

The TEAD reporter-containing human breast cancer cell line MCF7 (60618, BPS bioscience) was cultured according to the manufacturer's instructions. Cells were plated in 96-well white plates at a density of 30,000 per well and incubated overnight. The following day cells were treated with small molecules in 1% serum media (0.1% v/v DMSO). After 24 hours of the treatment, cell viability was evaluated by using CCK-8 reagent (CK04, Dojindo) and measuring absorbance at 450 nm. Then, the assay for luciferase activity was performed using Dual-Glo® Luciferase Assay System (Promega, E2940) following the manufacturer's instructions. Luminescence intensity was measured and the potency of the compounds was determined by IC₅₀ value generated from non-linear regression analysis of the dose-response curves.

IC₅₀ for luciferase activity are shown in the Table 1 below and designated within the following ranges. [+++: ≤0.1 μM, ++: >0.1 μM to ≤1 μM, +: >1 μM to ≤10 μM, −: >10 μM]

TABLE 1

| | Luciferase IC₅₀ |
|---|---|
| Example No | (μM) |
| 1 | +++ |
| 2 | ++ |
| 3 | +++ |
| 4 | − |
| 5 | ++ |
| 6 | − |
| 7 | ++ |
| 8 | + |
| 9 | +++ |
| 10 | + |
| 11 | + |
| 12 | ++ |
| 15 | +++ |
| 16 | ++ |
| 18 | + |
| 19 | +++ |
| 20 | + |
| 21 | +++ |
| 22 | ++ |
| 23 | +++ |
| 24 | ++ |
| 25 | +++ |
| 26 | ++ |
| 27 | ++ |
| 28 | ++ |
| 29 | − |
| 30 | +++ |
| 31 | ++ |
| 32 | +++ |
| 33 | + |
| 34 | +++ |
| 35 | ++ |
| 36 | ++ |
| 37 | ++ |
| 38 | + |
| 39 | − |
| 45 | ++ |
| 48 | + |
| 51 | + |
| 54 | +++ |
| 55 | ++ |
| 56 | + |
| 57 | + |
| 58 | ++ |
| 59 | ++ |
| 60 | + |
| 61 | + |
| 62 | − |
| 63 | +++ |
| 64 | +++ |
| 65 | − |
| 69 | + |
| 70 | + |
| 75 | − |
| 76 | +++ |
| 78 | ++ |
| 81 | ++ |
| 82 | ++ |
| 85 | ++ |
| 86 | ++ |
| 87 | ++ |
| 89 | ++ |
| 90 | + |
| 92 | +++ |
| 93 | ++ |
| 94 | ++ |
| 97 | ++ |
| 98 | + |
| 101 | + |
| 102 | + |
| 106 | ++ |
| 107 | +++ |
| 108 | ++ |
| 109 | + |
| 110 | ++ |
| 111 | ++ |
| 112 | − |
| 113 | − |
| 115 | − |
| 116 | ++ |
| 117 | ++ |
| 118 | + |
| 120 | + |
| 121 | ++ |
| 122 | +++ |
| 123 | +++ |
| 124 | +++ |
| 125 | + |
| 126 | − |
| 130 | ++ |
| 132 | + |
| 133 | + |
| 134 | ++ |
| 135 | + |
| 136 | +++ |
| 137 | +++ |
| 138 | +++ |

### Example A2: Proliferation Assay

The anti-proliferative activity of TEAD inhibitors was evaluated in cells with different properties for YAP; “YAP-driven” cells (MSTO-211H, NCI-H226) in which YAP located in the nucleus and “YAP-independent” cells (OCM1, NCI-H28) where YAP located in the cytoplasm. Also, MSTO-211H cell line has the LATS1 mutation, and NCI-H226 has the NF2 mutation; otherwise OCM1 has a BRAF mutation with inactive cytoplasmic YAP, and NCI-H28 has a WT form for NF2. TEAD inhibitors were evaluated with cell lines with these YAP profiling and mutational status.

2500-3000 cells were seeded with cell growth medium (as suggested from ATCC for each cell line) containing 10% serum in a black 96-well plate (Corning) with clear flat bottom for imaging at 50-60% density. The next day, replace it with cell starvation medium containing 1% serum. After one day of growth in the starvation medium, the cells were incubated for 3 days with TEAD inhibitors in a concentration range of 0.01-10 μM. After 3 days, EdU (Invitrogen) at a concentration of 10 UM was added to the starvation medium and incubated for an additional 24 hours. After removing the starvation medium remaining in each well, wash the wells with PBS. Cells were fixed with 4% paraformaldehyde (Electron microscopy sciences) at room temperature for 20 minutes. After removing the fix solution remaining in each well, wash the wells with PBS and the cells were permeabilized for 40 minutes by adding 0.5% triton X-100 (Sigma Aldrich). After removing the permeabilization solution remaining in each well, the EdU labeling was performed according to the instructions of the manufacturer's instructions (Click-iT™ Cell Reaction Buffer Kit, Invitrogen). After the Click reaction for EdU labeling, nuclei were stained with Hoechst 333 (Thermo Scientific) for total cell count analysis. After plate preparation for detection, Image acquisition was performed using the ImageXpress Micro XLS. The acquired images were analyzed using MetaXpress software (molecular devices). Through this experimental method, it was possible to confirm the inhibitory activity of TEAD inhibitors by detection EdU incorporated into proliferating cells.

IC₅₀ for cell proliferation inhibition activity are shown in the Table 2 below and designated within the following ranges. [+++: ≤0.1 μM, ++: >0.1 μM to ≤1 μM, +: >1 μM to ≤10μ, −: >10 μM]

TABLE 2

| Example No | MSTO-211H IC₅₀ (μM) | NCI-H226 IC₅₀ (μM) |
|---|---|---|
| 1 | ++ | +++ |
| 2 | +++ | +++ |
| 3 | +++ | ++ |
| 4 | +++ | +++ |
| 5 | ++ | |
| 10 | ++ | |
| 13 | + | |
| 14 | ++ | |
| 15 | +++ | +++ |
| 16 | +++ | +++ |
| 17 | ++ | +++ |
| 18 | +++ | +++ |
| 19 | ++ | +++ |
| 21 | ++ | ++ |
| 22 | ++ | ++ |
| 23 | ++ | |
| 24 | ++ | +++ |
| 25 | +++ | ++ |
| 26 | ++ | |
| 27 | ++ | |
| 28 | ++ | +++ |
| 30 | ++ | +++ |
| 31 | +++ | +++ |
| 32 | ++ | |
| 33 | +++ | |
| 34 | ++ | |
| 35 | ++ | |
| 36 | +++ | |
| 37 | +++ | |
| 40 | ++ | |
| 41 | +++ | |
| 42 | ++ | |
| 43 | ++ | |
| 44 | +++ | |
| 46 | ++ | |
| 47 | +++ | +++ |
| 49 | +++ | |
| 51 | +++ | |
| 52 | ++ | |
| 63 | +++ | +++ |
| 64 | +++ | |
| 66 | ++ | |
| 67 | + | |
| 68 | + | |
| 70 | + | |
| 77 | ++ | |
| 81 | +++ | |
| 85 | ++ | |
| 87 | +++ | +++ |
| 88 | ++ | |
| 89 | +++ | +++ |
| 92 | +++ | +++ |
| 93 | +++ | +++ |
| 95 | ++ | |
| 96 | ++ | |
| 97 | +++ | +++ |
| 98 | +++ | +++ |
| 100 | +++ | |
| 103 | ++ | |
| 104 | ++ | |
| 106 | +++ | |
| 107 | +++ | |
| 111 | +++ | |
| 118 | +++ | |
| 121 | +++ | |
| 122 | ++ | |
| 128 | ++ | ++ |
| 129 | ++ | |
| 130 | ++ | |
| 132 | ++ | |

1. A compound represented by formula (I), a pharmaceutically acceptable salt thereof, stereoisomer thereof, a tautomer thereof, a solvate thereof, a hydrate thereof, or a prodrug analog thereof: 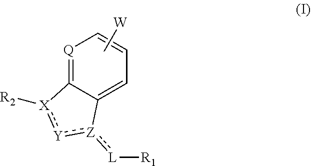
wherein:
each [symbol] of formula (I) is independently a single bond or a double bond,
L is absent, —N(R_{L})—, —N(R_{L})C(R_{L})₂—, —N(R_{L})C(═O)—, —C(R_{L})₂O—, —OC(R_{L})₂—, —C(R_{L})₂N(R_{L})—, —C(═O)—, —CH(OR_{L})—, ═CH— or is unbranched or branched C1-C3 alkylene or C2-C3 alkenylene, wherein at least one H of the unbranched or branched C1-C3 alkylene or C2-C3 alkenylene is independently unsubstituted or substituted with halogen, C1-C6 haloalkyl OR_{L} or N(R_{L})₂;
R_{L} is H or C1-C6 alkyl, wherein at least one H of the C1-C6 alkyl is independently unsubstituted or substituted with halogen;
Q is CH or N, wherein H of the CH is unsubstituted or substituted with halogen;
X is C or N;
Y is CR_{N}, C(═O) or N;
R_{N} is H or C1-C6 alkyl;
Z is C, N or CH;
W is 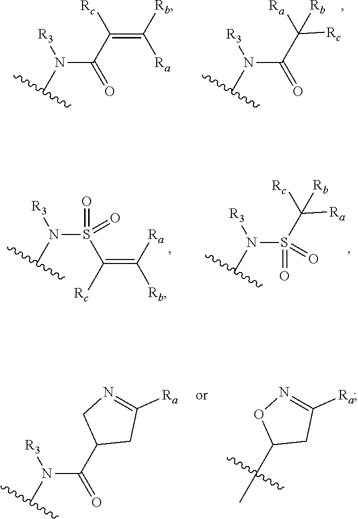
Rₐ, R_{b}, and R_{c} are each independently H, halogen, cyano, hydroxyl, C1-C3 alkyl-N(R₄)₂, or C1-C6 alkyl;
R₁ is H, C1-C3 alkyl, C3-C6 cycloalkyl, 4 to 6-membered heterocycloalkyl, C3-C6 cycloalkenyl, 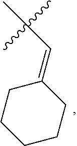 [SMILES: *C=C1CCCCC1]
aryl or heteroaryl, wherein at least one H of the C1-C3 alkyl, C3-C6 cycloalkyl, 4 to 6-membered heterocycloalkyl, C3-C6 cycloalkenyl, aryl or heteroaryl is independently unsubstituted or substituted with halogen, CN, —CH═CH—R₄, —(CH₂)₂—R₄, OR₄, SR₄, CH₂N(R₄)₂, C(═O)—R₄, COOR₄, CON(R₄)₂, C1-C10 alkyl, C1-C6 haloalkyl, C3-C6 cycloalkyl, 4 to 6-membered heterocycloalkyl, phenyl or phenyl substituted with halogen;
R₂ is H, unbranched or branched C1-C6 alkyl or C3-C6 cycloalkyl, wherein at least one H of the unbranched or branched C1-C6 alkyl or C3-C6 cycloalkyl is independently unsubstituted or substituted with deuterium, halogen or =0;
R₃ is H, unbranched or branched C1-C6 alkyl, C3-C6 cycloalkyl, C1-C6 alkyl-aryl or C1-C6 alkyl-heteroaryl, wherein at least one H of the C1-C6 alkyl, C3-C6 cycloalkyl, C1-C6 alkyl-aryl or C1-C6 alkyl-heteroaryl is independently unsubstituted or substituted with halogen, OR_{M}, N(R_{M})₂, COOH, COOR_{M}, C(═O)R_{M} or C(═O)N(R_{M})₂;
R_{M} is H, C1-C6 alkyl or C3-C6 cycloalkyl; and
R₄ is H, C1-C6 alkyl, C3-C6 cycloalkyl, aryl or C1-C6 alkyl-aryl, wherein at least one H of the C1-C6 alkyl, C3-C6 cycloalkyl, aryl or C1-C6 alkyl-aryl is independently unsubstituted or substituted with halogen.

2. The compound represented by formula (I), a pharmaceutically acceptable salt thereof, a stereoisomer thereof, a tautomer thereof, a solvate thereof, a hydrate thereof, or a prodrug analog thereof according to claim 1, wherein:
L is absent or ═CH— or is unbranched or branched C1-C3 alkylene or C2-C3 alkenylene, wherein at least one H of the unbranched or branched C1-C3 alkylene or C2-C3 alkenylene is independently unsubstituted or substituted with C1-C6 haloalkyl;
Q is CH or N, wherein H of the CH is unsubstituted or substituted with halogen;
X is C or N;
Y is CR_{N}, C(═O) or N;
R_{N} is H or C1-C6 alkyl;
Z is C, N or CH,
when [symbol] connecting Y to Z is a single bond and L is absent, unbranched or branched C1-C3 alkylene, C2-C3 alkenylene or C2-C3 alkynylene, Z is N or CH,
when [symbol] connecting Y to Z is a double bond and L is absent, unbranched or branched C1-C3 alkylene, C2-C3 alkenylene or C2-C3 alkynylene, Z is C, and
when [symbol] connecting Y to Z is a single bond and L is ═CH—, Z is C;
W is 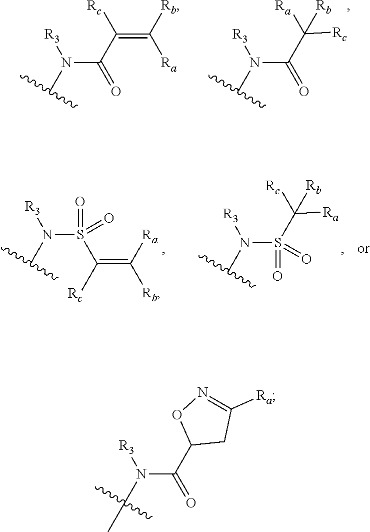
Rₐ, R_{b}, and R_{c} are each independently H, halogen, cyano, hydroxyl, C1-C3 alkyl-N(R₄)₂ or C1-C6 alkyl;
R₁ is H, C1-C3 alkyl, C3-C6 cycloalkyl, 4 to 6-membered heterocycloalkyl, C3-C6 cycloalkenyl, 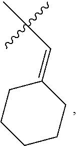 [SMILES: *C=C1CCCCC1]
aryl or heteroaryl, wherein at least one H of the C1-C3 alkyl, C3-C6 cycloalkyl, C3-C6 cycloalkenyl, aryl or heteroaryl is independently unsubstituted or substituted with halogen, CN, —CH═CH—R₄, —(CH₂)₂—R₄, OR₄, SR₄, CH₂N(R₄)₂, C(═O)—R₄, COOR₄, C1-C10 alkyl, C1-C6 haloalkyl, C3-C6 cycloalkyl, 4 to 6-membered heterocycloalkyl, phenyl or phenyl substituted with halogen;
R₂ is H or unbranched or branched C1-C6 alkyl or C3-C6 cycloalkyl, wherein at least one H of the unbranched or branched C1-C6 alkyl or C3-C6 cycloalkyl is independently unsubstituted or substituted with deuterium, halogen or ═O;
R₃ is H, unbranched or branched C1-C6 alkyl, C3-C6 cycloalkyl, benzyl or C1-C6 alkyl-benzyl, wherein at least one H of the unbranched or branched C1-C6 alkyl, C3-C6 cycloalkyl, benzyl or C1-C6 alkyl-benzyl is independently unsubstituted or substituted with COOH;
R₄ is H, C1-C6 alkyl, C3-C6 cycloalkyl, phenyl or benzyl, wherein at least one H of the C1-C6 alkyl, C3-C6 cycloalkyl, phenyl or benzyl is independently unsubstituted or substituted with halogen.

3. The compound represented by formula (I), a pharmaceutically acceptable salt thereof, a stereoisomer thereof, a tautomer thereof, a solvate thereof, a hydrate thereof, or a prodrug analog thereof according to claim 1, wherein:
L is absent, unbranched or branched C1-C3 alkylene or C2-C3 alkenylene;
X, Y, Z, Q, R₁, R₂ and W are the same as defined in claim 1.

4. The compound represented by formula (I), a pharmaceutically acceptable salt thereof, a stereoisomer thereof, a tautomer thereof, a solvate thereof, a hydrate thereof, or a prodrug analog thereof according to claim 1, wherein:
(i) X is N, Y is CR_{N} and Z is C,
(ii) X is N, Y is N and Z is C,
(iii) X is C, Y is CR_{N} and Z, or
(iv) X is N, Y is C(═O) and Z is C,
L, Q, R₁, R₂ and W are the same as defined in claim 1.

5. The compound represented by formula (I), a pharmaceutically acceptable salt thereof, a stereoisomer thereof, a tautomer thereof, a solvate thereof, a hydrate thereof, or a prodrug analog thereof according to claim 1, wherein the compound by represented by formula (I) is a compound represented by formula (II): 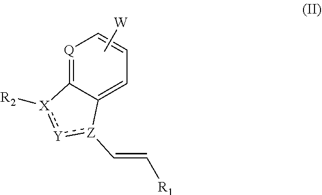
wherein:
Q is CH or N, wherein H of the CH is unsubstituted or substituted with halogen;
X is N;
Y is CR_{N} or N;
R_{N} is H or C1-C3 alkyl;
Z is C;
W is 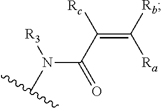
Rₐ, R_{b}, and R_{c} are each independently H, halogen, cyano, or C1-C6 alkyl;
R₁ is C1-C3 alkyl, C3-C6 cycloalkyl, 4 to 6-membered heterocycloalkyl, C3-C6 cycloalkenyl, aryl or heteroaryl, wherein at least one H of the C1-C3 alkyl, C3-C6 cycloalkyl, 4 to 6-membered heterocycloalkyl, C3-C6 cycloalkenyl, aryl or heteroaryl is independently unsubstituted or substituted with halogen, OR₄, unbranched or branched C1-C6 alkyl, COOR₄, C1-C6 haloalkyl, or phenyl;
R₂ is unbranched or branched C1-C6 alkyl or C3-C6 cycloalkyl, wherein at least one H of the unbranched or branched C1-C6 alkyl is independently unsubstituted or substituted with deuterium, halogen or =0;
R₃ is H, C1-C3 alkyl, benzyl, C1-C3 alkyl-heteroaryl or C1-C3 alkyl substituted with COOH; and
R₄ is C1-C3 alkyl.

6. The compound represented by formula (I), a pharmaceutically acceptable salt thereof, a stereoisomer thereof, a tautomer thereof, a solvate thereof, a hydrate thereof, or a prodrug analog thereof according to claim 1, wherein the compound represented by formula (I) is a compound represented by formula (III): 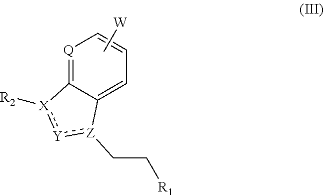
wherein:
Q is CH or N, wherein the CH is optionally substituted with halogen;
X is N;
Y is CR_{N};
R_{N} is H;
Z is C;
W is 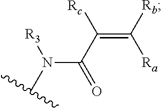
Rₐ, R_{b}, and R_{c} are H;
R₁ is C3-C6 cycloalkyl, C3-C6 cycloalkenyl or aryl, wherein at least one H of the C3-C6 cycloalkyl, C3-C6 cycloalkenyl or aryl is independently unsubstituted or substituted with halogen, OR₄, C1-C10 alkyl or C1-C6 haloalkyl;
R₂ is H or unbranched or branched C1-C6 alkyl or C3-C6 cycloalkyl;
R₃ is H, C1-C3 alkyl or benzyl;
R₄ is C1-C3 alkyl or benzyl, wherein at least one H of the benzyl is independently unsubstituted or substituted with halogen.

7. The compound represented by formula (I), a pharmaceutically acceptable salt thereof, a stereoisomer thereof, a tautomer thereof, a solvate thereof, a hydrate thereof, or a prodrug analog thereof according to claim 1, wherein the compound represented by formula (I) is a compound represented by formula (IV): 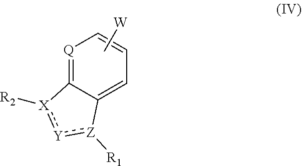
Q is CH or N, wherein H of the CH is unsubstituted or substituted with halogen;
X is N;
Y is CR_{N} or N;
R_{N} is H;
Z is C;
W is 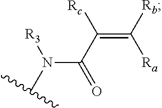
Rₐ, R_{b}, and R_{c} are each independently H or C1-C3 alkyl-N(R₄)₂;
R₁ is H, C3-C6 cycloalkyl, C3-C6 cycloalkenyl, 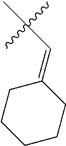 [SMILES: *C=C1CCCCC1]
or aryl, wherein at least one H of the C3-C6 cycloalkyl, C3-C6 cycloalkenyl or aryl is independently unsubstituted or substituted with halogen, CN, —CH═CH—R₄, —(CH₂)₂—R₄, OR₄, SR₄, CH₂N(R₄)₂, C(═O)—R₄, C1-C10 alkyl C1-C6 haloalkyl, C3-C6 cycloalkyl, 4 to 6-membered heterocycloalkyl, phenyl or phenyl substituted with halogen;
R₂ is H or unbranched or branched C1-C6 alkyl;
R₃ is H;
R₄ is H, C1-C3 alkyl, C3-C6 cycloalkyl, phenyl or benzyl, wherein at least one H of the C3-C6 cycloalkyl or benzyl is independently unsubstituted or substituted with halogen.

8. A compound, a pharmaceutically acceptable salt thereof, a stereoisomer thereof, a tautomer thereof, a solvate thereof, a hydrate thereof, or a prodrug analog thereof, wherein the compound is one selected from the group consisting of compounds represented by following 1 to 138:

| Compound | |
|---|---|
| No. | Structure |
| 1 | 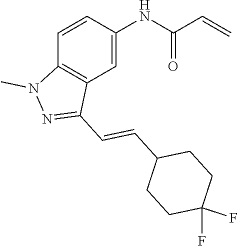 [SMILES: C=CC(=O)Nc1ccc2c(c1)c(/C=C/C1CCC(F)(F)CC1)nn2C] |
| 2 | 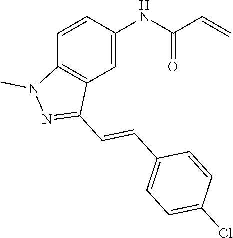 [SMILES: C=CC(=O)Nc1ccc2c(c1)c(/C=C/c1ccc(Cl)cc1)nn2C] |
| 3 | 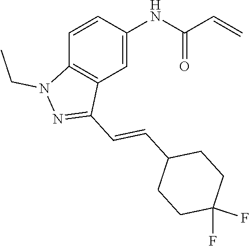 [SMILES: C=CC(=O)Nc1ccc2c(c1)c(/C=C/C1CCC(F)(F)CC1)nn2CC] |
| 4 | 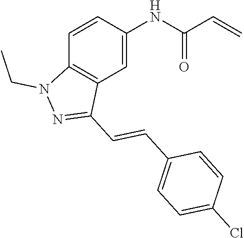 [SMILES: C=CC(=O)Nc1ccc2c(c1)c(/C=C/c1ccc(Cl)cc1)nn2CC] |
| 5 | 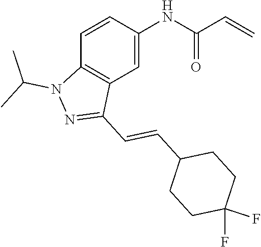 [SMILES: C=CC(=O)Nc1ccc2c(c1)c(/C=C/C1CCC(F)(F)CC1)nn2C(C)C] |
| 6 | 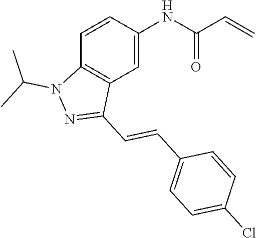 [SMILES: C=CC(=O)Nc1ccc2c(c1)c(/C=C/c1ccc(Cl)cc1)nn2C(C)C] |
| 7 | 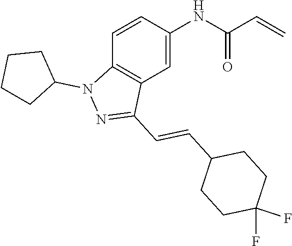 [SMILES: C=CC(=O)Nc1ccc2c(c1)c(/C=C/C1CCC(F)(F)CC1)nn2C1CCCC1] |
| 8 | 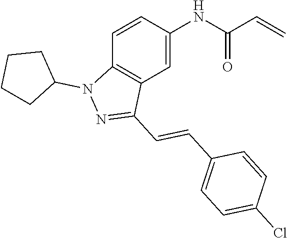 [SMILES: C=CC(=O)Nc1ccc2c(c1)c(/C=C/c1ccc(Cl)cc1)nn2C1CCCC1] |
| 9 | 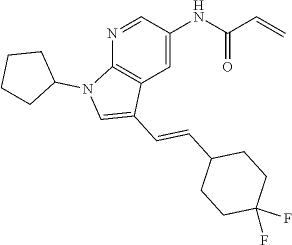 [SMILES: C=CC(=O)Nc1cnc2c(c1)c(/C=C/C1CCC(F)(F)CC1)cn2C1CCCC1] |
| 10 | 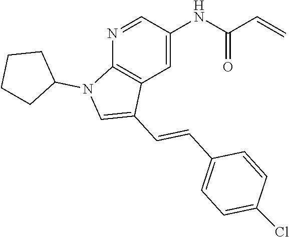 [SMILES: C=CC(=O)Nc1cnc2c(c1)c(/C=C/c1ccc(Cl)cc1)cn2C1CCCC1] |
| 11 | 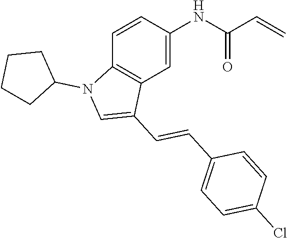 [SMILES: C=CC(=O)Nc1ccc2c(c1)c(/C=C/c1ccc(Cl)cc1)cn2C1CCCC1] |
| 12 | 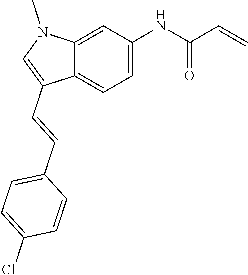 [SMILES: C=CC(=O)Nc1ccc2c(/C=C/c3ccc(Cl)cc3)cn(C)c2c1] |
| 13 | 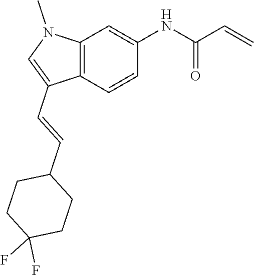 [SMILES: C=CC(=O)Nc1ccc2c(/C=C/C3CCC(F)(F)CC3)cn(C)c2c1] |
| 14 | 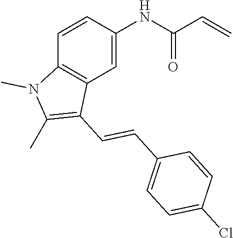 [SMILES: C=CC(=O)Nc1ccc2c(c1)c(/C=C/c1ccc(Cl)cc1)c(C)n2C] |
| 15 | 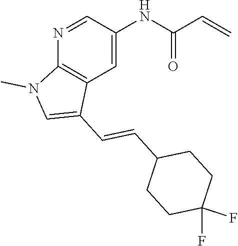 [SMILES: C=CC(=O)Nc1cnc2c(c1)c(/C=C/C1CCC(F)(F)CC1)cn2C] |
| 16 | 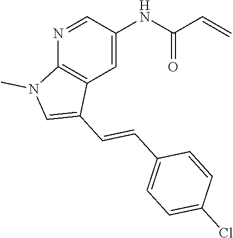 [SMILES: C=CC(=O)Nc1cnc2c(c1)c(/C=C/c1ccc(Cl)cc1)cn2C] |
| 17 | 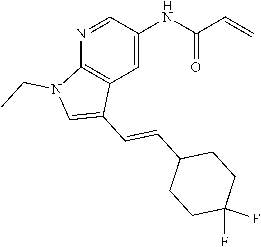 [SMILES: C=CC(=O)Nc1cnc2c(c1)c(/C=C/C1CCC(F)(F)CC1)cn2CC] |
| 18 | 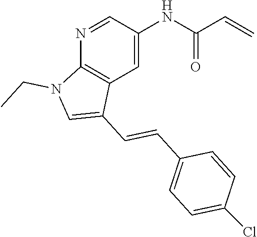 [SMILES: C=CC(=O)Nc1cnc2c(c1)c(/C=C/c1ccc(Cl)cc1)cn2CC] |
| 19 | 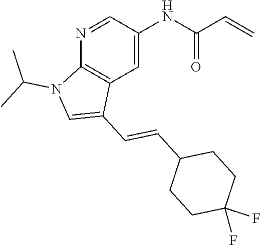 [SMILES: C=CC(=O)Nc1cnc2c(c1)c(/C=C/C1CCC(F)(F)CC1)cn2C(C)C] |
| 20 | 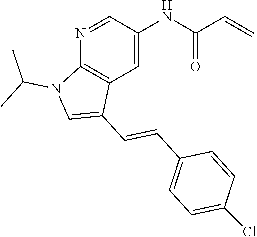 [SMILES: C=CC(=O)Nc1cnc2c(c1)c(/C=C/c1ccc(Cl)cc1)cn2C(C)C] |
| 21 | 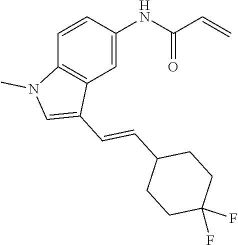 [SMILES: C=CC(=O)Nc1ccc2c(c1)c(/C=C/C1CCC(F)(F)CC1)cn2C] |
| 22 | 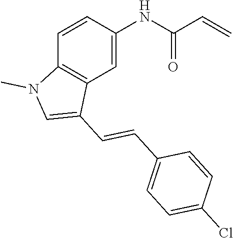 [SMILES: C=CC(=O)Nc1ccc2c(c1)c(/C=C/c1ccc(Cl)cc1)cn2C] |
| 23 | 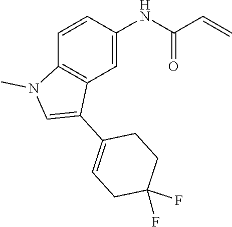 [SMILES: C=CC(=O)Nc1ccc2c(c1)c(C1=CCC(F)(F)CC1)cn2C] |
| 24 | 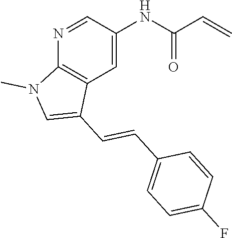 [SMILES: C=CC(=O)Nc1cnc2c(c1)c(/C=C/c1ccc(F)cc1)cn2C] |
| 25 | 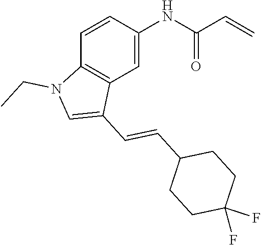 [SMILES: C=CC(=O)Nc1ccc2c(c1)c(/C=C/C1CCC(F)(F)CC1)cn2CC] |
| 26 | 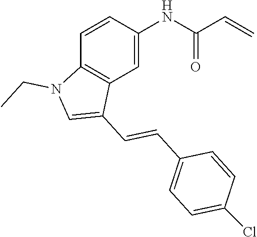 [SMILES: C=CC(=O)Nc1ccc2c(c1)c(/C=C/c1ccc(Cl)cc1)cn2CC] |
| 27 | 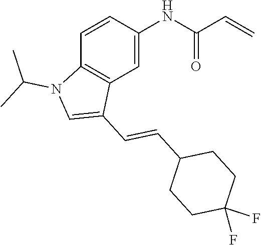 [SMILES: C=CC(=O)Nc1ccc2c(c1)c(/C=C/C1CCC(F)(F)CC1)cn2C(C)C] |
| 28 | 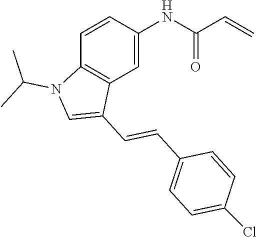 [SMILES: C=CC(=O)Nc1ccc2c(c1)c(/C=C/c1ccc(Cl)cc1)cn2C(C)C] |
| 29 | 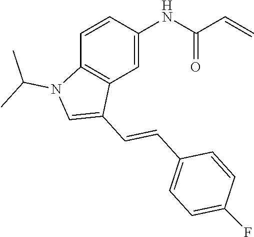 [SMILES: C=CC(=O)Nc1ccc2c(c1)c(/C=C/c1ccc(F)cc1)cn2C(C)C] |
| 30 | 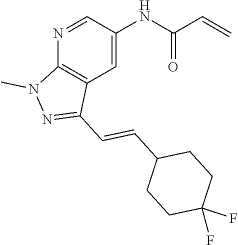 [SMILES: C=CC(=O)Nc1cnc2c(c1)c(/C=C/C1CCC(F)(F)CC1)nn2C] |
| 31 | 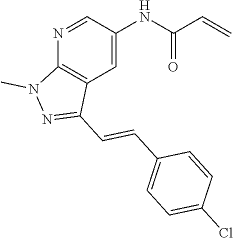 [SMILES: C=CC(=O)Nc1cnc2c(c1)c(/C=C/c1ccc(Cl)cc1)nn2C] |
| 32 | 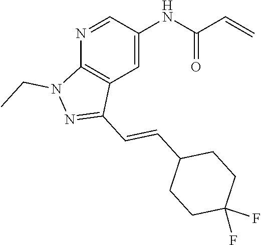 [SMILES: C=CC(=O)Nc1cnc2c(c1)c(/C=C/C1CCC(F)(F)CC1)nn2CC] |
| 33 | 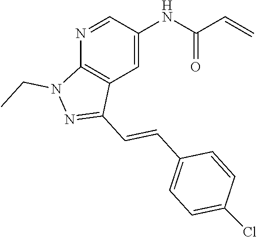 [SMILES: C=CC(=O)Nc1cnc2c(c1)c(/C=C/c1ccc(Cl)cc1)nn2CC] |
| 34 | 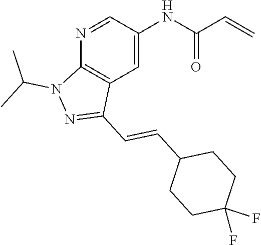 [SMILES: C=CC(=O)Nc1cnc2c(c1)c(/C=C/C1CCC(F)(F)CC1)nn2C(C)C] |
| 35 | 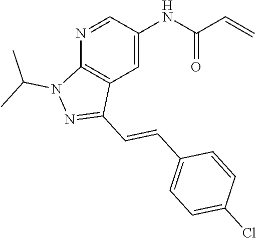 [SMILES: C=CC(=O)Nc1cnc2c(c1)c(/C=C/c1ccc(Cl)cc1)nn2C(C)C] |
| 36 | 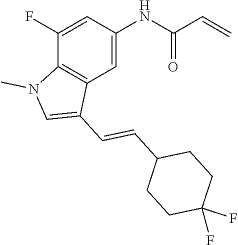 [SMILES: C=CC(=O)Nc1cc(F)c2c(c1)c(/C=C/C1CCC(F)(F)CC1)cn2C] |
| 37 | 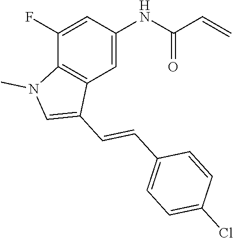 [SMILES: C=CC(=O)Nc1cc(F)c2c(c1)c(/C=C/c1ccc(Cl)cc1)cn2C] |
| 38 | 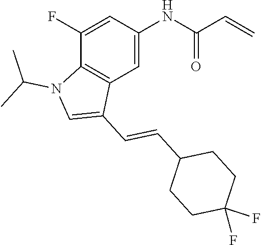 [SMILES: C=CC(=O)Nc1cc(F)c2c(c1)c(/C=C/C1CCC(F)(F)CC1)cn2C(C)C] |
| 39 | 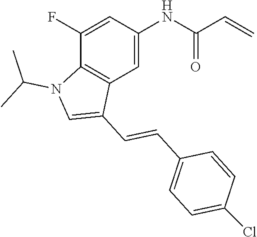 [SMILES: C=CC(=O)Nc1cc(F)c2c(c1)c(/C=C/c1ccc(Cl)cc1)cn2C(C)C] |
| 40 | 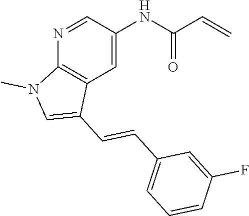 [SMILES: C=CC(=O)Nc1cnc2c(c1)c(/C=C/c1cccc(F)c1)cn2C] |
| 41 | 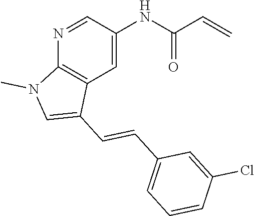 [SMILES: C=CC(=O)Nc1cnc2c(c1)c(/C=C/c1cccc(Cl)c1)cn2C] |
| 42 | 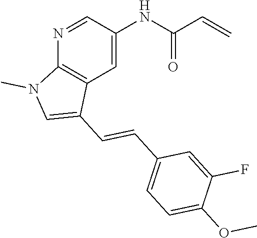 |
| 43 | 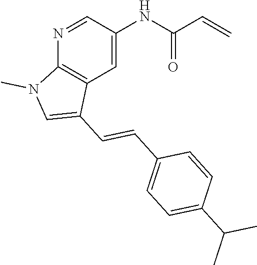 [SMILES: C=CC(=O)Nc1cnc2c(c1)c(/C=C/c1ccc(C(C)C)cc1)cn2C] |
| 44 | 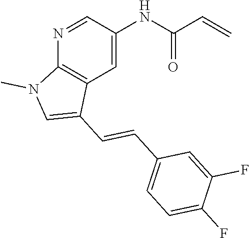 [SMILES: C=CC(=O)Nc1cnc2c(c1)c(/C=C/c1ccc(F)c(F)c1)cn2C] |
| 45 | 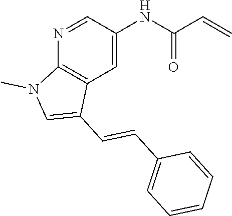 [SMILES: C=CC(=O)Nc1cnc2c(c1)c(/C=C/c1ccccc1)cn2C] |
| 46 | 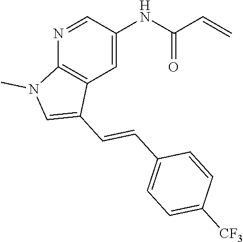 [SMILES: C=CC(=O)Nc1cnc2c(c1)c(/C=C/c1ccc(C(F)(F)F)cc1)cn2C] |
| 47 | 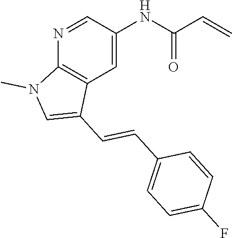 [SMILES: C=CC(=O)Nc1cnc2c(c1)c(/C=C/c1ccc(F)cc1)cn2C] |
| 48 | 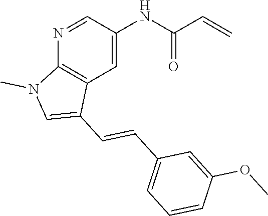 |
| 49 | 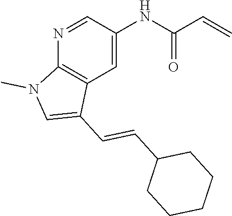 [SMILES: C=CC(=O)Nc1cnc2c(c1)c(/C=C/C1CCCCC1)cn2C] |
| 50 | 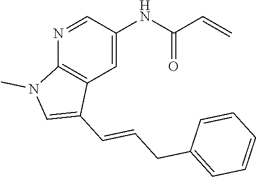 [SMILES: C=CC(=O)Nc1cnc2c(c1)c(/C=C/Cc1ccccc1)cn2C] |
| 51 | 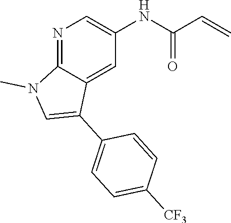 [SMILES: C=CC(=O)Nc1cnc2c(c1)c(-c1ccc(C(F)(F)F)cc1)cn2C] |
| 52 | 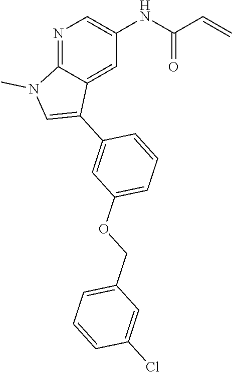 [SMILES: C=CC(=O)Nc1cnc2c(c1)c(-c1cccc(OCc3cccc(Cl)c3)c1)cn2C] |
| 53 | 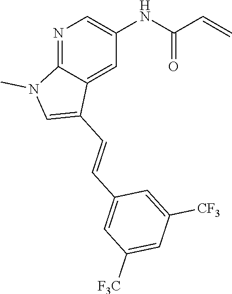 [SMILES: C=CC(=O)Nc1cnc2c(c1)c(/C=C/c1cc(C(F)(F)F)cc(C(F)(F)F)c1)cn2C] |
| 54 | 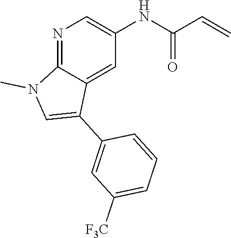 [SMILES: C=CC(=O)Nc1cnc2c(c1)c(-c1cccc(C(F)(F)F)c1)cn2C] |
| 55 | 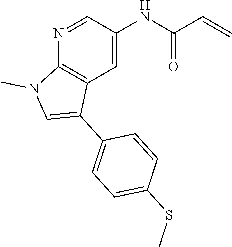 [SMILES: C=CC(=O)Nc1cnc2c(c1)c(-c1ccc(SC)cc1)cn2C] |
| 56 | 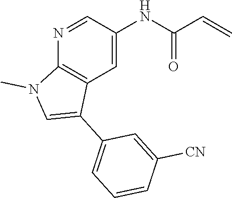 [SMILES: C=CC(=O)Nc1cnc2c(c1)c(-c1cccc(C#N)c1)cn2C] |
| 57 | 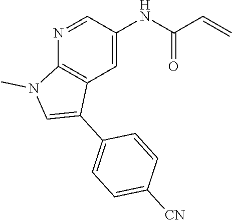 [SMILES: C=CC(=O)Nc1cnc2c(c1)c(-c1ccc(C#N)cc1)cn2C] |
| 58 | 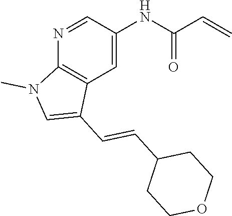 [SMILES: C=CC(=O)Nc1cnc2c(c1)c(/C=C/C1CCOCC1)cn2C] |
| 59 | 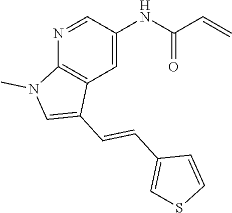 [SMILES: C=CC(=O)Nc1cnc2c(c1)c(/C=C/c1ccsc1)cn2C] |
| 60 | 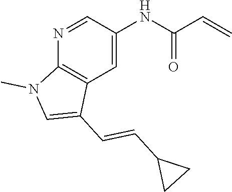 [SMILES: C=CC(=O)Nc1cnc2c(c1)c(/C=C/C1CC1)cn2C] |
| 61 | 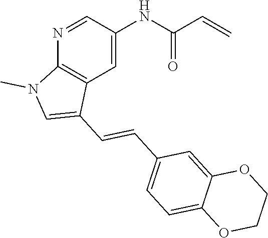 [SMILES: C=CC(=O)Nc1cnc2c(c1)c(/C=C/c1ccc3c(c1)OCCO3)cn2C] |
| 62 | 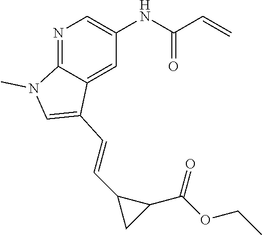 [SMILES: C=CC(=O)Nc1cnc2c(c1)c(/C=C/C1CC1C(=O)OCC)cn2C] |
| 63 | 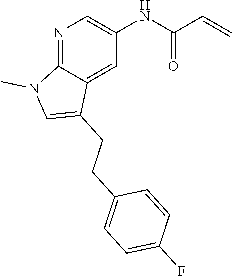 [SMILES: C=CC(=O)Nc1cnc2c(c1)c(CCc1ccc(F)cc1)cn2C] |
| 64 | 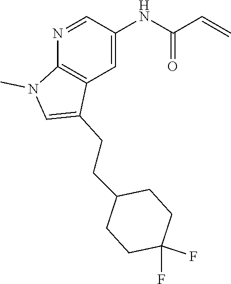 [SMILES: C=CC(=O)Nc1cnc2c(c1)c(CCC1CCC(F)(F)CC1)cn2C] |
| 65 | 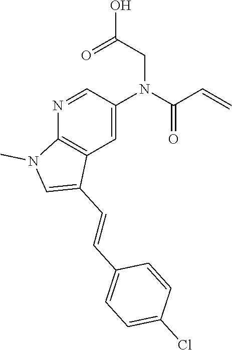 [SMILES: C=CC(=O)N(CC(=O)O)c1cnc2c(c1)c(/C=C/c1ccc(Cl)cc1)cn2C] |
| 66 | 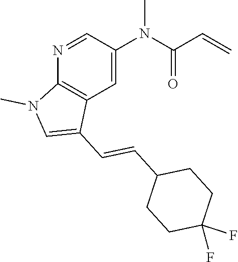 [SMILES: C=CC(=O)N(C)c1cnc2c(c1)c(/C=C/C1CCC(F)(F)CC1)cn2C] |
| 67 | 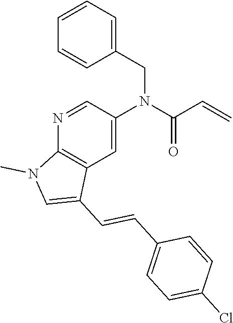 [SMILES: C=CC(=O)N(Cc1ccccc1)c1cnc2c(c1)c(/C=C/c1ccc(Cl)cc1)cn2C] |
| 68 | 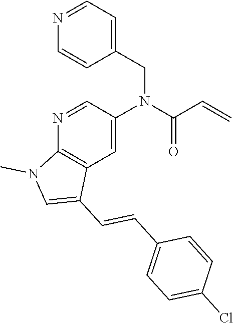 [SMILES: C=CC(=O)N(Cc1ccncc1)c1cnc2c(c1)c(/C=C/c1ccc(Cl)cc1)cn2C] |
| 69 | 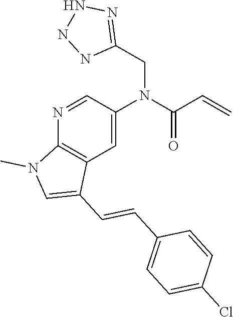 [SMILES: C=CC(=O)N(CC1=NNNN1)c1cnc2c(c1)c(/C=C/c1ccc(Cl)cc1)cn2C] |
| 70 | 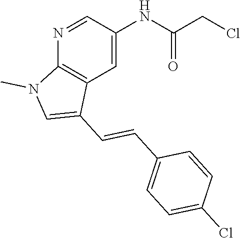 [SMILES: Cn1cc(/C=C/c2ccc(Cl)cc2)c2cc(NC(=O)CCl)cnc21] |
| 71 | 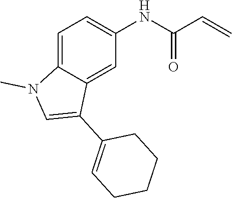 [SMILES: C=CC(=O)Nc1ccc2c(c1)c(C1=CCCCC1)cn2C] |
| 72 | 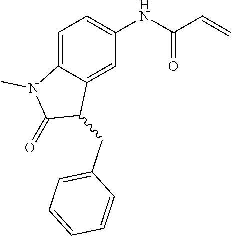 [SMILES: C=CC(=O)Nc1ccc2c(c1)C(Cc1ccccc1)C(=O)N2C] |
| 73 | 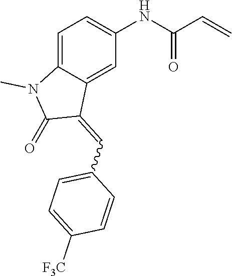 [SMILES: C=CC(=O)Nc1ccc2c(c1)C(=Cc1ccc(C(F)(F)F)cc1)C(=O)N2C] |
| 74 | 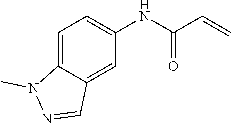 |
| 75 | 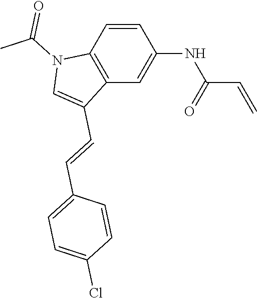 [SMILES: C=CC(=O)Nc1ccc2c(c1)c(/C=C/c1ccc(Cl)cc1)cn2C(C)=O] |
| 76 | 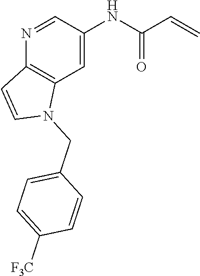 [SMILES: C=CC(=O)Nc1cnc2ccn(Cc3ccc(C(F)(F)F)cc3)c2c1] |
| 77 | 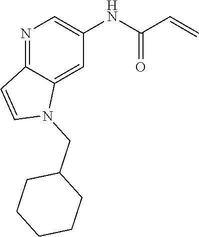 [SMILES: C=CC(=O)Nc1cnc2ccn(CC3CCCCC3)c2c1] |
| 78 | 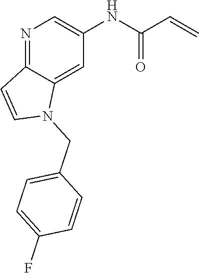 [SMILES: C=CC(=O)Nc1cnc2ccn(Cc3ccc(F)cc3)c2c1] |
| 79 | 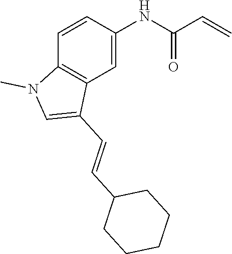 [SMILES: C=CC(=O)Nc1ccc2c(c1)c(/C=C/C1CCCCC1)cn2C] |
| 80 | 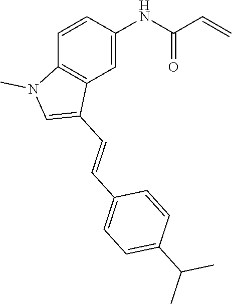 [SMILES: C=CC(=O)Nc1ccc2c(c1)c(/C=C/c1ccc(C(C)C)cc1)cn2C] |
| 81 | 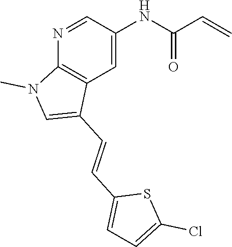 [SMILES: C=CC(=O)Nc1cnc2c(c1)c(/C=C/c1ccc(Cl)s1)cn2C] |
| 82 | 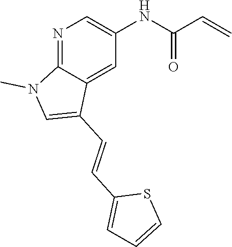 [SMILES: C=CC(=O)Nc1cnc2c(c1)c(/C=C/c1cccs1)cn2C] |
| 83 | 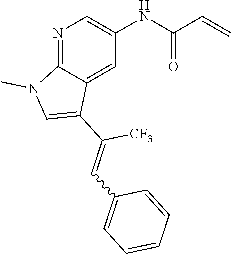 [SMILES: C=CC(=O)Nc1cnc2c(c1)c(C(=Cc1ccccc1)C(F)(F)F)cn2C] |
| 84 | 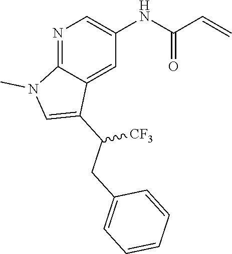 [SMILES: C=CC(=O)Nc1cnc2c(c1)c(C(Cc1ccccc1)C(F)(F)F)cn2C] |
| 85 | 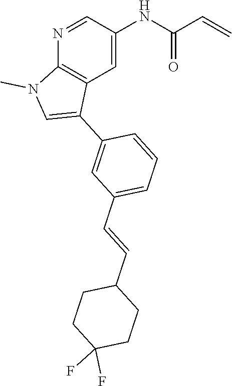 [SMILES: C=CC(=O)Nc1cnc2c(c1)c(-c1cccc(/C=C/C3CCC(F)(F)CC3)c1)cn2C] |
| 86 | 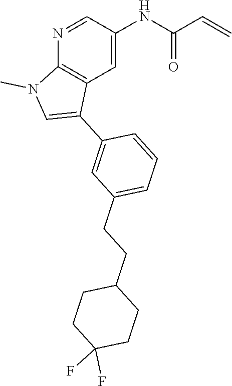 [SMILES: C=CC(=O)Nc1cnc2c(c1)c(-c1cccc(CCC3CCC(F)(F)CC3)c1)cn2C] |
| 87 | 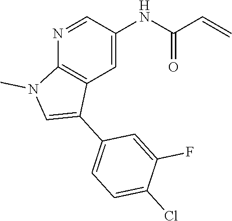 [SMILES: C=CC(=O)Nc1cnc2c(c1)c(-c1ccc(Cl)c(F)c1)cn2C] |
| 88 | 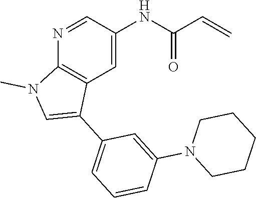 [SMILES: C=CC(=O)Nc1cnc2c(c1)c(-c1cccc(N3CCCCC3)c1)cn2C] |
| 89 | 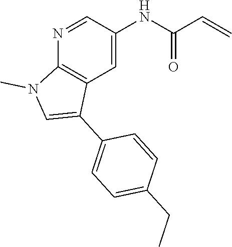 [SMILES: C=CC(=O)Nc1cnc2c(c1)c(-c1ccc(CC)cc1)cn2C] |
| 90 | 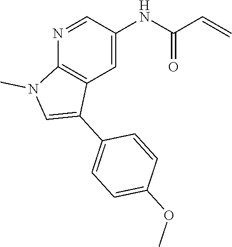 [SMILES: C=CC(=O)Nc1cnc2c(c1)c(-c1ccc(OC)cc1)cn2C] |
| 91 | 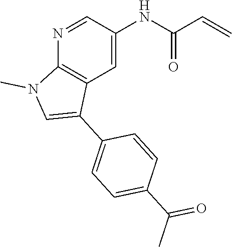 [SMILES: C=CC(=O)Nc1cnc2c(c1)c(-c1ccc(C(C)=O)cc1)cn2C] |
| 92 | 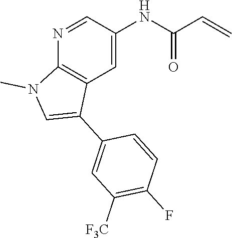 [SMILES: C=CC(=O)Nc1cnc2c(c1)c(-c1ccc(F)c(C(F)(F)F)c1)cn2C] |
| 93 | 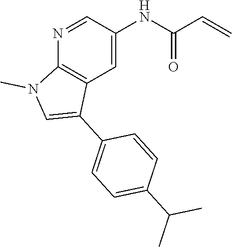 [SMILES: C=CC(=O)Nc1cnc2c(c1)c(-c1ccc(C(C)C)cc1)cn2C] |
| 94 | 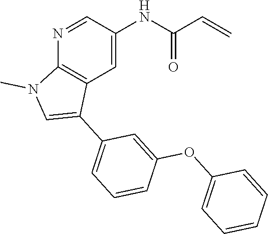 [SMILES: C=CC(=O)Nc1cnc2c(c1)c(-c1cccc(Oc3ccccc3)c1)cn2C] |
| 95 | 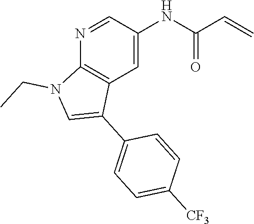 [SMILES: C=CC(=O)Nc1cnc2c(c1)c(-c1ccc(C(F)(F)F)cc1)cn2CC] |
| 96 | 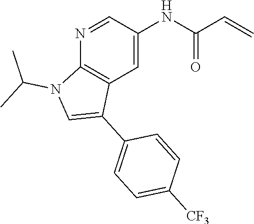 [SMILES: C=CC(=O)Nc1cnc2c(c1)c(-c1ccc(C(F)(F)F)cc1)cn2C(C)C] |
| 97 | 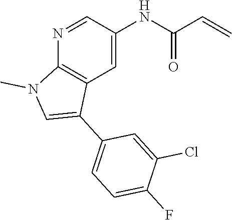 [SMILES: C=CC(=O)Nc1cnc2c(c1)c(-c1ccc(F)c(Cl)c1)cn2C] |
| 98 | 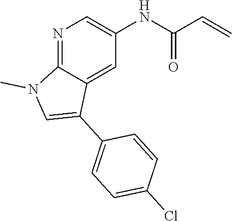 [SMILES: C=CC(=O)Nc1cnc2c(c1)c(-c1ccc(Cl)cc1)cn2C] |
| 99 | 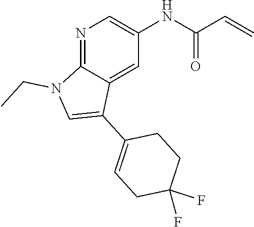 [SMILES: C=CC(=O)Nc1cnc2c(c1)c(C1=CCC(F)(F)CC1)cn2CC] |
| 100 | 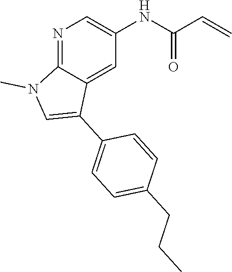 [SMILES: C=CC(=O)Nc1cnc2c(c1)c(-c1ccc(CCC)cc1)cn2C] |
| 101 | 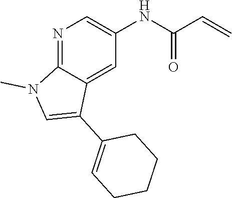 [SMILES: C=CC(=O)Nc1cnc2c(c1)c(C1=CCCCC1)cn2C] |
| 102 | 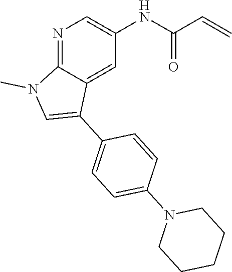 [SMILES: C=CC(=O)Nc1cnc2c(c1)c(-c1ccc(N3CCCCC3)cc1)cn2C] |
| 103 | 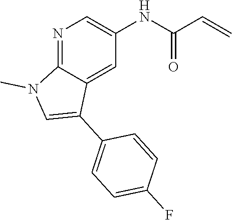 [SMILES: C=CC(=O)Nc1cnc2c(c1)c(-c1ccc(F)cc1)cn2C] |
| 104 | 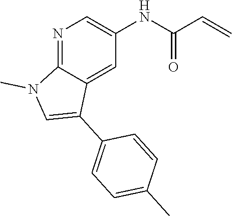 [SMILES: C=CC(=O)Nc1cnc2c(c1)c(-c1ccc(C)cc1)cn2C] |
| 105 | 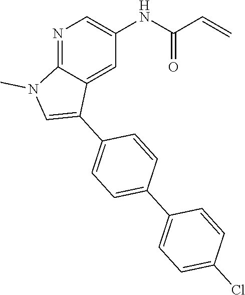 [SMILES: C=CC(=O)Nc1cnc2c(c1)c(-c1ccc(-c3ccc(Cl)cc3)cc1)cn2C] |
| 106 | 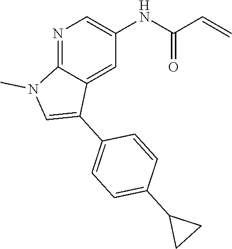 [SMILES: C=CC(=O)Nc1cnc2c(c1)c(-c1ccc(C3CC3)cc1)cn2C] |
| 107 | 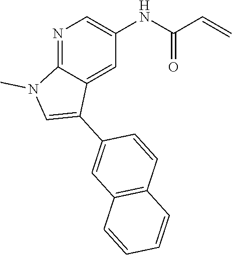 [SMILES: C=CC(=O)Nc1cnc2c(c1)c(-c1ccc3ccccc3c1)cn2C] |
| 108 | 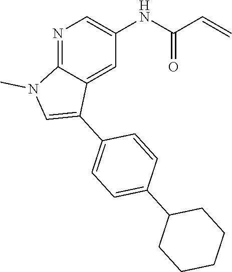 [SMILES: C=CC(=O)Nc1cnc2c(c1)c(-c1ccc(C3CCCCC3)cc1)cn2C] |
| 109 | 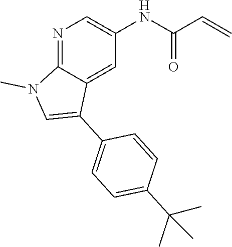 [SMILES: C=CC(=O)Nc1cnc2c(c1)c(-c1ccc(C(C)(C)C)cc1)cn2C] |
| 110 | 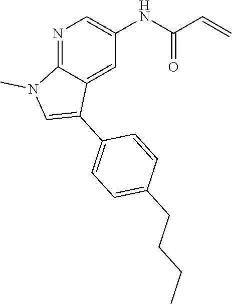 [SMILES: C=CC(=O)Nc1cnc2c(c1)c(-c1ccc(CCCC)cc1)cn2C] |
| 111 | 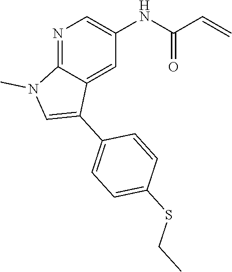 [SMILES: C=CC(=O)Nc1cnc2c(c1)c(-c1ccc(SCC)cc1)cn2C] |
| 112 | 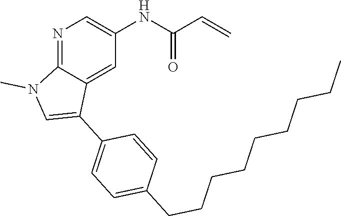 [SMILES: C=CC(=O)Nc1cnc2c(c1)c(-c1ccc(CCCCCCCCC)cc1)cn2C] |
| 113 | 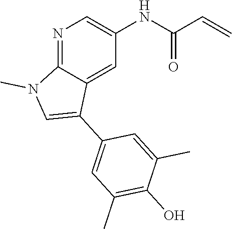 [SMILES: C=CC(=O)Nc1cnc2c(c1)c(-c1cc(C)c(O)c(C)c1)cn2C] |
| 114 | 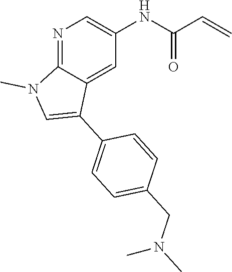 [SMILES: C=CC(=O)Nc1cnc2c(c1)c(-c1ccc(CN(C)C)cc1)cn2C] |
| 115 | 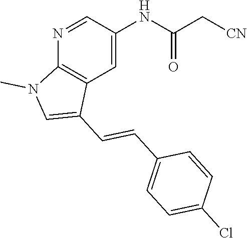 [SMILES: Cn1cc(/C=C/c2ccc(Cl)cc2)c2cc(NC(=O)CC#N)cnc21] |
| 116 | 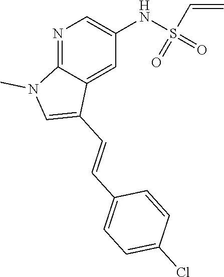 [SMILES: C=CS(=O)(=O)Nc1cnc2c(c1)c(/C=C/c1ccc(Cl)cc1)cn2C] |
| 117 | 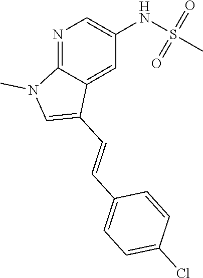 |
| 118 | 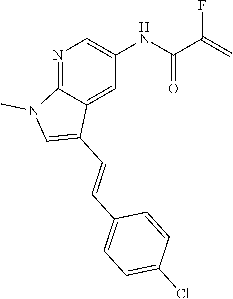 [SMILES: C=C(F)C(=O)Nc1cnc2c(c1)c(/C=C/c1ccc(Cl)cc1)cn2C] |
| 119 | 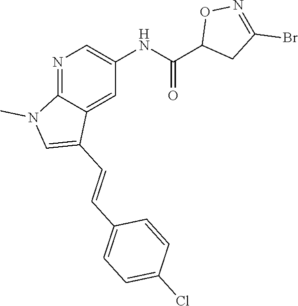 [SMILES: Cn1cc(/C=C/c2ccc(Cl)cc2)c2cc(NC(=O)C3CC(Br)=NO3)cnc21] |
| 120 | 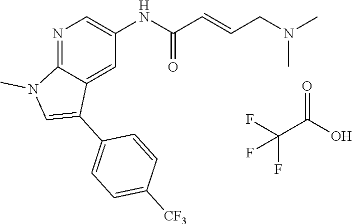 |
| 121 | 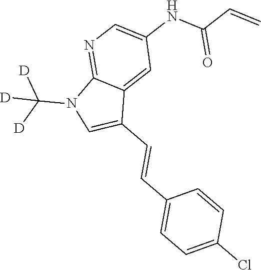 [SMILES: [2H]C([2H])([2H])n1cc(/C=C/c2ccc(Cl)cc2)c2cc(NC(=O)C=C)cnc21] |
| 122 | 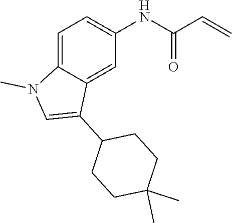 [SMILES: C=CC(=O)Nc1ccc2c(c1)c(C1CCC(C)(C)CC1)cn2C] |
| 123 | 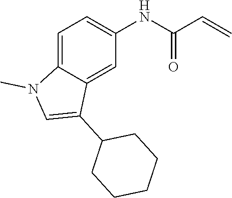 [SMILES: C=CC(=O)Nc1ccc2c(c1)c(C1CCCCC1)cn2C] |
| 124 | 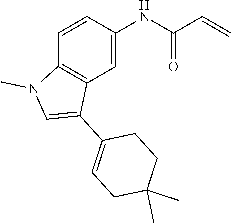 [SMILES: C=CC(=O)Nc1ccc2c(c1)c(C1=CCC(C)(C)CC1)cn2C] |
| 125 | 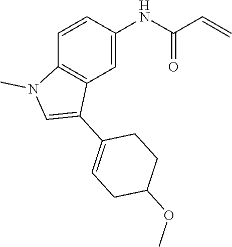 [SMILES: C=CC(=O)Nc1ccc2c(c1)c(C1=CCC(OC)CC1)cn2C] |
| 126 | 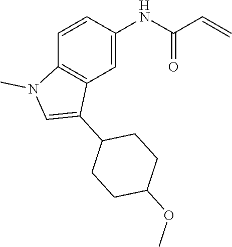 [SMILES: C=CC(=O)Nc1ccc2c(c1)c(C1CCC(OC)CC1)cn2C] |
| 127 | 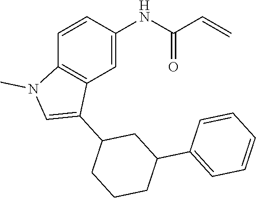 [SMILES: C=CC(=O)Nc1ccc2c(c1)c(C1CCCC(c3ccccc3)C1)cn2C] |
| 128 | 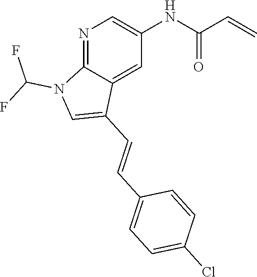 [SMILES: C=CC(=O)Nc1cnc2c(c1)c(/C=C/c1ccc(Cl)cc1)cn2C(F)F] |
| 129 | 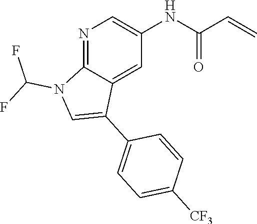 [SMILES: C=CC(=O)Nc1cnc2c(c1)c(-c1ccc(C(F)(F)F)cc1)cn2C(F)F] |
| 130 | 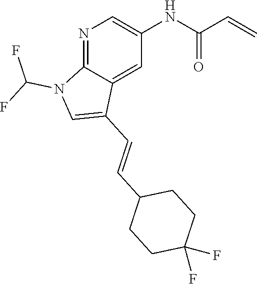 [SMILES: C=CC(=O)Nc1cnc2c(c1)c(/C=C/C1CCC(F)(F)CC1)cn2C(F)F] |
| 131 | 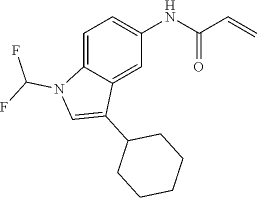 [SMILES: C=CC(=O)Nc1ccc2c(c1)c(C1CCCCC1)cn2C(F)F] |
| 132 | 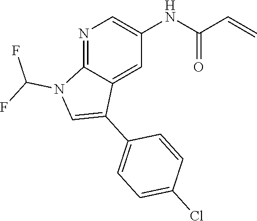 [SMILES: C=CC(=O)Nc1cnc2c(c1)c(-c1ccc(Cl)cc1)cn2C(F)F] |
| 133 | 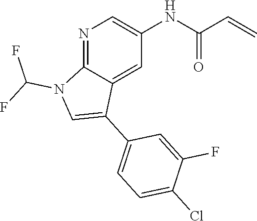 [SMILES: C=CC(=O)Nc1cnc2c(c1)c(-c1ccc(Cl)c(F)c1)cn2C(F)F] |
| 134 | 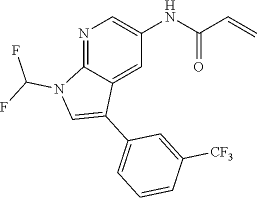 [SMILES: C=CC(=O)Nc1cnc2c(c1)c(-c1cccc(C(F)(F)F)c1)cn2C(F)F] |
| 135 | 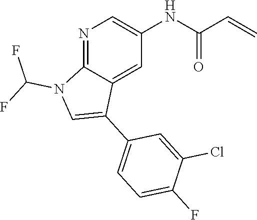 [SMILES: C=CC(=O)Nc1cnc2c(c1)c(-c1ccc(F)c(Cl)c1)cn2C(F)F] |
| 136 | 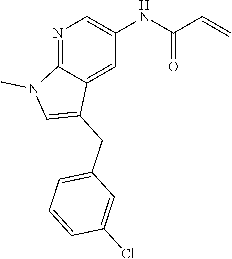 [SMILES: C=CC(=O)Nc1cnc2c(c1)c(Cc1cccc(Cl)c1)cn2C] |
| 137 | 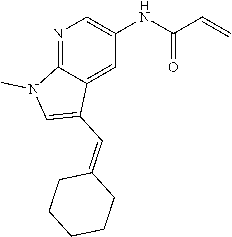 [SMILES: C=CC(=O)Nc1cnc2c(c1)c(C=C1CCCCC1)cn2C] |
| 138 | 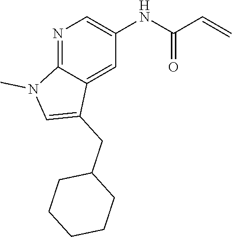 [SMILES: C=CC(=O)Nc1cnc2c(c1)c(CC1CCCCC1)cn2C] |

9. A pharmaceutical composition comprising the compound according to claim 1, a pharmaceutically acceptable salt thereof, stereoisomer thereof, a tautomer thereof, a solvate thereof, a hydrate thereof, or a prodrug analog thereof, as an effective component.

10. The pharmaceutical composition according to claim 9, wherein the pharmaceutical composition is used for preventing or treating YAP/TAZ-TEAD-mediated diseases or TEAD-dependent gene transcription-mediated diseases.

11. The pharmaceutical composition according to claim 10, wherein YAP/TAZ-TEAD-mediated diseases or TEAD-dependent gene transcription-mediated diseases comprise cancer.

12. A method for preventing or treating YAP/TAZ-TEAD-mediated diseases or TEAD-dependent gene transcription-mediated diseases, the method comprising administering a therapeutically effective amount of the compound according to claim 18, a pharmaceutically acceptable salt thereof, stereoisomer thereof, a tautomer thereof, a solvate thereof, a hydrate thereof, or a prodrug analog thereof.

13-14. (canceled)
